(19) United States
(12) Patent Application Publication
(10) Pub. No.: US 2026/0258382 A1
(43) Pub. Date: Sep. 3, 2026

(54) ULTRACOMPACT PRECISION EDITING SYSTEMS AND USES THEREOF

(71) Applicant: Mammoth Biosciences, Inc., Brisbane, CA (US)

(72) Inventors: Benjamin Julius RAUCH, San Francisco, CA (US); Aaron DELOUGHERY, San Francisco, CA (US); Fnu YUNANDA, Daly City, CA (US); Shravanti K. SURESH, San Francisco, CA (US); Sean CHEN, Brisbane, CA (US); Matan DRORY RETWITZER, Foster City, CA (US); William Douglass WRIGHT, Fairfield, CA (US); Wiputra Jaya HARTONO, San Francisco, CA (US); Lucas Benjamin HARRINGTON, San Francisco, CA (US); Wang-Ting LU, San Francisco, CA (US)

(21) Appl. No.: 19/334,209

(22) Filed: Sep. 19, 2025

Related U.S. Application Data
(63) Continuation of application No. PCT/US24/20492, filed on Mar. 19, 2024.
(60) Provisional application No. 63/453,442, filed on Mar. 20, 2023.
(60) Provisional application No. 63/496,622, filed on Apr. 17, 2023.
(60) Provisional application No. 63/500,566, filed on May 5, 2023.
(60) Provisional application No. 63/502,048, filed on May 12, 2023.
(60) Provisional application No. 63/514,299, filed on Jul. 18, 2023.
(60) Provisional application No. 63/594,393, filed on Oct. 30, 2023.
(60) Provisional application No. 63/608,136, filed on Dec. 8, 2023.
(60) Provisional application No. 63/622,444, filed on Jan. 18, 2024.

(51) Int. Cl. C12N 9/22; C12N 9/78; C12N 15/11; C12N 15/115; C12N 15/86; C12N 15/90

(52) U.S. Cl. CPC C12N 9/222; C12N 9/78; C12N 15/11; C12N 15/115; C12N 15/86; C12N 15/902; C12Y 305/04001; C12Y 305/04002; C12N 2310/16; C12N 2310/20; C12N 2750/14143

(57) ABSTRACT

Provided herein are compositions, systems, and methods comprising effector proteins, and uses thereof. These effector proteins may be characterized as CRISPR-associated (Cas) proteins. Various compositions, systems, and methods of the present disclosure may leverage the activities of these effector proteins for the editing, detecting and/or engineering of nucleic acids.

### CROSS-REFERENCED APPLICATIONS

This application is a continuation of International Patent Application No. PCT/US2024/020492, filed Mar. 19, 2024, which claims the benefit of priority to U.S. Provisional Application No. 63/453,442, filed on Mar. 20, 2023, U.S. Provisional Application No. 63/496,622, filed on Apr. 17, 2023, U.S. Provisional Application No. 63/500,566, filed on May 5, 2023, U.S. Provisional Application No. 63/502,048, filed on May 12, 2023, U.S. Provisional Application No. 63/514,299, filed on Jul. 18, 2023, U.S. Provisional Application No. 63/594,393, filed on Oct. 30, 2023, U.S. Provisional Application No. 63/608,136, filed on Dec. 8, 2023, and U.S. Provisional Application No. 63/622,444, filed on Jan. 18, 2024 the entire contents of each of which are incorporated herein by reference.

### INCORPORATION BY REFERENCE OF SEQUENCE LISTING

The instant application contains a Sequence Listing, which has been submitted via Patent Center. The Sequence Listing titled 203477-778301_US_SL.xml, which was created on Sep. 12, 2024, and is 374,085 bytes in size, is hereby incorporated by reference in its entirety.

### FIELD

The present disclosure relates generally to polypeptides, such as effector proteins, compositions of such polypeptides and guide nucleic acids, systems, and methods of using such polypeptides and compositions, including detecting and editing target nucleic acids.

### BACKGROUND

Clustered Regularly Interspaced Short Palindromic Repeats (CRISPR) and associated proteins (Cas proteins), sometimes referred to as a CRISPR/Cas system, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner. Native systems contain a CRISPR array, which includes direct repeats flanking short spacer sequences that, in part, guide Cas proteins to their targets. The discovery of CRISPR/Cas systems has revolutionized the field of genomic manipulation and engineering, and therapeutic applications of these systems are being explored.

### SUMMARY

The present disclosure provides for polypeptides, such as effector proteins, compositions, systems, and methods comprising the same, and uses thereof. In some instances, compositions, systems, and methods comprise guide nucleic acids or uses thereof. Compositions, systems, and methods disclosed herein may leverage nucleic acid modification activities, such as nucleic acid editing. Editing may comprise: insertion, deletion, substitution, or a combination thereof of one or more nucleotides in a target nucleic acid. In some embodiments, editing comprises modifying one or more nucleobases of a target nucleic acid. Editing may comprise cleavage activity, such as cis cleavage activity, nicking activity, and/or nuclease activity. In some instances, editing does not comprise generating a double stranded break in a target nucleic acid comprising DNA. In some instances, compositions, systems and methods are useful for the editing the sequence of target nucleic acids. In some instances, compositions, systems and methods are useful for the treatment of a disease or disorder. The disease or disorder may be associated with a target nucleic acid. The disease or disorder may be associated with one or more mutations in the target nucleic acid.

### Certain Embodiments

Provided herein are systems comprising one or more components, wherein the one or more components individually or collectively comprise: (a) an effector protein, or a nucleic acid encoding the effector protein, wherein the effector protein comprises an amino acid sequence that is at least 75%, at least 80%, at least 85%, at least 90%, at least 95%, at least 97%, or at least 98%, at least 99%, or less than 100% identical to the amino acid sequence recited in TABLE 1; (b) an effector partner, or a nucleic acid encoding the effector partner, wherein the effector partner is a base editing enzyme; and (c) an engineered guide nucleic acid or a nucleic acid that encodes the engineered guide nucleic acid, wherein the engineered guide nucleic acid comprises a first region and a second region, wherein the first region is capable of, at least partially, interacting with the effector protein, wherein the second region comprises a spacer sequence, and wherein the spacer sequence comprises a nucleic acid sequence that is capable of hybridizing to a target sequence in a target nucleic acid. In some embodiments, the first region, at least partially, interacts with the effector protein. In some embodiments, the spacer sequence comprises a nucleic acid sequence that hybridizes to a target sequence in a target nucleic acid. In some embodiments, the system is a base editor system that is capable of editing: (a) an adenine (A) to guanine (G); (b) cytosine (C) to thymine (T); (c) cytosine (C) to guanine (G); (d) uracil (U) to cytosine (C); (e) guanine (G) to adenine (A); (f) hydrolytic deamination of an adenine or adenosine, or methylation of cytosine; or (g) a combination thereof. In some embodiments, the system is a base editor system that edits: (a) an adenine (A) to guanine (G); (b) cytosine (C) to thymine (T); (c) cytosine (C) to guanine (G); (d) uracil (U) to cytosine (C); (e) guanine (G) to adenine (A); (f) hydrolytic deamination of an adenine or adenosine, or methylation of cytosine; or (g) a combination thereof. In some embodiments, the system is an adenosine base editor (ABE) system. In some embodiments, the system is a cytosine base editor (CBE) system. In some embodiments, the base editing enzyme comprises a deaminase or deaminase activity. In some embodiments, the effector protein, the base editing enzyme, or both are further fused to an enzyme selected from an endonuclease and a glycosylase. In some embodiments, the base editing enzyme comprises an amino acid sequence that is at least 75%, at least 80%, at least 85%, at least 90%, at least 95%, at least 97%, or at least 98%, at least 99%, or 100% identical to any one of the amino acid sequences of SEQ ID NO: 2-9. In some embodiments, the base editing enzyme comprises an amino acid sequence that is at least 75%, at least 80%, at least 85%, at least 90%, at least 95%, at least 97%, or at least 98%, at least 99%, or 100% identical to SEQ ID NO: 2. In some embodiments, the system comprises a fusion protein, or a nucleic acid encoding the fusion protein, wherein the fusion protein comprises the effector protein and the effector partner fused to each other. In some embodiments, the effector protein is linked to an N-terminus of the effector partner. In some embodiments, the effector protein is linked to a C-terminus of the effector partner. In some embodiments, the effector protein and the effector partner are directly fused to each other. In some embodiments, the effector protein and the effector partner are fused by a linker. In some embodiments, the effector protein provides cis cleavage activity. In some embodiments, the effector protein provides nickase activity. In some embodiments, the effector partner is capable of base editing of the target nucleic acid, wherein the target nucleic acid is a single stranded target nucleic acid. In some embodiments, the effector partner edits a base of the target nucleic acid, wherein the target nucleic acid is a single stranded target nucleic acid. In some embodiments, the effector protein is capable of cleaving the single stranded target nucleic acid. In some embodiments, the effector protein cleaves the single stranded target nucleic acid. In some embodiments, the effector partner is capable of base editing of the target nucleic acid, wherein the target nucleic acid is a double stranded target nucleic acid comprising a target strand and a non-target strand. In some embodiments, the effector partner edits a base of the target nucleic acid, wherein the target nucleic acid is a double stranded target nucleic acid comprising a target strand and a non-target strand. In some embodiments, the effector partner is capable of editing the target strand. In some embodiments, the effector partner edits the target strand. In some embodiments, the effector partner is capable of editing the non-target strand. In some embodiments, the effector partner edits the non-target strand. In some embodiments, the effector protein is capable of cleaving the target strand, the non-target strand, or both. In some embodiments, the effector protein cleaves the target strand, the non-target strand, or both. In some embodiments, the effector partner edits a base of the target strand, the non-target strand, or both. In some embodiments, the effector protein nicks the target strand or the non-target strand. In some embodiments, the effector partner edits a base of a non-nicked strand. In some embodiments, the system comprises a prime editing enzyme or a nucleic acid encoding the prime editing enzyme, wherein the prime editing enzyme catalyzes a reverse transcriptase reaction. In some embodiments, a nicked strand is corrected by reverse transcriptase editing. In some embodiments, the effector protein comprises at least one mutation that reduces its nuclease activity, relative to an otherwise identical polypeptide without the mutation, as measured in a cleavage assay. In some embodiments, the effector protein comprises one or more amino acid substitutions relative to an otherwise identical protein, wherein the one or more amino acid substitutions provide reduced catalytic activity relative to the otherwise identical protein. In some embodiments, the effector protein is a catalytically inactive effector protein. In some embodiments, the effector protein comprises one or more substitutions selected from positions D237, D418, and E335 relative to the amino acid sequence recited in TABLE 1. In some embodiments, the effector protein comprises one or more amino acid substitutions selected from D237A, D418A, D418N, E335A, and E335Q relative to the amino acid sequence recited in TABLE 1. In some embodiments, the effector protein comprises one or more amino acid substitutions independently at positions selected from A75, K58, 180, T84, K105, D171, N193, C202, S209, G210, A218, D220, E225, C246, K250, N286, M295, M298, A306, Y315, Q360, E362 and A393 relative to the amino acid sequence recited in TABLE 1. In some embodiments, the effector protein comprises one or more amino acid substitutions selected from I80R, T84R, K105R, G210R, C202R, A218R, D220R, E225R, C246R, and Q360R relative to the amino acid sequence recited in TABLE 1. In some embodiments, the effector protein comprises the amino acid substitution of D220R relative to the amino acid sequence recited in TABLE 1. In some embodiments, the effector protein comprising the amino acid substitution of D220R relative to the amino acid sequence recited in TABLE 1 further comprises at least one of the amino acid substitutions selected from L337A, T381A, S382A, C385A, F406A, N420A, and N424A. In some embodiments, the effector protein further comprises at least one of the amino acid substitutions selected from L337A, S382A and F406A. In some embodiments, the effector protein further comprises an amino acid substitution at S382A. In some embodiments, the effector protein comprises one or more amino acid substitutions selected from K58W, 180K, N193K, S209F, A218K, E225K, N286K, M295W, M298L, A306K, and Y315M relative to the amino acid sequence recited in TABLE 1. In some embodiments, the effector protein comprises one or more amino acid substitutions selected from A75F, T84R, K250N, D171K, N193K, C202K, C202R, S209F, S209Y, D220R, E225K, E225R, K250N, N286K, A306K, Y315M, Q360R, E362D, A393R and A393E relative to the amino acid sequence recited in TABLE 1. In some embodiments, the effector protein comprises one or more amino acid substitutions selected from: (a) D220R and D237A; (b) D220R and D237N; and (c) E335Q and optionally D220R, wherein the effector partner comprises an amino acid sequence that is at least at least 90% identical to the amino acid sequence of SEQ ID NO: 2.

Also provided herein are systems comprising one or more components, wherein the one or more components individually or collectively comprise: (a) an effector protein, or a nucleic acid encoding the effector protein, wherein the effector protein comprises an amino acid sequence that is at least 75%, at least 80%, at least 85%, at least 90%, at least 95%, at least 97%, or at least 98%, at least 99%, or less than 100% identical to the amino acid sequence recited in TABLE 1, wherein the effector protein is a catalytically inactive effector protein; (b) an effector partner, or a nucleic acid encoding the effector partner, wherein the effector partner is an adenosine base editing enzyme; and (c) an engineered guide nucleic acid or a nucleic acid that encodes the engineered guide nucleic acid, wherein the engineered guide nucleic acid comprises a first region and a second region, wherein the first region is capable of, at least partially, interacting with the effector protein, wherein the second region comprises a spacer sequence, and wherein the spacer sequence comprises a nucleic acid sequence that is capable of hybridizing to a target sequence in a target nucleic acid. In some embodiments, the first region, at least partially, interacts with the effector protein. In some embodiments, the spacer sequence comprises a nucleic acid sequence that hybridizes to a target sequence in a target nucleic acid. In some embodiments, the effector protein comprises one or more amino acid substitutions selected from D237A, D418A, D418N, E335A, and E335Q relative to the amino acid sequence recited in TABLE 1. In some embodiments, the effector protein comprises, wherein the effector protein comprises one or more amino acid substitutions independently at positions selected from A75, K58, 180, T84, K105, D171, N193, C202, S209, G210, A218, D220, E225, C246, K250, N286, M295, M298, A306, Y315, Q360, E362 and A393 relative to the amino acid sequence recited in TABLE 1. In some embodiments, the effector protein comprises one or more amino acid substitutions selected from I80R, T84R, K105R, G210R, C202R, A218R, D220R, E225R, C246R, and Q360R relative to the amino acid sequence recited in TABLE 1. In some embodiments, the effector protein comprises the amino acid substitution of D220R relative to the amino acid sequence recited in TABLE 1. In some embodiments, the effector protein comprises one or more amino acid substitutions selected from K58W, 180K, N193K, S209F, A218K, E225K, N286K, M295W, M298L, A306K, and Y315M relative to the amino acid sequence recited in TABLE 1. In some embodiments, the effector protein comprises one or more amino acid substitutions selected from A75F, T84R, K250N, D171K, N193K, C202K, C202R, S209F, S209Y, D220R, E225K, E225R, K250N, N286K, A306K, Y315M, Q360R, E362D, A393R and A393E relative to the amino acid sequence recited in TABLE 1. In some embodiments, the effector partner comprises an amino acid sequence that is at least 75%, at least 80%, at least 85%, at least 90%, at least 95%, at least 97%, or at least 98%, at least 99%, or 100% identical to the amino acid sequences of SEQ ID NO: 2. In some embodiments, the effector partner edits a base of the target nucleic acid, wherein the target nucleic acid is a single stranded target nucleic acid. In some embodiments, the effector partner edits a base of the target nucleic acid, wherein the target nucleic acid is a double stranded target nucleic acid comprising a target strand and a non-target strand. In some embodiments, the effector partner edits the target strand. In some embodiments, the effector partner edits the non-target strand. In some embodiments, the system comprises a fusion protein, or a nucleic acid encoding the fusion protein, wherein the fusion protein comprises the effector protein and the effector partner fused to each other. In some embodiments, the effector protein is linked to an N-terminus of the effector partner. In some embodiments, the effector protein comprises D220R and D237A amino acid substitutions, and wherein the effector partner comprises an amino acid sequence that is at least at least 90% identical to the amino acid sequence of SEQ ID NO: 2. In some embodiments, the effector protein comprises D220R and D237N amino acid substitutions, and wherein the effector partner comprises an amino acid sequence that is at least at least 90% identical to the amino acid sequence of SEQ ID NO: 2. In some embodiments, the effector protein comprises E335Q amino acid substitutions, and wherein the effector partner comprises an amino acid sequence that is at least at least 90% identical to the amino acid sequence of SEQ ID NO: 2. In some embodiments, the effector protein further comprises D220R amino acid substitution. In some embodiments, the effector protein is linked to a C-terminus of the effector partner. In some embodiments, the effector protein comprises D220R and D237A amino acid substitutions, and wherein the effector partner comprises an amino acid sequence that is at least at least 90% identical to the amino acid sequence of SEQ ID NO: 2. In some embodiments, the effector protein comprises D220R and D237N amino acid substitutions, and wherein the effector partner comprises an amino acid sequence that is at least at least 90% identical to the amino acid sequence of SEQ ID NO: 2. In some embodiments, the effector protein comprises E335Q amino acid substitutions, and wherein the effector partner comprises an amino acid sequence that is at least at least 90% identical to the amino acid sequence of SEQ ID NO: 2. In some embodiments, the effector protein further comprises D220R amino acid substitution.

Also provided are the systems described herein, wherein the systems further comprise one or more additional effector partners. In some embodiments, the one or more additional effector partners are fused to the fusion proteins or the effector partners described herein. In some embodiments, the one or more additional effector partners comprise a uracil glycosylase inhibitor, a ssDNA binding protein, a reverse transcriptase, a deaminase, a transcriptional activator, a transcriptional repressor, a functional domain thereof, or a combination thereof. In some embodiments, the one or more additional effector partners independently comprises an amino sequence that is at least 75%, at least 80%, at least 85%, at least 90%, at least 95%, at least 97%, or at least 98%, at least 99%, or 100% identical to any one of the amino acid sequences recited in TABLE 2. In some embodiments, the one or more additional effector partners are fused to the fusion protein. In some embodiments, the spacer sequence comprises a nucleotide sequence in a range of from 10 to 24 linked nucleotides. In some embodiments, the spacer sequence consists of a nucleotide sequence of 12, 13, 20, or 21 nucleotides. In some embodiments, the spacer sequence comprises a nucleotide sequence of 20 linked nucleotides. In some embodiments, the spacer sequence comprises a nucleotide sequence of 21 linked nucleotides. In some embodiments, the spacer sequence comprises a nucleotide sequence in a range of from 10 to 20 linked nucleotides. In some embodiments, the spacer sequence comprises a nucleotide sequence of 12 linked nucleotides. In some embodiments, the spacer sequence comprises a nucleotide sequence of 13 linked nucleotides. In some embodiments, the spacer sequence comprises a nucleotide sequence that is about 80% to about 95% complementary to the target sequence. In some embodiments, the spacer sequence comprises a nucleotide sequence that is at least 90% identical to any one of the nucleotide sequences recited in TABLE 21, TABLE 22, TABLE 23, TABLE 24, TABLE 25, TABLE 26, TABLE 27 and TABLE 28. In some embodiments, the first region comprises a repeat sequence that at least partially interacts with the effector protein. In some embodiments, the repeat sequence comprises a nucleotide sequence that is at least 75%, at least 80%, at least 85%, at least 90%, at least 95%, at least 97%, or at least 98%, at least 99%, or 100% identical to any one of the nucleotide sequences recited in TABLE 5. In some embodiments, the engineered guide nucleic acid is a crRNA. In some embodiments, the first region comprises an intermediary sequence that at least partially interacts with the effector protein. In some embodiments, the intermediary sequence comprises a nucleotide sequence that is at least 75%, at least 80%, at least 85%, at least 90%, at least 95%, at least 97%, or at least 98%, at least 99%, or 100% identical to any one of the nucleotide sequences recited in TABLE 6. In some embodiments, the first region comprises a handle sequence that at least partially interacts with the effector protein. In some embodiments, the handle sequence comprises a nucleotide sequence that is at least 75%, at least 80%, at least 85%, at least 90%, at least 95%, at least 97%, or at least 98%, at least 99%, or 100% identical to any one of the nucleotide sequences recited in TABLE 5, TABLE 6 and TABLE 8. In some embodiments, the engineered guide nucleic acid is a sgRNA. In some embodiments, the engineered guide nucleic acid comprises one or more phosphorothioate (PS) backbone modifications, one or more 2′-fluoro (2′-F) sugar modifications, one or more 2′-O-Methyl(2′OMe) sugar modifications, one or more GC covariation modifications, or combinations thereof. In some embodiments, the engineered guide nucleic acid comprises a chemical modification pattern represented in FIG. 43. In some embodiments, the target sequence is adjacent to a protospacer adjacent motif (PAM). In some embodiments, the effector protein recognizes the PAM. In some embodiments, the PAM comprises any one of the nucleotide sequences of TABLE 4. In some embodiments, the effector protein, the effector partner or a combination thereof further comprises one or more additional heterologous peptide that are heterologous to the effector protein, the effector partner, or the combination thereof, respectively. In some embodiments, the one or more additional heterologous peptides are located at N-terminus, C-terminus, or both of the effector protein, the effector partner, or the combination thereof. In some embodiments, the one or more heterologous peptide independently comprises any one of the amino acid sequences recited in TABLE 3. In some embodiments, the target sequence is within a human gene. In some embodiments, the target nucleic acid is any one of the genes set forth in TABLE 9. In some embodiments, the target nucleic acid is associated with any one of the diseases or disorders of TABLE 10. In some embodiments, the system comprises an expression vector, wherein the expression vector encodes the effector protein, the base editing enzyme, the engineered guide nucleic acid, the one or more additional effector partners, or a combination thereof. In some embodiments, the system comprises an expression vector, wherein the expression vector encodes the effector protein, the base editing enzyme, and the engineered guide nucleic acid. In some embodiments, the nucleic acid expression vector is a viral vector or a non-viral vector. In some embodiments, the viral vector is an adeno associated viral (AAV) vector. In some embodiments, the nucleic acids encoding the effector protein, the effector partner or combinations thereof comprises a messenger RNA. In some embodiments, the nucleic acids encoding the effector protein, the effector partner or combinations thereof are messenger RNAs. In some embodiments, the systems described herein comprise a lipid or a lipid nanoparticle. In some embodiments, the lipid nanoparticle is formulated according to any one of LNP formulations described in TABLE 20.

Also provided herein are libraries of nucleic acid expression vectors comprising at least one of the nucleic acid expression vectors described herein.

Also provided herein are compositions comprising one or more components of any one of the systems described herein.

Also provided herein are compositions comprising one or more components of any one of the systems described herein for use in therapy.

Also provided herein are pharmaceutical compositions comprising the systems or the compositions described herein and a pharmaceutically acceptable excipient.

Also provided herein are cells or progeny thereof comprising the systems described herein, the libraries of nucleic acid expression vectors described herein, the compositions described herein, or the pharmaceutical compositions described herein.

Also provided herein are systems comprising one or more components, wherein the one or more components individually or collectively comprise: (a) an effector protein, or a nucleic acid encoding the effector protein, wherein the effector protein comprises an amino acid sequence of SEQ ID NO: 1 having D220R and E335Q substitutions relative to SEQ ID NO: 1; (b) an effector partner, or a nucleic acid encoding the effector partner, wherein the effector partner is a base editing enzyme, wherein the base editing enzyme comprises an amino acid sequence of SEQ ID NO: 2, and wherein the N-terminus of the base editing enzyme is fused to C-terminus of the effector protein; and (c) an engineered guide nucleic acid or a nucleic acid that encodes the engineered guide nucleic acid, wherein the engineered guide nucleic acid comprises a first region and a second region, wherein the first region, at least partially, interacts with the effector protein, wherein the second region comprises a spacer sequence, wherein the spacer sequence comprises any one of nucleic acid sequences recited in TABLE 21, TABLE 22, TABLE 23, TABLE 24, TABLE 25, TABLE 26, TABLE 27 and TABLE 28.

Also provided herein are systems comprising one or more components, wherein the one or more components individually or collectively comprise: (a) an effector protein, or a nucleic acid encoding the effector protein, wherein the effector protein comprises any one of the amino acid sequences recited in TABLE 1.1; (b) an effector partner, or a nucleic acid encoding the effector partner, wherein the effector partner comprises any one of the amino acid sequences of TABLE 2; and (c) an engineered guide nucleic acid or a nucleic acid that encodes the engineered guide nucleic acid, wherein (i) the engineered guide nucleic acid, at least partially, interacts with a target nucleic acid, (ii) the target nucleic acid comprises a target strand or a non-target strand of the target nucleic acid, (iii) the effector protein nicks the target strand or the non-target strand, and (iv) the effector partner edits a non-nicked strand. In some embodiments, the effector protein comprises any one of the amino acid sequences of SEQ ID NOs: 379, 377 and 381. In some embodiments, the effector protein comprises an amino acid sequence of SEQ ID NO: 379. In some embodiments, a nicked strand is corrected by reverse transcriptase editing. In some embodiments, the effector partner is fused to the effector protein.

Also provided herein are methods of modifying a target nucleic acid of a cell, the method comprising contacting the cell with one or more of: (a) the systems described herein; (b) the libraries of nucleic acid expression vectors described herein; (c) the compositions described herein; or (d) the pharmaceutical compositions described herein, thereby modifying the target nucleic acid of the cell. In some embodiments, the target nucleic acid comprises a target strand that is being modified. In some embodiments, the target nucleic acid comprises a non-target strand that is being modified. In some embodiments, the target nucleic acid comprises one or more mutations associated with a disease. In some embodiments, the one or more mutations comprise a point mutation, a single nucleotide polymorphism (SNP), a chromosomal mutation, a copy number mutation, or any combination thereof. In some embodiments, the target nucleic acid is any one of the nucleic acids set forth in TABLE 9. In some embodiments, the target nucleic acid is associated with any one of the diseases set forth in TABLE 10. In some embodiments, the method comprises modifying a target strand of the target nucleic acid. In some embodiments, the method comprises modifying a non-target strand of the target nucleic acid.

Also provided herein are methods of modifying a target nucleic acid, the method comprising contacting the target nucleic acid with one or more of: (a) the systems described herein; (b) the libraries of nucleic acid expression vectors described herein; (c) the compositions described herein; or (d) the pharmaceutical compositions described herein, thereby modifying the target nucleic acid. In some embodiments, the target nucleic acid comprises a target strand that is being modified. In some embodiments, the target nucleic acid comprises a non-target strand that is being modified. In some embodiments, the method is performed in a cell. In some embodiments, the method is performed in vivo. In some embodiments, the method is performed ex vivo. In some embodiments, the method is performed in vitro. In some embodiments, the target nucleic acid comprises one or more mutations associated with a disease. In some embodiments, the one or more mutations comprise a point mutation, a single nucleotide polymorphism (SNP), a chromosomal mutation, a copy number mutation, or any combination thereof. In some embodiments, the target nucleic acid is any one of the nucleic acids set forth in TABLE 9. In some embodiments, the target nucleic acid is associated with any one of the diseases set forth in TABLE 10. In some embodiments, the method comprises modifying a target strand of the target nucleic acid. In some embodiments, the method comprises modifying a non-target strand of the target nucleic acid.

Also provided herein are methods of modifying a nucleobase of a target nucleic acid within a human gene or associated with expression of a human gene, the method comprising contacting the target nucleic acid with one or more of: (a) the systems described herein; (b) the libraries of nucleic acid expression vectors described herein; (c) the compositions described herein; or (d) the pharmaceutical compositions described herein, thereby modifying the nucleotide base. In some embodiments, the target nucleic acid comprises a target strand that is being modified. In some embodiments, the target nucleic acid comprises a non-target strand that is being modified. In some embodiments, the method is performed in a cell. In some embodiments, the method is performed in vivo. In some embodiments, the method is performed ex vivo. In some embodiments, the method is performed in vitro. In some embodiments, the target nucleic acid comprises a mutation associated with a disease. In some embodiments, the one or more mutations comprise a point mutation, a single nucleotide polymorphism (SNP), a chromosomal mutation, a copy number mutation, or any combination thereof. In some embodiments, the target nucleic acid is any one of the genes set forth in TABLE 9. In some embodiments, the target nucleic acid is associated with any one of the diseases set forth in TABLE 10. In some embodiments, the method comprises modifying a target strand of the target nucleic acid. In some embodiments, the method comprises modifying a non-target strand of the target nucleic acid.

Also provided herein are cells or progeny thereof contacted by: (a) the systems described herein; (b) the libraries of nucleic acid expression vectors described herein; (c) the compositions described herein; (d) the pharmaceutical compositions described herein; or (e) the methods described herein.

Also provided herein are cells or progeny thereof comprising a nucleotide base of a target nucleic acid modified by: (a) the systems described herein; (b) the libraries of nucleic acid expression vectors described herein; (c) the compositions described herein; or (d) the pharmaceutical compositions described herein; or (e) the methods described herein.

Also provided are cells or progeny thereof described herein, wherein the cell is a eukaryotic cell. In some embodiments, the cell or progeny thereof is a mammalian cell. In some embodiments, the cell is a human cell.

Also provided herein is a population of cells that comprises at least one cell as described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1B show schematics of activities for polypeptides. FIG. 1A shows an exemplary base editor comprising an effector protein (light grey) linked to a base editing enzyme (dark grey), wherein a guide nucleic acid hybridizes to a target strand (TS), the effector protein nicks a non-target strand (NTS), and a nucleobase modification occurs on the NTS. Nucleobase modifications may occur on the TS alternatively or additionally (not shown). FIG. 1B shows a numbering scheme for analyzing ABE-mediated base editing data provided herein. Positions are fixed counting from the 5′ direction on the NTS. For example, position 1 is the first nucleotide of the NTS that is 3′ of the PAM. In this example, NTS editing is reported as A>G editing percent in NTS and TS editing is reported as T>C editing percent in NTS.

FIGS. 2A-2C shows exemplary AAV constructs. FIG. 2A shows an exemplary AAV construct comprising an effector protein, an effector partner and a guide RNA according to an embodiment of the disclosure. FIG. 2B shows an exemplary AAV construct comprising a fusion protein and a guide RNA according to an embodiment of the disclosure, wherein the fusion protein comprises an effector protein and an effector partner. FIG. 2C shows an exemplary AAV construct comprising a fusion protein, an effector partner and a guide RNA according to an embodiment of the disclosure, wherein the fusion protein comprises an effector protein and a base editing enzyme.

FIG. 3 shows the nuclease activity of CasM.265466 with flexible PAM sequences, in accordance with an embodiment of the present disclosure.

FIG. 4 illustrates the effects of an arginine substitution on CasM.265466 nuclease activity for a target nucleic acid, in accordance with an embodiment of the present disclosure.

FIG. 5 illustrates the dose titration curves of CasM.265466 arginine mutants, in accordance with an embodiment of the present disclosure.

FIG. 6 illustrates % indel generated by D220R effector protein variant of CasM.265466 relative to corresponding wildtype CasM.265466 and Cas9 effector proteins.

FIG. 7 shows % indel generated at various dose (ng) for D220R and A306K variants relative to CasM.265466 and Cas9 effector proteins.

FIG. 8 shows the activity of the engineered variants relative to that of wildtype as fold change.

FIG. 9 shows gel electrophoresis analysis of cis cleavage activity by CasM.265466 protein and variants thereof.

FIG. 10 shows schematics of a fluorescence polarization assay using a duplex substrate and a non-paired DNA substrate.

FIG. 11 shows binding affinity curves for the wildtype CasM.265466 protein with non-paired DNA and normal duplex DNA.

FIGS. 12A-12B show binding affinity curves for the CasM.265466 variant effector proteins relative to corresponding wildtype CasM.265466 effector protein, wherein the polarization (mP) observed is plotted against concentration of the effector protein.

FIGS. 13A-13B show binding affinity curves for the variant D220R, relative to the corresponding wildtype CasM.265466 effector protein, wherein the polarization (mP) observed is plotted against concentration of the effector protein.

FIGS. 14A-14B show cis cleavage catalytic activity for the variant D220R relative to the wildtype effector protein.

FIG. 15 shows heat map color distribution of four engineered polypeptides, ABE8e_466-D220R-E335Q, 466-D220R-E335Q_ABE8e, ABE8e_466-E335Q and 466-E335Q_ABE8e, tested for editing window observed in the non-target strand.

FIG. 16 shows heat map color distribution of four engineered polypeptides, ABE8e_466-D220R-E335Q, 466-D220R-E335Q_ABE8e, ABE8e_466WT-E335Q and 466 WT-E335Q_ABE8e, tested for editing window observed in the target strand.

FIG. 17 summarizes result of engineered polypeptides, a265466-D220R-D237A_ABE8e, ABE8e_a265466-D220R-D237A, a265466-D220R-D237N_ABE8e, ABE8e_a265466-D220R-D 237N, a265466-D220R-E335Q_ABE8e and ABE8e_a265466-E335Q-D237N, mediated change in % base in the non-target strand.

FIG. 18 summarizes result of engineered polypeptides, a265466-D220R-D237A_ABE8e, ABE8e_a265466-D220R-D237A, a265466-D220R-D237N_ABE8e, ABE8e_a265466-D220R-D237N, a265466-D220R-E335Q_ABE8e and ABE8e_a265466-E335Q-D237N, mediated change in % base in the target strand.

FIG. 19 shows results of change in nuclease activity of the CasM.265466 WT effector protein (SEQ ID NO: 1) as a function of change in spacer sequence length of guide RNAs that are targeting a sequence within B2M gene.

FIG. 20 shows quantitative analysis of FIG. 19 data showing relative quantities of uncut, nicked or cleaved (double stranded break) target nucleic acid.

FIG. 21 shows results of an NGS analysis for CasM.265466 D220R effector protein system relative to Cas9 effector protein system, wherein the effector protein systems were tested for their ability to generate % indels in B2M gene, TRAC gene, and CIITA gene, individually or simultaneously (Triple edit 3×).

FIG. 22A-22C show results of an FACS analysis for CasM.265466 D220R effector protein system relative to Cas9 effector protein system targeting a gene of interest. Specifically, FIG. 22A shows results of an FACS analysis for CasM.265466 D220R effector protein system relative to Cas9 effector protein system, wherein the effector protein systems were tested for their ability to knockout B2M gene expression by targeting B2M gene individually (Single Edit) or simultaneous targeting B2M gene, TRAC gene, and CIITA gene (Triple Edit). FIG. 22B shows results of an FACS analysis for CasM.265466 D220R effector protein system relative to Cas9 effector protein system, wherein the effector protein systems were tested for their ability to knockout CD3 expression by targeting TRAC gene individually (Single Edit) or simultaneous targeting B2M gene, TRAC gene, and CIITA gene (Triple Edit). FIG. 22C shows results of an FACS analysis for CasM.265466 D220R effector protein system relative to Cas9 effector protein system, wherein the effector protein systems were tested for their ability to generate % indels in CIITA gene, wherein the effector protein systems were targeting CIITA gene individually (Single Edit), or B2M gene, TRAC gene and CIITA gene simultaneously (Triple Edit).

FIG. 23 shows results of translocation rates based on an dGH assay for CasM.265466 D220R effector protein system relative to Cas9 effector protein system, wherein the effector protein systems were tested for their ability to generate % indels in B2M gene, TRAC gene, and CIITA gene, individually (1×) or simultaneously (Triple edit 3×).

FIG. 24 shows results of reciprocal translocations rates based on dGH assay for CasM.265466 D220R effector protein system relative to Cas9 effector protein system, wherein the effector protein systems were tested for their ability to generate % indels in B2M gene, TRAC gene, and CIITA gene, individually (1×) or simultaneously (Triple edit 3×).

FIG. 25A-25C show results of cell counts, cell viability and cell numbers for CasM.265466 D220R effector protein system edited target nucleic acids relative to Cas9 effector protein system edited target nucleic acids. Specifically, FIG. 25A shows results of cell counts for CasM.265466 D220R effector protein system edited target nucleic acids relative to Cas9 effector protein system edited target nucleic acids (B2M gene, TRAC gene, and CIITA gene, individually (1×) or simultaneously (Triple edit 3×)). FIG. 25B shows results of cell viability for CasM.265466 D220R effector protein system edited target nucleic acids relative to Cas9 effector protein system edited target nucleic acids (B2M gene, TRAC gene, and CIITA gene, individually (1×) or simultaneously (Triple edit 3×)). FIG. 25C shows results of cell numbers for CasM.265466 D220R effector protein system edited target nucleic acids relative to Cas9 effector protein system edited target nucleic acids.

FIG. 26 shows results of an NGS analysis for CasM.265466 D220R effector protein system relative to Cas9 effector protein system, wherein the effector protein systems were tested for their ability to generate indels within target nucleic acids (B2M gene, TRAC gene, and CIITA gene, individually (Single Edit) or simultaneously (Triple Edit)).

FIG. 27A-27B show results of NGS analysis for CasM.265466 D220R effector protein system and Cas9 effector protein system, wherein the effector protein systems were tested for their ability to generate indels within target nucleic acids at 6 μg and 9 μg. Specifically, FIG. 27A shows results of an NGS analysis for CasM.265466 D220R effector protein system and Cas9 effector protein system, wherein the effector protein systems were tested for their ability to generate indels within target nucleic acids (B2M gene, TRAC gene, and CIITA gene, individually (Single) or simultaneously (Triple)) at 6 μg. FIG. 27B shows results of an NGS analysis for CasM.265466 D220R effector protein system and Cas9 effector protein system, wherein the effector protein systems were tested for their ability to generate indels within target nucleic acids (B2M gene, TRAC gene, and CIITA gene, individually (Single) or simultaneously (Triple)) at 9 μg. Results for indels generated by Cas9 and guide nucleic acids targeting CIITA gene are not shown.

FIG. 28A-28B show results of FACS analysis for CasM.265466 D220R effector protein system relative to Cas9 effector protein system, wherein the effector protein systems were tested for their ability to generate indels within target nucleic acids at 6 μg and 9 μg. Specifically, FIG. 28A shows results of FACS analysis for CasM.265466 D220R effector protein system relative to Cas9 effector protein system, wherein the effector protein systems were tested for their ability to generate indels within target nucleic acids (B2M gene, TRAC gene, and CIITA gene, individually (Single) or simultaneously (Triple)) at 6 μg. FIG. 28B show results of FACS analysis for CasM.265466 D220R effector protein system relative to Cas9 effector protein system, wherein the effector protein systems were tested for their ability to generate indels within target nucleic acids (B2M gene, TRAC gene, and CIITA gene, individually or simultaneously (Triple)) at 9 μg.

FIG. 29A-29B show results of cell counts analysis for CasM.265466 D220R effector protein system relative to Cas9 effector protein system, wherein the effector protein systems were tested for their ability to generate indels within target nucleic acids at 6 μg and 9 μg. Specifically, FIG. 29A shows results of cell counts analysis for CasM.265466 D220R effector protein system relative to Cas9 effector protein system, wherein the effector protein systems were tested for their ability to generate indels within target nucleic acids (B2M gene, TRAC gene, and CIITA gene, individually (Single Edit) or simultaneously (Triple Edit)) at 6 μg. FIG. 29B shows results of cell counts analysis for CasM.265466 D220R effector protein system relative to Cas9 effector protein system, wherein the effector protein systems were tested for their ability to generate indels within target nucleic acids (B2M gene, TRAC gene, and CIITA gene, individually (Single Edit) or simultaneously (Triple Edit)) at 9 μg.

FIG. 30A-30B show results of cell viability analysis for CasM.265466 D220R effector protein system relative to Cas9 effector protein system, wherein the effector protein systems were tested for their ability to generate indels within target nucleic acids at 6 μg and 9 μg. Specifically, FIG. 30A shows results of cell viability analysis for CasM.265466 D220R effector protein system relative to Cas9 effector protein system, wherein the effector protein systems were tested for their ability to generate indels within target nucleic acids (B2M gene, TRAC gene, and CIITA gene, individually (Single Edit) or simultaneously (Triple Edit)) at 6 μg. FIG. 30B shows results of cell viability analysis for CasM.265466 D220R effector protein system relative to Cas9 effector protein system, wherein the effector protein systems were tested for their ability to generate indels within target nucleic acids (B2M gene, TRAC gene, and CIITA gene, individually (Single Edit) or simultaneously (Triple Edit)) at 9 μg.

FIG. 31 shows results of an NGS analysis for in vivo target nucleic acid editing for CasM.265466 D220R effector protein system relative to Cas9 effector protein system.

FIG. 32 shows results of NGS study conducted for determining off-target activity. Eight off-target sites were identified and tested. For each off-target site, NGS study was conducted on non-treated (left) cells, cells treated with mRNA encoding CasM.265466 effector protein (middle), and cells treated with mRNA encoding D220R variant of CasM.265466 effector protein.

FIG. 33 shows results of NGS study conducted for determining indel activity of CasM.265466 effector protein variants targeting a target sequence within MLH1.

FIG. 34 shows results of effect of linker length on gene editing activity of engineered polypeptide comprising an adenine base editing enzyme. Specifically, FIG. 34 shows (a) % editing generated by each engineered polypeptide on the non-target strand and the target strand; and (b) ratio of peak % editing generated on the target strand (PeakTS %) and peak % editing generated on the non-target strand (PeakNTS %). A higher value of the ratio (PeakTS %:PeakNTS %) indicates higher preference for editing the target strand relative to the non-target strand.

FIG. 35 shows results of effect of spacer length on nicking activity of engineered polypeptide. Specifically, FIG. 35 shows: (1) % indel generated by each guide nucleic acid; (2) % substitution generated by each guide nucleic acid on a non-target strand and a target strand; and (3) a ratio of peak % editing generated on the target strand (PeakTS %) and peak % editing generated on the non-target strand (PeakNTS %). A higher value of the ratio (PeakTS %:PeakNTS %) indicates higher preference for editing the target strand relative to the non-target strand.

FIG. 36 shows results of gene editing activity of an engineered polypeptide comprising cytosine deaminase. Specifically, FIG. 36 shows: (1) % substitution generated by each guide nucleic acid; (2) % editing generated by each guide nucleic acid from C to T, C to G and C to A; and (3) % editing generated by each guide nucleic acid from A to G. An analysis of FIG. 36 indicates that cytosine base editing activity of cytosine deaminase can be used for gene knockout application.

FIG. 37 shows heat map color distribution of a representative engineered polypeptide comprising cytosine deaminase tested for editing window observed in the non-target strand.

FIG. 38 shows heat map color distribution of a representative engineered polypeptide comprising cytosine deaminase tested for editing window observed in the target strand.

FIGS. 39A-39B show the impact of spacer length on activities of a combination of a gRNA and a CasM.265466 effector protein variant in primary human hepatocytes. Specifically, FIG. 39A shows indel activity results from NGS analysis. FIG. 39B shows fluorescence results of luciferase assay.

FIGS. 40A-40B show the impact of spacer length on activities of a combination of a gRNA and a CasM.265466 effector protein variant in primary human hepatocytes. Specifically, FIG. 40A shows indel activity results from NGS analysis. FIG. 40B shows fluorescence results of luciferase assay.

FIG. 41 shows impact of 2′-O-Methyl(2-O-Me) sugar modifications in gRNA on activity of a combination of the gRNA and CasPhi.265466 effector protein in Hep3B cells.

FIG. 42 shows impact of phosphorothioate (PS) backbone modifications in gRNA on activity of a combination of the gRNA and CasPhi.265466 effector protein in Hep3B cells.

FIG. 43 shows various gRNA modifications that were tested in Hep3B cells. The modifications include one or more 2′-O-Methyl(2-O-Me) sugar modifications, shown as ▪, one or more phosphorothioate (PS) backbone modifications, shown as [symbol] , and G-C covariation modifications.

FIG. 44 shows impact of various gRNA modifications that were tested in Hep3B cells as described in FIG. 43.

FIGS. 45A-45C show impact of G-C covariation modifications of gRNA on activity of a combination of the gRNA and CasPhi.265466 effector protein over U-A covariations in Hep3B cells as summarized in FIG. 44. Specifically, FIG. 45A shows effect of G-C covariation modifications (right bar) over U-A covariations (left bar), where the gRNA was incubated with the Hep3B cells for 0 hours prior to transfection. FIG. 45B shows effect of G-C covariation modifications (right bar) over U-A covariations (left bar), where the gRNA was incubated with the Hep3B cells for 6 hours prior to transfection. FIG. 45C shows effect of G-C covariation modifications (right bar) over U-A covariations (left bar), where the gRNA was incubated with the Hep3B cells for 12 hours prior to transfection.

FIG. 46 shows target nucleic acid modifying activity of CasM.265466 variant fusions. Seven CasM.265466 variant fusions were tested. Normalized indel activity are shown in left bars. Normalized nickase scores are shown in right bars. Wildtype CasM.265466 was used as a positive control. dCasM.265466 (D220R/E335Q) was used as a negative control.

FIGS. 47A-47D show results of % indel generated, % target-strand editing observed, % non-target strand editing observed, and nickase score calculated, respectively, for each of the tested fusion protein systems. Each fusion protein system comprised an RNA encoding a CasM.265466 variant fused to a deaminase protein, and a guide RNA. Eight plasmids (PL28330, PL34536, PL34572, PL34590, PL34592, PL34612, PL34621 and PL28336) were used, wherein each plasmid encodes a different guide RNA. Eight CasM.265466 variants and two controls were tested. FIGS. 47A-47D include groups of 10 bars for each tested plasmid, wherein each bar shows results for CasM.265466 variants having the amino acid sequence of one of SEQ ID NOs: 379, 377, 381, 378, 380, 375, 382 or 383, a control dCasM.265466 D220R/E335Q, or a control CasM.265466 D220R (SEQ ID NO: 385) (left to right).

FIG. 48 shows results of % indel generated, % target-strand editing observed, % non-target strand editing observed, and ratio of % target-strand editing observed and % non-target strand editing observed for each of the tested fusion protein systems. Each fusion protein system comprised an RNA encoding a CasM.265466 variant fused to a deaminase protein, and a guide RNA. A plasmid (PL28330) encoding a guide RNA that targets MLH1 gene was used. Three CasM.265466 variants and four controls were tested. FIG. 48 includes groups of 5 bars for each fusion protein system, wherein each bar shows results for CasM.265466 variants having the amino acid sequence of one of SEQ ID NO: 379, 377 or 381, a control dCasM.265466 D220R/E335Q, a control CasM.265466 D220R (SEQ ID NO: 385), a control dCas9, or a control nCas9 (left to right).

FIG. 49 shows results of % indel generated, % target-strand editing observed, % non-target strand editing observed, and ratio of % target-strand editing observed and % non-target strand editing observed for each of the tested fusion protein systems. Each fusion protein system comprised an RNA encoding a CasM.265466 variant fused to a deaminase protein, and a guide RNA. A plasmid (PL34536) encoding a guide RNA that targets ANGPTL3 gene was used. Three CasM.265466 variants and four controls were tested. FIG. 49 includes groups of seven bars for each fusion protein system, wherein each bar shows results for each bar shows results for CasM.265466 variants having the amino acid sequence of one of SEQ ID NO: 379, 377 or 381, a control dCasM.265466 D220R/E335Q, a control CasM.265466 D220R (SEQ ID NO: 385), a control dCas9, or a control nCas9 (left to right).

FIG. 50 shows results of % indel generated, % target-strand editing observed, % non-target strand editing observed, and ratio of % target-strand editing observed and % non-target strand editing observed for each of the tested fusion protein systems. Each fusion protein system comprised an RNA encoding a CasM.265466 variant fused to a deaminase protein, and a guide RNA. A plasmid (PL34572) encoding a guide RNA that targets PCSK9 gene was used. Three CasM.265466 variants and two controls were tested. FIG. 50 includes groups of 5 bars for each fusion protein system, wherein each bar shows results for CasM.265466 variants having the amino acid sequence of one of SEQ ID NO: 379, 377 or 381, a control dCasM.265466 D220R/E335Q, or a control CasM.265466 D220R (SEQ ID NO: 385) (left to right).

FIG. 51 shows results of % indel generated, % target-strand editing observed, % non-target strand editing observed, and ratio of % target-strand editing observed and % non-target strand editing observed for each of the tested fusion protein systems. Each fusion protein system comprised an RNA encoding a CasM.265466 variant fused to a deaminase protein, and a guide RNA. A plasmid (PL28336) encoding a guide RNA that targets B2M gene was used. Three CasM.265466 variants and two controls were tested. FIG. 51 includes groups of 5 bars for each fusion protein system, wherein each bar shows results for CasM.265466 variants having the amino acid sequence of one of SEQ ID NO: 379, 377 or 381, a control dCasM.265466 D220R/E335Q, or a control CasM.265466 D220R (SEQ ID NO: 385) (left to right).

FIG. 52 shows results of % indel generated, % target-strand editing observed, % non-target strand editing observed, and ratio of % target-strand editing observed and % non-target strand editing observed for each of the tested fusion protein systems. Each fusion protein system comprised an RNA encoding a CasM.265466 variant fused to a deaminase protein, and a guide RNA. A plasmid (PL34590) encoding a guide RNA that targets LDHA gene was used. Three CasM.265466 variants and two controls were tested. FIG. 52 includes groups of 5 bars for each fusion protein system, wherein each bar shows results for CasM.265466 variants having the amino acid sequence of one of SEQ ID NO: 379, 377 or 381, a control dCasM.265466 D220R/E335Q, or a control CasM.265466 D220R (SEQ ID NO: 385) (left to right).

FIG. 53 shows results of % indel generated, % target-strand editing observed, % non-target strand editing observed, and ratio of % target-strand editing observed and % non-target strand editing observed for each of the tested fusion protein systems. Each fusion protein system comprised an RNA encoding a CasM.265466 variant fused to a deaminase protein, and a guide RNA. A plasmid (PL34592) encoding a guide RNA that targets LDHA gene was used. Three CasM.265466 variants and two controls were tested. FIG. 53 includes groups of 5 bars for each fusion protein system, wherein each bar shows results for CasM.265466 variants having the amino acid sequence of one of SEQ ID NO: 379, 377 or 381, a control dCasM.265466 D220R/E335Q, or a control CasM.265466 D220R (SEQ ID NO: 385) (left to right).

FIG. 54 shows results of % indel generated, % target-strand editing observed, % non-target strand editing observed, and ratio of % target-strand editing observed and % non-target strand editing observed for each of the tested fusion protein systems. Each fusion protein system comprised an RNA encoding a CasM.265466 variant fused to a deaminase protein, and a guide RNA. A plasmid (PL34612) encoding a guide RNA that targets HSD17B13 gene was used. Three CasM.265466 variants and two controls were tested. FIG. 54 includes groups of 5 bars for each fusion protein system, wherein each bar shows results for CasM.265466 variants having the amino acid sequence of one of SEQ ID NO: 379, 377 or 381, a control dCasM.265466 D220R/E335Q, or a control CasM.265466 D220R (SEQ ID NO: 385) (left to right).

FIG. 55 shows results of % indel generated, % target-strand editing observed, % non-target strand editing observed, and ratio of % target-strand editing observed and % non-target strand editing observed for each of the tested fusion protein systems. Each fusion protein system comprised an RNA encoding a CasM.265466 variant fused to a deaminase protein, and a guide RNA. A plasmid (PL34612) encoding a guide RNA that targets HSD17B13 gene was used. Three CasM.265466 variants and two controls were tested. FIG. 55 includes groups of 5 bars for each fusion protein system, wherein each bar shows results for CasM.265466 variants having the amino acid sequence of one of SEQ ID NO: 379, 377 or 381, a control dCasM.265466 D220R/E335Q, or a control CasM.265466 D220R (SEQ ID NO: 385) (left to right).

FIGS. 56A-56B show heatmap for % indel generated, and nickase score calculated, respectively, for each of the tested fusion protein systems. Each fusion protein system comprised an RNA encoding a CasM.265466 variant fused to a deaminase protein, and a guide RNA. Eight plasmids (PL28330, PL34536, PL34572, PL34590, PL34592, PL34612, PL34621 and PL28336) were used, wherein each plasmid encodes a different guide RNA. CasM.265466 variants of SEQ ID NO: 375 and 377-384 were tested. Two controls dCasM.265466 D220R/E335Q and CasM.265466 D220R (SEQ ID NO: 385) were used.

FIGS. 57A-57B show results of % indel generated, and nickase score calculated, respectively, for each of the tested fusion protein systems. Each fusion protein system comprised an RNA encoding a CasM.265466 variant fused to a deaminase protein, and a guide RNA. CasM.265466 variants of SEQ ID NO: 375, 377-381 and 384 were tested. Two controls dCasM.265466 D220R/E335Q and CasM.265466 D220R (SEQ ID NO: 385) were used. A plasmid (PL28330) targeting MLH1 gene was used. A concentration of the RNA encoding the CasM.265466 fusion protein was kept constant at 150 ng, whereas the plasmid encoding the guide RNA was varied from 25 ng to 150 ng. FIGS. 57A-57B include groups of 4 bars for each fusion protein system, wherein each bar shows results for plasmid concentration of 150 ng, 100 ng, 50 ng and 25 ng (left to right).

FIGS. 58A-58B show results of % indel generated, and nickase score calculated, respectively, for each of the tested fusion protein systems. Each fusion protein system comprised an RNA encoding a CasM.265466 variant fused to a deaminase protein, and a guide RNA. CasM.265466 variants of SEQ ID NO: 375, 377-381 and 384 were tested. Two controls dCasM.265466 D220R/E335Q and CasM.265466 D220R (SEQ ID NO: 385) were used. A plasmid (PL34536) targeting ANGPTL3 gene was used. A concentration of the RNA encoding the CasM.265466 fusion protein was kept constant at 150 ng, whereas the plasmid encoding the guide RNA was varied from 25 ng to 150 ng. FIGS. 58A-58B include groups of 4 bars for each fusion protein system, wherein each bar shows results for plasmid concentration of 150 ng, 100 ng, 50 ng and 25 ng (left to right).

FIG. 59 shows base editing on target strands and non-target strands of exemplary target nucleic acids ANGPTL3, APOC3, HAO1, LDHA, LPA, CNBP, PCSK9, and HSD17B13.

FIG. 60 shows that fusing an endonuclease to a CasM.265466-ABE8e fusion protein can increase editing of a target strand relative to a non-target strand of a double stranded target nucleic acid. Plasmid numbers listed in the legend correspond to data points shown in the figure from left to right in any individual figure panel.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any and all combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is optional and understood to mean the stated number and numbers+/−10% thereof, or 10% below the lower listed limit and 10% above the higher listed limit for the values listed for a range.

The terms, “% identical,” “% identity,” “percent identity,” and grammatical equivalents thereof, as used herein, in the context of an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The terms, “% complementary”, “% complementarity”, “percent complementary”, “percent complementarity” and grammatical equivalents thereof, as used interchangeably herein, in the context of two or more nucleic acid molecules, refer to the percent of nucleotides in two nucleotide sequences in said nucleic acid molecules of equal length that can undergo cumulative base pairing at two or more individual corresponding positions in an antiparallel orientation. Accordingly, the terms include nucleic acid sequences that are not completely complementary over their entire length, which indicates that the two or more nucleic acid molecules include one or more mismatches. A “mismatch” is present at any position in the two opposed nucleotides that are not complementary. The % complementary is calculated by dividing the total number of the complementary residues by the total number of the nucleotides in one of the equal length sequences and multiplying by 100. Complete or total complementarity describes nucleotide sequences in 100% of the residues of a nucleotide sequence are complementary to residues in a reference nucleotide sequence. “Partially complementarity” describes nucleotide sequences in which at least 20%, but less than 100%, of the residues of a nucleotide sequence are complementary to residues in a reference nucleotide sequence. In some instances, at least 50%, but less than 100%, of the residues of a nucleotide sequence are complementary to residues in a reference nucleotide sequence. In some instances, at least 70%, 80%, 90% or 95%, but less than 100%, of the residues of a nucleotide sequence are complementary to residues in a reference nucleotide sequence. “Noncomplementary” describes nucleotide sequences in which less than 20% of the residues of a nucleotide sequence are complementary to residues in a reference nucleotide sequence.

The term, “percent similarity,” or “% similarity,” as used herein, in the context of an amino acid sequence, refers to a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold ≥1.

The terms, “amplification,” “amplifying,” and grammatical equivalents thereof, as used herein, refer to a process by which a nucleic acid molecule is enzymatically copied to generate a plurality of nucleic acid molecules containing the same sequence as the original nucleic acid molecule or a distinguishable portion thereof.

The terms, “bind,” “binding,” “interact” and “interacting,” as used herein, refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

The term, “base editor,” as used herein, refers to a polypeptide or fusion protein comprising a base editing activity. The polypeptide with base editing activity may be referred to as an effector partner. The base editor can differ from a naturally occurring base editing enzyme. It is understood that any reference to a base editor herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some instances, the catalytically inactive effector protein is referred to as a catalytically inactive variant of an effector protein.

The term, “cis cleavage,” as used herein, refers to cleavage (hydrolysis of a phosphodiester bond) of a target nucleic acid by a complex of an effector protein and a guide nucleic acid (e.g., an RNP complex), wherein at least a portion of the guide nucleic acid is hybridized to at least a portion of the target nucleic acid. Cleavage may occur within or directly adjacent to the portion of the target nucleic acid that is hybridized to the portion of the guide nucleic acid.

The term, “codon optimized,” as used herein, refers to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon.

The terms, “complementary” and “complementarity,” as used herein, in the context of a nucleic acid molecule or nucleotide sequence, refer to the characteristic of a polynucleotide having nucleotides that can undergo cumulative base pairing with their Watson-Crick counterparts (C with G; or A with T/U) in a reference nucleic acid in antiparallel orientation. For example, when every nucleotide in a polynucleotide or a specified portion thereof forms a base pair with every nucleotide in an equal length sequence of a reference nucleic acid, that polynucleotide is said to be 100% complementary to the sequence of the reference nucleic acid. In a double stranded DNA or RNA sequence, the upper (sense) strand sequence is, in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the “reverse complement” sequence or the “reverse complementary” sequence is understood as the sequence of the lower strand in the direction of its 5′- to its 3′-end. Each nucleotide in a double stranded DNA or RNA molecule that is paired with its Watson-Crick counterpart can be referred to as its complementary nucleotide. The complementarity of modified or artificial base pairs can be based on other types of hydrogen bonding and/or hydrophobicity of bases and/or shape complementarity between bases.

The term, “cleavage assay,” as used herein, refers to an assay designed to visualize, quantitate or identify cleavage of a nucleic acid. In some instances, the cleavage activity may be cis cleavage activity.

The terms, “cleave,” “cleaving” and “cleavage,” as used herein, in the context of a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from another organism.

The term, “conservative substitution,” as used herein, refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

The terms, “CRISPR RNA” and “crRNA,” as used herein, refer to a type of guide nucleic acid that is RNA comprising a first sequence that is capable of hybridizing to a target sequence of a target nucleic acid and a second sequence that is capable of interacting with an effector protein either directly (by being bound by an effector protein) or indirectly (e.g., by hybridization with a second nucleic acid molecule that can be bound by an effector). The first sequence and the second sequence are directly connected to each other or by a linker.

The term, “detectable product,” as used herein, refers to a unit produced after the cleavage of a reporter that is capable of being discovered, identified, perceived or noticed. A detectable product can comprise a detectable label and/or moiety that emits a detectable signal. A detectable product may include other components that are not capable of being readily discovered, identified, perceived or noticed at the same time as the detectable signal. For example, a detectable product may comprise remnants of the reporter. Accordingly, in some instances, the detectable product comprises RNA and/or DNA.

The term, “detectable signal,” as used herein, refers to an act, event, physical quantity or impulse that can be detected using optical, fluorescent, chemiluminescent, electrochemical or other detection methods known in the art.

The term, “diseased cell,” as used herein, refers to a cell comprising pathway conditions or pathway systems that are not conducive to cell survival, tissue survival, systemic survival, or organism survival.

The term, “edited target nucleic acid,” as used herein, refers to a target nucleic acid, wherein the target nucleic acid has undergone an editing, for example, after contact with an effector protein. In some instances, the editing is an alteration in the sequence of the target nucleic acid. In some instances, the edited target nucleic acid comprises an insertion, deletion, or substitution of one or more nucleotides compared to the unedited target nucleic acid.

The term, “effector protein,” as used herein, refers to a protein, polypeptide, or peptide that is capable of interacting with a nucleic acid, such as a guide nucleic acid, to form a complex (e.g., a RNP complex), wherein the complex interacts with a target nucleic acid.

The term, “effector partner,” as used herein, refers to a protein, polypeptide or peptide that can, in combination with an effector protein, impart some function that can be used to effectuate modification(s) of a target nucleic acid described herein and/or change expression of the target nucleic acid or other nucleic acids associated with the target nucleic acid, when used in connection with compositions, systems and methods described herein.

The term, “engineered modification,” as used herein, refers to a structural change of one or more nucleic acid residues of a nucleotide sequence or one or more amino acid residue of an amino acid sequence. The engineered modifications of a nucleotide sequence can include chemical modification of one or more nucleobases, or a chemical change to the phosphate backbone, a nucleotide, a nucleobase or a nucleoside. The engineered modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “functional domain,” as used herein, refers to a region of one or more amino acids in a protein that is required for an activity of the protein, or the full extent of that activity, as measured in an in vitro assay. Activities include, but are not limited to nucleic acid binding, nucleic acid editing, nucleic acid modifying, nucleic acid cleaving, protein binding. The absence of the functional domain, including mutations of the functional domain, would abolish or reduce activity.

The term, “functional fragment,” as used herein, refers to a fragment of a protein that retains some function relative to the entire protein. Non-limiting examples of functions are nucleic acid binding, nucleic acid editing, protein binding, nuclease activity, nickase activity, deaminase activity, demethylase activity, or acetylation activity. A functional fragment may be a recognized functional domain, e.g., a catalytic domain. In some instances, the catalytic domain comprises a RuvC domain.

The term, “functional protein,” as used herein, refers to protein that retains at least some if not all activity relative to the wildtype protein. A functional protein can also include a protein having enhanced activity relative to the wildtype protein. Assays are known and available for detecting and quantifying protein activity, e.g., colorimetric and fluorescent assays. In some instances, a functional protein is a wildtype protein. In some instances, a functional protein is a functional portion of a wildtype protein.

The term, “fused,” as used herein, refers to at least two sequences that are connected together, such as by a covalent bond (e.g., an amide bond or a phosphodiester bond) or by a linker. The covalent bond can be formed by conjugation (e.g., chemical conjugation or enzymatic conjugation) reaction.

The term, “fusion protein,” as used herein, refers to a protein comprising at least two polypeptides. The fusion protein may comprise one or more effector proteins and effector partners. In some instances, an effector protein and effector partner are not found connected to one another as a native protein or complex that occurs together in nature.

The term, “genetic disease,” as used herein, refers to a disease, disorder, condition, or syndrome associated with or caused by one or more mutations in the DNA of an organism having the genetic disease.

The term, “guide nucleic acid,” as used herein, refers to a nucleic acid that, when in a complex with one or more polypeptides described herein (e.g., an RNP complex) can impart sequence selectivity to the complex when the complex interacts with a target nucleic acid. A guide nucleic acid may be referred to interchangeably as a guide RNA, however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof. A guide RNA may comprise DNA.

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary sequence, that is capable of being non-covalently bound by an effector protein. In some instances, the handle sequence further comprises a repeat sequence. In such instances, the intermediary sequence or a combination of the intermediary sequence and the repeat sequence is capable of being non-covalently bound by an effector protein.

The term, “heterologous,” as used herein, refers to at least two different polypeptide sequences that are not found similarly connected to one another in a native nucleic acid or protein. A protein that is heterologous to the effector protein is a protein that is not covalently linked by an amide bond to the effector protein in nature. In some instances, a protein is heterologous when the protein is not encoded by a species that encodes the protein. A guide nucleic acid may comprise “heterologous” sequences, which means that it includes a first sequence and a second sequence, wherein the first sequence and the second sequence are not found covalently linked by a phosphodiester bond in nature. Thus, the first sequence is considered to be heterologous with the second sequence, and the guide nucleic acid may be referred to as a heterologous guide nucleic acid. A heterologous system comprises at least one component that is not naturally occurring together with remaining components of the heterologous system.

The terms, “hybridize,” “hybridizable” and grammatical equivalents thereof, refer to a nucleotide sequence that is able to noncovalently interact, i.e., form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g. complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Green, M. and Sambrook, J., Molecular Cloning: A Laboratory Manual, Fourth Edition, Cold Spring Harbor Laboratory Press, Cold Spring Harbor (2012).

The term, “indel,” as used herein, refers to an insertion-deletion or indel mutation, which is a type of genetic mutation that results from the insertion and/or deletion of one or more nucleotide in a target nucleic acid. An indel can vary in length (e.g., 1 to 1,000 nucleotides in length) and be detected by any suitable method, including sequencing.

The term, “indel percentage,” as used herein, refers to a percentage of sequencing reads that show at least one nucleotide has been edited from the insertion and/or deletion of nucleotides regardless of the size of insertion or deletion, or number of nucleotides edited. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

The terms, “intermediary RNA” and “intermediary sequence,” as used herein, in a context of a single nucleic acid system, refers to a nucleotide sequence in a handle sequence, wherein the nucleotide sequence is capable of, at least partially, being non-covalently bound to an effector protein to form a complex (e.g., an RNP complex). An intermediary sequence is not a transactivating nucleic acid in systems, methods, and compositions described herein.

The term, “in vitro,” as used herein, refers to describing something outside an organism. An in vitro system, composition or method may take place in a container for holding laboratory reagents such that it is separated from the biological source from which a material in the container is obtained. In vitro assays can encompass cell-based assays in which living or dead cells are employed. In vitro assays can also encompass a cell-free assay in which no intact cells are employed. The term “in vivo” is used to describe an event that takes place within an organism. The term “ex vivo” is used to describe an event that takes place in a cell that has been obtained from an organism. An ex vivo assay is not performed on a subject. Rather, it is performed upon a sample separate from a subject.

The term, “length,” as used herein, is used to characterize the number of nucleotides forming polynucleotides or the number of amino acids forming polypeptides. Polynucleotide length may be expressed as kilobases (kb) or base pairs (bp). Thus, a length of 1 kb refers to a length of 1000 linked nucleotides, and a length of 500 bp refers to a length of 500 linked nucleotides. Similarly, a polypeptide having a length of 500 linked amino acids may also be simply described as having a length of 500 amino acids.

The term, “linker,” as used herein, refers to a molecule that links a first polypeptide to a second polypeptide (e.g., one or more amino acids) or a first nucleic acid to a second nucleic acid (e.g., one or more nucleotides). In some embodiments, the linker that links polynucleotides comprise a phosphodiester bond. In some embodiments, the linker that links polypeptides comprise an amide bond.

The term, “mutation,” as used herein, refers to an alteration that changes an amino acid residue or a nucleotide as described herein. Such an alteration can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation. Methods of mutating an amino acid residue or a nucleotide are well known.

The terms, “mutation associated with a disease” and “mutation associated with a genetic disorder,” as used herein, refer to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The term, “nickase,” as used herein, refers to an enzyme that possess catalytic activity for single stranded nucleic acid cleavage of a double stranded nucleic acid.

The term, “nickase activity,” as used herein, refers to catalytic activity that results in single stranded nucleic acid cleavage of a double stranded nucleic acid.

The terms, “non-naturally occurring” and “engineered,” as used herein, refer to indicate involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term, “NUC lobe,” as used herein, refers to the nuclease lobe which typically houses the RuvC domains. The NUC lob is connected to the REC lobe by a bridge helix.

The terms, “nuclease” and “endonuclease,” as used herein, refer to an enzyme which possesses catalytic activity for nucleic acid cleavage.

The term, “nuclease activity,” as used herein, refers to catalytic activity that results in nucleic acid cleavage (e.g., ribonuclease activity (ribonucleic acid cleavage), or deoxyribonuclease activity (deoxyribonucleic acid cleavage), etc.).

The term, “nucleic acid,” as used herein, refers to a polymer of nucleotides. A nucleic acid may comprise ribonucleotides, deoxyribonucleotides, combinations thereof, and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more engineered modifications, or both.

The term, “nucleic acid expression vector,” as used herein, refers to a plasmid that can be used to express a nucleic acid of interest, as well as viral expression vectors. In some instances, the nucleic acid expression vector is an adeno associated viral (AAV) vector.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

The terms, “nucleotide(s)” and “nucleoside(s)”, as used herein, in the context of a nucleic acid molecule having multiple residues, refer to describing the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

The term, “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington, The Science and Practice of Pharmacy 23rd Ed. Academic Press, 2021).

The terms, “polypeptide” and “protein,” as used herein, refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a heterologous peptide, such as an effector partner, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

The term, “prime editing enzyme”, as used herein, refers to a protein, polypeptide, or fragment thereof that is capable of catalyzing the editing (insertion, deletion, or base-to-base conversion) of a target nucleotide or nucleotide sequence in a nucleic acid.

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The terms, “protospacer adjacent motif” and “PAM,” as used herein, refer to a nucleotide sequence found in a target nucleic acid that directs an effector protein to bind or edit the target nucleic acid at a specific location. In some instances, a PAM is required for a complex of an effector protein and a guide nucleic acid (e.g., an RNP complex) to hybridize to and edit the target nucleic acid. In some instances, the complex does not require a PAM to edit the target nucleic acid.

The term, “REC domain,” as used herein, refers to domain in an a-helical recognition region or lobe. An effector protein may contain at least one REC domain (e.g., REC1, REC2) which generally helps to accommodate and stabilize the guide nucleic acid and target nucleic acid hybrid.

The term, “recombinant,” as used herein, in the context of proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term, “repeat sequence,” as used herein, refers to a sequence of nucleotides in a guide nucleic acid that is capable of, at least partially, interacting with an effector protein.

The terms, “reporter,” “reporter nucleic acid,” and “reporter molecule,” as used herein, are used interchangeably and refer to a non-target nucleic acid molecule that can provide a detectable signal upon cleavage by an effector protein. Examples of detectable signals and detectable moieties that generate detectable signals are provided herein.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “R-Loop” as used herein, refers to a three-stranded nucleic acid structure comprising a DNA: RNA hybrid and a displaced strand of DNA. For example, an R-Loop can be formed upon hybridization of a guide nucleic acid as described herein to a target sequence of a target nucleic acid.

The terms, “RuvC” and “RuvC domain,” as used herein, refer to a region of an effector protein that is capable of cleaving a target nucleic acid, and in certain instances, of processing a pre-crRNA. In some instances, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons.

The term, “sample,” as used herein, refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The terms, “single guide nucleic acid”, “single guide RNA” and “sgRNA,” as used herein, in the context of a single nucleic acid system, refers to a guide nucleic acid, wherein the guide nucleic acid is a single polynucleotide chain having all the required sequence for a functional complex with an effector protein (e.g., being bound by an effector protein, including in some instances activating the effector protein, and hybridizing to a target nucleic acid, without the need for a second nucleic acid molecule). For example, an sgRNA can have two or more linked guide nucleic acid components (e.g., an intermediary sequence, a repeat sequence, a spacer sequence and optionally a linker, or a handle sequence and a spacer sequence).

The term, “single nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein.

The term, “spacer sequence,” as used herein, refers to a nucleotide sequence in a guide nucleic acid that is capable of, at least partially, hybridizing to an equal length portion of a sequence (e.g., a target sequence) of a target nucleic acid. In other words, at least a portion of the spacer sequence hybridizes to the target sequence.

The term, “subject,” as used herein, refers to an animal. The subject may be a mammal. The subject may be a human. The subject may be diagnosed or at risk for a disease.

The term, “sufficiently complementary,” as used herein, refers to a first nucleotide sequence that is partially complementarity to a second nucleotide sequence while still allowing the first nucleotide sequence to hybridize to the second nucleotide sequence with enough affinity to permit a biological activity to occur. Depending on the context, a biological activity may be the formation of a complex between two or more components described herein, such as an effector protein and a guide nucleic acid. A biological activity may also be bringing one or more components described herein into proximity of another component, such as bringing an effector protein-guide nucleic acid complex into proximity of a target nucleic acid. A biological activity may additionally be permitting a component described herein to act on another component described herein, such as permitting an effector protein to cleave a target nucleic acid. In some instances, sequences are said to be sufficiently complementary when at least 85% of the residues of a nucleotide sequence are complementary to residues in a reference nucleotide sequence.

The term, “syndrome,” as used herein, refers to a group of symptoms which, taken together, characterize a condition.

The term, “target nucleic acid,” as used herein, refers to a nucleic acid that is selected as the nucleic acid for editing, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, in the context of a target nucleic acid, refers to a nucleotide sequence found within a target nucleic acid. Such a nucleotide sequence can, for example, hybridize to a respective length portion of a guide nucleic acid (i.e., the spacer sequence).

The term, “trans cleavage,” as used herein, in the context of cleavage (e.g., hydrolysis of a phosphodiester bond) of one or more target nucleic acids or non-target nucleic acids, or both, by an effector protein that is complexed with a guide nucleic acid and the target nucleic acid. Trans cleavage activity may be triggered by the hybridization of a guide nucleic acid to a target nucleic acid. The effector may cleave a target strand as well as non-target strand, wherein the target nucleic is a double stranded nucleic acid. Trans cleavage of the target nucleic acid may occur away from (e.g., not within or directly adjacent to) the portion of the target nucleic acid that is hybridized to the portion of the guide nucleic acid.

The term, “transcriptional activator,” as used herein, refers to a polypeptide or a fragment thereof that can activate or increase transcription of a target nucleic acid molecule.

The term, “transcriptional repressor,” as used herein, refers to a polypeptide or a fragment thereof that is capable of arresting, preventing, or reducing transcription of a target nucleic acid.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. The cell in which transgene expression occurs can be a target cell, such as a host cell.

The terms, “treatment” and “treating,” as used herein, refer to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “variant,” as used herein, refers to a form or version of a protein that differs from the wild-type protein. A variant may have a different function or activity relative to the wild-type protein.

The term, “viral vector,” as used herein, refers to a nucleic acid to be delivered into a host cell by a recombinantly produced virus or viral particle.

### Introduction

Disclosed herein are compositions, systems, and methods comprising at least one of: a polypeptide or a nucleic acid encoding the polypeptide; and a guide nucleic acid or a nucleic acid encoding the guide nucleic acid.

Polypeptides described herein may bind and, optionally, cleave nucleic acids in a sequence-specific manner. Polypeptides described herein may also cleave the target nucleic acid within a target sequence or at a position adjacent to the target sequence. In some embodiments, a polypeptide is activated when it binds a certain sequence of a nucleic acid described herein, allowing the polypeptide to cleave a region of a target nucleic acid that is near, but not adjacent to the target sequence. A polypeptide may be an effector protein, such as a CRISPR-associated (Cas) protein, which may bind a guide nucleic acid that imparts activity or sequence selectivity to the polypeptide. An effector protein may also be referred to as a programmable nuclease because the nuclease activity of the protein may be directed to different target nucleic acids by way of revising the guide nucleic acid that the protein binds.

In some embodiments, compositions, systems, and methods comprising guide nucleic acids comprise a first region or first sequence, at least a portion of which interacts with a polypeptide. In some embodiments, the first region or first sequence comprises a sequence that is similar or identical to an intermediary nucleic acid sequence, a handle, a repeat sequence, or a combination thereof. In some embodiments, the guide nucleic acid does not comprise an intermediary nucleic acid. In some embodiments, compositions, systems, and methods comprising guide nucleic acids comprise a second sequence that is at least partially complementary to a target nucleic acid, and which may be referred to as a spacer sequence.

Effector proteins disclosed herein may bind and/or cleave nucleic acids, including double stranded RNA (dsRNA), single-stranded RNA (ssRNA), double stranded DNA (dsDNA), and single-stranded DNA (ssDNA). Polypeptides disclosed herein may provide binding activity, cis cleavage activity, nickase activity, nuclease activity, or a combination thereof.

The compositions, systems, and methods described herein are non-naturally occurring. In some embodiments, compositions, systems, and methods comprise an engineered guide nucleic acid (also referred to herein as a guide nucleic acid) or a use thereof. In some embodiments, compositions, systems, and methods comprise an engineered protein or a use thereof. In some embodiments, compositions, systems, and methods comprise an isolated polypeptide or a use thereof. In general, compositions, methods, and systems described herein are not found in nature. In some embodiments, compositions, methods, and systems described herein comprise at least one non-naturally occurring component. For example, disclosed compositions, methods, and systems may comprise a guide nucleic acid, wherein the sequence of the guide nucleic acid is different or modified from that of a naturally-occurring guide nucleic acid.

In some embodiments, compositions, systems, and methods comprise at least two components that do not naturally occur together. For example, disclosed compositions, systems, and methods may comprise a guide nucleic acid comprising a first region, at least a portion of which, interacts with a polypeptide, and a second region that is at least partially complementary to a target sequence in a target nucleic acid, wherein the first region and second region do not naturally occur together and/or are heterologous to each other. Also, by way of non-limiting example, disclosed compositions, systems, and methods may comprise a guide nucleic acid and an effector protein that do not naturally occur together. Likewise, by way of non-limiting example, disclosed compositions, systems, and methods may comprise a ribonucleotide-protein (RNP) complex comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. The non-natural nucleotide sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. In some embodiments, compositions and systems comprise at least two components that do not occur together in nature, wherein the at least two components comprise at least one of an effector protein, an effector partner and a guide nucleic acid. Guide nucleic acids may comprise a first sequence (or a first region) and a second sequence (or a second region) that do not occur naturally together. For example, a guide nucleic acid may comprise a naturally-occurring repeat sequence and a spacer sequence that is complementary to a naturally-occurring eukaryotic sequence. The guide nucleic acid may comprise a repeat sequence that occurs naturally in an organism and a spacer sequence that does not occur naturally in that organism. A guide nucleic acid may comprise a first sequence (or a first region) that occurs in a first organism and a second sequence (or a second region) that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, the guide nucleic acid comprises two heterologous sequences arranged in an order or proximity that is not observed in nature. Therefore, compositions and systems described herein are not naturally occurring.

In some embodiments, compositions, systems, and methods described herein comprise a polypeptide (e.g., an effector protein, an effector partner, a fusion protein, or a combination thereof) that is similar to a naturally occurring effector protein. The polypeptide may lack a portion of the naturally occurring polypeptide. The polypeptide may comprise a mutation relative to the naturally-occurring polypeptide, wherein the mutation is not found in nature. The polypeptide may also comprise at least one additional amino acid relative to the naturally-occurring polypeptide. In some embodiments, the polypeptide may comprise a heterologous peptide. For example, the polypeptide may comprise an addition of a nuclear localization signal relative to the natural occurring polypeptide. In some embodiments, a nucleotide sequence encoding the polypeptide is codon optimized (e.g., for expression in a eukaryotic cell) relative to the naturally occurring sequence.

### Polypeptide Systems

Provided herein are compositions, systems and methods comprising a polypeptide or polypeptide system, wherein the polypeptide or polypeptide system described herein comprises one or more effector proteins or variants thereof, one or more effector partners or variants thereof, one or more linkers for peptides, or combinations thereof.

In some embodiments, the polypeptides described herein comprise modification activities. In some embodiments, the modification activity of the polypeptide described herein comprises cleavage activity, binding activity, insertion activity, substitution activity, and the like. In some embodiments, the modification activity of the polypeptide results in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, chemical modification of one or more nucleotides of a target nucleic acid with an alternative nucleotide, or combinations thereof. In some embodiments, the cleavage activity is a nicking activity.

### Effector Proteins

Provided herein are compositions, systems, and methods comprising an effector protein or a use thereof.

An effector protein provided herein interacts with a guide nucleic acid to form a complex. In some embodiments, the complex interacts with a target nucleic acid by guide RNA-target strand hybridization. In some embodiments, the complex interacts with a target nucleic acid, a non-target nucleic acid, or both. In some embodiments, an interaction between the complex and a target nucleic acid, a non-target nucleic acid, or both comprises one or more of: recognition of a protospacer adjacent motif (PAM) sequence within the target nucleic acid by the effector protein, hybridization of the guide nucleic acid to the target nucleic acid, modification of the target nucleic acid and/or the non-target nucleic acid by the effector protein, or combinations thereof. In some embodiments, recognition of a PAM sequence within a target nucleic acid directs the modification activity of an effector protein. In some embodiments, recognition of a PAM sequence adjacent to a target sequence of a target nucleic acid directs the modification activity of an effector protein.

In some embodiments, effector proteins disclosed herein provides cleavage activity, such as cis cleavage activity, nickase activity, nuclease activity, or a combination thereof. In some embodiments, effector proteins described herein edit a target nucleic acid, wherein the target nucleic acid comprises a target sequence and a non-target sequence. In some embodiments, the effector proteins edit the target nucleic acid by cis cleavage activity on the target sequence. Alternatively or additionally, in some embodiments, the effector proteins are not capable of editing a target nucleic acid by trans cleavage activity on the non-target sequence. In some embodiments, the effector proteins do not edit a target nucleic acid by trans cleavage activity on the non-target sequence. Effector proteins disclosed herein may cleave nucleic acids, including double stranded RNA (dsRNA), single stranded RNA (ssRNA), double stranded DNA (dsDNA), and single-stranded DNA (ssDNA). In some embodiments, effector proteins disclosed herein provides catalytic activity (e.g., cleavage activity, nickase activity, nuclease activity, or combinations thereof). In some embodiments, the catalytic activity of the effector protein is similar to that of a naturally-occurring effector protein, such as, for example, a naturally-occurring effector protein with reduced cleavage activity including cis cleavage activity.

In some embodiments, effector proteins disclosed herein provide nickase activity. In some embodiments, the effector protein provides catalytic activity (e.g., binding activity, nickase activity, or a combination thereof). In some embodiments, the effector protein nicks a target nucleic acid, wherein the target nucleic acid is a double stranded RNA (dsRNA), a double stranded DNA (dsDNA) or a combination thereof. In some embodiments, the effector protein nicks a target strand of the target nucleic acid. In some embodiments, the effector protein nicks a non-target strand of the target nucleic acid. In some embodiments, the effector protein cleaves a target nucleic acid, wherein the target nucleic acid is a single stranded RNA (ssRNA), a single stranded DNA (ssDNA), a double stranded RNA (dsRNA), a double stranded DNA (dsDNA) or a combination thereof.

In some embodiments, effector proteins described herein comprise one or more functional domains. Effector protein functional domains can include a protospacer adjacent motif (PAM)-interacting domain, an oligonucleotide-interacting domain, one or more recognition domains, a non-target strand interacting domain, and a RuvC domain. A PAM interacting domain can be a target strand PAM interacting domain (TPID) or a non-target strand PAM interacting domain (NTPID). In some embodiments, a PAM interacting domain, such as a TPID or a NTPID, on an effector protein describes a region of an effector protein that interacts with target nucleic acid. In some embodiments, the effector proteins comprise a RuvC domain. In some embodiments, a RuvC domain comprises with substrate binding activity, catalytic activity, or both. In some embodiments, the RuvC domain is defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein, but form a RuvC domain once the protein is produced and folds. In some embodiments, the RuvC domain described herein comprises variants thereof (e.g., one or more mutations including substitutions, additions, deletions (e.g., truncation), or combinations thereof. In some embodiments, effector proteins comprise one or more recognition domain (REC domain) with a binding affinity for a guide nucleic acid or for a guide nucleic acid-target nucleic acid heteroduplex. An effector protein may comprise a zinc finger domain. In some embodiments, the effector protein does not comprise an HNH domain.

An effector protein may be a CRISPR-associated (“Cas”) protein. An effector protein may be a modified effector protein having increased modification activity and/or increased substrate binding activity (e.g., substrate selectivity, specificity, and/or affinity). In some embodiments, the substrate can be a double-stranded RNA (dsRNA), single stranded RNA (ssRNA), double stranded DNA (dsDNA), or single-stranded DNA (ssDNA). An effector protein may function as a single protein, including a single protein that is capable of binding (or binds) to a guide nucleic acid and editing a target nucleic acid. Alternatively, an effector protein may function as part of a multiprotein complex.

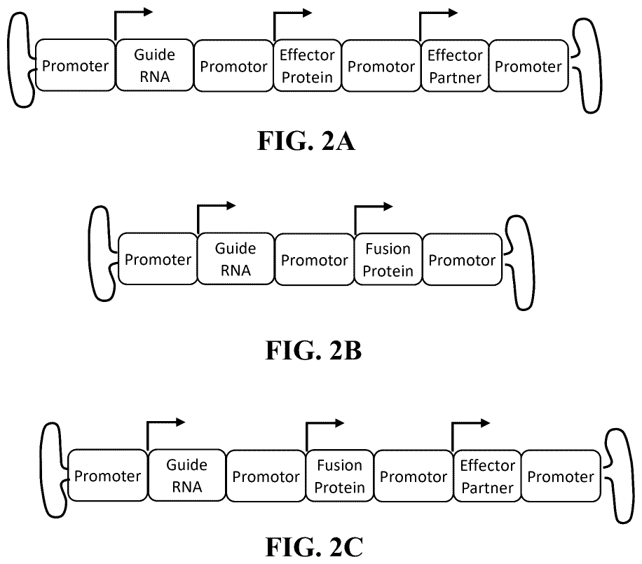

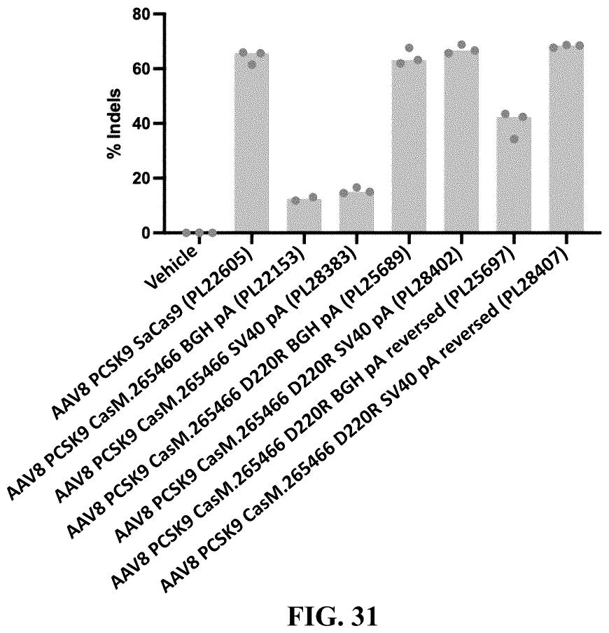

TABLE 1 provides an illustrative amino acid sequence of an effector protein, CasM.265466 that may be modified for use in the compositions, systems and methods described herein. CasM.265466 and engineered variants thereof have demonstrated robust editing in various cell types (e.g., T cells, HSCs and hepatocytes), low off-target activity (e.g., less than 2% indels in off target sites), and fewer translocations in multiplex edited cells than Cas9. CasM.265466 and engineered variants thereof have also demonstrated robust editing in vivo, >40% indels (see, e.g., Example 20 and FIG. 31). Additionally, CasM.265466 and variants thereof are about 450 amino acids or less in length, allowing for a sequence encoding CasM.265466, a sequence encoding a base editing enzyme, and at least one guide nucleic acid to be packaged into a single AAV vector (see, e.g., FIG. 2A-2C).

In some embodiments, compositions, systems and methods described herein comprise an effector protein, or a nucleic acid encoding the effector protein, wherein the amino acid sequence of the effector protein comprises at least about 200 contiguous amino acids or more of the amino acid sequence recited in TABLE 1. In some embodiments, the amino acid sequence of an effector protein provided herein comprises at least 200, at least 220, at least 240, at least 260, at least 280, at least 300, at least 320, at least 340, at least 360, at least 380, at least 400 contiguous amino acids, at least 420 contiguous amino acids, at least 440 contiguous amino acids, of the amino acid sequence of TABLE 1.

In some embodiments, compositions, systems and methods described herein comprise an effector protein or a nucleic acid encoding the effector protein, wherein the effector protein comprises a portion of the amino acid sequence recited in TABLE 1. In some embodiments, the effector protein comprises a portion of the amino acid sequence recited in TABLE 1, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of the amino acid sequence recited in TABLE 1. In some embodiments, the effector protein comprises a portion of the amino acid sequence recited in TABLE 1, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of the amino acid sequence recited in TABLE 1.

In some embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, or at least 99%, but less than 100% identical to the amino acid sequence as recited in TABLE 1. In some embodiments, an effector protein provided herein comprises an amino acid sequence that is at least 65% but less than 100% identical to the amino acid sequence as recited in TABLE 1. In some embodiments, an effector protein provided herein comprises an amino acid sequence that is at least 70% but less than 100% identical to the amino acid sequence as recited in TABLE 1. In some embodiments, an effector protein provided herein comprises an amino acid sequence that is at least 75% but less than 100% identical to the amino acid sequence as recited in TABLE 1. In some embodiments, an effector protein provided herein comprises an amino acid sequence that is at least 80% but less than 100% identical to the amino acid sequence as recited in TABLE 1. In some embodiments, an effector protein provided herein comprises an amino acid sequence that is at least 85% but less than 100% identical to the amino acid sequence as recited in TABLE 1. In some embodiments, an effector protein provided herein comprises an amino acid sequence that is at least 90% but less than 100% identical to the amino acid sequence as recited in TABLE 1. In some embodiments, an effector protein provided herein comprises an amino acid sequence that is at least 95% but less than 100% identical to the amino acid sequence as recited in TABLE 1. In some embodiments, an effector protein provided herein comprises an amino acid sequence that is at least 97% but less than 100% identical to the amino acid sequence as recited in TABLE 1. In some embodiments, an effector protein provided herein comprises an amino acid sequence that is at least 98% but less than 100% identical to the amino acid sequence as recited in TABLE 1. In some embodiments, an effector protein provided herein comprises an amino acid sequence that is at least 99% but less than 100% identical to the amino acid sequence as recited in TABLE 1. In some embodiments, an effector protein provided herein comprises an amino acid sequence that is less than 100% identical to the amino acid sequence as recited in TABLE 1.

In some embodiments, compositions, systems, and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar, but not the same, to the amino acid sequence as recited in TABLE 1. In some embodiments, an effector protein provided herein comprises an amino acid sequence that is at least 80% similar, but not the same, to the amino acid sequence as recited in TABLE 1. In some embodiments, an effector protein provided herein comprises an amino acid sequence that is at least 85% similar, but not the same, to the amino acid sequence as recited in TABLE 1. In some embodiments, an effector protein provided herein comprises an amino acid sequence that is at least 90% identical, but not the same, to the amino acid sequence as recited in TABLE 1. In some embodiments, an effector protein provided herein comprises an amino acid sequence that is at least 95% similar, but not the same, to the amino acid sequence as recited in TABLE 1. In some embodiments, an effector protein provided herein comprises an amino acid sequence that is at least 97% similar, but not the same, to the amino acid sequence as recited in TABLE 1. In some embodiments, an effector protein provided herein comprises an amino acid sequence that is at least 98% similar, but not the same, to the amino acid sequence as recited in TABLE 1. In some embodiments, an effector protein provided herein comprises an amino acid sequence that is at least 99% similar, but not the same, to the amino acid sequence as recited in TABLE 1. In some embodiments, an effector protein provided herein comprises an amino acid sequence that is 100% similar, but not the same, to the amino acid sequence as recited in TABLE 1.

In some embodiments, compositions, systems, and methods described herein comprise an effector protein, or a nucleic acid encoding the effector protein, wherein the effector protein comprises one or more amino acid alterations relative to the amino acid sequence recited in TABLE 1. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to the amino acid sequence recited in TABLE 1. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more amino acid alterations relative to the amino acid sequence recited in TABLE 1. In some embodiments, the one or more alterations comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid alterations relative to the amino acid sequence recited in TABLE 1. In some embodiments, the one or more alterations comprises one, two, three, four, five, six, seven, eight, nine, ten, or more amino acid alterations relative to the amino acid sequence recited in TABLE 1. In some embodiments, the one or more amino acid alterations comprises substitutions (e.g., conservative substitutions, non-conservative substitutions), deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to the amino acid sequence recited in TABLE 1. In some embodiments, the effector protein comprises one or more alterations selected from positions D237, D418, and E335 relative to the amino acid sequence recited in TABLE 1. In some embodiments, the effector protein comprises one or more alterations independently selected at positions 58, 80, 84, 105, 193, 202, 209, 210, 218, 220, 225, 246, 286, 295, 298, 306, 315, 360, or a combination thereof relative to the corresponding reference amino acid sequence of TABLE 1. In some embodiments, the effector protein comprises one or more amino acid alterations independently at the positions selected from A75, K58, 180, T84, K105, D171, N193, C202, S209, G210, A218, D220, E225, C246, K250, N286, M295, M298, A306, Y315, Q360, E362, A393 or a combination thereof.

In some embodiments, compositions, systems, and methods described herein comprise an effector protein, or a nucleic acid encoding the effector protein, wherein the effector protein comprises one or more substitutions relative to the amino acid sequence recited in TABLE 1. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to the amino acid sequence recited in TABLE 1. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more substitutions relative to the amino acid sequence recited in TABLE 1. In some embodiments, the one or more substitutions comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid substitutions relative to the amino acid sequence recited in TABLE 1. In some embodiments, the one or more substitutions comprise one, two, three, four, five, six, seven, eight, nine, ten or more substitutions relative to the amino acid sequence recited in TABLE 1.

In some embodiments, the effector proteins described herein comprise one or more substitutions independently selected from substitutions at position 58, 75, 80, 84, 105, 171, 193, 202, 209, 210, 218, 220, 225, 246, 250, 286, 295, 298, 306, 315, 360, 362 and 393 relative to the amino acid sequence of TABLE 1. In some embodiments, the effector protein comprises one or more amino acid substitutions independently selected from substitutions at position A75, K58, 180, T84, K105, D171, N193, C202, S209, G210, A218, D220, E225, C246, K250, N286, M295, M298, A306, Y315, Q360, E362 and A393 relative to the amino acid sequence recited in TABLE 1. In some embodiments, the effector protein comprises one or more amino acid substitutions independently selected from K58W, I80K, N193K, S209F, A218K, E225K, N286K, M295W, M298L, A306K and Y315M relative to the amino acid sequence recited in TABLE 1. In some embodiments, the one or more amino acid substitutions effects change in catalytic activity of the effector protein. In some embodiments, the one or more amino acid substitutions effects increase in catalytic activity of the effector protein.

In some embodiments, the effector proteins described herein comprise one or more amino acid substitutions with a positively charged amino acid residues. In some embodiments, the positively charged amino acid residue is independently selected from Lys (K), Arg (R), and His (H). In some embodiments, the effector protein comprising one or more substitutions with the positively charged substitutions effects change in catalytic activity of the effector protein. In some embodiments, the effector protein comprising one or more substitutions with the positively charged substitutions effects increase in catalytic activity of the effector protein. In some embodiments, the effector protein comprises one or more amino acid substitutions independently selected from I80R, T84R, K105R, G210R, C202R, A218R, D220R, E225R, C246R and Q360R relative to the amino acid sequence recited in TABLE 1.

In some embodiments, an effector protein comprising one or more substitutions relative to the amino acid sequence recited in TABLE 1 effects change in catalytic activity of the effector protein. In some embodiments, the one or more substitutions improves catalytic activity of the effector protein relative to the reference effector protein comprising the amino acid sequence recited in TABLE 1. In some embodiments, at least one substitution, at least two substitutions, at least three substitutions, or more substitutions improve catalytic activity of the effector protein relative to the reference effector protein comprising the amino acid sequence recited in TABLE 1. In some embodiments, the effector protein comprises at least one amino acid substitution selected from substitutions at positions A75, T84, K250, D171, N193, C202, S209, D220, E225, K250, N286, A306, Y315, Q360, E362 and A393 relative to the amino acid sequence recited in TABLE 1. In some embodiments, the effector protein comprises at least one amino acid substitution selected from A75F, T84R, K250N, D171K, N193K, C202K, C202R, S209F, S209Y, D220R, E225K, E225R, K250N, N286K, A306K, Y315M, Q360R, E362D, A393R and A393E relative to the amino acid sequence recited in TABLE 1. In some embodiments, the effector protein comprises a substitution selected from A75F, T84R, K250N, D171K, N193K, C202K, C202R, S209F, S209Y, D220R, E225K, E225R, K250N, N286K, A306K, Y315M, Q360R, E362D, A393R and A393E relative to the amino acid sequence recited in TABLE 1.

In some embodiments, the effector protein comprises at least two amino acid substitutions selected from a substitutions at positions A75, T84, K250, D171, N193, C202, S209, D220, E225, K250, N286, A306, Y315, L337, Q360, E362 T381, S382, C385, A393, F406, N420, and N424 relative to the amino acid sequence recited in TABLE 1. In some embodiments, the effector protein comprises at least two amino acid substitutions independently selected from A75F, T84R, K250N, D171K, N193K, C202K, C202R, S209F, S209Y, D220R, E225K, E225R, K250N, N286K, A306K, Y315M, Q360R, E362D, A393R and A393E relative to the amino acid sequence recited in TABLE 1. In some embodiments, the at least two amino acid substitutions comprise a combination of substitutions at positions selected from: T84 and D171; T84 and C202; T84 and S209; T84 and E225; T84 and K250; T84 and N286; T84 and A306; T84 and Y315; T84 and E362; T84 and A393; D171 and C202; D171 and D220; D171 and E225; D171 and Q360; N193 and D220; C202 and D220; C202 and S209; C202 and E225; C202 and K250; C202 and Y315; C202 and E362; C202 and A393; S209 and E225; S209 and D220; S209 and Q360; D220 and K250; D220 and N286; D220 and A306; D220 and Y315; D220 and L337; D220 and E362; D220 and T381; D220 and S382; D220 and C385; D220 and A393; D220 and F406; D220 and N420; D220 and N424; E225 and K250; E225 and A306; N286 and Q360; A306 and Q360; Y315 and Q360; and Q360 and A393 relative to the amino acid sequence recited in TABLE 1. In some embodiments, the at least two amino acid substitutions comprise a combination of substitutions selected from: T84R and D171K; T84R and C202K; T84R and S209Y; T84R and E225K; T84R and K250N; T84R and N286K; T84R and A306K; T84R and Y315M; T84R and E362D; T84R and A393E; D171K and C202R; D171K and D220R; D171K and E225R; D171K and Q360R; N193K and D220R; C202R and D220R; C202R and S209F; C202R and S209Y; C202R and E225K; C202R and K250N; C202R and Y315M; C202R and E362D; C202R and A393E; S209F and E225R; S209F and D220R; S209F and Q360R; S209Y and D220R; S209Y and Q360R; D220R and K250N; D220R and N286K; D220R and A306K; D220R and Y315M; D220R and L337A; D220R and E362D; D220R and T381A; D220R and S382A; D220R and C385A; D220R and A393R; D220R and F406A; D220R and N420A; D220R and N424A; E225R and K250N; E225R and A306K; N286K and Q360R; A306K and Q360R; Y315M and Q360R; and Q360R and A393E relative to the amino acid sequence recited in TABLE 1. In some embodiments, the at least two amino acid substitutions comprise a combination of substitutions selected from: E225R/A306K, C202R/A306K, D220R/K250N, and D220R/A306K relative to the amino acid sequence recited in TABLE 1. In some embodiments, the at least two amino acid substitutions comprise a combination of substitutions selected from: D220R/L337A, D220R/T381A, D220R/S382A, D220R/C385A, D220R/F406A, D220R/N420A, and D220R/N424A relative to the amino acid sequence recited in TABLE 1.

In some embodiments, the effector protein comprises at least three amino acid substitutions selected from a substitutions at positions A75, T84, K250, D171, N193, C202, S209, D220, E225, K250, N286, A306, Y315, Q360, E362 and A393 relative to the amino acid sequence recited in TABLE 1. In some embodiments, the effector protein comprises at least three amino acid substitutions independently selected from A75F, T84R, K250N, D171K, N193K, C202K, C202R, S209F, S209Y, D220R, E225K, E225R, K250N, N286K, A306K, Y315M, Q360R, E362D, A393R and A393E relative to the amino acid sequence recited in TABLE 1. In some embodiments, the at least three amino acid substitutions comprise a combination of substitutions at positions selected from: A75, D220 and Q360; N193, D220 and Q360; S209, D220 and Q360; D220, K250 and Q360; D220, A306 and Q360; D220, Q360 and E362; and D220, Q360 and A393 relative to the amino acid sequence recited in TABLE 1. In some embodiments, the at least three amino acid substitutions comprise a combination of substitutions selected from: A75F, D220R and Q360R; N193K, D220R and Q360R; S209F, D220R and Q360R; D220R, K250N and Q360R; D220R, A306K and Q360R; D220R, Q360R and E362D; and D220R, Q360R and A393E relative to the amino acid sequence recited in TABLE 1.

An effector protein that has decreased catalytic activity may be referred to as catalytically or enzymatically inactive, catalytically or enzymatically dead, as a dead protein or a dCas protein. In some embodiments, such a protein comprises an enzymatically inactive domain (e.g., inactive nuclease domain or a domain with reduced nuclease activity). For example, a nuclease domain (e.g., RuvC domain) of an effector protein may be deleted or mutated relative to a wildtype counterpart so that it is no longer functional or comprises reduced nuclease activity. In some embodiments, a catalytically inactive effector protein binds to a guide nucleic acid and/or a target nucleic acid but does not cleave the target nucleic acid. In some embodiments, a catalytically inactive effector protein associates with a guide nucleic acid to activate or repress transcription of a target nucleic acid. In some embodiments, a catalytically inactive effector protein is fused to an effector partner to form a fusion protein. In some embodiments, the effector partner that confers an alternative activity to an effector protein activity. Accordingly, in some embodiments, the fusion proteins are capable of having (or have) some function or activity not provided by the effector protein. Such fusion proteins are described herein and throughout. In some embodiments, the effector protein or a fusion protein thereof comprises one or more amino acid substitutions relative to an otherwise identical protein, wherein the one or more amino acid substitutions provide reduced catalytic activity relative to the otherwise identical protein.

In some embodiments, the dCas proteins described herein comprise one or more substitutions independently selected from substitutions at positions D237, D418 and E335 relative to the amino acid sequence recited in TABLE 1. In some embodiments, the dCas protein comprises one or more amino acid substitutions independently selected from D237A, D418A, D418N, E335A, and E335Q relative to the amino acid sequence recited in TABLE 1. In some embodiments, the dCas protein comprises at least one amino acid substitutions of D237A relative to the amino acid sequence recited in TABLE 1. In some embodiments, the dCas protein comprises at least one amino acid substitutions of D237N relative to the amino acid sequence recited in TABLE 1. In some embodiments, the dCas protein comprises at least one amino acid substitutions of E335Q relative to the amino acid sequence recited in TABLE 1.

In some embodiments, the effector proteins described herein comprises two substitutions, wherein one substitution is selected from D237A, D418A, D418N, E335A, and E335Q, and the other substitution is selected from I80R, T84R, K105R, G210R, C202R, A218R, D220R, E225R, C246R, Q360R, K58W, I80K, N193K, S209F, A218K, E225K, N286K, M295W, M298L, A306K and Y315M relative to the amino acid sequence recited in TABLE 1.

In some embodiments, the effector proteins described herein is a dCas protein comprising one or more substitutions independently selected from D237A, D418A, D418N, E335A, and E335Q, and wherein the dCas protein further comprising a substitution with a positively charged amino acid residues. In some embodiments, the positively charged amino acid residue is independently selected from Lys (K), Arg (R), and His (H). In some embodiments, the dCas protein comprising one or more substitutions with the positively charged substitutions effects change in binding activity of the effector protein. In some embodiments, the effector protein comprising one or more substitutions with the positively charged substitutions effects increase in binding activity of the effector protein. In some embodiments, the one or more positively charged substitutions comprises I80R, T84R, K105R, G210R, C202R, A218R, D220R, E225R, C246R, Q360R, or a combination thereof relative to the amino acid sequence recited in TABLE 1. For example, in some embodiments, the effector protein comprises D237A and D220R amino acid substitutions relative to the amino acid sequence recited in TABLE 1. In some embodiments, the effector protein comprises D237N and D220R amino acid substitutions relative to the amino acid sequence recited in TABLE 1. In some embodiments, the effector protein comprises E335Q and D220R amino acid substitutions relative to the amino acid sequence recited in TABLE 1.

In some embodiments, compositions, systems, and methods described herein comprise an effector protein, or a nucleic acid encoding the effector protein, wherein the effector protein comprises one or more substitutions relative to the amino acid sequence recited in TABLE 1. In some embodiments, the one or more substitutions comprise one or more conservative substitutions, one or more non-conservative substitutions, or combinations thereof.

In some embodiments, compositions, systems, and methods described herein comprise an effector protein, or a nucleic acid encoding the effector protein, wherein the effector protein comprises one or more conservative substitutions relative to the amino acid sequence recited in TABLE 1. In some embodiments, the one or more conservative substitutions comprises at least one, at least two, at least three, at least four, at least five, at least six, at least seven, at least eight, at least nine, at least ten, at least twelve, at least sixteen, at least twenty, or more conservative substitutions relative to the amino acid sequence recited in TABLE 1. In some embodiments, the one or more conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more conservative substitutions relative to the amino acid sequence recited in TABLE 1. In some embodiments, the one or more conservative substitutions comprise one, two, three, four, five, six, seven, eight, nine, ten or more conservative substitutions relative to the amino acid sequence recited in TABLE 1. In some embodiments, compositions, systems, and methods described herein comprise a variant of an effector protein, or a nucleic acid encoding the same, wherein the variant comprises 1, 2, 3, 4, 5, 6, 7, 8, 9 or 10 conservative amino acid substitution relative to the amino acid sequence recited in TABLE 1. In some embodiments, the variant as described herein further comprises an additional one or more alterations, such as non-conservative substitutions, relative to the amino acid sequence recited in TABLE 1. For example, in some embodiments, the variant comprises an amino acid sequence that is identical to any one of the amino acid sequences as recited in TABLE 1.1. In some embodiments the variant comprises an amino acid sequence that is identical to any one of SEQ ID NOs: 379, 377 and 381. In some embodiments, the variant comprises an amino acid sequence that is identical to SEQ ID NO: 379.

In some embodiments, compositions, systems, and methods described herein comprise an effector protein, or a nucleic acid encoding the effector protein, wherein the effector protein comprises one or more non-conservative substitutions relative to the amino acid sequence recited in TABLE 1. In some embodiments, the one or more non-conservative substitutions comprises at least one, at least two, at least three, at least four, at least five, at least six, at least seven, at least eight, at least nine, at least ten, at least twelve, at least sixteen, at least twenty, or more non-conservative substitutions relative to the amino acid sequence recited in TABLE 1. In some embodiments, the one or more non-conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more non-conservative substitutions relative to the amino acid sequence recited in TABLE 1. In some embodiments, the one or more non-conservative substitutions comprise one, two, three, four, five, six, seven, eight, nine, ten or more non-conservative substitutions relative to the amino acid sequence recited in TABLE 1. In some embodiments, compositions, systems, and methods described herein comprise a variant of an effector protein, or a nucleic acid encoding the same, wherein the variant comprises 1, 2, 3, 4, 5, 6, 7, 8, 9 or 10 non-conservative amino acid substitutions relative to the amino acid sequence recited in TABLE 1. In some embodiments, the variant as described herein further comprises an additional one or more alterations, such as conservative substitutions, relative to the amino acid sequence recited in TABLE 1.

In some embodiments, the effector proteins described herein generate more indels on a target strand of a target nucleic acid relative to a non-target strand of the target nucleic acid. In some embodiments, the effector proteins generate less indel in non-target strand relative to a corresponding control effector protein comprising an amino acid sequence of SEQ ID NO: 1 or 385. In some embodiments, the effector proteins generate at least 5%, at least 10%, at least 15%, at least 20%, at least 25%, at least 30%, at least 35%, at least 40%, at least 45%, at least 50%, at least 55%, or at least 60% less indel in non-target strand relative to a corresponding control effector protein comprising an amino acid sequence of SEQ ID NO: 1 or 385. In some embodiments, the effector proteins generate more indels on target strand relative to a corresponding control effector protein comprising an amino acid sequence of SEQ ID NO: 1 or 385. In some embodiments, the effector proteins generate at least 5%, at least 10%, at least 15%, at least 20%, at least 25%, at least 30%, at least 35%, at least 40%, at least 45%, at least 50%, at least 55%, or at least 60% more indels on target strand relative to a corresponding control effector protein comprising an amino acid sequence of SEQ ID NO: 1 or 385. In some embodiments, a ratio of indels generated by the effector proteins on the target strand as compared to the non-target strand is higher relative to the same ratio of indels generated by a corresponding control effector protein comprising an amino acid sequence of SEQ ID NO: 1 or 385. In some embodiments, a ratio of indels generated by the effector proteins on the target strand as compared to the non-target strand is at least 5%, at least 10%, at least 15%, at least 20%, at least 25%, at least 30%, at least 35%, at least 40%, at least 45%, at least 50%, at least 55%, or at least 60% higher relative to the same ratio of indels generated by a corresponding control effector protein comprising an amino acid sequence of SEQ ID NO: 1 or 385.

### Effector Partners

Provided herein are compositions, systems, and methods comprising one or more effector partners or uses thereof. In some embodiments, the effector partner is a heterologous protein. In some embodiments, the effector partner edits a base (or a nucleobase) of a target strand, a non-target strand, or both. In some embodiments, the effector partner is fused or linked to an effector protein or a fusion protein thereof. In some embodiments, the effector partner is fused or linked to another effector partner or a fusion protein thereof. In some embodiments, the effector protein nicks a target strand or a non-target strand, wherein the effector partner edits a non-nicked strand. In some embodiments, a nicked strand is corrected by reverse transcriptase editing. In the context of a fusion protein, a person skilled in the art would recognize that reference to the effector partner and the effector protein being linked to each other is equivalent to the effector partner and the effector protein being fused to each other. Accordingly, in the context of fusion protein, reference to an effector partner and an effector protein being linked to each other and an effector partner and an effector protein being fused to each other are used interchangeably. In some embodiments, the amino terminus of the effector partner is linked to the carboxy terminus of the effector protein directly or by the linker. In some embodiments, the carboxy terminus of the effector partner is linked to the amino terminus of the effector protein directly or by the linker. In some embodiments, the effector partner is functional when the effector protein is coupled to a guide nucleic acid. In some embodiments, the effector partner is functional when the effector protein is coupled to a target nucleic acid. In some embodiments, the guide nucleic acid imparts sequence specific activity to the effector partner. By way of non-limiting example, the effector protein comprises a catalytically inactive effector protein (e.g., a catalytically inactive variant of an effector protein described herein).

In some embodiments, the effector partner described herein does not comprise an effector protein described herein. In some embodiments, the effector partner is capable of imparting (or imparts) a function or activity that is not provided by an effector protein. In some embodiments, the effector partner is capable of forming (or forms) a multimeric protein with another effector partner. In some embodiments, the multimeric protein is a heteromeric protein. In some embodiments, the multimeric protein is a homomeric protein.

In some embodiments, the effector partner described herein comprises an effector protein. Accordingly, in some embodiments, the effector partner is capable of forming (or forms) a multimeric protein with the effector protein. In some embodiments, the multimeric protein is a heteromeric protein.

In some embodiments, the effector partner directly or indirectly modifies a target nucleic acid. Modifications can be of a nucleobase, nucleotide, or nucleotide sequence of a target nucleic acid. In some embodiments, the effector partner interacts with additional proteins, or functional fragments thereof, to make modifications to a target nucleic acid. In some embodiments, modification of a target nucleic acid comprises introducing or removing epigenetic modification(s). In other embodiments, the effector partner modifies proteins associated with a target nucleic acid. In some embodiments, an effector partner modulates transcription (e.g., inhibits transcription, increases transcription) of a target nucleic acid. In yet another example, an effector partner may directly or indirectly inhibit, reduce, activate or increase expression of a target nucleic acid.

In some embodiments, the effector partner described herein comprises modification activities. In some embodiments, the modification activities comprise a cleavage activity, binding activity, insertion activity, substitution activity, and the like. Modification activity of an effector partner may result in: chemical modification of one or more nucleotides of a target nucleic acid into an alternate nucleotide, substitution of one or more nucleotides of a target nucleic acid with an alternative nucleotide, more than one of the foregoing, or any combination thereof. A target nucleic acid comprises a target strand and a non-target strand. Accordingly, in some embodiments, the effector partner edits a target strand and/or a non-target strand of a target nucleic acid. In some embodiments, an ability of an effector partner to modify a target nucleic acid depends upon the effector protein being complexed with a guide nucleic acid, the guide nucleic acid being hybridized to a target sequence of the target nucleic acid, the distance between the target sequence and a PAM sequence, concentration of the effector partner near to the target nucleic acid, distance between the effector protein and the effector partner, or combinations thereof.

In some embodiments, the systems described herein comprising effector partners and effector proteins, wherein the system modified a target nucleic acid comprising a target strand and a non-target strand. In some embodiments, the effector partner modifies the target strand or the non-target strand. In some embodiments, the effector protein nicks the target strand or the non-target strand. In some embodiments, the effector partner modifies the target strand, and the effector protein nicks the non-target strand. In some embodiments, the effector partner modifies the target strand, and the effector protein is a dCas protein. In some embodiments, the effector partner modifies the target strand, and the effector protein cleaves the target nucleic acid. In some embodiments, the effector partner modifies the non-target strand, and the effector protein nicks the non-target strand. In some embodiments, the effector partner modifies the non-target strand, and the effector protein is a dCas protein. In some embodiments, the effector partner modifies the non-target strand, and the effector protein cleaves the target nucleic acid. In some embodiments, the effector protein comprises amino acid substitutions of D220R, D237A, or a combination thereof. In some embodiments, the effector protein comprises amino acid substitutions of D220R, D237N, or a combination thereof. In some embodiments, the effector protein comprises amino acid substitutions of D220R, E335Q, or a combination thereof. In some embodiments, the effector partner can be any of the effector partners described herein.

TABLE 2 provides illustrative sequences of exemplary effector partners that are useful in the compositions, systems and methods described herein.

In some embodiments, compositions, systems and methods described herein comprise an effector partner, or a nucleic acid encoding the effector partner, wherein the amino acid sequence of the effector partner comprises at least 200 contiguous amino acids or more of the amino acid sequence recited in TABLE 2. In some embodiments, the amino acid sequence of an effector partner provided herein comprises at least 200, at least 220, at least 240, at least 260, at least 280, at least 300, at least 320, at least 340, at least 360, at least 380, at least 400 contiguous amino acids, at least 420 contiguous amino acids, at least 440 contiguous amino acids, at least 460 contiguous amino acids, at least 480 contiguous amino acids, at least 500 contiguous amino acids, at least 520 contiguous amino acids, at least 540 contiguous amino acids, at least 560 contiguous amino acids, at least 580 contiguous amino acids, at least 600 contiguous amino acids, at least 620 contiguous amino acids, at least 640 contiguous amino acids, at least 660 contiguous amino acids, at least 680 contiguous amino acids, at least 700 contiguous amino acids, at least 720 contiguous amino acids, at least 760 contiguous amino acids, at least 800 contiguous amino acids, at least 840 contiguous amino acids, at least 880 contiguous amino acids, at least 920 contiguous amino acids, at least 960 contiguous amino acids, or at least 1,000 contiguous amino acids, or more of the amino acid sequence of TABLE 2.

In some embodiments, compositions, systems and methods described herein comprise an effector partner or a nucleic acid encoding the effector partner, wherein the effector partner comprises a portion of the amino acid sequence recited in TABLE 2. In some embodiments, the effector partner comprises a portion of the amino acid sequence recited in TABLE 2, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of the amino acid sequence recited in TABLE 2. In some embodiments, the effector partner comprises a portion of the amino acid sequence recited in TABLE 2, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of the amino acid sequence recited in TABLE 2.

In some embodiments, compositions, systems, and methods described herein comprise an effector partner, or a nucleic acid encoding the effector partner, wherein the effector partner comprises an amino acid sequence that is at least 65%, at least 70%, at least 75%, at least 80%, at least 85%, at least 90%, at least 95%, at least 97%, at least 98%, or at least 99%, or 100% identical to the amino acid sequence as recited in TABLE 2. In some embodiments, an effector partner provided herein comprises an amino acid sequence that is at least 65% identical to the amino acid sequence as recited in TABLE 2. In some embodiments, an effector partner provided herein comprises an amino acid sequence that is at least 70% identical to the amino acid sequence as recited in TABLE 2. In some embodiments, an effector partner provided herein comprises an amino acid sequence that is at least 75% identical to the amino acid sequence as recited in TABLE 2. In some embodiments, an effector partner provided herein comprises an amino acid sequence that is at least 80% identical to the amino acid sequence as recited in TABLE 2. In some embodiments, an effector partner provided herein comprises an amino acid sequence that is at least 85% identical to the amino acid sequence as recited in TABLE 2. In some embodiments, an effector partner provided herein comprises an amino acid sequence that is at least 90% identical to the amino acid sequence as recited in TABLE 2. In some embodiments, an effector partner provided herein comprises an amino acid sequence that is at least 95% identical to the amino acid sequence as recited in TABLE 2. In some embodiments, an effector partner provided herein comprises an amino acid sequence that is at least 97% identical to the amino acid sequence as recited in TABLE 2. In some embodiments, an effector partner provided herein comprises an amino acid sequence that is at least 98% identical to the amino acid sequence as recited in TABLE 2. In some embodiments, an effector partner provided herein comprises an amino acid sequence that is at least 99% identical to the amino acid sequence as recited in TABLE 2. In some embodiments, an effector partner provided herein comprises an amino acid sequence that is 100% identical to the amino acid sequence as recited in TABLE 2. In some embodiments, an effector partner provided herein comprises an amino acid sequence that is at least 65%, at least 70%, at least 75%, at least 80%, at least 85%, at least 90%, at least 95%, at least 97%, at least 98%, or at least 99%, or 100% identical to any one of the amino acid sequences of SEQ ID NO: 2 and 391-393.

In some embodiments, compositions, systems, and methods described herein comprise an effector partner, or a nucleic acid encoding the effector partner, wherein the effector partner comprises an amino acid sequence that is at least 80%, at least 85%, at least 90%, at least 95%, at least 97%, at least 98%, at least 99%, or 100% similar to the amino acid sequence as recited in TABLE 2. In some embodiments, an effector partner provided herein comprises an amino acid sequence that is at least 80% similar to the amino acid sequence as recited in TABLE 2. In some embodiments, an effector partner provided herein comprises an amino acid sequence that is at least 85% similar to the amino acid sequence as recited in TABLE 2. In some embodiments, an effector partner provided herein comprises an amino acid sequence that is at least 90% identical to the amino acid sequence as recited in TABLE 2. In some embodiments, an effector partner provided herein comprises an amino acid sequence that is at least 95% similar to the amino acid sequence as recited in TABLE 2. In some embodiments, an effector partner provided herein comprises an amino acid sequence that is at least 97% similar to the amino acid sequence as recited in TABLE 2. In some embodiments, an effector partner provided herein comprises an amino acid sequence that is at least 98% similar to the amino acid sequence as recited in TABLE 2. In some embodiments, an effector partner provided herein comprises an amino acid sequence that is at least 99% similar to the amino acid sequence as recited in TABLE 2. In some embodiments, an effector partner provided herein comprises an amino acid sequence that is 100% similar to the amino acid sequence as recited in TABLE 2. In some embodiments, an effector partner provided herein comprises an amino acid sequence that is at least 65%, at least 70%, at least 75%, at least 80%, at least 85%, at least 90%, at least 95%, at least 97%, at least 98%, or at least 99%, or 100% similar to any one of the amino acid sequences of SEQ ID NO: 2 and 391-393.

In some embodiments, compositions, systems, and methods described herein comprise an effector partner, or a nucleic acid encoding the effector partner, wherein the effector partner comprises one or more amino acid alterations relative to the amino acid sequence recited in TABLE 2. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to the amino acid sequence recited in TABLE 2. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more amino acid alterations relative to the amino acid sequence recited in TABLE 2. In some embodiments, the one or more alterations comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid alterations relative to the amino acid sequence recited in TABLE 2. In some embodiments, the one or more alterations comprises one, two, three, four, five, six, seven, eight, nine, ten, or more amino acid alterations relative to the amino acid sequence recited in TABLE 2. In some embodiments, the one or more amino acid alterations comprises substitutions (e.g., conservative substitutions, non-conservative substitutions), deletions, or combinations thereof. In some embodiments, an effector partner or a nucleic acid encoding the effector partner comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to the amino acid sequence recited in TABLE 2.

In some embodiments, compositions, systems, and methods described herein comprise an effector partner, or a nucleic acid encoding the effector partner, wherein the effector partner comprises one or more substitutions relative to the amino acid sequence recited in TABLE 2. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to the amino acid sequence recited in TABLE 2. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more substitutions relative to the amino acid sequence recited in TABLE 2. In some embodiments, the one or more substitutions comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid substitutions relative to the amino acid sequence recited in TABLE 2. In some embodiments, the one or more substitutions comprise one, two, three, four, five, six, seven, eight, nine, ten or more substitutions relative to the amino acid sequence recited in TABLE 2. In some embodiments, the one or more substitutions comprise one or more conservative substitutions, one or more non-conservative substitutions, or combinations thereof.

In some embodiments, compositions, systems, and methods described herein comprise an effector partner, or a nucleic acid encoding the effector partner, wherein the effector partner comprises one or more conservative substitutions relative to the amino acid sequence recited in TABLE 2. In some embodiments, the one or more conservative substitutions comprises at least one, at least two, at least three, at least four, at least five, at least six, at least seven, at least eight, at least nine, at least ten, at least twelve, at least sixteen, at least twenty, or more conservative substitutions relative to the amino acid sequence recited in TABLE 2. In some embodiments, the one or more conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more conservative substitutions relative to the amino acid sequence recited in TABLE 2. In some embodiments, the one or more conservative substitutions comprise one, two, three, four, five, six, seven, eight, nine, ten or more conservative substitutions relative to the amino acid sequence recited in TABLE 2. In some embodiments, compositions, systems, and methods described herein comprise a variant of an effector partner, or a nucleic acid encoding the same, wherein the variant comprises 1, 2, 3, 4, 5, 6, 7, 8, 9 or 10 conservative amino acid substitutions relative to the amino acid sequence recited in TABLE 2. In some embodiments, the variant of effector partner as described herein further comprises an additional one or more alterations, such as non-conservative substitutions, relative to the amino acid sequence recited in TABLE 2.

In some embodiments, compositions, systems, and methods described herein comprise an effector partner, or a nucleic acid encoding the effector partner, wherein the effector partner comprises one or more non-conservative substitutions relative to the amino acid sequence recited in TABLE 2. In some embodiments, the one or more non-conservative substitutions comprises at least one, at least two, at least three, at least four, at least five, at least six, at least seven, at least eight, at least nine, at least ten, at least twelve, at least sixteen, at least twenty, or more non-conservative substitutions relative to the amino acid sequence recited in TABLE 2. In some embodiments, the one or more non-conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, sixteen to twenty, or more non-conservative substitutions relative to the amino acid sequence recited in TABLE 2. In some embodiments, the one or more non-conservative substitutions comprise one, two, three, four, five, six, seven, eight, nine, ten or more non-conservative substitutions relative to the amino acid sequence recited in TABLE 2. In some embodiments, compositions, systems, and methods described herein comprise a variant of an effector partner, or a nucleic acid encoding the same, wherein the variant comprises 1, 2, 3, 4, 5, 6, 7, 8, 9 or 10 non-conservative amino acid substitutions relative to the amino acid sequence recited in TABLE 2. In some embodiments, the variant as described herein further comprises an additional one or more alterations, such as conservative substitutions, relative to the amino acid sequence recited in TABLE 2.

### Multimeric Complex Formation Modification Activity

In some embodiments, an effector partner inhibits the formation of a multimeric complex of an effector protein. Alternatively, the effector partner promotes the formation of a multimeric complex of the effector protein. In some embodiments, two of more effector partners forms a multimeric complex with the effector protein. In some embodiments, two of more effector partners forms a multimeric complex with the effector partner. In some embodiments, the effector partner comprises a Calcineurin A tag, which promotes formation of a multimeric complex (e.g., dimer) in the presence of Tacrolimus (FK506). In some embodiments, the effector partner comprises a SpyTag configured to dimerize or associate with another protein in a multimeric complex.

### Prime Editing System

In some embodiments, an effector partner and/or a fusion protein can comprise a prime editing enzyme. When used herein, a prime editing enzyme can describe a protein, polypeptide, or fragment thereof that is capable of catalyzing (or catalyzes) the modification (insertion, deletion, or base-to-base conversion) of a target nucleotide or nucleotide sequence in a nucleic acid. A prime editing enzyme capable of catalyzing (or catalyzes) such a reaction includes a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K, and W313F relative to wildtype M-MLV RT enzyme. Accordingly, in some embodiments, the prime editing systems described herein comprise prime editing enzymes or nucleic acids encoding the prime editing enzymes, wherein the prime editing enzymes catalyze a reverse transcriptase reaction.

A prime editing enzyme may require a prime editing guide RNA (pegRNA) to catalyze the modification. Such a pegRNA can be capable of identifying (or identifies) the nucleotide or nucleotide sequence in the target nucleic acid to be edited and encoding the new genetic information that replaces the targeted nucleotide or nucleotide sequence in the nucleic acid. A prime editing enzyme may require a pegRNA and a single guide RNA to catalyze the modification. In some embodiments, the target nucleic acid is a dsDNA molecule. In some embodiments, the pegRNA comprises a guide RNA comprising a first region that is bound by the effector protein, and a second region comprising a spacer sequence that is complementary to a target sequence of the target dsDNA molecule; a template RNA comprising a primer binding sequence that hybridizes to a primer sequence of the target dsDNA molecule that is formed when target nucleic acid is cleaved, and a template sequence that is complementary to at least a portion of the target sequence of the target dsDNA molecule with the exception of at least one nucleotide. In some embodiments, the spacer sequence is complementary to the target sequence on the target strand of the dsDNA molecule. In some embodiments, the spacer sequence is complementary to the target sequence on the non-target strand of the dsDNA molecule. In some instances, the primer binding sequence hybridizes to a primer sequence on the non-target strand of the target dsDNA molecule. In some instances, the primer binding sequence hybridizes to a primer sequence on the target strand of the target dsDNA molecule. In some instances, the target strand is cleaved. In some instances, the non-target strand is cleaved.

### Nucleic Acid Modification Activity

In some embodiments, effector partners have enzymatic activity that modifies a nucleic acid, such as a target nucleic acid. In some embodiments, the target nucleic acid comprises or consists of a ssRNA, dsRNA, ssDNA, or a dsDNA. Examples of enzymatic activity that modifies the target nucleic acid include, but are not limited to: nuclease activity, which comprises the enzymatic activity of an enzyme which allows the enzyme to cleave the phosphodiester bonds between the nucleotide subunits of nucleic acids, such as that provided by a restriction enzyme, or a nuclease (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; helicase activity; photolyase activity; and glycosylase activity.

In some embodiments, effector partners target a ssRNA, dsRNA, ssDNA, or a dsDNA. In some embodiments, effector partners target ssRNA. Non-limiting examples of effector partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that an effector partner may include an entire protein, or in some embodiments, includes a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, protein binding or combinations thereof. The absence of the functional domain, including mutations of the functional domain, would abolish or reduce activity.

Accordingly, effector partners may comprise a protein or domain thereof selected from: deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); and other suitable domains that affect nucleic acid modifications.

In some embodiments, effector partner comprises a chromatin-modifying enzyme. In some embodiments, the effector partner chemically modifies a target nucleic acid, for example by methylating, demethylating, or acetylating the target nucleic acid in a sequence specific or non-specific manner.

### Base Editing Enzymes

In some embodiments, effector partners edit a nucleobase of a target nucleic acid. Such a effector partner may be referred to as a base editing enzyme. In some embodiments, a base editing enzyme variant that differs from a naturally occurring base editing enzyme, but it is understood that any reference to a base editing enzyme herein also refers to a base editing enzyme variant. In some embodiments, the base editing enzyme edits a base on a target strand of the target nucleic acid. In some embodiments, the base editing enzyme edits a base on a non-target strand of the target nucleic acid.

In some embodiments, a base editor is a system comprising an effector protein and a base editing enzyme. In some embodiments, the base editor is capable of modifying (or modifies) a target nucleic acid, wherein the target nucleic acid comprises a target strand and a non-target strand. In some embodiments, the effector protein nicks one of the target strand or the non-target strand, wherein the base editing enzyme edits a non-nicked strand. In some embodiments, a nicked strand is corrected by a reverse transcriptase enzyme. For example, in some embodiments, the base editing enzyme modifies a base of the target strand, and the effector protein nicks the non-target strand. Accordingly, in some embodiments, the non-target strand is corrected by the reverse transcriptase enzyme. In some embodiments, the base editing enzyme modifies a base of the target strand, and the effector protein is a dCas protein. In some embodiments, the base editing enzyme modifies a base of the target strand, and the effector protein cleaves the target nucleic acid. In some embodiments, the base editing enzyme modifies a base of the non-target strand, and the effector protein nicks a non-target strand. In some embodiments, the base editing enzyme modifies a base of the non-target strand, and the effector protein is a dCas protein. In some embodiments, the base editing enzyme modifies a base of the non-target strand, and the effector protein cleaves the target nucleic acid. For example, in some embodiments, the dCas protein comprises one or more amino acid substitutions of D237A, D237N, and E335Q relative to the amino acid sequence recited in TABLE 1. In some embodiments, the dCas protein further comprises one or more substitutions with positively charged amino acid as described herein. In some embodiments, the positively charged amino acid substitution comprises an amino acid substitution of D220R relative to the amino acid sequence recited in TABLE 1.

In some embodiments, the base editor described herein comprises a base editing enzyme and an effector protein as independent components. In some embodiments, the base editor comprises a fusion protein comprising a base editing enzyme fused or linked to an effector protein. Without being bound by theory, it is predicted that the binding of an effector protein at the target locus allows the fused effector partner to induce base changes on both, target strand and non-target strand, of a double stranded nucleic acid, wherein the effector protein having nickase activity nicks one of the two stands of the double stranded nucleic acid which would bias DNA repair machinery to “correct” the base edit on the nicked strand, keeping the not-nicked strand edit. Accordingly, in some embodiments, the effector protein nicks one of the target strand or the non-target strand, wherein the base editing enzyme edits a non-nicked strand. In some embodiments, a nicked strand is corrected by a reverse transcriptase enzyme. In some embodiments, the amino terminus of the effector partner is linked to the carboxy terminus of the effector protein by the linker. In some embodiments, the carboxy terminus of the effector partner is linked to the amino terminus of the effector protein by the linker. The base editor may be functional when the effector protein is coupled to a guide nucleic acid. The base editor may be functional when the effector protein is coupled to a target nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. Accordingly, in some embodiments, the base editing enzyme comprises a deaminase or deaminase activity. Additional base editors are described herein.

In some embodiments, base editing enzymes are capable of catalyzing (or catalyzes) editing (e.g., a chemical modification) of a nucleobase of a nucleic acid molecule, such as DNA or RNA (single stranded or double stranded). In some embodiments, a base editing enzyme, and therefore a base editor, is capable of converting (or converts) an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In the context of base editing, a person skilled in the art would recognize that reference to the nucleobase (e.g., adenine) or nucleotide (e.g., adenosine) that is being modified by the base editor or base editing enzyme is the nucleobase of the molecule. Accordingly, in the context of base editing, reference to a nucleobase and nucleotide are used interchangeably. In some embodiments, base editing enzymes edit a nucleobase on a ssDNA. In some embodiments, base editing enzymes edit a nucleobase on both strands of dsDNA. In some embodiments, base editing enzymes edit a nucleobase of an RNA.

In some embodiments, a base editing enzyme itself does or does not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editing enzyme having the deaminase enzyme activity. In some embodiments, base editing systems for improved efficiency in eukaryotic cells comprise a base editing enzyme, and a catalytically inactive effector protein that generates a nick in the non-edited strand and induce repair of the non-edited strand using the edited strand as a template.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3: eaao4774 (2017), and Rees et al., Nat Rev Genet. 2018 December; 19(12):770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editing enzymes comprise a DNA glycosylase inhibitor (e.g., an uracil glycosylase inhibitor (UGI) or uracil N-glycosylase (UNG)). In some embodiments, the effector partner is a deaminase, e.g., ADAR1/2, ADAR-2, AID, or any functional variant thereof.

In some embodiments, the base editor is a cytosine base editor (CBE), wherein the base editing enzyme is a cytosine base editing enzyme. In some embodiments, the cytosine base editing enzyme, and therefore CBE, converts a cytosine to a thymine. In some embodiments, a cytosine base editing enzyme accepts ssDNA as a substrate but is not capable of cleaving (or cleaves) dsDNA, wherein the CBE comprises a catalytically inactive effector protein. In some embodiments, a cytosine base editing enzyme is capable of introducing (or introduces) a premature stop codon into a target nucleic acid. Accordingly, in some embodiments, a cytosine base editing enzyme is useful in gene knockout application. In some embodiments, when bound to its cognate DNA, the catalytically inactive effector protein of the CBE performs local denaturation of the DNA duplex to generate an R-loop in which the DNA strand not paired with a guide nucleic acid exists as a disordered single-stranded bubble. In some embodiments, the catalytically inactive effector protein generated ssDNA R-loop enables the CBE to perform efficient and localized cytosine deamination in vitro. In some embodiments, deamination activity is exhibited in a window of about 4 to about 10 base pairs. In some embodiments, the catalytically inactive effector protein presents a target site to the cytosine base editing enzyme in high effective molarity, which enables the CBE to deaminate cytosines located in a variety of different sequence motifs, with differing efficacies. In some embodiments, the CBE is capable of mediating (or mediates) RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C·G-to-G·C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, the effector partner comprises a uracil glycosylase inhibitor (UGI). In some embodiments, the CBEs described herein comprise UGI. In some embodiments, base excision repair (BER) of U·G in DNA is initiated by a UNG, which recognizes a U·G mismatch and cleaves the glyosidic bond between a uracil and a deoxyribose backbone of DNA. In some embodiments, BER results in the reversion of the U·G intermediate created by the cytosine base editing enzyme back to a C·G base pair. In some embodiments, the UNG is inhibited by fusion of a UGI. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG.

In some embodiments, the CBEs described herein mediates efficient base editing in bacterial cells and moderately efficient editing in mammalian cells, enabling conversion of a C·G base pair to a T·A base pair through a U·G intermediate. In some embodiments, the CBE is modified to increase base editing efficiency while editing more than one strand of DNA.

In some embodiments, the CBEs described herein nicks a non-edited DNA strand. In some embodiments, the non-edited DNA strand nicked by the CBE biases cellular repair of a U·G mismatch to favor a U·A outcome, elevating base editing efficiency.

In some embodiments, a base editor described herein comprising one or more base editing enzymes (e.g., APOBEC1, nickase, and UGI) efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment is capable of excising (excises) a uracil residue from DNA by cleaving an N-glycosidic bond.

In some embodiments, the effector partner comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, the base editor is a cytosine base editor, wherein the based editing enzyme is a cytosine base editing enzyme. In some embodiments, the cytosine base editing enzyme is a cytidine deaminase. In some embodiments, the base editor comprising the cytidine deaminase is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9 (A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE), wherein the base editing enzyme is a cytosine to guanine base editing enzyme. In some embodiments, the cytosine to guanine base editing enzyme and, therefore, the CGBE, converts a cytosine to a guanine.

In some embodiments, a base editor is an adenine base editor (ABE), wherein the base editing enzyme is an adenine base editing enzyme. In some embodiments, the adenine base editing enzyme and, therefore, the ABE converts an adenine to a guanine. In some embodiments, the adenine base editing enzyme converts an A·T base pair to a G.C base pair. In some embodiments, the adenine base editing enzyme converts a target A·T base pair to G.C in vivo or in vitro. In some embodiments, the adenine base editing enzymes provided herein reverse spontaneous cytosine deamination, which has been linked to pathogenic point mutations. In some embodiments, the adenine base editing enzymes provided herein enable correction of pathogenic SNPs (~47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing (or pairs) with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max), and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, the ABE described herein is capable of targeting (targets) polyA signals, splice site acceptors, and start codons. In some embodiments, the ABE cannot create stop codons for knock-down.

In some embodiments, an adenine base editing enzyme is an adenosine deaminase. Non-limiting exemplary adenosine base editors suitable for use herein include ABE9. In some embodiments, the ABE comprises an engineered adenosine deaminase enzyme capable of acting (acts) on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, the base editor comprises an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises TadA.

In some embodiments, a base editing enzyme is a deaminase dimer. In some embodiments, the ABE comprises the effector protein, the adenine base editing enzyme and the deaminase dimer. In some embodiments, the deaminase dimer comprises an adenosine deaminase. In some embodiments, the deaminase dimer comprises TadA and a suitable adenine base editing enzyme including an: ABE8e, ABE8.20m, APOBEC3A, Anc APOBEC (a.k.a. AncBE4Max), BtAPOBEC2, and variants thereof. In some embodiments, the adenine base editing enzyme is fused to amino-terminus or the carboxy-terminus of TadA.

In some embodiments, a base editor is an RNA base editor, wherein the base editing enzyme is an RNA base editing enzyme. In some embodiments, the RNA base editing enzyme comprises an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editing enzymes, and therefore base editors, are used for treating a subject having or a subject suspected of having a disease related to a gene of interest. In some embodiments, base editing enzymes, and therefore base editors, are useful for treating a disease or a disorder caused by a point mutation in a gene of interest. In some embodiments, compositions, systems, and methods described herein comprise a base editor and a guide nucleic acid, wherein the base editor comprises an effector protein and a base editing enzyme, and wherein the guide nucleic acid directs the base editor to a sequence in a target gene.

### Protein Modification Activity

In some embodiments, an effector partner provides enzymatic activity that modifies a protein associated with a target nucleic acid. The protein may be a histone, an RNA binding protein, or a DNA binding protein. Examples of such protein modification activities include: methyltransferase activity, such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity; phosphatase activity; ubiquitin ligase activity; deubiquitinating activity; adenylation activity; deadenylation activity; SUMOylating activity; deSUMOylating activity; ribosylation activity; deribosylation activity; myristoylation activity; and demyristoylation activity.

### CRISPRa Fusions and CRISPRi Fusions

In some embodiments, effector partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, effector partners that increase or decrease transcription include a transcription activator domain or a transcription repressor domain, respectively.

In some embodiments, effector partners activate or increase expression of a target nucleic acid. In some embodiments, effector partners increase expression of the target nucleic acid relative to its expression in the absence of the effector partners. Relative expression, including transcription and RNA levels, may be assessed, quantified, and compared, e.g., by RT-qPCR. In some embodiments, effector partners comprise a transcriptional activator. In some embodiments, the transcriptional activators promote transcription by: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof.

Non-limiting examples of effector partners that promote or increase transcription include: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable effector partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, effector partners inhibit or reduce expression of a target nucleic acid. In some embodiments, effector partners reduce expression of the target nucleic acid relative to its expression in the absence of the effector partners. Relative expression, including transcription and RNA levels, may be assessed, quantified, and compared, e.g., by RT-qPCR. In some embodiments, effector partners comprise a transcriptional repressor. In some embodiments, the transcriptional repressors inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of effector partners that decrease or inhibit transcription include: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable effector partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

In some embodiments, fusion proteins comprising the described effector partners and an effector protein are referred to as CRISPRa fusions, wherein the effector partners activate or increase expression of a target nucleic acid. In some embodiments, fusion proteins comprising the described effector partners and an effector protein are referred to as CRISPRi fusions, wherein the effector partners inhibit or reduce expression of a target nucleic acid. In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function), and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, effector partner comprises an RNA splicing factor. The RNA splicing factor may be used (in whole or as fragments thereof) for modular organization, with separate sequence-specific RNA binding modules and splicing effector domains. In some embodiments, the RNA splicing factors comprise members of the Serine/Arginine-rich (SR) protein family containing N-terminal RNA recognition motifs (RRMs) that bind to exonic splicing enhancers (ESEs) in pre-mRNAs and C-terminal RS domains that promote exon inclusion. In some embodiments, a hnRNP Al binds to exonic splicing silencers (ESSs) through its RRM domains and inhibits exon inclusion through a C-terminal Glycine-rich domain. In some embodiments, the RNA splicing factors regulate alternative use of splice site (ss) by binding to regulatory sequences between two alternative sites. For example, in some embodiments, ASF/SF2 recognize ESEs and promote the use of intron proximal sites, whereas hnRNP Al binds to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. Long splicing isoform Bcl-xL is a potent apoptosis inhibitor expressed in long-lived postmitotic cells and is up-regulated in many cancer cells, protecting cells against apoptotic signals. Short isoform Bcl-xS is a pro-apoptotic isoform and expressed at high levels in cells with a high turnover rate (e.g., developing lymphocytes). A ratio of the two Bcl-x splicing isoforms is regulated by multiple có-elements that are located in either core exon region or exon extension region (i.e., between the two alternative 5′ splice sites). For more examples, see WO2010075303, which is hereby incorporated by reference in its entirety.

### Recombinases

In some embodiments, effector partners comprise a recombinase. In some embodiments, a recombinase system comprises effector proteins described herein and the recombinase. In some embodiments, the effector proteins have reduced nuclease activity or no nuclease activity. In some embodiments, the recombinase is a site-specific recombinase.

In some embodiments, the recombinase system comprises a catalytically inactive effector protein, wherein the recombinase can be a site-specific recombinase. Such systems can be used for site-directed transgene insertion. Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include: Bxb1, wBeta, BL3, phiR4, A118, TG1, MR11, phi370, SPBc, TP901-1, phiRV, FC1, K38, phiBT1, and phiC31. Further discussion and examples of suitable recombinase effector partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

### Linkers for Peptides

In some embodiments, a linker comprises a bond or molecule that links a first polypeptide to a second polypeptide. Accordingly, in some embodiments, effector proteins, effector partners, or combinations thereof are connected by linkers. The linker may comprise or consist of a covalent bond. The linker may comprise or consist of a chemical group. In some embodiments, the linker comprises an amino acid. In some embodiments, a peptide linker comprises at least two amino acids linked by an amide bond. In general, the linker connects a terminus of the effector protein to a terminus of the effector partner. In some embodiments, carboxy terminus of the effector protein is linked to the amino terminus of the fusion effector. In some embodiments, carboxy terminus of the effector partner is linked to the amino terminus of the effector protein. In some embodiments, the effector protein and the effector partner are directly linked by a covalent bond.

In some embodiments, linkers comprise one or more amino acids. In some embodiments, linker is a protein. In some embodiments, a terminus of the effector protein is linked to a terminus of the effector partner through an amide bond. In some embodiments, a terminus of the effector protein is linked to a terminus of the effector partner through a peptide bond. In some embodiments, linkers comprise an amino acid. In some embodiments, linkers comprise a peptide. In some embodiments, an effector protein is coupled to an effector partner by a linker protein. In some embodiments, the linker comprises any of a variety of amino acid sequences. In some embodiments, the linker comprises a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, linked amino acids described herein comprise at least two amino acids linked by an amide bond.

Linkers may be produced by using synthetic, linker-encoding oligonucleotides to couple proteins, or may be encoded by a nucleic acid sequence encoding a fusion protein (e.g., an effector protein coupled to an effector partner). In some embodiments, the linker is from 1 to 300, from 1 to 250, from 1 to 200, from 1 to 150, from 1 to 100, from 1 to 50, from 1 to 25, from 1 to 10, from 10 to 300, from 10 to 250, from 10 to 200, from 10 to 150, from 10 to 100, from 10 to 50, from 10 to 25, from 25 to 300, from 25 to 250, from 25 to 200, from 25 to 150, from 25 to 100, from 25 to 50, from 50 to 300, from 50 to 250, from 50 to 200, from 50 to 150, from 50 to 100, from 100 to 300, from 100 to 250, from 100 to 200, from 100 to 150, from 150 to 300, from 150 to 250, from 150 to 200, from 200 to 300, from 200 to 250, or from 250 to 300 amino acids in length. In some embodiments, the linker is from 1 to 100 amino acids in length. In some embodiments, the linker is more 100 amino acids in length. In some embodiments, the linker is from 10 to 27 amino acids in length. In some embodiments, linker proteins include glycine polymers (G)n, glycine-serine polymers (including, for example, (GS)n, (GSGGS)n, (GGSGGS)n, and (GGGS)n, where n is an integer of at least one), glycine-alanine polymers, and alanine-serine polymers. TABLE 7.1 lists non-limiting exemplary polypeptide linker sequences. In some embodiments, the linker comprises one or more repeats a tri-peptide GGS. In some embodiments, the linker is a GS-rich linker. In some embodiments, the GS-rich linker comprises a peptide having two amino acids (2aa), three amino acids (3aa), five amino acids (5aa), ten amino acids (10aa), twenty amino acids (20aa), or forty amino acids (40aa). In some embodiments, the linker is an XTEN linker. In some embodiments, the XTEN linker is an XTEN80 linker. In some embodiments, the XTEN linker is an XTEN40 linker. In some embodiments, the XTEN linker is an XTEN20 linker. In some embodiments, the XTEN20 has an acid sequence linker amino of GSGGSPAGSPTSTEEGTSESATPGSG (SEQ ID NO: 217). In some embodiments, the XTEN linker is an XTEN10 linker.

In some embodiments, a polypeptide described herein comprises an activity (e.g., a binding activity, a catalytic activity, or a combination thereof) for a target nucleic acid comprising a target strand and a non-target strand. In some embodiments, a length of the linker effects preference of the polypeptide for the activity on the target strand relative to the activity on the non-target strand. In some embodiments, a length of the linker effects preference of the polypeptide for the activity on the target strand relative to the activity on the non-target strand, wherein the polypeptide comprises C-terminus of an effector protein described herein linked by the linker to an effector protein described herein. In some embodiments, a shorter length of the linker (e.g., up to one amino acid, up to two amino acids, up to three amino acids, up to four amino acids, up to five amino acids, up to six amino acids, up to seven amino acids, up to eight amino acids, up to nine amino acids, or up to ten amino acids) favors activity of the polypeptide on the target strand relative to activity on the non-target strand, wherein the polypeptide comprises C-terminus of an effector protein described herein linked by the linker to an effector protein described herein.

In some embodiments, a length of a linker effects activity of the polypeptide described herein. In some embodiments, a length of the linker effects activity of the polypeptide, wherein the polypeptide comprises N-terminus of an effector protein described herein linked by the linker to an effector protein described herein.

In some embodiments, linkers do not comprise an amino acid. In some embodiments, linkers do not comprise a peptide. In some embodiments, linkers comprise a nucleotide, a polynucleotide, a polymer, or a lipid. In some embodiments, linker is a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

### Engineered Proteins

In some embodiments, proteins (e.g., effector protein, effector partner) described herein have been modified (also referred to as an engineered protein). In some embodiments, a modification of the proteins includes addition of one or more amino acids, deletion of one or more amino acids, substitution of one or more amino acids, or combinations thereof. In some embodiments, the proteins disclosed herein are engineered proteins. Unless otherwise indicated, reference to the proteins throughout the present disclosure include engineered proteins thereof.

In some embodiments, proteins (e.g., effector protein, effector partner) described herein can be modified with the addition of one or more heterologous peptides. In some embodiments, the protein modified with the addition of one or more heterologous peptides is referred to herein as a fusion protein. Such fusion proteins are described herein and throughout.

In some embodiments, a heterologous peptide comprises a subcellular localization signal. In some embodiments, a subcellular localization signal can be a nuclear localization signal (NLS). In some embodiments, the NLS facilitates localization of a nucleic acid, protein, or small molecule to the nucleus, when present in a cell that contains a nuclear compartment. TABLE 3 lists exemplary NLS sequences. In some embodiments, the subcellular localization signal is a nuclear export signal (NES), a sequence to keep the protein retained in the cytoplasm, a mitochondrial localization signal for targeting to the mitochondria, a chloroplast localization signal for targeting to a chloroplast, an ER retention signal, and the like. In some embodiments, the protein described herein is not modified with a subcellular localization signal so that the protein is not targeted to the nucleus, which can be advantageous depending on the circumstance (e.g., when the target nucleic acid is an RNA that is present in the cytosol).

In some embodiments, a heterologous peptide comprises a chloroplast transit peptide (CTP), also referred to as a chloroplast localization signal or a plastid transit peptide, which targets the protein to a chloroplast. Chromosomal transgenes from bacterial sources requires a sequence encoding a CTP sequence fused to a sequence encoding an expressed protein (e.g., effector protein, effector partner) if the expressed protein is to be compartmentalized in the plant plastid (e.g., chloroplast). The CTP may be removed in a processing step during translocation into the plastid. Accordingly, localization of the protein to a chloroplast is often accomplished by means of operably linking a polynucleotide sequence encoding a CTP sequence to the 5′ region of a polynucleotide encoding the exogenous protein.

In some embodiments, the heterologous peptide is an endosomal escape peptide (EEP). An EEP is an agent that quickly disrupts the endosome in order to minimize the amount of time that a delivered molecule, such protein, spends in the endosome-like environment, and to avoid getting trapped in the endosomal vesicles and degraded in the lysosomal compartment. An exemplary EEP is recited in TABLE 3.

In some embodiments, the heterologous peptide is a cell penetrating peptide (CPP), also known as a Protein Transduction Domain (PTD). A CPP may facilitate traversing a lipid bilayer, micelle, cell membrane, organelle membrane, or vesicle membrane.

Further suitable heterologous peptide include, but are not limited to, proteins (or fragments/domains thereof) that are boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

In some embodiments, a heterologous peptide comprises a protein tag. In some embodiments, the protein tag is referred to as purification tag or a fluorescent protein. The protein tag may be detectable for use in detection of the protein and/or purification of the protein. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6×His tag (SEQ ID NO: 387); a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

A heterologous peptide may be located at or near the amino terminus (N-terminus) of the protein (e.g., effector protein, effector partner) disclosed herein. A heterologous peptide may be located at or near the carboxy terminus (C-terminus) of the proteins disclosed herein. In some embodiments, a heterologous peptide is located internally in the protein described herein (i.e., is not at the N- or C-terminus of the protein described herein) at a suitable insertion site.

In some embodiments, protein (e.g., effector protein, effector partner) described herein comprises 1, 2, 3, 4, 5, 6, 7, 8, 9, 10, or more heterologous peptide at or near the N-terminus, 1, 2, 3, 4, 5, 6, 7, 8, 9, 10, or more heterologous peptide at or near the C-terminus, or a combination of these (e.g., one or more heterologous peptide at the amino-terminus and one or more heterologous peptide at the carboxy terminus). When more than one heterologous peptide is present, each may be selected independently of the others, such that a single heterologous peptide may be present in more than one copy and/or in combination with one or more other heterologous peptide present in one or more copies. In some embodiments, a heterologous peptide is considered near the N- or C-terminus when the nearest amino acid of the heterologous peptide is within about 1, 2, 3, 4, 5, 10, 15, 20, 25, 30, 40, 50, or more amino acids along the polypeptide chain from the N- or C-terminus.

In some embodiments, a heterologous peptide described herein comprises a heterologous peptide sequence recited in TABLE 3. In some embodiments, proteins described herein comprise any one of the proteins (e.g., effector proteins, effector partners, fusion proteins, or combinations thereof) described herein fused to one or more of the sequences recited in TABLE 3. In some embodiments, a heterologous peptide described herein is an effector partner as described en supra.

In some embodiments, proteins (e.g., effector protein, effector partner, fusion protein) described herein are encoded by a codon optimized nucleic acid. In some embodiments, a nucleic acid sequence encoding the protein described herein, is codon optimized. In some embodiments, the proteins described herein is codon optimized for expression in a specific cell, for example, a bacterial cell, a plant cell, a eukaryotic cell, an animal cell, a mammalian cell, or a human cell. In some embodiments, the effector protein is codon optimized for a human cell. In some embodiments, the effector partner is codon optimized for a human cell.

### Fusion Proteins

In some embodiments, compositions, systems, and methods comprise a fusion protein or uses thereof. A fusion protein generally comprises at least one effector protein, at least one effector partner, or a combination thereof. For example, in some embodiments, the effector protein, the fusion protein thereof, or the combination thereof is further fused to an enzyme selected from an endonuclease and a glycosylase. Similarly, in some embodiments, the effector partner, the fusion protein thereof, or the combination thereof are further fused to an enzyme selected from an endonuclease and a glycosylase. Alternatively, in some embodiments, the effector protein, the effector partner, the fusion protein thereof, or the combinations thereof are independently fused to one or more enzymes selected from an endonuclease and a glycosylase. In some embodiments, the effector partner is fused or linked to the effector protein. In some embodiments, the effector partner is fused to the N-terminus of the effector protein. In some embodiments, the effector partner is fused to the C-terminus of the effector protein.

In some embodiments, the fusion proteins are multimeric proteins. In some embodiments, the multimeric protein is a homomeric protein. In some embodiments, the multimeric protein is a heteromeric protein. In some embodiments, the fusion protein comprising the effector partner is an effector protein. Accordingly, in such embodiments, the fusion protein can comprise at least two effector proteins that are same. In some embodiments, the fusion protein comprises at least two effector proteins that are different. Unless otherwise indicated, reference to effector proteins throughout the present disclosure include fusion proteins described herein.

In some embodiments, the fusion protein described herein comprises the effector protein that is located at the N-terminus of the effector partner. In some embodiments, the fusion protein described herein comprises the effector protein that is located at the C-terminus of the effector partner. In some embodiments, the effector protein comprises amino acid substitutions of D220R, D237A, or combination thereof. In some embodiments, the effector protein comprises amino acid substitutions of D220R, D237N, or combination thereof. In some embodiments, the effector protein comprises amino acid substitutions of D220R, E335Q, or combination thereof.

In some embodiments, the fusion protein complexes with a guide nucleic acid and the complex interacts with the target nucleic acid, a non-target nucleic acid, or both. In some embodiments, the interaction comprises one or more of: recognition of a protospacer adjacent motif (PAM) sequence within the target nucleic acid by the effector protein, hybridization of the guide nucleic acid to the target nucleic acid, nicking of the target nucleic acid, modification of the target nucleic acid and/or the non-target nucleic acid by the fusion protein, or combinations thereof. In some embodiments, recognition of a PAM sequence within a target nucleic acid directs the modification activity of a fusion protein.

Modification activity of a fusion protein described herein may be nickase activity, binding activity, substitution activity, and the like. Modification activity of a fusion protein may result in: nicking of a target nucleic acid, chemical modification of one or more nucleotides of a target nucleic acid into an alternative nucleotide, substitution of one or more nucleotides of a target nucleic acid with an alternative nucleotide, more than one of the foregoing, or any combination thereof. In some embodiments, an ability of a fusion protein to edit a target nucleic acid depends upon the effector protein being complexed with a guide nucleic acid, the guide nucleic acid being hybridized to a target sequence of the target nucleic acid, or combinations thereof. A target nucleic acid comprises a target strand and a non-target strand. Accordingly, in some embodiments, the fusion protein edits a target strand and/or a non-target strand of a target nucleic acid.

In some embodiments, the fusion protein described herein comprises a fusion protein, wherein the fusion protein affects formation of a multimeric complex of the fusion protein. By way of non-limiting example, the fusion protein comprises an effector protein described herein and an effector partner comprising a Calcineurin A tag, wherein the fusion protein dimerizes in the presence of Tacrolimus (FK506). Also, by way of non-limiting example, the fusion protein comprises an effector protein described herein and a SpyTag configured to dimerize or associate with another effector protein in a multimeric complex. Multimeric complex formation is further described herein.

In some embodiments, the fusion proteins described herein comprises the effector protein described herein and the base editing enzyme described herein. In some embodiments, the fusion proteins are capable of editing (edits) a base on a non-target strand of the target nucleic acid. In some embodiments, the fusion proteins are capable of editing (edits) a base on a target strand of the target nucleic acid. In some embodiments, the fusion proteins are provided with an additional effector partner comprising a ssDNA binding protein. In some embodiments, the ssDNA binding protein prevents non-target strand editing by the fusion protein.

In some embodiments, the fusion proteins described herein generate more indels on a target strand of a target nucleic acid relative to a non-target strand of the target nucleic acid. In some embodiments, the fusion proteins generate less indel in non-target strand relative to a corresponding control fusion protein comprising an amino acid sequence of SEQ ID NO: 1 or 385. In some embodiments, the fusion proteins generate at least 5%, at least 10%, at least 15%, at least 20%, at least 25%, at least 30%, at least 35%, at least 40%, at least 45%, at least 50%, at least 55%, or at least 60% less indel in non-target strand relative to a corresponding control fusion protein comprising an amino acid sequence of SEQ ID NO: 1 or 385. In some embodiments, the fusion proteins generate more indels on target strand relative to a corresponding control fusion protein comprising an amino acid sequence of SEQ ID NO: 1 or 385. In some embodiments, the fusion proteins generate at least 5%, at least 10%, at least 15%, at least 20%, at least 25%, at least 30%, at least 35%, at least 40%, at least 45%, at least 50%, at least 55%, or at least 60% more indels on target strand relative to a corresponding control fusion protein comprising an amino acid sequence of SEQ ID NO: 1 or 385. In some embodiments, a ratio of indels generated by the fusion proteins on the target strand as compared to the non-target strand is higher relative to the same ratio of indels generated by corresponding control fusion protein comprising an amino acid sequence of SEQ ID NO: 1 or 385. In some embodiments, a ratio of indels generated by the fusion proteins on the target strand as compared to the non-target strand is at least 5%, at least 10%, at least 15%, at least 20%, at least 25%, at least 30%, at least 35%, at least 40%, at least 45%, at least 50%, at least 55%, or at least 60% higher relative to the same ratio of indels generated by corresponding control fusion protein comprising an amino acid sequence of SEQ ID NO: 1 or 385.

### Multimeric Complexes

Compositions, systems, and methods of the present disclosure may comprise a multimeric complex or uses thereof, wherein the multimeric complex comprises one or more polypeptides (e.g., effector proteins, effector partners, fusion proteins, or combinations thereof) that non-covalently interact with one another. In some embodiments, the polypeptide functions as part of a multiprotein complex, including, for example, a complex having two or more polypeptides, including two or more of the same polypeptides (e.g., dimer or multimer). The polypeptide, when functioning in a multiprotein complex, may have only one functional activity (e.g., binding to a guide nucleic acid), while other polypeptides present in the multiprotein complex are capable of (or have) the other functional activity (e.g., editing a target nucleic acid). In some embodiments, the polypeptide, when functioning in a multiprotein complex, has differing and/or complementary functional activity to other polypeptides in the multiprotein complex. In some embodiments, the polypeptide is modified to have increased substrate binding activity (e.g., substrate selectivity, specificity, and/or affinity) relative to an unmodified counterpart wildtype polypeptide. In some embodiments, the substrate can be a double-stranded RNA (dsRNA), single stranded RNA (ssRNA), double stranded DNA (dsDNA), or single-stranded DNA (ssDNA).

A multimeric complex may comprise enhanced modification activity relative to the modification activity of a monomeric form thereof. For example, a multimeric complex comprising two polypeptides (e.g., in dimeric form) comprises greater nucleic acid binding affinity than that of either of the polypeptides provided in monomeric form. A multimeric complex may comprise one or more polypeptides fused to form a fusion protein, wherein the fusion protein is capable of (or have) different activity than that of the one or more polypeptides. In another example, a multimeric complex comprises at least two polypeptides, wherein the multimeric complex comprises greater nucleic acid binding affinity and/or modification activity than that of either of the polypeptide provided in monomeric form. A multimeric complex may have an affinity for a target sequence of a target nucleic acid and is capable of (or have) catalytic activity (e.g., nicking, substituting or otherwise editing the nucleic acid) at or near the target sequence. Multimeric complexes may be activated when complexed with a guide nucleic acid. Multimeric complexes may be activated when complexed with a target nucleic acid. Multimeric complexes may be activated when complexed with a guide nucleic acid, a target nucleic acid, or a combination thereof. In some embodiments, the multimeric complex nicks the target nucleic acid.

Various aspects of the present disclosure include compositions and methods comprising multiple polypeptides (e.g., effector proteins, effector partners, fusion proteins, or combinations thereof), and uses thereof, respectively. For example, in some embodiments, two polypeptides are provided each targeting different nucleic acid sequences. Two polypeptides may target different types of nucleic acids (e.g., a first polypeptide may target double- and single-stranded nucleic acids, and a second polypeptide may only target single-stranded nucleic acids). Two polypeptides may provide different types of activities (e.g., nucleic acid modification activity, nucleic acid expression modification activity). It is understood that when discussing the use of more than one polypeptide in compositions, systems, and methods provided herein, the multimeric complex form is also described.

In some embodiments, multimeric complexes comprise at least one polypeptide (e.g., effector protein, effector partner, or fusion protein) as described herein. In some embodiments, the multimeric complex is a dimer comprising a first polypeptide and a second polypeptide. In some embodiments, the first polypeptide and the second polypeptide comprise identical amino acid sequences. In some embodiments, the first polypeptide and the second polypeptide comprise amino acid sequences that are at least 90%, at least 92%, at least 94%, at least 96%, at least 98% identical, at least 99%, or 100% identical to each other. In some embodiments, the first polypeptide and the second polypeptide comprise amino acid sequences that are at least 90%, at least 92%, at least 94%, at least 96%, at least 98% identical, at least 99%, or 100% similar to each other.

In some embodiments, the multimeric complex is a heterodimeric complex comprising at least two polypeptides (e.g., effector proteins, effector partners, fusion proteins, or combinations thereof) of different amino acid sequences. In some embodiments, the at least two polypeptides comprise two, three, four, five, six, seven, eight, nine, or ten polypeptides. In some embodiments, the multimeric complex is a heterodimeric complex comprising a first polypeptide and a second polypeptide, wherein the amino acid sequence of the first polypeptide is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second polypeptide.

In some embodiments, the multimeric complex described herein is capable of targeting (targets) polyA signals, splice site acceptors, and start codons. In some embodiments, the multimeric complex cannot create stop codons for knock-down. In some embodiments, the multimeric complex is a dimer comprising fusion protein described herein. In some embodiments, the fusion protein comprises the effector protein described herein and the effector partner described herein. In some embodiments, the dimer is formed due to non-covalent interactions between the effector proteins of monomers. In some embodiments, N- and C-termini of “formerly active” monomer is closer to 5′ region of non-target strand, while the termini of the “other” monomer is closer to 3′ region, which results in a larger editing window of the multimeric complex having a larger editing window on the non-target strand. In some embodiments, the multimeric complex has a lower editing window for a target strand due to in accessibility for the effector partner.

### Synthesis, Isolation and Assaying

Polypeptides (e.g., effector proteins, effector partners, and fusion proteins) of the present disclosure may be synthesized, using any suitable method. In some embodiments, the polypeptides are produced in vitro or by eukaryotic cells or by prokaryotic cells. In some embodiments, the polypeptides are further processed by unfolding (e.g., heat denaturation, dithiothreitol reduction, etc.) and are further refolded, using any suitable method. In some embodiments, the nucleic acid(s) encoding the polypeptides described herein, the recombinant nucleic acid(s) described herein, the vectors described herein are produced in vitro or in vivo by eukaryotic cells or by prokaryotic cells.

Any suitable method of generating and assaying the polypeptides (e.g., effector proteins, effector partners, and fusion proteins) described herein may be used. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Fourth Ed., Cold Spring Harbor Laboratory, Cold Spring Harbor (2012); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing the polypeptide is to express recombinant nucleic acids encoding the polypeptide in a suitable microbial organism, such as a bacterial cell, a yeast cell, or other suitable cell, using methods well known in the art. Exemplary methods are also described in the Examples provided herein.

In some embodiments, a polypeptide provided herein is an isolated polypeptide (e.g., effector protein, effector partner, and fusion protein). In some embodiments, the polypeptide is isolated and purified for use in compositions, systems, and/or methods described herein. In some embodiments, methods described here include the step of isolating polypeptides described herein. Any suitable method to provide isolated polypeptides described herein may be used in the present disclosure, for example, recombinant expression systems, precipitation, gel filtration, ion-exchange, reverse-phase and affinity chromatography, and the like. Other well-known methods are described in Deutscher et al., Guide to Protein Purification: Methods in Enzymology, 2nd edition, Vol. 463, (Academic Press, (2009)). Alternatively, the isolated polypeptides of the present disclosure can be obtained using well-known recombinant methods (see, e.g., Sambrook et al., Molecular Cloning: A Laboratory Manual, Fourth Ed., Cold Spring Harbor Laboratory, Cold Spring Harbor (2012); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

In some embodiments, compositions, systems, and methods described herein further comprise a purification tag that can be attached to a polypeptide (e.g., effector protein, effector partner, and fusion protein), or a nucleic acid encoding the purification tag that can be attached to a nucleic acid encoding the polypeptide as described herein. In some embodiments, the purification tag comprises an amino acid sequence which can attach or bind with high affinity to a separation substrate and assist in isolating the polypeptide of interest from its environment, which is its biological source, such as a cell lysate. Attachment of the purification tag may be at the N or C terminus of the polypeptide. Furthermore, an amino acid sequence recognized by a protease or a nucleic acid encoding for an amino acid sequence recognized by a protease, such as TEV protease or the HRV3C protease may be inserted between the purification tag and the polypeptide, such that biochemical cleavage of the sequence with the protease after initial purification liberates the purification tag. Purification and/or isolation may be performed through high performance liquid chromatography (HPLC), exclusion chromatography, gel electrophoresis, affinity chromatography, or other purification technique. Non-limiting examples of purification tags are as described herein.

In some embodiments, polypeptides (e.g., effector proteins, effector partners, and fusion proteins) described herein are isolated from cell lysate. In some embodiments, the compositions described herein comprise 20% or more by weight, 75% or more by weight, 95% or more by weight, or 99.5% or more by weight of the polypeptide, related to the method of preparation of compositions described herein and its purification thereof, wherein percentages are upon total polypeptide content in relation to contaminants. Thus, in some embodiments, a composition comprising the polypeptide is at least 80% pure, at least 85% pure, at least 90% pure, at least 95% pure, at least 98% pure, or at least 99% pure (e.g., free of contaminants, non-engineered proteins or other macromolecules, etc.) relative to the polypeptide.

### Protospacer Adjacent Motif (PAM) Sequences

Polypeptide (e.g., effector protein, effector partner, and fusion protein) of the present disclosure may bind, nick and/or modify a target nucleic acid within or near a protospacer adjacent motif (PAM) sequence of the target nucleic acid. In some embodiments, the target nucleic acid is a double stranded nucleic acid comprising a target strand and a non-target strand. In some embodiments, binding, nicking and/or modifying occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, polypeptides described herein recognize a PAM sequence. In some embodiments, recognizing a PAM sequence comprises interacting with a sequence adjacent to the PAM. In some embodiments, a target nucleic acid comprises a target sequence that is adjacent to a PAM sequence. In some embodiments, the polypeptide does not require a PAM to bind, nick and/or modify a target nucleic acid. In some embodiments, the polypeptide does not cleave the target nucleic acid, wherein the polypeptide comprises a dCas protein.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP binds, cleaves and/or modifies the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, the PAM sequence is located 5′ of the target sequence on the non-target strand. In some embodiments, such a PAM described herein is directly adjacent to the target sequence on the target strand or the non-target strand. In some embodiments, an RNP binds, nicks and/or modifies the target strand or the non-target strand. In some embodiments, an RNP recognizes the PAM sequence, hybridizes to a target sequence of the target nucleic acid, and optionally modifies the target nucleic acid. In some embodiments, the RNP binds, nicks and/or modifies the target nucleic acid, wherein the RNP has recognized the PAM sequence, is hybridized to the target sequence of the target nucleic acid, and optionally modifies the target nucleic acid.

In some embodiments, a polypeptide (e.g., an effector protein described herein, an effector partner described herein) or a multimeric complex thereof, recognizes a PAM on a target nucleic acid. In some embodiments, multiple polypeptides of the multimeric complex recognize a PAM on a target nucleic acid. In some embodiments, at least two of the multiple polypeptides recognize the same PAM sequence. In some embodiments, at least two of the multiple polypeptides recognize different PAM sequences. In some embodiments, only one polypeptide of the multimeric complex recognizes a PAM on a target nucleic acid.

An effector protein of the present disclosure, or a multimeric complex thereof, may bind, cleave, nick, or modify a target nucleic acid within or near a protospacer adjacent motif (PAM) sequence of the target nucleic acid. In some embodiments, binding, cleavage, nicking and/or modification occurs within 1, 2, 3, 4, 5, 6, 7, 8, 9 or 10 nucleotides of a 5′ or 3′ terminus of a PAM sequence.

In some embodiments, a PAM sequence provided herein comprises any one of the nucleotide sequences recited in TABLE 4. PAMs used in compositions, systems, and methods herein are further described throughout the application. In some embodiments, the PAM is 5′-TNTR-3′ (SEQ ID NO: 65), wherein R is selected from A and G, and N is any nucleotide. In some embodiments, the PAM is 5′-NNTN-3′ (SEQ ID NO: 66), wherein N is any nucleotide. In some embodiments, the PAM is a PAM selected from PAMs represented by 5′-NNTN-3′ (SEQ ID NO: 66), wherein Nis any nucleotide. In some embodiments, the PAM is a PAM selected from PAMs represented by 5′-TNTR-3′ (SEQ ID NO: 65), wherein R is selected from A and G, and N is any nucleotide.

In some embodiments, compositions, methods and systems described herein do not comprise a PAM sequence. In some embodiments, polypeptides polypeptide (e.g., effector protein, effector partner, and fusion protein) do not recognize a PAM sequence. In some embodiments, compositions, methods and systems described herein comprise a protospacer-flanking site (PFS) sequence. A PFS sequence may be useful for the detection and/or modification of RNA.

### Nucleic Acid Systems

### Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid. Guide nucleic acids are also referred to herein as “guide RNA.” A guide nucleic acid, as well as any components thereof (e.g., spacer sequence, repeat sequence, linker nucleotide sequence, handle sequence, intermediary sequence etc.) may comprise one or more deoxyribonucleotides, ribonucleotides, biochemically or chemically modified nucleotides (e.g., one or more engineered modifications as described herein), or any combinations thereof. Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which can be a nucleotide sequence for use in a guide nucleic acid as described herein. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion. In some embodiments, a nucleotide “U” is a uracil or a IN-Methyl-Pseudouridine.

A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). A guide nucleic acid may be chemically synthesized or recombinantly produced by any suitable methods. Guide nucleic acids and portions thereof may be found in or identified from a CRISPR array present in the genome of a host organism or cell.

In general, the guide nucleic acid comprises a nucleotide sequence that is at least 80%, at least 85%, at least 90%, at least 95%, at least 98%, at least 99%, or 100% complementary to the target sequence. In general, a portion of the guide nucleic acid (i.e., the spacer sequence) has a degree of complementarity to the target sequence and hybridizes to the target sequence. In some embodiments, the guide nucleic acid comprises at least 10 contiguous nucleotides that are complementary to the target sequence in the target nucleic acid. In some embodiments, guide nucleic acid comprises a spacer sequence that is at least 80%, at least 85%, at least 90%, at least 95%, at least 98%, at least 99%, or 100% complementary to the target sequence.

In general, a guide nucleic acid comprises a first region that is not complementary to a target nucleic acid (FR) and a second region is complementary to the target nucleic acid (SR), wherein the FR and the SR are heterologous to each other. In some embodiments, FR is located 5′ to SR (FR-SR). In some embodiments, SR is located 5′ to FR (SR-FR). In some embodiments, the FR comprises one or more repeat sequence, handle sequence, intermediary sequence, or combinations thereof. In some embodiments, at least a portion of the FR interacts or binds to an effector protein. In some embodiments, the SR comprises a spacer sequence, wherein the spacer sequence can interact in a sequence-specific manner with (e.g., has complementarity with, or can hybridize to a target sequence in) a target nucleic acid.

In some embodiments, the first region, the second region, or both are about 8 nucleic acids, about 10 nucleic acids, about 12 nucleic acids, about 14 nucleic acids, about 16 nucleic acids, about 18 nucleic acids, about 20 nucleic acids, about 22 nucleic acids, about 24 nucleic acids, about 26 nucleic acids, about 28 nucleic acids, about 30 nucleic acids, about 32 nucleic acids, about 34 nucleic acids, about 36 nucleic acids, about 38 nucleic acids, about 40 nucleic acids, about 42 nucleic acids, about 44 nucleic acids, about 46 nucleic acids, about 48 nucleic acids, or about 50 nucleic acids long.

In some embodiments, the first region, the second region, or both are from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, or from about 8 to about 50 nucleic acids long.

In some embodiments, the first region, the second region, or both comprise a GC content of about 1%, about 5%, about 10%, about 15%, about 20%, about 25%, about 30%, about 40%, about 50%, about 60%, about 70%, about 80%, about 90%, about 95%, or about 99%. In some embodiments, the first region, the second region, or both comprise a GC content of from about 1% to about 95%, from about 5% to about 90%, from about 10% to about 80%, from about 15% to about 70%, from about 20% to about 60%, from about 25% to about 50%, or from about 30% to about 40%.

In some embodiments, the first region, the second region, or both have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region, the second region, or both have a melting temperature of from about 35° C. to about 40° C., from about 35° C. to about 45° C., from about 35° C. to about 50° C., from about 35° C. to about 55° C., from about 35° C. to about 60° C., from about 35° C. to about 65° C., from about 35° C. to about 70° C., from about 35° C. to about 75° C., from about 35° C. to about 80° C., or from about 35° C. to about 85° C.

In some embodiments, the compositions, systems, and methods of the present disclosure further comprise an additional nucleic acid, wherein a portion of the additional nucleic acid at least partially hybridizes to the first region of the guide nucleic acid. In some embodiments, the additional nucleic acid is at least partially hybridized to the 5′ end of the second region of the guide nucleic acid. In some embodiments, an unhybridized portion of the additional nucleic acid, at least partially, interacts with an effector protein or polypeptide.

The guide nucleic acid may also form complexes as described through herein. For example, a guide nucleic acid hybridizes to another nucleic acid, such as target nucleic acid, or a portion thereof. In another example, a guide nucleic acid complexes with an effector protein. In such embodiments, a guide nucleic acid-effector protein complex is described herein as an RNP. In some embodiments, when in a complex, at least a portion of the complex binds, recognizes, and/or hybridizes to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that, in some embodiments, a RNP hybridizes to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid comprises or forms intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by hybridizing to different target sequences. In some embodiments, a first guide nucleic acid hybridizes within a location of the target nucleic acid that is different from where a second guide nucleic acid hybridizes the target nucleic acid. In some embodiments, the first loci and the second loci of the target nucleic acid are located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid are located between 100 and 200, 200 and 300, 300 and 400, 400 and 500, 500 and 600, 600 and 700, 700 and 800, 800 and 900 or 900 and 1000 nucleotides apart. In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, compositions, systems, and methods comprising multiple guide nucleic acids or uses thereof comprise multiple effector proteins, wherein the effector proteins are identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, signal, and the like. In some embodiments, a target sequence is a eukaryotic sequence.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

In some embodiments, guide nucleic acids comprise one or more nucleotide sequences as described herein (e.g., TABLE 5, TABLE 6, and TABLE 8). Such nucleotide sequences described herein (e.g., TABLE 5, TABLE 6, and TABLE 8) may be described as a nucleotide sequence of either DNA or RNA, however, no matter the form of the sequence described, it is readily understood that such nucleotide sequences may be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLE 5, TABLE 6, and TABLE 8) also discloses the complementary nucleotide sequence, the reverse nucleotide sequence, and the reverse complement nucleotide sequence, any one of which may be a nucleotide sequence for use in a guide nucleic acid as described herein. In some embodiments, guide nucleic acid sequence(s) comprises one or more nucleotide alterations at one or more positions in any one of the sequences described herein. Alternative nucleotides may be any one or more of A, C, G, T or U, or a deletion, or an insertion.

In some embodiments, the guide nucleic acid comprises a nucleotide sequence that is capable of hybridizing (hybridizes) to a target sequence in a target nucleic acid, wherein the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the guide nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

In some embodiments, guide nucleic acids described herein comprise an MS2 aptamer sequence. In some embodiments, the MS2 aptamer sequence comprises a nucleotide sequence that is at least 65%, at least 70%, at least 75%, at least 80%, at least 85%, at least 90%, at least 95%, or 100% identical to ACAUGAGGAUCACCCAUGU (SEQ ID NO: 218). In some embodiments, proteins described herein are fused to an MS2 coat protein (MCP) or an MCP domain that is capable of binding (or binds) the MS2 aptamer sequence, thereby bringing the protein to the guide nucleic acid. In some embodiments, the protein could be used as an effector partner described herein. In some embodiments, the protein is an exonuclease. In some embodiments, the MS2 aptamer sequence comprises a nucleotide sequence that is at least 65%, at least 70%, at least 75%, at least 80%, at least 85%, at least 90%, at least 95%, or 100% similar to ACAUGAGGAUCACCCAUGU (SEQ ID NO: 218). In some embodiments, the effector partner comprises one or more of the MCP domain and sbcB exonuclease. The exemplary sequences for MCP domain and sbcB exonuclease are recited in TABLE 9. In some embodiments, the effector partner comprises an MCP domain, wherein the MCP domain comprises an amino acid sequence that is at least 65%, at least 70%, at least 75%, at least 80%, at least 85%, at least 90%, at least 95%, at least 99%, or 100% identical to SEQ ID NO: 220, and an sbcB exonuclease having at least at least 65%, at least 70%, at least 75%, at least 80%, at least 85%, at least 90%, at least 95%, at least 99%, or 100% identical to SEQ ID NO: 221, wherein the effector partner binds the MS2 aptamer sequence and the sbcB exonuclease has exonuclease activity. In some embodiments, the effector partner comprises an MCP domain, wherein the MCP domain comprises an amino acid sequence that is at least 65%, at least 70%, at least 75%, at least 80%, at least 85%, at least 90%, at least 95%, at least 99%, or 100% similar to SEQ ID NO: 220, wherein the effector partner binds the MS2 aptamer sequence.

### Repeat Sequences

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that interacts with an effector protein. In some embodiments, a repeat sequence is connected to another sequence of a guide nucleic acid, such as an intermediary sequence, that is capable of non-covalently interacting (or non-covalently interacts) with an effector protein. In some embodiments, a repeat sequence includes a nucleotide sequence that is capable of forming (or forms) a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which comprises a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence includes a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events hybridizes over one or more segments such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of any one of the repeat sequences in TABLE 5. In some embodiments, the repeat sequence is at least 85% identical to any one of nucleotide sequences recited in TABLE 5. In some embodiments, a repeat sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of any one of the nucleotide sequences recited in TABLE 5.

In some embodiments, a repeat sequence comprises one or more nucleotide alterations at one or more positions in the nucleotide sequence recited in TABLE 5. Alternative nucleotides can be any one or more of A, C, G, T or U, or a deletion, or an insertion.

### Spacer Sequences

Guide nucleic acids described herein may comprise one or more spacer sequences. In some embodiments, a spacer sequence is capable of hybridizing (or hybridizes) to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence functions to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to at least about 25, or at least about 15 to about 25 linked nucleotides. In some embodiments, the spacer sequence comprises 15-28 linked nucleotides. In some embodiments, a spacer sequence comprises 10-26, 10-26, 10-24, 10-22, 10-20, 10-18, 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, the spacer sequence comprises 10, 11, 12, 13, 14, 15, 16, 17, 18, 19, 20, 21, 22, 23, 24, 25, 26, 27, 28, 29, 30, or more nucleotides. In some embodiments, the spacer sequence comprises a nucleotide sequence of 10 to 24 linked nucleotides. In some embodiments, the spacer sequence comprises a nucleotide sequence of 11 to 14 linked nucleotides. In some embodiments, the spacer sequence comprises a nucleotide sequence of 21 linked nucleotides. In some embodiments, the spacer sequence comprises a nucleotide sequence of 20 linked nucleotides. In some embodiments, the spacer sequence comprises a nucleotide sequence of 12 linked nucleotides. In some embodiments, the spacer sequence comprises a nucleotide sequence of 13 linked nucleotides.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence is capable of hybridizing (or hybridizes) to an equal length portion of a target nucleic acid (e.g., a target sequence). In some embodiments, a target nucleic acid, such as DNA or RNA, is a cancer gene or gene associated with a genetic disorder, or an amplicon thereof, as described herein. In some embodiments, a target nucleic acid is a gene selected from TABLE 9. In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid selected from TABLE 9. In some embodiments, a spacer sequence comprises a nucleotide sequence that is complementary to a target sequence of a target nucleic acid selected from TABLE 9 in a range of from 65% to 100%, from 70% to 100%, from 75% to 100%, from 80% to 100%, from 85% to 100%, from 90% to 100%, from 95% to 100%, from 65% to 95%, from 70% to 95%, from 75% to 95%, from 80% to 95%, from 85% to 95%, from 90% to 95%, from 65% to 90%, from 70% to 90%, from 75% to 90%, from 80% to 90%, or from 85% to 90%. In some embodiments, a target nucleic acid is a nucleic acid associated with a disease or syndrome recited in TABLE 10. In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid associated with a disease or syndrome recited in TABLE 10. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing (or hybridizes) to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

In some embodiments, a spacer length effects activity (e.g., a binding activity, a catalytic activity, or a combination thereof) for a target nucleic acid. In some embodiments, the target nucleic acid comprises a target strand and a non-target strand. In some embodiments, a guide RNA having a spacer length of 10-14 nucleotides relative to a guide RNA having a spacer length of 17-22 nucleotides favors nicking activity over double strand break activity. In some embodiments, a guide RNA having a spacer length of 10-14 nucleotides relative to a guide RNA having a spacer length of 17-22 nucleotides favors nicking of a target strand relative to nicking of a non-target strand. In some embodiments, a guide RNA having a spacer length of about 10-14 nucleotides reduces target nucleic acid editing efficiency relative to a guide RNA having a spacer length of 17-22 nucleotides.

It is understood that the spacer sequence of a spacer sequence need not be 100% complementary to that of a target sequence of a target nucleic acid to hybridize or hybridize specifically to the target sequence. For example, the spacer sequence comprises at least one alteration, such as a substituted or modified nucleotide, that is not complementary to the corresponding nucleotide of the target sequence.

In some embodiments, a spacer sequence comprises a nucleotide sequence that hybridizes to an equal length portion of any one of the genes selected from ANGPTL3, APOC3, PCSK9, LDHA, HSD17B13, HAO1, CNBP and LPA. In some embodiments, a spacer sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of any one of the spacer sequences in TABLE 21, TABLE 22, TABLE 23, TABLE 24, TABLE 25, TABLE 26, TABLE 27 and TABLE 28. In some embodiments, the spacer sequence is at least 85% identical to any one of nucleotide sequences recited in TABLE 21, TABLE 22, TABLE 23, TABLE 24, TABLE 25, TABLE 26, TABLE 27 and TABLE 28. In some embodiments, a spacer sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of any one of the nucleotide sequences recited in TABLE 21, TABLE 22, TABLE 23, TABLE 24, TABLE 25, TABLE 26, TABLE 27 and TABLE 28.

In some embodiments, a spacer sequence comprises one or more nucleotide alterations at one or more positions in the nucleotide sequence recited in TABLE 21, TABLE 22, TABLE 23, TABLE 24, TABLE 25, TABLE 26, TABLE 27 and TABLE 28. Alternative nucleotides can be any one or more of A, C, G, T or U, or a deletion, or an insertion.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises any one of the nucleotide sequences recited in TABLE 7.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Intermediary Sequences

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region hybridizes to the 3′ region. In some embodiments, the 5′ region of the intermediary sequence does not hybridize to the 3′ region. In some embodiments, the intermediary RNA comprises an MS2 aptamer sequence. In some embodiments, the MS2 aptamer sequence is located within the intermediary RNA. In some embodiments, the MS2 aptamer sequence is located between 5′ end and 3′ end of the intermediary RNA.

In some embodiments, the hairpin region comprises a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

In some embodiments, an intermediary sequence comprises a nucleotide sequence that is at least 65%, at least 70%, at least 75%, at least 80%, at least 85%, at least 90%, at least 95%, at least 97%, or at least 98%, at least 99%, or 100% identical to any one of the intermediary sequences in TABLE 6. In some embodiments, an intermediary sequence comprises at least 5, at least 6, at least 7, at least 8, at least 9, at least 10, at least 20, at least 30, at least 40, at least 50, at least 60, at least 70, at least 80, at least 90, at least 100, at least 110, at least 120, at least 130, or at least 140 contiguous nucleotides of any one of the intermediary sequences recited in TABLE 6.

### Handle Sequences

Guide nucleic acids described herein may comprise one or more handle sequences. In some embodiments, the handle sequence comprises an intermediary sequence. In such instances, at least a portion of an intermediary sequence non-covalently bonds with an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such instances, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided in TABLE 7. In some embodiments, the linker comprises a sequence of 5′-GAAA-3′. In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

A handle sequence may comprise or form a secondary structure (e.g., one or more hairpin loops) that facilitates the binding of an effector protein to a guide nucleic acid and/or modification activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). An effector protein may recognize a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, a handle sequence comprises a nucleotide sequence that is at least 65%, at least 70%, at least 75%, at least 80%, at least 85%, at least 90%, at least 95%, at least 97%, or at least 98%, at least 99%, or 100% identical to any one of the handle sequences in TABLE 8. In some embodiments, a handle sequence comprises at least 5, at least 6, at least 7, at least 8, at least 9, at least 10, at least 20, at least 30, at least 40, at least 50, at least 60, at least 70, at least 80, at least 90, at least 100, at least 110, at least 120, at least 130, or at least 140 contiguous nucleotides of any one of the handle sequences recited in TABLE 8.

In some embodiments, compositions, systems and methods of the disclosure comprises a guide nucleic acid comprising a handle sequence comprises an MS2 aptamer sequence. In some embodiments, the MS2 aptamer sequence comprises a nucleotide sequence that is at least 65%, at least 70%, at least 75%, at least 80%, at least 85%, at least 90%, at least 95%, or 100% identical to ACAUGAGGAUCACCCAUGU (SEQ ID NO: 218). In some embodiments, the handle sequence comprising the MS2 aptamer sequence comprises a nucleotide sequence that is at least 65%, at least 70%, at least 75%, at least 80%, at least 85%, at least 90%, at least 95%, or 100% identical to any one of SEQ ID NO: 118 through SEQ ID NO: 150. In some embodiments, the handle sequence comprises the intermediary RNA, the repeat sequence, and the MS2 aptamer sequence. In some embodiments, the handle sequence comprises, from 5′ to 3′ direction, the MS2 aptamer sequence, the intermediary RNA, and the repeat sequence. In some embodiments, the handle sequence comprises, from 5′ to 3′ direction, the intermediary RNA, the MS2 aptamer sequence, and the repeat sequence.

### Single Nucleic Acid Systems

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) of the guide nucleic acid non-covalently interacts with the one or more polypeptides described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

In contrast to the single nucleic system, in some embodiments, a dual nucleic acid system comprises a crRNA and a transactivating RNA (tracrRNA), wherein the tracrRNA is capable of hybridizing (or hybridizes), at least partially, to a crRNA to form a tracrRNA-crRNA duplex. The tracrRNA-crRNA duplex is capable of interacting (or interacts) with an effector protein to form a complex (e.g., an RNP complex). In some embodiments, a tracrRNA or a tracrRNA-crRNA duplex transactivates the effector protein, and thereby, enabling the effector protein to have a binding and/or nuclease activity on a target nucleic acid.

crRNA

In some embodiments, a guide nucleic acid comprises a crRNA. In some embodiments, the guide nucleic acid is the crRNA. In general, a crRNA comprises a first region (FR) and a second region (SR), wherein the FR of the crRNA comprises a repeat sequence, and the SR of the crRNA comprises a spacer sequence. In some embodiments, at least a portion of the first region hybridizes to a target sequence of a target nucleic acid and at least a portion of the second region interacts with an effector protein either directly (by being bound by an effector protein) or indirectly (e.g., by hybridization with a second nucleic acid molecule that can be bound by an effector). In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting (or connects) a crRNA to an effector protein. In some embodiments, a single nucleic acid system comprises a guide nucleic acid comprising a crRNA linked to another nucleotide sequence that is capable of being non-covalently bond (or bound) by an effector protein. In such embodiments, a repeat sequence of a crRNA can be linked to an intermediary sequence. In some embodiments, a single nucleic acid system comprises a guide nucleic acid comprising a crRNA and an intermediary sequence.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

In some embodiments, a crRNA sequence comprises a repeat sequence that is at least 65%, at least 70%, at least 75%, at least 80%, at least 85%, at least 90%, at least 95%, at least 97%, or at least 98%, at least 99%, or 100% identical to any one of the sequences set forth in TABLE 5. In some embodiments, a crRNA sequence comprises at least 5, at least 6, at least 7, at least 8, at least 9, or at least 10 contiguous nucleotides of any one of the repeat sequences recited in TABLE 5.

sgRNA

In some embodiments, a guide nucleic acid comprises a sgRNA. In some embodiments, a guide nucleic acid is a sgRNA. In some embodiments, a sgRNA comprises a first region (FR) and a second region (SR), wherein the FR comprises a handle sequence and the SR comprises a spacer sequence. In some embodiments, the handle sequence and the spacer sequences are directly connected to each other (e.g., covalent bond (phosphodiester bond)). In some embodiments, the handle sequence and the spacer sequence are connected by a linker.

In some embodiments, a sgRNA comprises one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g, covalently linked, such as through a phosphodiester bond) In some embodiments, a repeat sequence and a spacer sequence are linked in an sgRNA by any suitable linker, examples of which are provided herein.

In some embodiments, a sgRNA sequence comprises a nucleotide sequence that is at least 65%, at least 70%, at least 75%, at least 80%, at least 85%, at least 90%, at least 95%, at least 97%, or at least 98%, at least 99%, or 100% identical to any one of the nucleotide sequences recited in TABLE 5, TABLE 6, and TABLE 8. In some embodiments, a sgRNA sequence comprises a repeat sequence comprising at least 5, at least 6, at least 7, at least 8, at least 9, or at least 10 contiguous nucleotides of any one of the nucleotide sequences recited in TABLE 5. In some embodiments, a sgRNA sequence comprises an intermediary sequence comprising at least 5, at least 6, at least 7, at least 8, at least 9, or at least 10 contiguous nucleotides of any one of the nucleotide sequences recited in TABLE 6. In some embodiments, a sgRNA sequence comprises a handle sequence comprising at least 5, at least 6, at least 7, at least 8, at least 9, or at least 10 contiguous nucleotides of any one of the nucleotide sequences recited in TABLE 8.

In some embodiments, compositions, systems and methods of the disclosure comprises an sgRNA comprising a spacer sequence further comprises an MS2 aptamer sequence. In some embodiments, the MS2 aptamer sequence comprises a nucleotide sequence that is at least 65%, at least 70%, at least 75%, at least 80%, at least 85%, at least 90%, at least 95%, or 100% identical to ACAUGAGGAUCACCCAUGU (SEQ ID NO: 218). In some embodiments, the MS2 aptamer sequence is 5′ to the spacer sequence. In some embodiments, the MS2 aptamer sequence is 3′ to the spacer sequence. In some embodiments, the sgRNA comprises a nucleotide sequence that is at least 65%, at least 70%, at least 75%, at least 80%, at least 85%, at least 90%, at least 95%, or 100% identical to any one of the sequences recited in TABLE 17, wherein the aptamer sequence comprises a nucleotide sequence that is at least 65%, at least 70%, at least 75%, at least 80%, at least 85%, at least 90%, at least 95%, or 100% identical to ACAUGAGGAUCACCCAUGU (SEQ ID NO: 218).

### Engineered Modifications

Polypeptides (e.g., effector proteins) and nucleic acids (e.g., engineered guide nucleic acids) can be further modified as described herein. Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues (e.g., phosphotyrosine, phosphoserine, or phosphothreonine).

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, e.g., cysteines may be used to make thioethers, histidines for linking to a metal ion complex, carboxyl groups for forming amides or esters, amino groups for forming amides, and the like.

Modifications can further include changing of nucleic acids described herein (e.g., engineered guide nucleic acids) to provide the nucleic acid with a new or enhanced feature, such as improved stability. Such modifications of a nucleic acid include a base editing, a base modification, a backbone modification, a sugar modification, or combinations thereof. In some embodiments, the modifications can be of one or more nucleotides, nucleosides, or nucleobases in a nucleic acid.

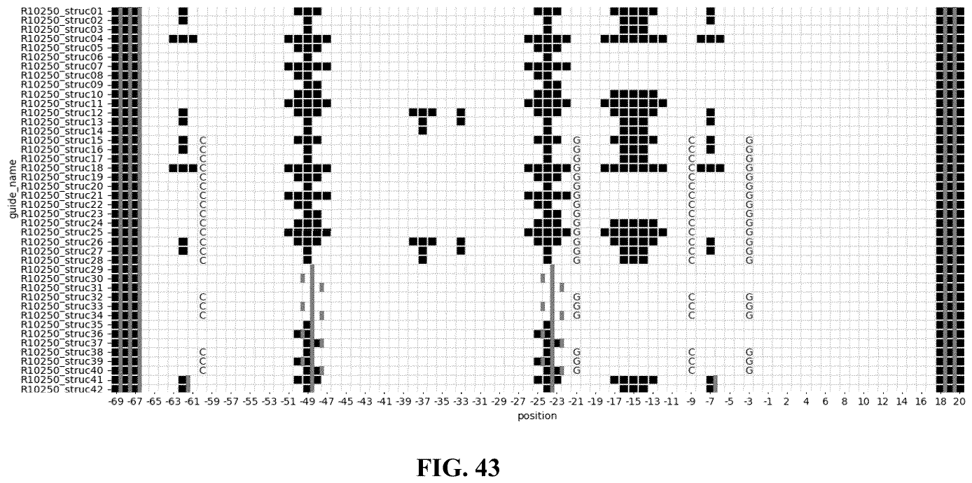

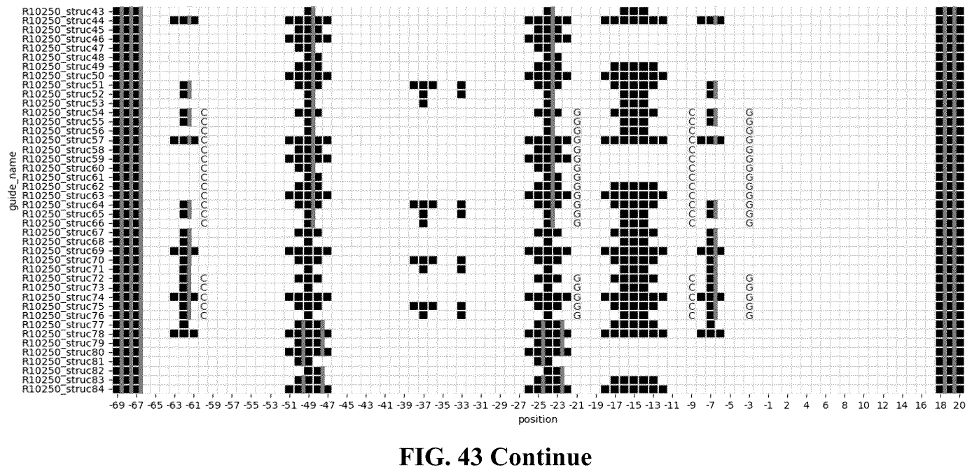

In some embodiments, nucleic acids (e.g., nucleic acids encoding effector proteins, engineered guide nucleic acids, or nucleic acids encoding engineered guide nucleic acids) described herein comprise one or more modifications comprising: 2′O-methyl modified nucleotides (e.g., 2′-O-Methyl (2-O-Me) sugar modifications); 2′ fluoro modified nucleotides (e.g., 2′-fluoro (2′-F) sugar modifications); locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof. In some embodiments, a guide nucleic acid has a modification pattern as depicted in FIG. 43 and described in Example 38.

In some embodiments, repeat sequences described herein comprise at least three nucleotides that are modified. In some embodiments, repeat sequences described herein comprise at least three consecutive nucleotides from the 5′-end of the guide nucleic acid that are modified. In some embodiments, spacer sequences described herein comprise at least three nucleotides that are modified. In some embodiments, spacer sequences described herein comprise at least three consecutive nucleotides from the 3′-end of the guide nucleic acid that are modified. In some embodiments, guide nucleic acids described herein comprise at least three nucleotides that are modified. In some embodiments, guide nucleic acids described herein comprise at least six nucleotides that are modified.

In some embodiments, guide nucleic acids described herein comprise one or more 2′-O-Methyl (2-O-Me) sugar modifications, one or more phosphorothioate (PS) backbone modifications, one or more U-A>G-C covariations, or combinations thereof. In some embodiments, the one or more 2-O-Me sugar modification, PS backbone modification, one or more U-A>G-C covariations, or combinations thereof are contained within a portion of a guide nucleic acid (e.g., repeat sequence, intermediary sequence, handle sequence) that at least partially interacts with an effector protein described herein. In some embodiments, the one or more U-A>G-C covariations contained within the portion of a guide nucleic acid that at least partially interacts with an effector protein described herein modifies the interaction between the effector protein and the guide nucleic acid. In some embodiments, the one or more U-A>G-C covariations increases the interaction between the effector protein and the guide nucleic acid relative to a guide nucleic acid not having the one or more U-A>G-C covariations. Alternatively, in some embodiments, the one or more U-A>G-C covariations decreases the interaction between the effector protein and the guide nucleic acid relative to a guide nucleic acid not having the one or more U-A>G-C covariations. Such modifications in interaction, in some embodiments, result in modifications of activities of the effector protein and guide nucleic acid complex. In some embodiments, the one or more 2-O-Me sugar modification, PS backbone modification, or combinations thereof are contained within a portion of a guide nucleic acid (e.g., spacer sequence) that at least partially interacts with a target sequence of a target nucleic acid.

### Nucleic Acid Modification Systems & Nucleic Acid Expression Modification Systems

Provided herein are systems for modifying target nucleic acids, modifying expression of target nucleic acids, modifying expression and/or functionality of proteins associated with the target nucleic acids or expression of the target nucleic acids, modifying other nucleic acids associated with the target nucleic acids or expression of the target nucleic acids, or combinations thereof. In some embodiments, the systems comprise one or more effector proteins described herein or nucleic acids encoding the effector proteins, one or more guide nucleic acids or nucleic acids encoding the guide nucleic acids, and one or more effector partners or nucleic acids encoding the effector partners.

The modification of the target nucleic acid generated by the system described herein may, as a non-limiting example, result in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization). Accordingly, in some embodiments, provided herein are methods of editing a target nucleic acid using the systems described herein.

Also provided herein are methods of modulating expression of a target nucleic acid using the systems described herein. Further provided herein are methods of modulating the activity of a translation product of a target nucleic acid using the systems described herein.

In some embodiments, an ability of the system described herein to edit a target nucleic acid depends upon the effector protein being complexed with a guide nucleic acid, the guide nucleic acid being hybridized to a target sequence of the target nucleic acid, the distance between the target sequence and a PAM sequence, or combinations thereof. In some embodiments, an ability of the system described herein to edit a target nucleic acid depends upon distance between the effector partner and the target nucleic acid, distance between the effector partner and the effector protein, or a combination thereof. In some embodiments, the effector protein is fused to the effector partner. In some embodiments, the effector protein is fused to the effector partner by a linker. Accordingly, in some embodiments, the ability of the system described herein to edit a target nucleic acid depends upon a size of the linker. In some embodiments, the linker described herein comprises an amino acid sequence ranging from 1 to 300, from 1 to 250, from 1 to 200, from 1 to 150, from 1 to 100, from 1 to 50, from 1 to 25, from 1 to 10, from 10 to 300, from 10 to 250, from 10 to 200, from 10 to 150, from 10 to 100, from 10 to 50, from 10 to 25, from 25 to 300, from 25 to 250, from 25 to 200, from 25 to 150, from 25 to 100, from 25 to 50, from 50 to 300, from 50 to 250, from 50 to 200, from 50 to 150, from 50 to 100, from 100 to 300, from 100 to 250, from 100 to 200, from 100 to 150, from 150 to 300, from 150 to 250, from 150 to 200, from 200 to 300, from 200 to 250, or from 250 to 300. A target nucleic acid comprises a target strand and a non-target strand. Accordingly, in some embodiments, the system edits a target strand and/or a non-target strand of a target nucleic acid. In some embodiments, the target nucleic acid can be a single-stranded DNA (ssDNA), double-stranded DNA (dsDNA), single-stranded RNA (ssRNA), double-stranded RNA (dsRNA), or a combination thereof.

### Vectors and Multiplexed Expression Vectors

Compositions, systems, and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises a polypeptide(s) (e.g., effector protein(s), effector partner(s), fusion protein(s), or combinations thereof), guide nucleic acid(s), and target nucleic acid(s). In some embodiments, the component comprises a nucleic acid encoding the polypeptide, and a guide nucleic acid or a nucleic acid encoding the guide nucleic acid. In some embodiments, a vector is part of a vector system. The vector system may comprise a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., a polypeptide (e.g., an effector protein, an effector partner, a fusion protein, or a combination thereof) a guide nucleic acid, and/or a target nucleic acid) are each encoded by different vectors of the system.

In some embodiments, a vector comprises a nucleotide sequence encoding one or more polypeptides (e.g., effector proteins, effector partners, fusion proteins, or combinations thereof) as described herein. In some embodiments, the one or more polypeptides comprise at least two polypeptides. In some embodiments, the at least two polypeptides are the same. In some embodiments, the at least two polypeptides are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more polypeptides.

In some embodiments, a vector encodes one or more of any system components, including but not limited to polypeptides (e.g., effector proteins, effector partners, fusion proteins, or combinations thereof), guide nucleic acids, and target nucleic acids as described herein. In some embodiments, a system component encoding sequence is operably linked to a promoter that is operable in a target cell, such as a eukaryotic cell. In some embodiments, a vector encodes 1, 2, 3, 4 or more of any system components. For example, a vector encodes two or more guide nucleic acids, wherein each guide nucleic acid comprises a different sequence. A vector may encode the polypeptide and the guide nucleic acid.

In some embodiments, a vector comprises one or more guide nucleic acids, or a nucleotide sequence encoding the one or more guide nucleic acids as described herein. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector comprises or encodes one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector comprises or encodes for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can include a promoter-a regulatory region on a nucleic acid, such as a DNA sequence, capable of initiating (or initiates) transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes a polypeptide (e.g., an effector protein, an effector partner, a fusion protein, or a combination thereof) to a cell may include nucleic acid sequences that encode for selectable markers in the target cells, so as to identify cells that have taken up the guide nucleic acid and/or the polypeptide.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, TRE, UAS, Ac5, Polyhedrin, CaMKIIa, GAL1-10, H1, TEF1, GDS, ADH1, CaMV35S, HSV TK, Ubi, U6, MNDU3, MSCV, MND and CAG.

In some embodiments, some promoters (e.g., U6, enhanced U6, Hl and 7SK) prefers the nucleic acid being transcribed having “g” nucleotide at the 5′ end of the coding sequence. Accordingly, when such coding sequence is expressed, it comprises an additional “g” nucleotide at 5′ end. In some embodiments, vectors provided herein comprise a promotor driving expression or transcription of any one of the guide nucleic acids described herein (e.g., TABLE 5, TABLE 6 and TABLE 8) further comprises “g” nucleotide at 5′ end of the guide nucleic acid, wherein the promotor is selected from U6, enhanced U6, Hl and 7SK.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing a polypeptide (e.g., an effector protein, an effector partner, a fusion protein, or a combination thereof) is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA. In some embodiments, a vector comprising the recombinant nucleic acid as described herein, wherein the vector is a viral vector, an adeno associated viral (AAV) vector, a retroviral vector, or a lentiviral vector. In some embodiments, a vector described herein or a recombinant nucleic acid described herein is comprised in a cell. In some embodiments, a recombinant nucleic acid integrated into a genomic DNA sequence of the cell, wherein the cell is a eukaryotic cell or a prokaryotic cell.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more polypeptides (e.g., effector proteins, effector partners, fusion proteins, or combinations thereof), and one or more guide nucleic acids or a nucleic acid encoding the one or more guide nucleic acid, are provided in the single expression vector. In some embodiments, components, such as the guide nucleic acid and the polypeptide, are encoded by the same vector. In some embodiments, an effector protein (or a nucleic acid encoding same) and/or an engineered guide nucleic acid (or a nucleic acid that, when transcribed, produces same) are not co-administered with an effector partner (or a nucleic acid encoding the same) and/or a fusion protein (or a nucleic acid encoding the same) in a single vehicle. In some embodiments, an effector protein (or a nucleic acid encoding same), an engineered guide nucleic acid (or a nucleic acid that, when transcribed, produces same), an effector partner (or a nucleic acid encoding the same), and/or a fusion protein (or a nucleic acid encoding the same) are administered in one or more or two or more vehicles, such as one or more, or two or more expression vectors.

In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more polypeptides (e.g., effector proteins, effector partners, fusion proteins, or combinations thereof), and one or more guide nucleic acids or a nucleic acid encoding the one or more guide nucleic acids, or a variant thereof is provided in a different vector. In some embodiments, the nucleic acid encoding the polypeptide, and a guide nucleic acid or a nucleic acid encoding the guide nucleic acid are provided in different vectors. In some embodiments, the effector partner is encoded by a different vector than the vector encoding the effector protein and the guide nucleic acid.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate a nucleic acid (e.g., DNA or RNA) encoding one or more of the components as described herein. In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28 (3): 146-157). In some embodiments, compositions and methods comprise a lipid, polymer, nanoparticle, or a combination thereof, or use thereof, to introduce one or more effector proteins, one or more guide nucleic acids, one or more effector partners, one or more fusion proteins or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino) propyl)benzene-1,3,5-tricarboxamide (TT3), 1,2-Dioleoyl-sn-glycero-3-phosphocholine (DOPC), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), derivatives, analogs, or variants thereof.

In some embodiments, the LNP comprises one or more ionizable lipid. Such ionizable lipids include, but are not limited to: 4-(dimethylamino)-butanoic acid, (10Z,13Z)-1-(9Z,12Z)-9,12-octadecadien-1-yl-10,13-nonadecadien-1-yl ester (DLin-MC3-DMA, CAS No. 1224606-06-7); N,N-dimethyl-2,2-di-(9Z,12Z)-9,12-octadecadien-1-yl-1,3-dioxolane-4-ethanamine (DLin-KC2-DMA, CAS No. 1190197-97-7); 8-[(2-hydroxyethyl) [6-oxo-6-(undecyloxy) hexyl]amino]-octanoic acid, 1-octylnonyl ester (SM-102, CAS No. 2089251-47-6); 8-[(2-hydroxyethyl) [8-(nonyloxy)-8-oxooctyl]amino]-octanoic acid, 1-octylnonyl ester (Lipid 5, CAS No. 2089251-33-0); 1,1′-[2-[4-[2-[2-[bis(2-hydroxydodecyl)amino]ethyl](2-hydroxydodecyl)amino]ethyl]-1-piperazinyl]ethyl]imino]bis-2-dodecanol (C12-200, CAS No. 1220890-25-4); 2-hexyl-decanoic acid, 1,1′-[[(4-hydroxybutyl)imino]di-6,1-hexanediyl]ester (ALC-0315, CAS No. 2036272-55-4); 9,12-octadecadienoic acid, (9Z,12Z)-1,1′,1″,1′″-[(3,6-dioxo-2,5-piperazinediyl)bis(4,1-butanediylnitrilodi-4,1-butanediyl)]ester (OF-C4-Deg-Lin, CAS No. 1853203-01-6); bis(2-(dodecyldisulfaneyl)ethyl) 3,3′-((3-methyl-9-oxo-10-oxa-13,14-dithia-3,6-diazahexacosyl) azanediyl)dipropionate (BAMEA-O16B, CAS No. 2490668-30-7); 3,6-bis[4-[bis[(9Z,12Z)-2-hydroxy-9,12-octadecadien-1-yl]amino]butyl]-2,5-piperazinedione (OF-02, CAS No. 1883431-67-1); tetrakis(8-methylnonyl) 3,3′,3″,3″-(((methylazanediyl)bis(propane-3,1-diyl))bis(azanetriyl))tetrapropionate (3060i10, CAS No. 2322290-93-5); tetrakis(2-(octyldisulfaneyl)ethyl) 3,3′,3″,3′″-(((methylazanediyl)bis(propane-3,1-diyl))bis(azanetriyl))tetrapropionate (306-012B, CAS No. 2566523-06-4); bis(2-butyloctyl) 10-(N-(3-(dimethylamino)propyl)nonanamido)nonadecanedioate (Lipid A9, CAS No. 2036272-50-9); Arcturus Lipid 2,2 (8,8) 4C CH3 (ATX-0114, CAS No. 2230647-28-4)); di((Z)-non-2-en-1-yl) 8,8′-((2-((2-(dimethylamino)ethyl)thio)acetyl)azanediyl)dioctanoate (ATX-001, CAS No. 1777792-33-2); di((Z)-non-2-en-1-yl) 8,8′-((((2-(dimethylamino)ethyl)thio) carbonyl) azanediyl)dioctanoate (ATX-002, CAS No. 1777792-34-3); Genevant CL1 (CAS No. 1450888-71-7); LP01; hexa (octan-3-yl) 9,9′,9″,9′″,9″ “,9”-((((benzene-1,3,5-tricarbonyl)yris(azanediyl))tris(propane-3,1-diyl)) tris(azanetriyl))hexanonanoate (FTT5); 5A2-SC8 (CAS No. 1857341-90-2); COATSOME® SS-OP; derivatives; analogs; or variants thereof. In some embodiments, the LNP comprise a combination of two, three, four, five or more of the foregoing ionizable lipids.

In some embodiments, the LNP has a negative net overall charge prior to complexation with one or more of a guide nucleic acid, a nucleic acid encoding the one or more guide nucleic acid, a nucleic acid encoding a polypeptide (e.g., an effector protein, an effector partner, a fusion protein, or a combination thereof). In some embodiments, the inner core is a hydrophobic core. In some embodiments, the one or more of a guide nucleic acid, the nucleic acid encoding the one or more guide nucleic acid, the nucleic acid encoding the polypeptide forms a complex with one or more of the cationic lipids and the ionizable lipids. In some embodiments, the nucleic acid encoding the polypeptide or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

In some embodiments, the LNP described herein comprises nucleic acids (e.g., DNA or RNA) encoding an effector protein described herein, an effector partner described herein, a fusion protein described herein, a guide nucleic acid described herein, or combinations thereof. In some embodiments, the LNP comprises an mRNA that produces an effector protein described herein, an effector partner described herein, or a fusion protein described herein when translated. In some embodiments, the LNP comprises chemically modified guide nucleic acids.

In some embodiments, LNPs described herein comprises one or more ionizable lipids, phospholipids, cholesterols, and PEG lipids. In some embodiments, the LNP comprises one or more ionizable lipids at a molar ratio ranging from 20 to 60, from 25 to 60, from 30 to 60, from 35 to 60, from 40 to 60, from 45 to 60, from 50 to 60, from 55 to 60, from 20 to 55, from 25 to 55, from 30 to 55, from 35 to 55, from 40 to 55, from 45 to 55, or from 50 to 55. In some embodiments, the LNP comprises one or more cholesterols at a molar ratio ranging from 5 to 20, from 7 to 20, from 5 to 17, or from 7 to 17. In some embodiments, the LNP comprises one or more cholesterols at a molar ratio ranging from 30 to 65, from 35 to 65, from 40 to 65, from 45 to 65, from 50 to 65, from 55 to 65, from 60 to 65, from 30 to 60, from 35 to 60, from 40 to 60, from 45 to 60, from 50 to 60, or from 55 to 60. In some embodiments, the LNP comprises one or more PEG lipids at a molar ratio ranging from 0.5 to 5, from 1.5 to 5, from 2.5 to 5, from 0.5 to 4, from 1.5 to 4, from 2.5 to 4, from 0.5 to 3, from 1.5 to 3, or from 2.5 to 3. In some embodiments, the LNPs are formulated according to any one of the LNP formulations described in TABLE 20.

### Delivery of Viral Vectors

In some embodiments, a vector described herein comprises a viral vector. In some embodiments, the viral vector comprises a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein may be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector is genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more polypeptides (e.g., effector proteins, effector partners, fusion proteins, or combinations thereof), a nucleic acid encoding the one or more polypeptides comprising heterologous peptide (e.g., a nuclear localization signal (NLS), polyA tail), one or more guide nucleic acids, a nucleic acid encoding the one or more guide nucleic acids, respective promoter(s), or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing AAV delivery vectors herein comprise packaging a nucleic acid encoding a polypeptide (e.g., an effector protein, an effector partner, a fusion protein, or a combination thereof) and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging the polypeptide encoding nucleic acid into the AAV particle, thereby generating an AAV delivery vector. In some embodiments, promoters, stuffer sequences, and any combination thereof are packaged in the AAV vector. In some examples, the AAV vector packages 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes are not the same. In some examples, the Rep gene and ITR from a first AAV serotype (e.g., AAV2) are used in a capsid from a second AAV serotype (e.g., AAV9), wherein the first and second AAV serotypes are not the same. As a non-limiting example, a hybrid AAV serotype comprising the AAV2 ITRs and AAV9 capsid protein is indicated AAV2/9. In some examples, the hybrid AAV delivery vector comprises an AAV2/1, AAV2/2, AAV 2/4, AAV2/5, AAV2/8, or AAV2/9 vector.

### Producing AAV Particles

In some embodiments, AAV particles described herein are recombinant AAV (rAAV). In some embodiments, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a variant thereof, or a combination thereof. In some embodiments, rAAV virus particles can be produced in the mammalian cell culture system by providing the rAAV plasmid to the mammalian cell. In some embodiments, producing rAAV virus particles in a mammalian cell comprises transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, the insect cell for producing rAAV viral particles comprises a Sf9 cell. In some embodiments, production of rAAV virus particles in insect cells comprise baculovirus. In some embodiments, production of rAAV virus particles in insect cells comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### Target Nucleic Acids

Disclosed herein are compositions, systems and methods for detecting and/or editing a target nucleic acid. In some embodiments, the target nucleic acid is a double stranded nucleic acid. In some embodiments, the target nucleic acid is a single stranded nucleic acid. Alternatively, or in combination, the target nucleic acid is a double stranded nucleic acid and is prepared into single stranded nucleic acids before or upon contacting an RNP. In some embodiments, the single stranded nucleic acid comprises a RNA, wherein the RNA comprises a mRNA, a rRNA, a tRNA, a non-coding RNA, a long non-coding RNA, a microRNA (miRNA), and a single-stranded RNA (ssRNA). In some embodiments, the target nucleic acid is complementary DNA (cDNA) synthesized from a single-stranded RNA template in a reaction catalyzed by a reverse transcriptase. In some embodiments, the target nucleic acid comprises an RNA, a DNA, or combination thereof. In some embodiments, guide nucleic acids described herein hybridize to a portion of the target nucleic acid. In some embodiments, the target nucleic acid is from a virus, a parasite, or a bacterium described herein.

In some embodiments, a target nucleic acid comprising a target sequence comprises a PAM sequence. In some embodiments, the PAM sequence is adjacent to the target sequence. In some embodiments, the PAM sequence is 3′ to the target sequence. In some embodiments, the PAM sequence is directly 3′ to the target sequence. In some embodiments, the PAM sequence 5′ to the target sequence. In some embodiments, the PAM sequence is directly 5′ to the target sequence. In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a polypeptide system described herein.

In some embodiments, a target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides. In some embodiments, the target sequence in the target nucleic acid comprises at least 10 contiguous nucleotides that are complementary to the guide nucleic acid or engineered guide nucleic acid.

In some embodiments, compositions, systems, and methods described herein comprise a target nucleic acid that is responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the sequence of the target nucleic acid. In some embodiments, the change comprises an insertion, deletion, or substitution of one or more nucleotides compared to the target nucleic acid that has not undergone any modification.

In some embodiments, the target nucleic acid comprises a nucleic acid sequence from a pathogen responsible for a disease. Non-limiting examples of pathogens are bacteria, a virus and a fungus. In some embodiments, the target sequence is a portion of a nucleic acid from a virus or a bacterium or other agents responsible for a disease in the sample. The target sequence, in some embodiments, is a portion of a nucleic acid from a sexually transmitted infection or a contagious disease, in the sample. The target sequence, in some embodiments, is a portion of a nucleic acid from an upper respiratory tract infection, a lower respiratory tract infection, or a contagious disease, in the sample. The target sequence, in some embodiments, is a portion of a nucleic acid from a hospital acquired infection or a contagious disease, in the sample. The target sequence, in some embodiments, is a portion of a nucleic acid from sepsis, in the sample. In some embodiments, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. A pathogenic virus can be a DNA virus or an RNA virus. Pathogenic viruses include but are not limited to respiratory viruses, adenoviruses, parainfluenza viruses, severe acute respiratory syndrome (SARS), coronavirus (e.g., SARS-CoV), MERS, gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection), hepatic viral diseases (e.g., hepatitis A, B, C, D, E), cutaneous viral diseases (e.g., warts (including genital, anal), herpes (including oral, genital, anal), molluscum contagiosum), hemmorhagic viral diseases (e.g., Ebola, Lassa fever, dengue fever, yellow fever, Marburg hemorrhagic fever, Crimean-Congo hemorrhagic fever), neurologic viruses (e.g., polio, viral meningitis, viral encephalitis, rabies), sexually transmitted viruses (e.g., HIV, HPV, and the like), Adenovirus, coronavirus (i.e., a virus that causes COVID-19), Coronavirus HKU1, Coronavirus NL63, Coronavirus 229E, Coronavirus OC43, Severe Acute Respiratory Syndrome Coronavirus 2 (SARS-CoV-2), Human Metapneumovirus (hMPV), Human Rhinovirus/Enterovirus, influenza virus, Influenza A, Influenza A/H1, Influenza A/H3, Influenza A/H1-2009, Influenza B, Influenza C, Parainfluenza Virus 1, Parainfluenza Virus 2, Parainfluenza Virus 3, Parainfluenza Virus 4, Respiratory Syncytial Virus), human immunodeficiency virus (e.g., HIV), human papillomavirus (e.g., HPV), chlamydia, gonorrhea, syphilis, trichomoniasis, sexually transmitted infection, malaria, Dengue fever, Ebola, chikungunya, leishmaniasis, Orthopoxvirus (e.g., monkeypox virus, cowpox virus, camelpox virus, horsepox virus, vaccinia virus, and variola virus), West Nile virus, herpes virus, yellow fever virus, Hepatitis Virus C, Hepatitis Virus A, Hepatitis Virus B, papillomavirus, and the like. Pathogens include, e.g., HIV virus, Bordetella parapertussis, Bordetella pertussis, Chlamydia pneumoniae, Mycoplasma pneumoniae, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. In some embodiments, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target sequence is comprised in a sample. In some embodiments, the sample used for genetic disorder testing, cancer testing, or cancer risk testing can comprise at least one target sequence or target nucleic acid segment that can bind to a guide nucleic acid of the reagents described herein. In some embodiments, the sample used comprises a target sequence or target nucleic acid of a gene recited in TABLE 9. In some embodiments, the sample used comprises a target sequence or target nucleic acid of a gene selected from ANGPTL3, APOC3, PCSK9, LDHA, HSD17B13, HAO1, CNBP and LPA.

In some embodiments, the sample used for phenotyping testing can comprise at least one target nucleic acid segment that can bind to a guide nucleic acid of the reagents described herein. The target nucleic acid segment, in some cases, is a portion of a nucleic acid from a gene associated with a phenotypic trait.

In some embodiments, the sample used for genotyping testing can comprise at least one target nucleic acid segment that can bind to a guide nucleic acid of the reagents described herein. The target nucleic acid segment, in some cases, is a portion of a nucleic acid from a gene associated with a genotype.

In some embodiments, the target nucleic acid comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. A polypeptide (e.g., an effector protein, an effector partner, a fusion protein, or a combination thereof) of the disclosure may cleave the viral nucleic acid. In some embodiments, the target nucleic acid comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some embodiments, is a portion of a nucleic acid from a virus or a bacterium or other agents responsible for a disease in the plant (e.g., a crop). In some embodiments, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that are targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, a target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are recited in TABLE 9. Nucleic acid sequences of target nucleic acids and/or corresponding genes are readily available in public databases as known and used in the art. In some embodiments, the target nucleic acid is selected from TABLE 9. In some embodiments, the target nucleic acid comprises one or more target sequences. In some embodiments, the one or more target sequence is within any one of the target nucleic acids recited in TABLE 9. In some embodiments, the target nucleic acid is an amplicon of at least a portion of any one of the genes selected from ANGPTL3, APOC3, PCSK9, LDHA, HSD17B13, HAO1, CNBP and LPA. In some embodiments, the target nucleic acid comprises at least a portion of any one of the genes selected from ANGPTL3, APOC3, PCSK9, LDHA, HSD17B13, HAO1, CNBP and LPA. Accordingly, in some embodiments, a target sequence of the target nucleic acid is within any one of the genes selected from ANGPTL3, APOC3, PCSK9, LDHA, HSD17B13, HAO1, CNBP and LPA.

In some embodiments, the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell. In some embodiments, the target nucleic acid is isolated from a population of cells.

Nucleic acids, such as DNA and pre-mRNA, described herein can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In some embodiments, nucleic acids can contain one or more elements that act as a signal during transcription, splicing, and/or translation. In some embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids also comprise an untranslated region (UTR), such as a 5′ UTR or a 3′ UTR. An exemplary 5′ sequence a polynucleotide UTR comprises sequence of AGGAGAGAAAAGAAGAGUAAGAAGAAAUAUAAGAGCCACC (SEQ ID NO: 369). An exemplary 3′ UTR sequence comprises a polynucleotide sequence of UUAAUUAAGCUGCCUUCUGCGGGGCUUGCCUUCUGGCCAUGCCCUUCUUCUCUCCCUU

GCACCUGUACCUCUUGGUCUUUGAAUAAAGCCUGAGUAGGAAG (SEQ ID NO: 370). In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within about 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems, and methods described herein comprise an edited target nucleic acid which can describe a target nucleic acid wherein the target nucleic acid has undergone a change, for example, after contact with a polypeptide (e.g., an effector protein, an effector partner, a fusion protein, or a combination thereof). In some embodiments, the editing is an alteration in the sequence of the target nucleic acid. In some embodiments, the edited target nucleic acid comprises an insertion, deletion, or replacement of one or more nucleotides compared to the unedited target nucleic acid. In some embodiments, the editing is a mutation.

### Mutations

In some embodiments, target nucleic acids described herein comprise a mutation. In some embodiments, a composition, system or method described herein can be used to edit a target nucleic acid comprising a mutation such that the mutation is edited to be the wild-type nucleotide or nucleotide sequence. In some embodiments, a composition, system or method described herein can be used to detect a target nucleic acid comprising a mutation. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

Non-limiting examples of mutations are insertion-deletion (indel), a point mutation, single nucleotide polymorphism (SNP), a chromosomal mutation, a copy number mutation or variation, and frameshift mutations. In some embodiments, an indel mutation is an insertion or deletion of one or more nucleotides. In some embodiments, a point mutation comprises a substitution, insertion, or deletion. In some embodiments, a frameshift mutation occurs when the number of nucleotides in the insertion/deletion is not divisible by three, and it occurs in a protein coding region. In some embodiments, a chromosomal mutation can comprise an inversion, a deletion, a duplication, or a translocation of one or more nucleotides. In some embodiments, a copy number variation can comprise a gene amplification or an expanding trinucleotide repeat. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, the target nucleic acid comprises one or more mutations. In some embodiments, the target nucleic acid comprises one or more, two or more, three or more, four or more, five or more, six or more, seven or more, eight or more, nine or more, or ten or more mutations as compared to the unmutated target nucleic acid. In some embodiments, the target nucleic acid comprises a sequence comprising one or more, two or more, three or more, four or more, five or more, six or more, seven or more, eight or more, nine or more, or ten or more mutations as compared to the wildtype sequence. In some embodiments, the target nucleic acid comprises a mutation associated with a disease or disorder.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP, or a deletion, is encoded in the sequence of a target nucleic acid from the germline of an organism or is encoded in a target nucleic acid from a diseased cell.

In some embodiments, the mutation is associated with a disease, such as a genetic disorder. In some embodiments, the mutation is encoded in the sequence of a target nucleic acid from the germline of an organism or is encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation. In some embodiments, a target nucleic acid described herein comprises a mutation associated with a disease, wherein the target nucleic acid is any one of the target nucleic acids recited in TABLE 9. In some embodiments, a target nucleic acid described herein comprises a mutation associated with a disease, wherein the disease is any one of the diseases recited in TABLE 10.

### Compositions

Disclosed herein are compositions comprising one or more polypeptides (e.g., effector proteins, effector partners, fusion proteins, or combinations thereof) described herein or nucleic acids encoding the one or more polypeptides, one or more guide nucleic acids described herein or nucleic acids encoding the one or more guide nucleic acids described herein, or combinations thereof. In some embodiments, one or more of a repeat sequence, a handle sequence, and intermediary sequence of the one or more guide nucleic acids are capable of interacting (or interacts) with the one or more of the effector proteins. In some embodiments, spacer sequences of the one or more guide nucleic acids hybridizes with a target sequence of a target nucleic acid. In some embodiments, the compositions are capable of cleaving (or cleaves) a target strand, a non-target strand, or both of a target nucleic acid. In some embodiments, the compositions are not capable of cleaving (or cleaves) a target strand, a non-target strand, or both of a target nucleic acid. In some embodiments, the compositions are capable of modifying (or modifies) a target strand or a non-target strand of a target nucleic acid. In some embodiments, the compositions are capable of modifying (or modifies) expression of the target nucleic acids, proteins associated with the expression of the target nucleic acids, other nucleic acids associated with the target nucleic acids, or combinations thereof. In some embodiments, the compositions are capable of editing (or edits) a target nucleic acid in a cell or a subject. In some embodiments, the compositions are capable of editing (or edits) a target nucleic acid or the expression thereof in a cell, in a tissue, in an organ, in vitro, in vivo, or ex vivo. In some embodiments, the compositions are capable of editing (or edits) a target nucleic acid in a sample comprising the target nucleic.

In some embodiments, compositions described herein comprise plasmids described herein, viral vectors described herein, non-viral vectors described herein, or combinations thereof. In some embodiments, the compositions described herein comprise the viral vectors. In some embodiments, compositions described herein comprise an AAV. In some embodiments, the compositions described herein comprise liposomes (e.g., cationic lipids or neutral lipids), dendrimers, lipid nanoparticle (LNP), or cell-penetrating peptides. In some embodiments, the compositions described herein comprise an LNP.

### Pharmaceutical Compositions

Described herein are formulations of introducing compositions or components of a system described herein to a host.

In some embodiments, compositions described herein are pharmaceutical compositions. In some embodiments, the pharmaceutical compositions comprise compositions described herein or systems described herein. In some embodiments, the pharmaceutical composition comprises a pharmaceutically acceptable salt, one or more of a vehicle, adjuvant, excipient, or carrier, such as a filler, disintegrant, a surfactant, a binder, a lubricant, or combinations thereof. Remington: The Science and Practice of Pharmacy, 21st edition, 2005, ed. D. B. Troy, Lippincott Williams & Wilkins, Philadelphia; Encyclopedia of Pharmaceutical Technology, eds. J. Swarbrick and J. C. Boylan, 1988-1999, Marcel Dekker, New York; and Encyclopedia of Pharmaceutical Technology, eds. J. Swarbrick, 2015, CRC Press, Boca Raton disclose various carriers used in formulating pharmaceutically acceptably compositions and known techniques for the preparation thereof. Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺ SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

### Systems

Disclosed herein, in some aspects, are systems for modifying or editing a target nucleic acid. In some embodiments, systems comprise one or more components. In some embodiments, the one or more components are provided as a single composition. In some embodiments, the system comprises at least two components each independently or collectively comprising one or more compositions described herein. In some embodiments, the one or more components independently or collectively comprise at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids. In some embodiments, the system comprises an expression vector, wherein the expression vector encodes the effector protein, the base editing enzyme, and the guide nucleic acid. In some embodiments, the nucleic acid expression vector is a viral vector or a non-viral vector. In some embodiments, the viral vector is an adeno associated viral (AAV) vector. In some embodiments, the nucleic acids encoding the effector protein, the effector partner or the combination thereof are messenger RNAs. In some embodiments, the systems comprise a lipid or a lipid nanoparticle. In some embodiments, the lipid nanoparticle is formulated according to any one of LNP formulations described in TABLE 20.

In some embodiments, the system described herein edits the target nucleic acid. In some embodiments, the system edits at least one base of the target nucleic acid, wherein the target nucleic acid is a single stranded target nucleic acid. In some embodiments, the system cleaves the single stranded target nucleic acid. In some embodiments, the system edits at least one base of the target nucleic acid, wherein the target nucleic acid is a double stranded target nucleic acid comprising a target strand and a non-target strand. In some embodiments, the system edits the target strand. In some embodiments, the system edits the non-target strand. In some embodiments, the system cleaves the target strand, the non-target strand, or both.

### Additional System Components

In some embodiments, systems include a reagent, a solution, a buffer, and a support medium, or combinations thereof. In some embodiments, systems comprise compositions, a solution, a buffer, a reagent, a support medium, or combinations thereof. In some embodiments, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, syringes, and test tubes. In some embodiments, the containers are formed from a variety of materials such as glass, plastic, or polymers. In some embodiments, the system or systems described herein contain packaging materials. Examples of packaging materials include, but are not limited to, pouches, blister packs, bottles, tubes, bags, containers, bottles, and any packaging material suitable for intended mode of use.

In some embodiments, systems described herein include labels listing contents and/or instructions for use, or package inserts with instructions for use. In some embodiments, the systems include a set of instructions and/or a label is on or associated with the container. In some embodiments, the label is on a container when letters, numbers or other characters forming the label are attached, molded, or etched into the container itself; a label is associated with a container when it is present within a receptacle or carrier that also holds the container (e.g., as a package insert). In some embodiments, the label is used to indicate that the contents are to be used for a specific therapeutic application. In some embodiments, the label indicates directions for use of the contents, such as in the methods described herein. In some embodiments, after packaging the formed product and wrapping or boxing to maintain a sterile barrier, the product is terminally sterilized by heat sterilization, gas sterilization, gamma irradiation, or by electron beam sterilization. Alternatively, in some embodiments, the product is prepared and packaged by aseptic processing.

In some embodiments, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

Methods and Formulations for Introducing System Components and Compositions into a Target Cell

Disclosed herein, in some aspects, are systems and methods for introducing systems and components of such systems into a target cell. Such systems may comprise, as described herein, one or more components having any one of the polypeptides (e.g., effector proteins, effector partners, fusion proteins, or combination thereof) or a nucleic acid comprising a nucleotide sequence encoding same. In some embodiments, such systems comprise, as described herein, one or more components having a guide nucleic acid or a nucleic acid comprising a nucleotide sequence encoding same. In some embodiments, systems comprise one or more components having a guide nucleic acid and an additional nucleic acid. Systems and components thereof may be used to introduce the polypeptides, guide nucleic acids, or combinations thereof into a target cell. Such methods may be used to modify or edit a target nucleic acid. In some embodiments, systems comprise the polypeptide, one or more guide nucleic acids, and a reagent for facilitating the introduction of the polypeptide and the one or more guide nucleic acids. In some embodiments, system components for the methods comprise a solution, a buffer, a reagent for facilitating the introduction of the polypeptide and the one or more guide nucleic acids, or combinations thereof. A guide nucleic acid (or a nucleic acid comprising a nucleotide sequence encoding same) and/or a polypeptide (e.g., effector protein, effector partner, fusion protein, or combination thereof) (or a nucleic acid comprising a nucleotide sequence encoding same) described herein may be introduced into a host cell by any of a variety of well-known methods. As a non-limiting example, the guide nucleic acid and/or polypeptide may be combined with a lipid. As another non-limiting example, the guide nucleic acid and/or polypeptide may be combined with a particle or formulated into a particle.

### Methods for Introducing System Components and Compositions to a Host

Described herein are methods of introducing various components described herein to a host. A host may be any suitable host, such as a host cell. When described herein, a host cell may be an in vivo, ex vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells may be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell may be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method may be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X (12) 00283-9. doi: 10.1016/j.addr.2012.09.023), and the like. In some embodiments, the nucleic acid and/or protein(s) are introduced into a disease cell comprised in a pharmaceutical composition comprising the guide nucleic acid, the polypeptide, a pharmaceutically acceptable excipient, or combinations thereof.

In some embodiments, molecules of interest, such as nucleic acids of interest, are introduced to a host. In some embodiments, polypeptides are introduced to a host. In some embodiments, vectors, such as lipid particles and/or viral vectors, are introduced to a host. Introduction may be for contact with a host or for assimilation into the host, for example, introduction into a host cell.

In some embodiments, described herein are methods of introducing one or more nucleic acids, such as a nucleic acid encoding a guide nucleic acid, a nucleic acid that, when transcribed, produces an engineered guide nucleic acid, or combinations thereof, into a host cell. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell may occur in any culture media and under any culture conditions that promote the survival of the cells. Introducing one or more nucleic acids into a host cell may be carried out in vivo or ex vivo. Introducing one or more nucleic acids into a host cell may be carried out in vitro.

In some embodiments, polypeptides (e.g., effector proteins, effector partners, fusion proteins, or combination thereof) are provided as RNA. The RNA may be provided by direct chemical synthesis or may be transcribed in vitro from a DNA (e.g., encoding the polypeptide). Once synthesized, the RNA may be introduced into a cell by way of any suitable technique for introducing nucleic acids into cells (e.g., microinjection, electroporation, transfection, etc.). In some embodiments, introduction of one or more nucleic acids is through the use of a vector and/or a vector system, accordingly, in some embodiments, compositions and system described herein comprise a vector and/or a vector system.

Vectors may be introduced directly to a host. In some embodiments, host cells are contacted with one or more vectors as described herein, and in some embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein may also be introduced directly to a host. For example, an engineered guide nucleic acid may be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins, effector partners, fusion proteins, or combination thereof) described herein may also be introduced directly to a host. In some embodiments, polypeptides described herein are modified to promote introduction to a host. For example, polypeptides described herein are modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g. from 1 to 10 glycine residues. In some embodiments, the cleavage of the polypeptide is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. In another example, the polypeptide is modified to improve stability. For example, the polypeptides are PEGylated, where the polyethyleneoxy group provides for enhanced lifetime in the blood stream. Polypeptides may also be modified to promote uptake by a host, such as a host cell. For example, a polypeptide described herein is fused to a polypeptide permeant domain to promote uptake by a host cell. Any suitable permeant domains may be used in the non-integrating polypeptides of the present disclosure, including peptides, peptidomimetics, and non-peptide carriers. Examples include penetratin, a permeant peptide derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site may be determined by suitable methods.

### Formulations for Introducing System Components and Compositions to a Host

Described herein are formulations of introducing compositions or components of a system described herein to a host. In some embodiments, such formulations, systems and compositions described herein comprise polypeptides (e.g., effector proteins, effector partners, fusion proteins, or combination thereof) and a carrier (e.g., excipient, diluent, vehicle, or filling agent). In some aspects of the present disclosure, the polypeptides are provided in a pharmaceutical composition comprising the polypeptides and any pharmaceutically acceptable excipient, carrier, or diluent.

### Methods of Modifying a Nucleic Acid

Provided herein are compositions, methods, and systems for modifying (e.g., editing) target nucleic acids. In general, modifying refers to changing the physical composition of a target nucleic acid. However, compositions, methods, and systems disclosed herein may also be capable of modifying (or modifies) target nucleic acids, such as making epigenetic modifications of target nucleic acids, which does not change the nucleotide sequence of the target nucleic acids per se. Polypeptides (e.g., effector proteins, effector partners, fusion proteins, or combination thereof), compositions and systems described herein may be used for modifying a target nucleic acid, which includes editing a target nucleic acid sequence. Modifying a target nucleic acid may comprise one or more of: cleaving the target nucleic acid, deleting one or more nucleotides of the target nucleic acid, inserting one or more nucleotides into the target nucleic acid, mutating one or more nucleotides of the target nucleic acid, or otherwise changing one or more nucleotides of the target nucleic acid. Modifying a target nucleic acid may comprise one or more of: methylating, demethylating, deaminating, or oxidizing one or more nucleotides of the target nucleic acid. In some embodiments, the target nucleic acid is selected from the group consisting of B2M gene, TRAC gene and CIITA gene. In some embodiments, the methods described herein edit the target nucleic acid without resulting in translocation or chromosomal rearrangements in a cell. Alternatively, in some embodiments, the methods described herein edit the target nucleic acid with fewer translocations or chromosomal rearrangements relative to editing of the target nucleic acid by a Cas9 effector protein. In some embodiments, the methods comprise editing of more than one target sequences by the compositions or systems described herein, wherein the more than one target sequences are edited simultaneously or sequentially. In some embodiments, the more than one target sequences are within the same target nucleic acid. Alternatively, in some embodiments, at least two of the more than on target sequences are in different target nucleic acids.

Compositions, methods, and systems described herein may modify a coding portion of a gene, a non-coding portion of a gene, or a combination thereof. Modifying at least one gene using the compositions, methods or systems described herein may reduce or increase expression of one or more genes. In some embodiments, the compositions, methods or systems reduce expression of one or more genes by at least 10%, at least 20%, at least 30%, at least 40%, at least 50%, at least 60%, at least 70%, at least 80%, at least 90%, or at least 95%. In some embodiments, the compositions, methods or systems remove all expression of a gene, also referred to as genetic knock out. In some embodiments, the compositions, methods or systems increase expression of one or more genes by at least 10%, at least 20%, at least 30%, at least 40%, at least 50%, at least 60%, at least 70%, at least 80%, at least 90%, or at least 100%.

In some embodiments, the compositions, methods or systems comprise a nucleic acid expression vector, or use thereof, to introduce polypeptides (e.g., effector proteins, effector partners, fusion proteins, or combination thereof), guide nucleic acid, or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce the polypeptide, guide nucleic acid, or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of modifying may comprise contacting a target nucleic acid with one or more components, compositions or systems described herein. In some embodiments, a method of modifying comprises contacting a target nucleic acid with at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids. In some embodiments, a method of modifying comprises contacting a target nucleic acid with a composition described herein comprising at least one of: a) one or more polypeptides (e.g., effector proteins, effector partners, fusion proteins, or combination thereof), or one or more nucleic acids encoding the one or more polypeptides; or b) one or more guide nucleic acids, or one or more nucleic acids encoding one or more guide nucleic acids; in a composition. In some embodiments, a method of modifying as described herein produces a modified target nucleic acid.

Editing a target nucleic acid sequence may introduce a mutation (e.g., point mutations, deletions) in a target nucleic acid relative to a corresponding wildtype nucleotide sequence. Editing may remove or correct a disease-causing mutation in a nucleic acid sequence to produce a corresponding wildtype nucleotide sequence. Editing a target nucleic acid sequence may remove/correct point mutations, deletions, null mutations, or tissue-specific mutations in a target nucleic acid. Editing a target nucleic acid sequence may be used to generate gene knock-out, gene knock-in, gene editing, gene tagging, or a combination thereof. Methods of the disclosure may be targeted to any locus in a genome of a cell.

Modifying may comprise single stranded cleavage, double stranded cleavage, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer sequence. In some embodiments, polypeptides (e.g., effector proteins, effector partners, fusion proteins, or combination thereof) introduce a single-stranded break in a target nucleic acid to produce a cleaved nucleic acid. In some embodiments, the polypeptides are capable of introducing (or introduces) a break in a single stranded RNA (ssRNA). The polypeptides may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel may vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given polypeptide.

In some embodiments, methods of modifying described herein cleave a target nucleic acid at one or more locations to generate a cleaved target nucleic acid. In some embodiments, the cleaved target nucleic acid undergoes recombination (e.g., NHEJ or HDR). In some embodiments, cleavage in the target nucleic acid may be repaired (e.g., by NHEJ or HDR), such that the repair results in an indel in the target nucleic acid at or near the site of the cleavage site.

In some embodiments, wherein the compositions, systems, and methods of the present disclosure restore a wild-type reading frame. A wild-type reading frame may be a reading frame that produces at least a partially, or fully, functional protein. A non-wild-type reading frame may be a reading frame that produces a non-functional or partially non-functional protein.

Accordingly, in some embodiments, compositions, systems, and methods described herein may edit 1 to 1,000 nucleotides or any integer in between, in a target nucleic acid. In some embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, may be edited by the compositions, systems, and methods described herein. In some embodiments, 1, 2, 3, 4, 5, 6, 7, 8, 9, 10, or more nucleotides may be edited by the compositions, systems, and methods described herein. In some embodiments, 10, 20, 30, 40, 50, 60, 70, 80 90, 100 or more nucleotides, or any integer in between, may be edited by the compositions, systems, and methods described herein. In some embodiments, 100, 200, 300, 400, 500, 600, 700, 800, 900 or more nucleotides, or any integer in between, may be edited by the compositions, systems, and methods described herein.

Methods may comprise use of two or more polypeptides (e.g., effector proteins, effector partners, fusion proteins, or combination thereof). An illustrative method for introducing a break in a target nucleic acid comprises contacting the target nucleic acid with: (a) a first engineered guide nucleic acid comprising a region that binds to a first polypeptide described herein; and (b) a second engineered guide nucleic acid comprising a region that binds to a second polypeptide described herein, wherein the first engineered guide nucleic acid comprises an additional region that hybridizes to the target nucleic acid and wherein the second engineered guide nucleic acid comprises an additional region that hybridizes to the target nucleic acid. In some embodiments, the first and second polypeptide are identical. In some embodiments, the first and second polypeptide are not identical.

In some embodiments, editing a target nucleic acid comprises genome editing. Genome editing may comprise editing a genome, chromosome, plasmid, or other genetic material of a cell or organism.

In some embodiments, the genome, chromosome, plasmid, or other genetic material of the cell or organism is modified in vivo or ex vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid is edited in vitro using a composition described herein and introduced into a cell or organism.

In some embodiments, editing a target nucleic acid comprises deleting a sequence from a target nucleic acid. For example, a mutated sequence or a sequence associated with a disease is removed from a target nucleic acid. In some embodiments, editing a target nucleic acid comprises replacing a sequence in a target nucleic acid with a second sequence. For example, a mutated sequence or a sequence associated with a disease is replaced with a second sequence lacking the mutation or that is not associated with the disease. In some embodiments, editing a target nucleic acid comprises deleting or replacing a sequence comprising markers associated with a disease or disorder.

In some embodiments, methods comprise modifying a nucleotide base of an uncleaved target nucleic acid. The modification may be done at a specified (e.g., polypeptide targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting an uncleaved strand of the target nucleic acid with a base editing enzyme, thereby favoring the uncleaved strand of the target nucleic acid to be used as a template during base mismatch repair. In some embodiments, the cleaved target nucleic acid is cleaved at two locations. In such embodiments, the methods comprise contacting a target nucleic acid with a polypeptide described herein, thereby introducing a single-stranded break in the target nucleic acid; contacting the target nucleic acid with a second polypeptide described herein, to generate a second cleavage site in the target nucleic acid, contacting an uncleaved strand of the target nucleic acid with a base editing enzyme, thereby favoring the uncleaved strand of the target nucleic acid to be used as a template during base mismatch repair. An intermediate step may include HDR or NHEJ mediated repair.

In some embodiments, methods comprise editing a target nucleic acid with two or more polypeptides (e.g., effector proteins, effector partners, fusion proteins, or combination thereof). Editing a target nucleic acid may comprise introducing a single-stranded break in a target nucleic acid. In some embodiments, a break is introduced by contacting a target nucleic acid with the polypeptide and guide nucleic acid. The guide nucleic acid may bind to the polypeptide and hybridize to a region of the target nucleic acid, thereby recruiting the polypeptide to the region of the target nucleic acid. Binding of the polypeptide to the guide nucleic acid and the region of the target nucleic acid may activate a second polypeptide. In some embodiments, the second polypeptide introduces a base pair modification in the region of the target nucleic acid. In some embodiments, editing a target nucleic acid comprises introducing a break in a first region of the target nucleic acid and a base pair modification in a second region of the target nucleic acid. The first polypeptide may introduce a break in a first strand at the first region of the target nucleic acid, and the second polypeptide may introduce a base pair modification in a second strand at the second region of the target nucleic acid during base mismatch repair.

In some embodiments, methods comprise editing a target nucleic acid with two or more polypeptides (e.g., effector proteins, effector partners, fusion proteins, or combination thereof). Editing a target nucleic acid may comprise introducing a two or more single-stranded breaks in a target nucleic acid. In some embodiments, a break is introduced by contacting a target nucleic acid with a first polypeptide and a guide nucleic acid. The guide nucleic acid may bind to the first polypeptide and hybridize to a first region of the target nucleic acid, thereby recruiting the first polypeptide to the first region of the target nucleic acid. Binding of the effector protein to the guide nucleic acid and the region of the target nucleic acid may activate the second polypeptide, and the second polypeptide may introduce a base pair modification. The events lead to activation of a third polypeptide, and the third polypeptide may introduce a second break (e.g., a single stranded break) in a third region of the target nucleic acid. In some embodiments, a segment of the target nucleic acid between the first break and the second break may be removed, thereby favoring a use of uncleaved strand of the target nucleic acid as a template sequence during base mismatch repair.

Methods, systems and compositions described herein may edit a target nucleic acid wherein such editing may result in one or more indels. In some embodiments, where compositions, systems, and/or methods described herein effect one or more indels, the impact on the transcription and/or translation of the target nucleic acid is predicted depending on: 1) the amount of indels generated; and 2) the location of the indel on the target nucleic acid. For example, as described herein, in some embodiments, if the amount of indels is not divisible by three, and the indels occur within or along a protein coding region, then the edit or mutation may be a frameshift mutation. In some embodiments, if the amount of indels is divisible by three, then a frameshift mutation is not affected, but a splicing disruption mutation and/or sequence skip mutation is effected, such as an exon skip mutation. In some embodiments, if the amount of indels is not evenly divisible by three, then a frameshift mutation is affected.

Methods, systems and compositions described herein may edit a target nucleic acid wherein such editing may be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity is detected by next generation sequencing of one or more target loci of a target nucleic acid where indel percentage is calculated as the fraction of sequencing reads containing insertions or deletions relative to an unedited reference sequence. In some embodiments, methods, systems, and compositions comprising polypeptides (e.g., effector proteins, effector partners, fusion proteins, or combination thereof) and guide nucleic acid described herein exhibit about 0.0001% to about 65% or more indel activity upon contact to a target nucleic acid compared to a target nucleic acid non-contacted with compositions, systems, or by methods described herein. For example, methods, systems, and compositions comprising polypeptides (e.g., effector proteins, effector partners, fusion proteins, or combination thereof) and guide nucleic acid described herein exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing of a target nucleic acid as described herein effects one or more mutations comprising splicing disruption mutations, frameshift mutations (e.g., 1+ or 2+ frameshift mutation), sequence deletion, sequence skipping, sequence reframing, sequence knock-in, or any combination thereof. In some embodiments, the splicing disruption can be an editing that disrupts a splicing of a target nucleic acid or a splicing of a sequence that is transcribed from a target nucleic acid relative to a target nucleic acid without the splicing disruption. In some embodiments, the frameshift mutation can be an editing that alters the reading frame of a target nucleic acid relative to a target nucleic acid without the frameshift mutation. In some embodiments, the frameshift mutation can be a +2 frameshift mutation, wherein a reading frame is edited by 2 bases. In some embodiments, the frameshift mutation can be a +1 frameshift mutation, wherein a reading frame is edited by 1 base. In some embodiments, the frameshift mutation is an editing that alters the number of bases in a target nucleic acid so that it is not divisible by three. In some embodiments, the frameshift mutation can be an editing that is not a splicing disruption. In some embodiments a sequence as described in reference to the sequence deletion, sequence skipping, sequence reframing, and sequence knock-in can be a DNA sequence, a RNA sequence, an edited DNA or RNA sequence, a mutated sequence, a wild-type sequence, a coding sequence, a non-coding sequence, an exonic sequence (exon), an intronic sequence (intron), or any combination thereof. In some embodiments, the sequence deletion is an editing where one or more sequences in a target nucleic acid are deleted relative to a target nucleic acid without the sequence deletion. In some embodiments, the sequence deletion can result in or effect a splicing disruption or a frameshift mutation. In some embodiments, the sequence deletion result in or effect a splicing disruption. In some embodiments, the sequence skipping is an editing where one or more sequences in a target nucleic acid are skipped upon transcription or translation of the target nucleic acid relative to a target nucleic acid without the sequence skipping. In some embodiments, the sequence skipping can result in or effect a splicing disruption or a frameshift mutation. In some embodiments, the sequence skipping can result in or effect a splicing disruption. In some embodiments, the sequence reframing is an editing where one or more bases in a target are edited so that the reading frame of the sequence is reframed relative to a target nucleic acid without the sequence reframing. In some embodiments, the sequence reframing can result in or effect a splicing disruption or a frameshift mutation. In some embodiments, the sequence reframing can result in or effect a frameshift mutation. In some embodiments, the sequence knock-in is an editing where one or more sequences is inserted into a target nucleic acid relative to a target nucleic acid without the sequence knock-in. In some embodiments, the sequence knock-in can result in or effect a splicing disruption or a frameshift mutation. In some embodiments, the sequence knock-in can result in or effect a splicing disruption.

In some embodiments, editing of a target nucleic acid can be locus specific, wherein compositions, systems, and methods described herein can edit a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing of a specific locus can affect any one of a splicing disruption, frameshift (e.g., 1+ or 2+ frameshift), sequence deletion, sequence skipping, sequence reframing, sequence knock-in, or any combination thereof. In some embodiments, editing of a target nucleic acid can be locus specific, modification specific, or both. In some embodiments, editing of a target nucleic acid can be locus specific, modification specific, or both, wherein compositions, systems, and methods described herein comprise polypeptides (e.g., effector proteins, effector partners, fusion proteins, or combination thereof) described herein and a guide nucleic acid described herein.

Methods of editing a target nucleic acid or modulating the expression of a target nucleic acid are performed in vivo or ex vivo. Methods of editing a target nucleic acid or modulating the expression of a target nucleic acid are performed in vitro. For example, a plasmid is edited in vitro using a composition described herein and introduced into a cell or organism. Methods of editing a target nucleic acid or modulating the expression of a target nucleic acid are performed ex vivo. For example, methods comprise obtaining a cell from a subject, editing a target nucleic acid in the cell with methods described herein, and returning the cell to the subject.

In some embodiments, methods of modifying described herein comprise contacting a target nucleic acid with one or more components, compositions or systems described herein. In some embodiments, the one or more components, compositions or systems described herein comprise at least one of: a) one or more polypeptides (e.g., effector proteins, effector partners, fusion proteins, or combination thereof), or one or more nucleic acids encoding the one or more polypeptides; and b) one or more guide nucleic acids, or one or more nucleic acids encoding the one or more guide nucleic acids. In some embodiments, the one or more polypeptides introduce a single-stranded break or a double-stranded break in a target nucleic acid comprising a target strand and a non-target strand. In some embodiments, the one or more polypeptides introduce the single-stranded break in the target strand. In some embodiments, the one or more polypeptides introduce the single-stranded break in the non-target strand. In some embodiments, the one or more polypeptides introduce a base pair modification in the target nucleic acid. In some embodiments, the one or more polypeptides introduce the base pair modification in the target strand. In some embodiments, the one or more polypeptides introduce the base pair modification in the non-target strand. In some embodiments, methods of modifying described herein produce a modified target nucleic acid comprising an engineered nucleic acid sequence that expresses polypeptide having new activity as compared to an unmodified target nucleic acid, or alters expression of an endogenous polypeptide as compared to an unmodified target nucleic acid.

In some embodiments, methods of modifying described herein comprise using one or more guide nucleic acids or uses thereof, wherein the methods modify a target nucleic acid at a single location. In some embodiments, the methods comprise contacting an RNP comprising polypeptides (e.g., effector proteins, effector partners, fusion proteins, or combination thereof) and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, null mutations, or tissue-specific mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid. In some embodiments, the methods comprise using a system comprising an effector protein, an effector partner, and two guide nucleic acids, wherein two RNPs cleave the target nucleic acid at the same location, wherein a first RNP comprises the effector protein and a first guide nucleic acid, wherein a second RNP comprises the effector protein and a second guide nucleic acid, and wherein the effector partner is a base editing enzyme. In some embodiments, methods comprising using a system comprising a first effector protein, an effector partner and a second effector protein, and two guide nucleic acids, wherein both RNPs cleave the target nucleic acid at the same location, wherein a first RNP comprises the first effector protein and a first target nucleic acid, wherein a second RNP comprises the second effector protein and a second target nucleic acid, and wherein the effector partner is a base editing enzyme.

In some embodiments, methods of modifying described herein comprise using one or more guide nucleic acids or uses thereof, wherein the methods modify a target nucleic acid at two different locations. In some embodiments, the methods introduce two cleavage sites in the target nucleic acid, wherein a first cleavage site and a second cleavage site comprise one or more nucleotides therebetween. In some embodiments, the methods cause deletion of the one or more nucleotides. In some embodiments, the deletion restores a wild-type reading frame. In some embodiments, the wild-type reading frame produces at least a partially functional protein. In some embodiments, the deletion causes a non-wild-type reading frame. In some embodiments, a non-wild-type reading frame produces a partially functional protein or non-functional protein. In some embodiments, the at least partially functional protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% activity compared to a corresponding wildtype protein. In some embodiments, the methods comprise using a system comprising an effector protein, an effector partner, and two guide nucleic acids, wherein two RNPs cleave the target nucleic acid at different locations, wherein a first RNP comprises the effector protein and a first guide nucleic acid, wherein a second RNP comprises the effector protein and a second guide nucleic acid, and wherein the effector partner is a base editing enzyme. In some embodiments, methods comprising using a system comprising a first effector protein, an effector partner and a second effector protein, and two guide nucleic acids, wherein both RNPs cleave the target nucleic acid at different locations, wherein a first RNP comprises the first effector protein and a first target nucleic acid, wherein a second RNP comprises the second effector protein and a second target nucleic acid, and wherein the effector partner is a base editing enzyme.

### Genetically Modified Cells and Organisms

Methods of editing described herein may be employed to generate a genetically modified cell. In some embodiments, the cell is a eukaryotic cell (e.g., a mammalian cell) or a prokaryotic cell (e.g., an archaeal cell). In some embodiments, the cell is derived from a multicellular organism and cultured as a unicellular entity. In some embodiments, the cell comprises a heritable genetic modification, such that progeny cells derived therefrom comprise the heritable genetic mutation. In some embodiments, the cell is progeny of a genetically modified cell comprising a genetic modification of the genetically modified parent cell. In some embodiments, the genetically modified cell comprises a deletion, insertion, mutation, or non-native sequence relative to a wild-type version of the cell or the organism from which the cell was derived. In some embodiments, the methods modify the cell without resulting in translocations or chromosomal rearrangements in the cell. In some embodiments, the methods modify the cell with fewer translocations or chromosomal rearrangements in the cell as compared to modification with a Cas9 effector protein.

Methods of editing described herein may be performed in a cell. In some embodiments, the cell is in vivo. In some embodiments, the cell is ex vivo. In some embodiments, the cell is an isolated cell. In some embodiments, the cell is inside of an organism. In some embodiments, the cell is an organism. In some embodiments, the cell is in a cell culture. In some embodiments, the cell is one of a collection of cells. In some embodiments, the cell is a mammalian cell or derived there from. In some embodiments, the cell is a rodent cell or derived there from. In some embodiments, the cell is a human cell or derived there from. In some embodiments, the human cell is a T cell. In some embodiments, the human cell is a hematopoietic stem cell. In some embodiments, the cell is a eukaryotic cell or derived there from. In some embodiments, the cell is a progenitor cell or derived there from. In some embodiments, the cell is a pluripotent stem cell or derived there from. In some embodiments, the cell is an induced pluripotent stem cell or derived there from. In some embodiments, the cell is an animal cell or derived there from. In some embodiments, the cell is an invertebrate cell or derived there from. In some embodiments, the cell is a vertebrate cell or derived there from. In some embodiments, the cell is from a specific organ or tissue. In some embodiments, the cell is a hepatocyte.

In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow. In some embodiments, the tissue comprises muscle. In some embodiments, the muscle comprises a skeletal muscle. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

Methods of editing described herein may comprise contacting cells with compositions or systems described herein. For example, in some embodiments, the methods comprise contacting the cells with a nucleic acid encoding the effector protein at a dose of 3 μg to 10 μg (e.g., 3 μg, 6 μg, 9 μg or 10 μg), and a nucleic acid encoding the guide nucleic acid at a dose of 500 μM. In some embodiments, the contacting comprises electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

Methods of editing described herein may be performed in a subject. In some embodiments, the methods comprise administering compositions described herein to the subject. In some embodiments, the subject is a human. In some embodiments, the subject is a mammal (e.g., rat, mouse, cow, dog, pig, sheep, horse). In some embodiments, the subject is a vertebrate or an invertebrate. In some embodiments, the subject is a laboratory animal. In some embodiments, the subject is a patient. In some embodiments, the subject is at risk of developing, suffering from, or displaying symptoms of a disease. In some embodiments, the subject has a mutation associated with a gene described herein. In some embodiments, the subject displays symptoms associated with a mutation of a gene described herein.

### Methods of Treating a Disease or Disorder

Described herein are methods for treating a disease in a subject by contacting a target nucleic acid with a composition or system described herein, wherein the target nucleic acid is associated with a gene or expression of a gene related to the disease. In some embodiments, methods comprise treating, preventing, or inhibiting a disease or disorder associated with a mutation or aberrant expression of a gene. In some embodiments, methods for treating a disease or disorder comprise methods of editing a nucleic acid described herein. In some embodiments, compositions or systems described herein are for use in a method for treating a disease. In some embodiments, compositions or systems described herein are for use in the manufacture of a medicament for treating a disease.

Methods may comprise administration of a composition(s) or component(s) of a system described herein. In some embodiments, the composition(s) or component(s) of the system comprises use of a recombinant nucleic acid (DNA or RNA), administered for the purpose to edit a nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. By way of non-limiting example, the composition(s) comprises pharmaceutical compositions described herein. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

In some embodiments, treating, preventing, or inhibiting disease or disorder in a subject comprises contacting a target nucleic acid associated with a particular ailment with a composition described herein. In some aspects, the methods of treating, preventing, or inhibiting a disease or disorder involves removing, editing, modifying, replacing, transposing, or affecting the regulation of a genomic sequence of a patient in need thereof. In some embodiments, the methods of treating, preventing, or inhibiting a disease or disorder involves modulating gene expression.

In some embodiments, the compositions described herein are for use in therapy. In some embodiments, the compositions described herein are for use in treating a disease or condition described herein. Also provided is the use of the compositions described herein in the manufacture of a medicament. Also provided is the use of the compositions described herein in the manufacture of a medicament for therapeutic and/or prophylactic treatment of a disease or condition described herein.

In some embodiments, the compositions and systems described herein are for use in therapy. In some embodiments, the compositions and systems described herein are for use in treating a disease or condition described herein. Also provided is the use of the compositions and systems described herein in the manufacture of a medicament. Also provided is the use of the compositions and systems described herein in the manufacture of a medicament for therapeutic and/or prophylactic treatment of a disease or condition described herein.

In some embodiments, the guide nucleic acids described herein are for use in therapy. In some embodiments, the guide nucleic acids described herein are for use in treating a disease or condition described herein. Also provided is the use of the guide nucleic acids described herein in the manufacture of a medicament. Also provided is the use of the guide nucleic acids described herein in the manufacture of a medicament for therapeutic and/or prophylactic treatment of a disease or condition described herein.

Described herein are compositions, systems and methods for treating a disease in a subject by editing a target nucleic acid associated with a gene or expression of a gene related to the disease. For example, in some embodiments, the editing comprises knock-out of a gene comprising the target nucleic acid. In some embodiments, the compositions, systems and methods comprise LNPs, wherein the LNPs comprise the effector proteins described herein or nucleic acids encoding the effector proteins, the effector partners described herein or nucleic acids encoding the effector partners, the fusion proteins described herein or nucleic acids encoding the fusion proteins, or combinations thereof. In some embodiments, the LNPs comprise chemically modified guide nucleic acids. In some embodiments, the LNPs described herein are used for delivering the compositions, or one or more components of the systems described herein to a specific organ (e.g., liver). Alternatively, in some embodiments, the compositions, systems and methods comprise AAV particles, wherein the AAV particles comprise nucleic acids encoding the effector proteins described herein, the effector partners described herein, the fusion proteins described herein, or combinations thereof. In some embodiments, the AAV particles comprise nucleic acids encoding guide nucleic acids described herein. In some embodiments, the AAV particles described herein are used for delivering the compositions, or one or more components of the systems described herein to a specific cells (e.g., nerve cells or muscle cells). In some embodiments, methods comprise administering a composition or cell described herein to a subject. By way of non-limiting example, the disease comprises a cancer, an ophthalmological disorder, a neurological disorder, a neurodegenerative disease, a blood disorder, a metabolic disorder, or a combination thereof. The disease may be an inherited disorder, also referred to as a genetic disorder. The disease may be the result of an infection or associated with an infection. Also, by way of non-limiting example, the compositions are pharmaceutical compositions described herein.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include but are not limited to the diseases and syndromes listed in TABLE 10.

In some embodiments, compositions and methods edit at least one gene associated with a disease described herein or the expression thereof. In some embodiments, the disease is Alzheimer's disease and the gene is selected from APP, BACE-1, PSD95, MAPT, PSEN1, PSEN2, and APOE&4. In some embodiments, the disease is Parkinson's disease and the gene is selected from SNCA, GDNF, and LRRK2. In some embodiments, the disease is congenital muscular dystrophy 1A (MDC1A) and the gene is LAMA1 or LAMA2. In some embodiments, the disease is Ullrich Congenital Muscular Dystrophy (UCMD) and the gene is selected from COL6A1, COL6A2 and COL6A3. In some embodiments, the disease is Limb Girdle Muscular Dystrophies (LGMD1B, LGMD2A, LGMD2B) and the gene is selected from LMNA, DYSF, and CAPN3. In some embodiments, the disease is Nemaline Myopathy and the gene is selected from ACTA1, NEB, TPM2, TPM3, TNNT1, TNNT3, TNNI2 and LMOD3. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C9ORF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo (a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease is congenital adrenal hyperplasia and the gene is CAH1. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMRI. In some embodiments, the disease comprises Fuchs corneal dystrophy and the gene is selected from ZEBI, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease is cardiovascular disease and/or lipodystrophies and the gene is ANGPTL3. In some embodiments, the disease is cardiovascular disease and/or lipodystrophies and the gene is PCSK9. In some embodiments, the disease is cardiovascular disease and/or lipodystrophies and the gene is TTR. In some embodiments, the disease is severe hypertriglyceridemia (SHTG) and the gene is APOCIII or ANGPTL4. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from HSD17B13, PSD3, GPAM, CIDEB, DGAT2 and PNPLA3. In some embodiments, the disease is NASH/cirrhosis and the gene is MARCI. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease is ornithine transcarbamylase deficiency (OTCD) and the gene is OTC. In some embodiments, the disease is congenital adrenal hyperplasia (CAH) and the gene is CYP21A2. In some embodiments, the disease is atherosclerotic cardiovascular disease (ASCVD) and the gene is LPA. In some embodiments, the disease is hepatitis B virus infection (CHB) and the gene is HBV covalently closed circular DNA (cccDNA). In some embodiments, the disease is citrullinemia type I and the gene is ASSI. In some embodiments, the disease is citrullinemia type I and the gene is SLC25A13. In some embodiments, the disease is citrullinemia type I and the gene is ASSI. In some embodiments, the disease is arginase-1 deficiency and the gene is ARGI. In some embodiments, the disease is carbamoyl phosphate synthetase I deficiency and the gene is CPS1. In some embodiments, the disease is argininosuccinic aciduria and the gene is ASL. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot-Marie-Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden thrombophilia and the gene is F5. In some embodiments, the disease is fabry disease and the gene is GLA. In some embodiments, the disease is facioscapulohumeral muscular dystrophy and the gene is FSHD1. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease is hereditary angioedema and the gene is SERPING1 or KLKB1. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHII, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COLIA1, COLIA2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAGI, and JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease is thrombophilia due to antithrombin III deficiency and the gene is SERPINC1. In some embodiments the disease is spinal muscular atrophy and the gene is SMN1. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, the disease is cancer. Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer; extrahepatic (cholangiocarcinoma); bladder cancer; bone osteosarcoma/malignant fibrous histiocytoma; brain cancer (adult/childhood); brain tumor; cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver cancer); Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenström; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer; pituitary tumor, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or combinations thereof. Non-limiting examples of genes comprising a mutation associated with cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cal, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLDI, POLE, POTI, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and Cdk20. Non-limiting examples of tumor suppressor genes are TP53, RB1, and PTEN.

### Infections

Described herein are compositions, systems, and methods for treating an infection in a subject. Infections may be caused by a pathogen (e.g., bacteria, viruses, fungi, and parasites). Compositions, systems, and methods may modify a target nucleic acid associated with the pathogen or parasite causing the infection. In some embodiments, the target nucleic acid is in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, compositions, systems or methods described herein treat an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis Virus A, Hepatitis Virus B, Hepatitis Virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, compositions, systems or methods described herein treat an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### Illustrative Embodiments

Embodiment 1. A system comprising one or more components, wherein the one or more components individually or collectively comprise:
(a) an effector protein, or a nucleic acid encoding the effector protein, wherein the effector protein comprises an amino acid sequence that is at least 75%, at least 80%, at least 85%, at least 90%, at least 95%, at least 97%, or at least 98%, at least 99%, or less than 100% identical to the amino acid sequence recited in TABLE 1;
(b) an effector partner, or a nucleic acid encoding the effector partner, wherein the effector partner is a base editing enzyme; and
(c) an engineered guide nucleic acid or a nucleic acid that encodes the engineered guide nucleic acid, wherein the engineered guide nucleic acid comprises a first region and a second region, wherein the first region, at least partially, interacts with the effector protein, wherein the second region comprises a spacer sequence, and wherein the spacer sequence comprises a nucleic acid sequence hybridizes to a target sequence in a target nucleic acid.

Embodiment 2. The system of Embodiment 1, wherein the system is a base editor system for editing:
(a) an adenine (A) to guanine (G);
(b) cytosine (C) to thymine (T);
(c) cytosine (C) to guanine (G);
(d) uracil (U) to cytosine (C);
(e) guanine (G) to adenine (A);
(f) hydrolytic deamination of an adenine or adenosine, or methylation of cytosine; or
(g) a combination thereof.

Embodiment 3. The system of Embodiment 1 or 2, wherein the system is an adenosine base editor (ABE) system.

Embodiment 4. The system of Embodiment 1 or 2, wherein the system is a cytosine base editor (CBE) system.

Embodiment 5. The system of Embodiment 1 or 2, wherein the base editing enzyme comprises a deaminase or deaminase activity.

Embodiment 6. The system of Embodiment 1 or 2, wherein the effector protein, the base editing enzyme, or both are further fused to an enzyme selected from an endonuclease and a glycosylase.

Embodiment 7. The system of any one of Embodiments 1-6, wherein the base editing enzyme comprises an amino acid sequence that is at least 75%, at least 80%, at least 85%, at least 90%, at least 95%, at least 97%, or at least 98%, at least 99%, or 100% identical to any one of the amino acid sequences of SEQ ID NOS: 2-9.

Embodiment 8. The system of any one of Embodiments 1-7, wherein the base editing enzyme comprises an amino acid sequence that is at least 75%, at least 80%, at least 85%, at least 90%, at least 95%, at least 97%, or at least 98%, at least 99%, or 100% identical to SEQ ID NO: 2.

Embodiment 9. The system of any one of Embodiments 1-8, wherein the system comprises a fusion protein, or a nucleic acid encoding the fusion protein, wherein the fusion protein comprises the effector protein and the effector partner fused to each other.

Embodiment 10. The system of Embodiment 9, wherein the effector protein is linked to an N-terminus of the effector partner.

Embodiment 11. The system of Embodiment 9, wherein the effector protein is linked to a C-terminus of the effector partner.

Embodiment 12. The system of any one of Embodiments 9-11, wherein the effector protein and the effector partner are directly fused to each other.

Embodiment 13. The system of any one of Embodiments 9-12, wherein the effector protein and the effector partner are fused by a linker.

Embodiment 14. The system of any one of Embodiments 1-13, wherein the effector protein provides cis cleavage activity.

Embodiment 15. The system of any one of Embodiments 1-14, wherein the effector partner edits a base of the target nucleic acid, wherein the target nucleic acid is a single stranded target nucleic acid.

Embodiment 16. The system of Embodiment 15, wherein the effector protein cleaves the single stranded target nucleic acid.

Embodiment 17. The system of any one of Embodiments 1-14, wherein the effector protein provides nickase activity.

Embodiment 18. The system of any one of Embodiments 1-14 and 17, wherein the effector partner edits a nucleobase of the target nucleic acid, wherein the target nucleic acid is a double stranded target nucleic acid comprising a target strand and a non-target strand.

Embodiment 19. The system of Embodiment 18, wherein the effector partner edits the target strand.

Embodiment 20. The system of Embodiment 18, wherein the effector partner edits the non-target strand.

Embodiment 21. The system of any one of Embodiments 18-20, wherein the effector protein cleaves the target strand, the non-target strand, or both.

Embodiment 22. The system of Embodiment 21, wherein the effector partner edits a base of the target strand, the non-target strand, or both.

Embodiment 23. The system of any one of Embodiments 18-20, wherein the effector protein nicks the target strand or the non-target strand.

Embodiment 24. The system of Embodiment 23, wherein the effector partner edits a base of a non-nicked strand.

Embodiment 25. The system of Embodiment 23 or 24, wherein the system comprises a prime editing enzyme or a nucleic acid encoding the prime editing enzyme, wherein the prime editing enzyme catalyzes a reverse transcriptase reaction.

Embodiment 26. The system of Embodiment 25, wherein a nicked strand is corrected by reverse transcriptase editing.

Embodiment 27. The system of any one of Embodiments 1-26, wherein the effector protein comprises at least one mutation that reduces its nuclease activity, relative to an otherwise identical polypeptide without the mutation, as measured in a cleavage assay.

Embodiment 28. The system of any one of Embodiments 1-27, wherein the effector protein comprises one or more amino acid substitutions relative to an otherwise identical protein, wherein the one or more amino acid substitutions provide reduced catalytic activity relative to the otherwise identical protein.

Embodiment 29. The system of Embodiment 27 or 28, wherein the effector protein is a catalytically inactive effector protein.

Embodiment 30. The system of any one of Embodiments 27-29, wherein the one or more substitutions are selected from positions D237, D418, and E335 relative to the amino acid sequence recited in TABLE 1.

Embodiment 31. The system of any one of Embodiments 27-30, wherein the one or more amino acid substitutions are selected from D237A, D418A, D418N, E335A, and E335Q relative to the amino acid sequence recited in TABLE 1.

Embodiment 32. The system of any one of Embodiments 1-31 wherein the effector protein comprises one or more amino acid substitutions independently at positions selected from A75, K58, 180, T84, K105, D171, N193, C202, S209, G210, A218, D220, E225, C246, K250, N286, M295, M298, A306, Y315, Q360, E362 and A393 relative to the amino acid sequence recited in TABLE 1.

Embodiment 33. The system of Embodiment 32, wherein the effector protein comprises the effector protein comprising one or more amino acid substitutions selected from I80R, T84R, K105R, G210R, C202R, A218R, D220R, E225R, C246R, and Q360R relative to the amino acid sequence recited in TABLE 1.

Embodiment 34. The system of Embodiment 33, wherein the effector protein comprises the amino acid substitution of D220R relative to the amino acid sequence recited in TABLE 1.

Embodiment 35. The system of Embodiment 34, wherein the effector protein further comprises at least one of the amino acid substitutions selected from L337A, T381A, S382A, C385A, F406A, N420A, and N424A.

Embodiment 36. The system of Embodiment 34, wherein the effector protein further comprises at least one of the amino acid substitutions selected from L337A, S382A and F406A.

Embodiment 37. The system of Embodiment 34, wherein the effector protein further comprises an amino acid substitution at S382A.

Embodiment 38. The system of any one of Embodiments 1-32, wherein the effector protein one or more amino acid substitutions selected from K58W, 180K, N193K, S209F, A218K, E225K, N286K, M295W, M298L, A306K, and Y315M relative to the amino acid sequence recited in TABLE 1.

Embodiment 39. The system of any one of Embodiments 1-32, wherein the effector protein comprises one or more amino acid substitutions selected from A75F, T84R, K250N, D171K, N193K, C202K, C202R, S209F, S209Y, D220R, E225K, E225R, K250N, N286K, A306K, Y315M, Q360R, E362D, A393R and A393E relative to the amino acid sequence recited in TABLE 1.

Embodiment 40. A system comprising one or more components, wherein the one or more components individually or collectively comprise:
(a) an effector protein, or a nucleic acid encoding the effector protein, wherein the effector protein comprises an amino acid sequence that is at least 75%, at least 80%, at least 85%, at least 90%, at least 95%, at least 97%, or at least 98%, at least 99%, or less than 100% identical to the amino acid sequence recited in TABLE 1, wherein the effector protein is a catalytically inactive effector protein;
(b) an effector partner, or a nucleic acid encoding the effector partner, wherein the effector partner is an adenosine base editing enzyme; and
(c) an engineered guide nucleic acid or a nucleic acid that encodes the engineered guide nucleic acid, wherein the engineered guide nucleic acid comprises a first region and a second region, wherein the first region, at least partially, interacts with the effector protein, wherein the second region comprises a spacer sequence, and wherein the spacer sequence comprises a nucleic acid sequence that hybridizes to a target sequence in a target nucleic acid.

Embodiment 41. The system of Embodiment 40, wherein the effector protein comprises one or more amino acid substitutions selected from D237A, D418A, D418N, E335A, and E335Q relative to the amino acid sequence recited in TABLE 1.

Embodiment 42. The system of Embodiment 40 or 41, wherein the effector protein comprises, wherein the effector protein comprises one or more amino acid substitutions independently selected at positions from A75, K58, 180, T84, K105, D171, N193, C202, S209, G210, A218, D220, E225, C246, K250, N286, M295, M298, A306, Y315, Q360, E362 and A393 relative to the amino acid sequence recited in TABLE 1.

Embodiment 43. The system of Embodiment 42, wherein the effector protein comprises the effector protein comprising one or more amino acid substitutions selected from I80R, T84R, K105R, G210R, C202R, A218R, D220R, E225R, C246R, and Q360R relative to the amino acid sequence recited in TABLE 1.

Embodiment 44. The system of Embodiment 43, wherein the effector protein comprises the amino acid substitution of D220R relative to the amino acid sequence recited in TABLE 1.

Embodiment 45. The system of Embodiment 42, wherein the effector protein comprises the effector protein comprising one or more amino acid substitutions selected from K58W, 180K, N193K, S209F, A218K, E225K, N286K, M295W, M298L, A306K, and Y315M relative to the amino acid sequence recited in TABLE 1.

Embodiment 46. The system of Embodiment 42, wherein the effector protein comprises the effector protein comprising one or more amino acid substitutions selected from A75F, T84R, K250N, D171K, N193K, C202K, C202R, S209F, S209Y, D220R, E225K, E225R, K250N, N286K, A306K, Y315M, Q360R, E362D, A393R and A393E relative to the amino acid sequence recited in TABLE 1.

Embodiment 47. The system of any one of Embodiments 40-46, wherein the effector partner comprises an amino acid sequence that is at least 75%, at least 80%, at least 85%, at least 90%, at least 95%, at least 97%, or at least 98%, at least 99%, or 100% identical to the amino acid sequence of SEQ ID NO: 2.

Embodiment 48. The system of any one of Embodiments 40-47, wherein the effector partner edits a base of the target nucleic acid, wherein the target nucleic acid is a single stranded target nucleic acid.

Embodiment 49. The system of any one of Embodiments 40-47, wherein the effector partner edits a base of the target nucleic acid, wherein the target nucleic acid is a double stranded target nucleic acid comprising a target strand and a non-target strand.

Embodiment 50. The system of Embodiment 49, wherein the effector partner edits the target strand.

Embodiment 51. The system of Embodiment 49, wherein the effector partner edits the non-target strand.

Embodiment 52. The system of any one of Embodiments 40-51, wherein the system comprises a fusion protein, or a nucleic acid encoding the fusion protein, wherein the fusion protein comprises the effector protein and the effector partner fused to each other.

Embodiment 53. The system of Embodiment 52, wherein the effector protein is linked to the N-terminus the effector partner.

Embodiment 54. The system of Embodiment 53, wherein the effector protein comprises D220R and D237A amino acid substitutions, and wherein the effector partner comprises an amino acid sequence that is at least at least 90% identical to the amino acid sequence of SEQ ID NO: 2.

Embodiment 55. The system of Embodiment 53, wherein the effector protein comprises D220R and D237N amino acid substitutions, and wherein the effector partner comprises an amino acid sequence that is at least at least 90% identical to the amino acid sequence of SEQ ID NO: 2.

Embodiment 56. The system of Embodiment 53, wherein the effector protein comprises E335Q amino acid substitutions, and wherein the effector partner comprises an amino acid sequence that is at least at least 90% identical to the amino acid sequence of SEQ ID NO: 2.

Embodiment 57. The system of Embodiment 56, wherein the effector protein further comprises D220R amino acid substitution.

Embodiment 58. The system of Embodiment 52, wherein the effector protein is linked to the C-terminus of the effector partner.

Embodiment 59. The system of Embodiment 58, wherein the effector protein comprises D220R and D237A amino acid substitutions, and wherein the effector partner comprises an amino acid sequence that is at least at least 90% identical to the amino acid sequence of SEQ ID NO: 2.

Embodiment 60. The system of Embodiment 58, wherein the effector protein comprises D220R and D237N amino acid substitutions, and wherein the effector partner comprises an amino acid sequence that is at least at least 90% identical to the amino acid sequence of SEQ ID NO: 2.

Embodiment 61. The system of Embodiment 58, wherein the effector protein comprises E335Q amino acid substitutions, and wherein the effector partner comprises an amino acid sequence that is at least at least 90% identical to the amino acid sequence of SEQ ID NO: 2.

Embodiment 62. The system of Embodiment 61, wherein the effector protein further comprises D220R amino acid substitution.

Embodiment 63. The system of any one of Embodiments 1-62, wherein the system further comprises one or more additional effector partners.

Embodiment 64. The system of Embodiment 1-62, wherein the system further comprises one or more additional effector partners, optionally, fused to the fusion protein or the effector partner.

Embodiment 65. The system of Embodiment 63 or 64, wherein the one or more additional effector partners comprise a uracil glycosylase inhibitor, a ssDNA binding protein, a reverse transcriptase, a deaminase, a transcriptional activator, a transcriptional repressor, a functional domain thereof, or a combination thereof.

Embodiment 66. The system of any one of Embodiments 63-65, wherein the one or more additional effector partners independently comprises an amino sequence that is at least 75%, at least 80%, at least 85%, at least 90%, at least 95%, at least 97%, or at least 98%, at least 99%, or 100% identical to any one of the amino acid sequences recited in TABLE 2.

Embodiment 67. The system of any one of Embodiments 64-66, wherein the one or more additional effector partners are fused to the fusion protein.

Embodiment 68. The system of any one of Embodiments 1-67, wherein the spacer sequence comprises a nucleotide sequence in a range of from 10 to 24 linked nucleotides.

Embodiment 69. The system of any one of Embodiments 1-68, wherein the spacer sequence consists of a nucleotide sequence of 12, 13, 20, or 21 nucleotides.

Embodiment 70. The system of any one of Embodiments 1-69, wherein the spacer sequence comprises a nucleotide sequence of 20 linked nucleotides.

Embodiment 71. The system of any one of Embodiments 1-69, wherein the spacer sequence comprises a nucleotide sequence of 21 linked nucleotides.

Embodiment 72. The system of any one of Embodiments 1-69, wherein the spacer sequence comprises a nucleotide sequence of 12 linked nucleotides.

Embodiment 73. The system of any one of Embodiments 1-69, wherein the spacer sequence comprises a nucleotide sequence of 13 linked nucleotides.

Embodiment 74. The system of any one of Embodiments 1-73, wherein the spacer sequence comprises a nucleotide sequence that is about 80% to about 95% complementary to the target sequence.

Embodiment 75. The system of any one of Embodiments 1-74, wherein the spacer sequence comprises a nucleotide sequence that is at least 90% identical to any one of the nucleotide sequences recited in TABLE 21, TABLE 22, TABLE 23, TABLE 24, TABLE 25, TABLE 26, TABLE 27 and TABLE 28.

Embodiment 76. The system of any one of Embodiments 1-75, wherein the first region comprises a repeat sequence that at least partially interacts with the effector protein.

Embodiment 77. The system of Embodiment 76, wherein the repeat sequence comprises a nucleotide sequence that is at least 75%, at least 80%, at least 85%, at least 90%, at least 95%, at least 97%, or at least 98%, at least 99%, or 100% identical to any one of the nucleotide sequences recited in TABLE 5.

Embodiment 78. The system of any one of Embodiments 1-77, wherein the engineered guide nucleic acid is a crRNA.

Embodiment 79. The system of any one of Embodiments 1-75, wherein the first region comprises an intermediary sequence that at least partially interacts with the effector protein.

Embodiment 80. The system of Embodiment 79, wherein the intermediary sequence comprises a nucleotide sequence that is at least 75%, at least 80%, at least 85%, at least 90%, at least 95%, at least 97%, or at least 98%, at least 99%, or 100% identical to any one of the nucleotide sequences recited in TABLE 6.

Embodiment 81. The system of any one of Embodiments 1-75 and 79-80, wherein the first region comprises a handle sequence that at least partially interacts with the effector protein.

Embodiment 82. The system of Embodiment 81, wherein the handle sequence comprises a nucleotide sequence that is at least 75%, at least 80%, at least 85%, at least 90%, at least 95%, at least 97%, or at least 98%, at least 99%, or 100% identical to any one of the nucleotide sequences recited in TABLE 5, TABLE 6 and TABLE 8.

Embodiment 83. The system of any one of Embodiments 1-75 and 79-82, wherein the engineered guide nucleic acid is a sgRNA.

Embodiment 84. The system of any one of Embodiments 1-83, wherein the engineered guide nucleic acid comprises one or more phosphorothioate (PS) backbone modifications, one or more 2′-fluoro (2′-F) sugar modifications, one or more 2′-O-Methyl(2′OMe) sugar modifications, one or more GC covariation modifications, or combinations thereof.

Embodiment 85. The system of any one of Embodiments 1-84, wherein the engineered guide nucleic acid comprises a chemical modification pattern represented in FIG. 43.

Embodiment 86. The system of any one of Embodiments 1-85, wherein the target sequence is adjacent to a protospacer adjacent motif (PAM).

Embodiment 87. The system of Embodiment 86, wherein the effector protein recognizes the PAM.

Embodiment 88. The system of Embodiment 86 or 87, wherein the PAM comprises any one of the nucleotide sequences of TABLE 4.

Embodiment 89. The system of any one of Embodiment 1-88, wherein the effector protein, the effector partner or a combination thereof further comprises one or more additional heterologous peptides that are heterologous to the effector protein, the effector partner, or the combination thereof, respectively.

Embodiment 90. The system of Embodiment 89, wherein the one or more additional heterologous peptides are located at N-terminus, C-terminus, or both of the effector protein, the effector partner, or the combination thereof.

Embodiment 91. The system of Embodiment 89 or 90, wherein the one or more heterologous peptide independently comprises any one of the amino acid sequences recited in TABLE 3.

Embodiment 92. The system of any one of Embodiment 1-91, wherein the target sequence is within a human gene.

Embodiment 93. The system of any one of Embodiment 1-92, wherein the target nucleic acid is any one of the genes set forth in TABLE 9.

Embodiment 94. The system of any one of Embodiment 1-93, wherein the target nucleic acid is associated with any one of the diseases or disorders of TABLE 10.

Embodiment 95. The system of any one of Embodiments 1-94, wherein the system comprises an expression vector, wherein the expression vector encodes the effector protein, the base editing enzyme, the engineered guide nucleic acid, the one or more additional effector partners, or a combination thereof.

Embodiment 96. The system of Embodiment 95, wherein the nucleic acid expression vector is a viral vector or a non-viral vector.

Embodiment 97. The system of Embodiment 96, wherein the viral vector is an adeno associated viral (AAV) vector.

Embodiment 98. The system of any one of Embodiments 1-97, wherein the nucleic acids encoding the effector protein, the effector partner or combinations thereof comprises messenger RNAs.

Embodiment 99. The system of any one of Embodiments 1-98 comprising a lipid or a lipid nanoparticle.

Embodiment 100. The system of Embodiment 99, wherein the lipid nanoparticle is formulated according to any one of LNP formulations described in TABLE 20.

Embodiment 101. A system comprising one or more components, wherein the one or more components individually or collectively comprise:
(a) An effector protein, or a nucleic acid encoding the effector protein, wherein the effector protein comprises an amino acid sequence of SEQ ID NO: 1 having D220R and E335Q substitutions relative to SEQ ID NO: 1;
(b) an effector partner, or a nucleic acid encoding the effector partner, wherein the effector partner is a base editing enzyme, wherein the base editing enzyme comprises an amino acid sequence of SEQ ID NO: 2, and wherein the N-terminus of the base editing enzyme is fused to C-terminus of the effector protein; and
(c) an engineered guide nucleic acid or a nucleic acid that encodes the engineered guide nucleic acid, wherein the engineered guide nucleic acid comprises a first region and a second region, wherein the first region, at least partially, interacts with the effector protein, wherein the second region comprises a spacer sequence, wherein the spacer sequence comprises any one of nucleic acid sequences recited in TABLE 21, TABLE 22, TABLE 23, TABLE 24, TABLE 25, TABLE 26, TABLE 27 and TABLE 28.

Embodiment 102. A system comprising one or more components, wherein the one or more components individually comprise:
(a) an effector protein, or a nucleic acid encoding the effector protein, wherein the effector protein comprises any one of the amino acid sequences recited in TABLE 1.1;
(b) an effector partner, or a nucleic acid encoding the effector partner, wherein the effector partner comprises any one of the amino acid sequences of TABLE 2; and
(c) an engineered guide nucleic acid or a nucleic acid that encodes the engineered guide nucleic acid, wherein:

the engineered guide nucleic acid, at least partially, interacts with a target nucleic acid, the target nucleic acid comprises a target strand or a non-target strand of the target nucleic acid, the effector protein nicks the target strand or the non-target strand, and the effector partner edits a non-nicked strand.

Embodiment 103. The system of Embodiment 102, wherein the effector protein comprises any one of the amino acid sequences of SEQ ID NOs: 379, 377 and 381.

Embodiment 104. The system of Embodiment 102 or 103, wherein the effector protein comprises an amino acid sequence of SEQ ID NO: 379.

Embodiment 105. The system of any one of Embodiments 102-104, wherein a nicked strand is corrected by reverse transcriptase editing.

Embodiment 106. The system of any one of Embodiments 102-105, wherein the effector partner is fused to the effector protein.

Embodiment 107. A library of nucleic acid expression vectors comprising at least one of the nucleic acid expression vectors of Embodiments 95-98.

Embodiment 108. A composition comprising one or more components of any one of Embodiments 1-106.

Embodiment 109. A composition comprising one or more components of any one of Embodiments 1-106 for use in therapy.

Embodiment 110. A pharmaceutical composition comprising:
(a) the system of any one of Embodiments 1-106 or the composition of Embodiment 108 or 109; and
(b) a pharmaceutically acceptable excipient.

Embodiment 111. A cell or progeny thereof comprising the system of any one of Embodiments 1-106, the library of nucleic acid expression vectors of Embodiment 107, the composition of Embodiment 108 or 109, or the pharmaceutical composition of Embodiment 110.

Embodiment 112. A method of modifying a target nucleic acid of a cell, the method comprising contacting the cell with one or more of:
(a) the system of any one of Embodiments 1-106;
(b) the library of nucleic acid expression vectors of Embodiment 107;
(c) the composition of Embodiment 108 or 109; or
(d) the pharmaceutical composition of Embodiment 110, thereby modifying the target nucleic acid of the cell.

Embodiment 113. A method of modifying a target nucleic acid, the method comprising contacting the target nucleic acid with one or more of:
(a) the system of any one of Embodiments 1-106;
(b) the library of nucleic acid expression vectors of Embodiment 107;
(c) the composition of Embodiment 108 or 109; or
(d) the pharmaceutical composition of Embodiment 110, thereby modifying the target nucleic acid.

Embodiment 114. A method of modifying a nucleobase of a target nucleic acid within a human gene or associated with expression of a human gene, the method comprising contacting the target nucleic acid with one or more of:
(a) the system of any one of Embodiments 1-106;
(b) the library of nucleic acid expression vectors of Embodiment 107;
(c) the composition of Embodiment 108 or 109; or
(d) the pharmaceutical composition of Embodiment 110, thereby modifying the target nucleic acid.

Embodiment 115. A method of modifying a target nucleic acid of a cell, the method comprising contacting the cell with one or more of:
(a) the system of any one of Embodiments 1-106;
(b) the composition of Embodiment 108 or 109; or
(c) the pharmaceutical composition of Embodiment 110, thereby modifying the target nucleic acid of the cell.

Embodiment 116. A method of modifying a target nucleic acid, the method comprising contacting the target nucleic acid with one or more of:
(a) the system of any one of Embodiments 1-106;
(b) the composition of Embodiment 108 or 109; or
(c) the pharmaceutical composition of Embodiment 110, thereby modifying the target nucleic acid.

Embodiment 117. A method of modifying a nucleobase of a target nucleic acid within a human gene or associated with expression of a human gene, the method comprising contacting the target nucleic acid with one or more of:
(a) the system of any one of Embodiments 1-106;
(b) the composition of Embodiment 108 or 109; or
(c) the pharmaceutical composition of Embodiment 110, thereby modifying the target nucleic acid.

Embodiment 118. The method of any one of Embodiments 112-117 comprising modifying a target strand of the target nucleic acid.

Embodiment 119. The method of any one of Embodiments 112-117 comprising modifying a non-target strand of the target nucleic acid.

Embodiment 120. The method of any one of Embodiments 112-117, wherein the method is performed in a cell.

Embodiment 121. The method of any one of Embodiments 112-117, wherein the method is performed in vivo.

Embodiment 122. The method of any one of Embodiments 112-117, wherein the method is performed ex vitro.

Embodiment 123. The method of any one of Embodiments 112-117, wherein the method is performed in vitro.

Embodiment 124. The method of any one of Embodiments 112-123, wherein the target nucleic acid comprises one or more mutations associated with a disease.

Embodiment 125. The method of Embodiment 124, wherein the one or more mutations comprise a point mutation, a single nucleotide polymorphism (SNP), a chromosomal mutation, a copy number mutation, or any combination thereof.

Embodiment 126. The method of Embodiment 124 or 125, wherein the target nucleic acid is any one of the genes set forth in TABLE 9.

Embodiment 127. The method of any one of Embodiments 124-126, wherein the target nucleic acid is associated with any one of the diseases set forth in TABLE 10.

Embodiment 128. A cell or progeny thereof contacted by:
(a) the system of any one of Embodiments 1-106;
(b) the library of nucleic acid expression vectors of Embodiment 107;
(c) the composition of Embodiment 108 or 109;
(d) the pharmaceutical composition of Embodiment 110; or
(e) the method of any one of Embodiments 112-127.

Embodiment 129. A cell or progeny thereof comprising a nucleotide base of a target nucleic acid modified by:
(a) the system of any one of Embodiments 1-106;
(b) the library of nucleic acid expression vectors of Embodiment 107;
(c) the composition of Embodiment 108 or 109;
(d) the pharmaceutical composition of Embodiment 110; or
(e) the method of any one of Embodiments 112-127.

Embodiment 130. The cell or progeny thereof of any one of Embodiments 111 and 128-129, wherein the cell is a eukaryotic cell.

Embodiment 131. The cell or progeny thereof of any one of Embodiments 111 and 128-130, wherein the cell is a mammalian cell.

Embodiment 132. The cell or progeny thereof of any one of Embodiments 111 and 128-131, wherein the cell is a human cell.

Embodiment 133. A population of cells that comprises at least one cell of any one of Embodiments 111 and 128-132.

Embodiment 134. A method of inserting a double stranded DNA donor nucleic acid (dsDNA donor) into a target nucleic acid, comprising contacting the target nucleic acid with an effector protein or a nucleic acid encoding the same, and the dsDNA donor, wherein the effector protein comprises an amino acid sequence that is at least 70%, at least 75%, at least 80%, at least 85%, at least 90%, at least 95%, at least 97%, at least 98%, at least 99%, or 100% identical to of one or more nucleotides.

Embodiment 135. The method of Embodiment 134, wherein the method does not comprise contacting the target nucleic acid with an additional enzyme.

Embodiment 136. The method of Embodiment 134, wherein the method does not comprise contacting the target nucleic acid with a reverse transcriptase, DNA repair protein or combination thereof.

Embodiment 137. The method of any one of embodiments 134-136, comprising contacting the target nucleic acid with an inhibitor of non-homologous end joining repair.

### SEQUENCES AND TABLES

TABLE 1 provides illustrative amino acid sequences of effector proteins that are useful in the compositions, systems and methods described herein.

TABLE 1
EXEMPLARY AMINO ACID SEQUENCE(S) OF EFFECTOR PROTEIN

| | SEQ | |
|---|---|---|
| | ID | |
| Name | NO: | Sequence |
| CasM.265466 | 1 | MSVLTRKVQLIPVGDKEERDRVYKYLRDGIEAQNRAMNLYMSGLYFAA |
| | | INEASKEDRKELNQLYSRIATSSKGSAYTTDIEFPTGLASTSTLSMAVRQD |
| | | FTKSLKDGLMYGRVSLPTYRKDNPLFVDVRFVALRGTKQKYNGLYHEY |
| | | KSHTEFLDNLYSSDLKVYIKFANDITFQVIFGNPRKSSALRSEFQNIFEEY |
| | | YKVCQSSIQFSGTKIILNMAMDIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRCSCCGYED |
| | | AGNRPKKEKGQAYFKCLKCGEEMNADFNAARNIAMSTEFQSGKKTKK |
| | | QKKEQHENK |

TABLE 1.1 provides illustrative amino acid sequences of effector protein variants that are useful in the compositions, systems and methods described herein.

TABLE 1.1
EXEMPLARY VARIANT AMINO ACID SEQUENCE(S) OF EFFECTOR
PROTEIN

| | SEQ | |
|---|---|---|
| | ID | |
| Name | NO: | Sequence |
| CasM. | 375 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| N420A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRCSCCGYED |
| | | AGNRPKKEKGQAYFKCLKCGEEMNADFAAARNIAMSTEFQSGKKTKK |
| | | QKKEQHENK |
| CasM. | 376 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| N424A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRCSCCGYED |
| | | AGNRPKKEKGQAYFKCLKCGEEMNADFNAARAIAMSTEFQSGKKTKK |
| | | QKKEQHENK |
| CasM. | 377 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| F406A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRCSCCGYED |
| | | AGNRPKKEKGQAYAKCLKCGEEMNADFNAARNIAMSTEFQSGKKTKK |
| | | QKKEQHENK |
| CasM. | 378 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| C385A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRASCCGYED |
| | | AGNRPKKEKGQAYFKCLKCGEEMNADFNAARNIAMSTEFQSGKKTKK |
| | | QKKEQHENK |
| CasM. | 379 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| S382A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTAQRCSCCGYED |
| | | AGNRPKKEKGQAYFKCLKCGEEMNADFNAARNIAMSTEFQSGKKTKK |
| | | QKKEQHENK |
| CasM. | 380 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| T381A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHASQRCSCCGYED |
| | | AGNRPKKEKGQAYFKCLKCGEEMNADFNAARNIAMSTEFQSGKKTKK |
| | | QKKEQHENK |
| CasM. | 381 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| L337A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENAEGIRDDV |
| | | KNEWLLSNWSYYQLQQYITYKAKTYGIEVRKINPYHTSQRCSCCGYED |
| | | AGNRPKKEKGQAYFKCLKCGEEMNADFNAARNIAMSTEFQSGKKTKK |
| | | QKKEQHENK |
| CasM. | 382 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| N336A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLEALEGIRDDV |
| | | KNEWLLSNWSYYQLQQYITYKAKTYGIEVRKINPYHTSQRCSCCGYED |
| | | AGNRPKKEKGQAYFKCLKCGEEMNADFNAARNIAMSTEFQSGKKTKK |
| | | QKKEQHENK |
| CasM. | 383 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| Y379A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPAHTSQRCSCCGYED |
| | | AGNRPKKEKGQAYFKCLKCGEEMNADFNAARNIAMSTEFQSGKKTKK |
| | | QKKEQHENK |
| CasM. | 384 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| N424A/ | | FTKSLKDGLMYGRVSLPTYRKDNPLFVDVRFVALRGTKQKYNGLYHEY |
| D220R | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRCSCCGYED |
| | | AGNRPKKEKGQAYFKCLKCGEEMNADFNAARAIAMSTEFQSGKKTKK |
| | | QKKEQHENK |
| CasM. | 385 | MSVLTRKVQLIPVGDKEERDRVYKYLRDGIEAQNRAMNLYMSGLYFAA |
| 265466 | | INEASKEDRKELNQLYSRIATSSKGSAYTTDIEFPTGLASTSTLSMAVRQD |
| D220R | | FTKSLKDGLMYGRVSLPTYRKDNPLFVDVRFVALRGTKQKYNGLYHEY |
| | | KSHTEFLDNLYSSDLKVYIKFANDITFQVIFGNPRKSSALRSEFQNIFEEY |
| | | YKVCQSSIQFSGTKIILNMAMRIPDKEIELDEDVCVGVDLGIAIPAVCALN |
| | | KNRYSRVSIGSKEDFLRVRTKIRNQRKRLQTNLKSSNGGHGRKKKMKP |
| | | MDRFRDYEANWVQNYNHYVSRQVVDFAVKNKAKYINLENLEGIRDDV |
| | | KNEWLLSNWSYYQLQQYITYKAKTYGIEVRKINPYHTSQRCSCCGYED |
| | | AGNRPKKEKGQAYFKCLKCGEEMNADFNAARNIAMSTEFQSGKKTKK |
| | | QKKEQHENK |

TABLE 2 provides illustrative sequences of exemplary effector partners that are useful in the compositions, systems and methods described herein.

TABLE 2
Exemplary Amino Acid Sequence(s) of Effector Partners

| Nickname | SEQ | |
|---|---|---|
| (typical | ID | |
| function) | NO: | Sequence |
| ABE8e | 2 | SEVEFSHEYWMRHALTLAKRARDEREVPVGAVLVLNNRVIGEGWN |
| (base editing | | RAIGLHDPTAHAEIMALRQGGLVMQNYRLIDATLYVTFEPCVMCAG |
| enzyme) | | AMIHSRIGRVVFGVRNSKRGAAGSLMNVLNYPGMNHRVEITEGILA |
| | | DECAALLCDFYRMPRQVFNAQKKAQSSIN |
| ABE8e-TadA | 3 | SEVEFSHEYWMRHALTLAKRARDEREVPVGAVLVLNNRVIGEGWN |
| (base editing | | RAIGLHDPTAHAEIMALRQGGLVMQNYRLIDATLYVTFEPCVMCAG |
| enzyme) | | AMIHSRIGRVVFGVRNSKRGAAGSLMNVLNYPGMNHRVEITEGILA |
| | | DECAALLCDFYRMPRQVFNAQKKAQSSINSGGSSGGSSGSETPGTSE |
| | | SATPESSGGSSGGSSEVEFSHEYWMRHALTLAKRAWDEREVPVGAV |
| | | LVHNNRVIGEGWNRPIGRHDPTAHAEIMALRQGGLVMQNYRLIDAT |
| | | LYVTLEPCVMCAGAMIHSRIGRVVFGARDAKTGAAGSLMDVLHHPG |
| | | MNHRVEITEGILADECAALLSDFFRMRRQEIKAQKKAQSSTD |
| TadA-ABE8e | 4 | SEVEFSHEYWMRHALTLAKRAWDEREVPVGAVLVHNNRVIGEGWN |
| (base editing | | RPIGRHDPTAHAEIMALRQGGLVMQNYRLIDATLYVTLEPCVMCAG |
| enzyme) | | AMIHSRIGRVVFGARDAKTGAAGSLMDVLHHPGMNHRVEITEGILA |
| | | DECAALLSDFFRMRRQEIKAQKKAQSSTDSGGSSGGSSGSETPGTSES |
| | | ATPESSGGSSGGSSEVEFSHEYWMRHALTLAKRARDEREVPVGAVL |
| | | VLNNRVIGEGWNRAIGLHDPTAHAEIMALRQGGLVMQNYRLIDATL |
| | | YVTFEPCVMCAGAMIHSRIGRVVFGVRNSKRGAAGSLMNVLNYPG |
| | | MNHRVEITEGILADECAALLCDFYRMPRQVFNAQKKAQSSIN |
| ABE8.20m | 5 | SEVEFSHEYWMRHALTLAKRARDEREVPVGAVLVLNNRVIGEGWN |
| (base editing | | RAIGLHDPTAHAEIMALRQGGLVMQNYRLYDATLYSTFEPCVMCAG |
| enzyme) | | AMIHSRIGRVVFGVRNAKTGAAGSLMDVLHHPGMNHRVEITEGILA |
| | | DECAALLCRFFRMPRRVFNAQKKAQSSTD |
| APOBEC3A | 6 | EASPASGPRHLMDPHIFTSNFNNGIGRHKTYLCYEVERLDNGTSVKM |
| (base editing | | DQHRGFLHNQAKNLLCGFYGRHAELRFLDLVPSLQLDPAQIYRVTW |
| enzyme or | | FISWSPCFSWGCAGEVRAFLQENTHVRLRIFAARIYDYDPLYKEALQ |
| deaminase) | | MLRDAGAQVSIMTYDEFKHCWDTFVDHQGCPFQPWDGLDEHSQAL |
| | | SGRLRAILQNQGN |
| Anc APOBEC | 7 | SSETGPVAVDPTLRRRIEPHEFEVFFDPRELRKETCLLYEIKWGTSHKI |
| (a.k.a. | | WRHSSKNTTKHVEVNFIEKFTSERHFCPSTSCSITWFLSWSPCGECSK |
| AncBE4Max) | | AITEFLSQHPNVTLVIYVARLYHHMDQQNRQGLRDLVNSGVTIQIMT |
| (editing enzyme | | APEYDYCWRNFVNYPPGKEAHWPRYPPLWMKLYALELHAGILGLPP |
| editor or | | CLNILRRKQPQLTFFTIALQSCHYQRLPPHILWATGLK |
| deaminase) | | |
| BtAPOBEC2 | 8 | MAQKEEAAAAAEPASQNGEEVENLEDPEKLKELIELPPFEIVTGERLP |
| (base editing | | AHYFKFQFRNVEYSSGRNKTFLCYVVEAQSKGGQVQASRGYLEDEH |
| enzyme or | | ATNHAEEAFFNSIMPTFDPALRYMVTWYVSSSPCAACADRIVKTLNK |
| deaminase) | | TKNLRLLILVGRLFMWEEPEIQAALRKLKEAGCRLRIMKPQDFEYIW |
| | | QNFVEQEEGESKAFEPWEDIQENFLYYEEKLADILK |
| M-MLV RT or | 9 | TLNIEDEYRLHETSKEPDVSLGSTWLSDFPQAWAETGGMGLAVRQA |
| mutant thereof | | PLIIPLKATSTPVSIKQYPMSQEARLGIKPHIQRLLDQGILVPCQSPWNT |
| (D200N/L603W/ | | PLLPVKKPGTNDYRPVQDLREVNKRVEDIHPTVPNPYNLLSGLPPSH |
| T330P/T306K/ | | QWYTVLDLKDAFFCLRLHPTSQPLFAFEWRDPEMGISGQLTWTRLPQ |
| W313F | | GFKNSPTLFNEALHRDLADFRIQHPDLILLQYVDDLLLAATSELDCQQ |
| mutations) | | GTRALLQTLGNLGYRASAKKAQICQKQVKYLGYLLKEGQRWLTEA |
| (prime editing) | | RKETVMGQPTPKTPRQLREFLGKAGFCRLFIPGFAEMAAPLYPLTKP |
| | | GTLFNWGPDQQKAYQEIKQALLTAPALGLPDLTKPFELFVDEKQGY |
| | | AKGVLTQKLGPWRRPVAYLSKKLDPVAAGWPPCLRMVAAIAVLTK |
| | | DAGKLTMGQPLVILAPHAVEALVKQPPDRWLSNARMTHYQALLLD |
| | | TDRVQFGPVVALNPATLLPLPEEGLQHNCLDILAEAHGTRPDLTDQP |
| | | LPDADHTWYTDGSSLLQEGQRKAGAAVTTETEVIWAKALPAGTSAQ |
| | | RAELIALTQALKMAEGKKLNVYTDSRYAFATAHIHGEIYRRRGWLTS |
| | | EGKEIKNKDEILALLKALFLPKRLSIIHCPGHQKGHSAEARGNRMAD |
| | | QAARKAAITETPDTSTLLIENSSP |
| TET1 | 10 | MASNFTQFVLVDNGGTGDVTVAPSNFANGVAEWISSNSRSQAYKVT |
| (CRISPRa) | | CSVRQSSAQKRKYTIKVEVPKVATQTVGGVELPVAAWRSYLNMELT |
| | | IPIFATNSDCELIVKAMQGLLKDGNPIPSAIAANSGIYSAGGGGSGGG |
| | | GSGGGGSPKKKRKVEASGGGSGGGSGGGSGEAAPCDCDGGTQKEK |
| | | GPYYTHLGAGPSVAAVRELMETRFGQKGKAIRIEKIVFTGKEGKSSQ |
| | | GCPVAKWVIRRSGPEEKLICLVRERVDHHCSTAVIVVLILLWEGIPRL |
| | | MADRLYKELTENLRSYSGHPTDRRCTLNKKRTCTCQGIDPKTCGASF |
| | | SFGCSWSMYFNGCKFGRSENPRKFRLAPNYPLHNYYKRITGMSSEGS |
| | | DVKTGWIIPDRKTLISREEKQLEKNLQELATVLAPLYKQMAPVAYQN |
| | | QVEYEEVAGDCRLGNEEGRPFSGVTCCMDFCAHSHKDIHNMHNGST |
| | | VVCTLIRADGRDTNCPEDEQLHVLPLYRLADTDEFGSVEGMKAKIKS |
| | | GAIQVNGPTRKRRLRFTEPVPRCGKRAKMKQNHNKSGSHNTKSFSS |
| | | ASSTSHLVKDESTDFCPLQASSAETSTCTYSKTASGGFAETSSILHCT |
| | | MPSGAHSGANAAAGECTGTVQPAEVAAHPHQSLPTADSPVHAEPLT |
| | | SPSEQLTSNQSNQQLPLLSNSQKLASCQVEDERHPEADEPQHPEDDN |
| | | LPQLDEFWSDSEEIYADPSFGGVAIAPIHGSVLIECARKELHATTSLRS |
| | | PKRGVPFRVSLVFYQHKSLNKPNHGFDINKIKCKCKKVTKKKPADRE |
| | | CPDVSPEANLSHQIPSRVASTLTRDNVVTVSPYSLTHVAGPYNRWVA |
| | | AADYKDDDDK |
| TET2 | 11 | QSQPGHNQMLRPIKTEPVSKPSSYRYPLSPPQENMSSRIKQEISSPSRD |
| (CRISPRa) | | NGQPKSIIETMEQHLKQFQLKSLCDYKALTLKSQKHVKVPTDIQAAE |
| | | SENHARAAEPQATKSTDCSVLDDVSESDTPGEQSQNGKCEGCNPDK |
| | | DEAPYYTHLGAGPDVAAIRTLMEERYGEKGKAIRIEKVIYTGKEGKS |
| | | SQGCPIAKWVYRRSSEEEKLLCLVRVRPNHTCETAVMVIAIMLWDGI |
| | | PKLLASELYSELTDILGKCGICTNRRCSQNETKKKQSPPRNCCCQGEN |
| | | PETCGASFSFGCSWSMYYNGCKFARSKKPRKFRLHGAEPKEEERLGS |
| | | HLQNLATVIAPIYKKLAPDAYNNQVEFEHQAPDCCLGLKEGRPFSGV |
| | | TACLDFSAHSHRDQQNMPNGSTVVVTLNREDNREVGAKPEDEQFHV |
| | | LPMYIIAPEDEFGSTEGQEKKIRMGSIEVLQSFRRRRVIRIGELPKSCK |
| | | KKAEPKKAKTKKAARKHSSLENCSSRTEKGKSSSHTKLMENASHMK |
| | | QMTAQPQLSGPVIRQPPTLQRHLQQGQRPQQPQPPQPQPQTTPQPQP |
| | | QPQHIMPGNSQSVGSHCSGSTSVYTRQPTPHSPYPSSAHTSDIYGDTN |
| | | HVNFYPTSSHASGSYLNPSNYMNPYLGLLNQNNQYAPFPYNGSVPV |
| | | DNGSPFLGSYSPQAQSRDLHRYPNQDHLTNQNLPPIHTLHQQTFGDS |
| | | PSKYLSYGNQNMQRDAFTTNSTLKPNVHHLATFSPYPTPKMDSHFM |
| | | GAASRSPYSHPHTDYKTSEHHLPSHTVYSYTAAASGSSSSHAFHNKE |
| | | NDNIANGLSRVLPGFNHDRTASAQELLYSLTGSSQEKQPEVSGQDAA |
| | | AVQEIEYWSDSEHNFQDPCIGGVAIAPTHGSILIECAKCEVHATTKVN |
| | | DPDRNHPTRISLVLYRHKNLFLPKHCLALWEAKMAEKARKEEECGK |
| | | NGSDHVSQKNHGKQEKREPTGPQEPSYLRFIQSLAENTGSVTTDSTV |
| | | TTSPYAFTQVTGPYNTFV |
| P300 (CRISPRa) | 12 | IFKPEELRQALMPTLEALYRQDPESLPFRQPVDPQLLGIPDYFDIVKSP |
| | | MDLSTIKRKLDTGQYQEPWQYVDDIWLMFNNAWLYNRKTSRVYKY |
| | | CSKLSEVFEQEIDPVMQSLGYCCGRKLEFSPQTLCCYGKQLCTIPRDA |
| | | TYYSYQNRYHFCEKCFNEIQGESVSLGDDPSQPQTTINKEQFSKRKN |
| | | DTLDPELFVECTECGRKMHQICVLHHEIIWPAGFVCDGCLKKSARTR |
| | | KENKFSAKRLPSTRLGTFLENRVNDFLRRQNHPESGEVTVRVVHASD |
| | | KTVEVKPGMKARFVDSGEMAESFPYRTKALFAFEEIDGVDLCFFGM |
| | | HVQEYGSDCPPPNQRRVYISYLDSVHFFRPKCLRTAVYHEILIGYLEY |
| | | VKKLGYTTGHIWACPPSEGDDYIFHCHPPDQKIPKPKRLQEWYKKM |
| | | LDKAVSERIVHDYKDIFKQATEDRLTSAKELPYFEGDFWPNVLEESIK |
| | | ELEQEEEERKREENTSNESTDVTKGDSKNAKKKNNKKTSKNKSSLSR |
| | | GNKKKPGMPNVSNDLSQKLYATMEKHKEVFFVIRLIAGPAANSLPPI |
| | | VDPDPLIPCDLMDGRDAFLTLARDKHLEFSSLRRAQWSTMCMLVEL |
| | | HTQSQD |
| VPR (CRISPRa) | 13 | DALDDFDLDMLGSDALDDFDLDMLGSDALDDFDLDMLGSDALDDF |
| | | DLDMLINSRSSGSPKKKRKVGSQYLPDTDDRHRIEEKRKRTYETFKSI |
| | | MKKSPFSGPTDPRPPPRRIAVPSRSSASVPKPAPQPYPFTSSLSTINYDE |
| | | FPTMVFPSGQISQASALAPAPPQVLPQAPAPAPAPAMVSALAQAPAP |
| | | VPVLAPGPPQAVAPPAPKPTQAGEGTLSEALLQLQFDDEDLGALLGN |
| | | STDPAVFTDLASVDNSEFQQLLNQGIPVAPHTTEPMLMEYPEAITRLV |
| | | TGAQRPPDPAPAPLGAPGLPNGLLSGDEDFSSIADMDFSALLGSGSGS |
| | | RDSREGMFLPKPEAGSAISDVFEGREVCQPKRIRPFHPPGSPWANRPL |
| | | PASLAPTPTGPVHEPVGSLTPAPVPQPLDPAPAVTPEASHLLEDPDEET |
| | | SQAVKALREMADTVIPQKEEAAICGQMDLSHPPPRGHLDELTTTLES |
| | | MTEDLNLDSPLTPELNEILDTFLNDECLLHAMHISTGLSIFDTSLF |
| DNMT3A | 14 | TYGLLRRREDWPSRLQMFFANNHDQEFDPPKVYPPVPAEKRKPIRVL |
| (CRISPRi) | | SLFDGIATGLLVLKDLGIQVDRYIASEVCEDSITVGMVRHQGKIMYV |
| | | GDVRSVTQKHIQEWGPFDLVIGGSPCNDLSIVNPARKGLYEGTGRLF |
| | | FEFYRLLHDARPKEGDDRPFFWLFENVVAMGVSDKRDISRFLESNPV |
| | | MIDAKEVSAAHRARYFWGNLPGMNRPLASTVNDKLELQECLEHGRI |
| | | AKFSKVRTITTRSNSIKQGKDQHFPVFMNEKEDILWCTEMERVFGFP |
| | | VHYTDVSNMSRLARQRLLGRSWSVPVIRHLFAPLKEYFACV |
| DNMT3L | 15 | MAAIPALDPEAEPSMDVILVGSSELSSSVSPGTGRDLIAYEVKANQRN |
| (CRISPRi) | | IEDICICCGSLQVHTQHPLFEGGICAPCKDKFLDALFLYDDDGYQSYC |
| | | SICCSGETLLICGNPDCTRCYCFECVDSLVGPGTSGKVHAMSNWVCY |
| | | LCLPSSRSGLLQRRRKWRSQLKAFYDRESENPLEMFETVPVWRRQPV |
| | | RVLSLFEDIKKELTSLGFLESGSDPGQLKHVVDVTDTVRKDVEEWGP |
| | | FDLVYGATPPLGHTCDRPPSWYLFQFHRLLQYARPKPGSPRPFFWMF |
| | | VDNLVLNKEDLDVASRFLEMEPVTIPDVHGGSLQNAVRVWSNIPAIR |
| | | SRHWALVSEEELSLLAQNKQSSKLAAKWPTKLVKNCFLPLREYFKY |
| | | FST |
| VP64 | 16 | DALDDFDLDMLGSDALDDFDLDMLGSDALDDFDLDMLGSDALDDF |
| (CRISPRa) | | DLDML |
| EZH2 | 17 | GQTGKKSEKGPVCWRKRVKSEYMRLRQLKRFRRADEVKTMFSSNR |
| (CRISPRi) | | QKILERTETLNQEWKQRRIQPVHIMTSVSSLRGTRECSVTSDLDFPAQ |
| | | VIPLKTLNAVASVPIMYSWSPLQQNFMVEDETVLHNIPYMGDEVLDQ |
| | | DGTFIEELIKNYDGKVHGDRECGFINDEIFVELVNALGQYNDDDDDD |
| | | DGDDPDEREEKQKDLEDNRDDKETCPPRKFPADKIFEAISSMFPDKG |
| | | TAEELKEKYKELTEQQLPGALPPECTPNIDGPNAKSVQREQSLHSFHT |
| | | LFCRRCFKYDCFLHPFHATPNTYKRKNTETALDNKPCGPQCYQHLEG |
| | | AKEFAAALTAERIKTPPKRPGGRRRGRLPNNSSRPSTPTISVLESKDTD |
| | | SDREAGTETGGENNDKEEEEKKDETSSSSEANSRCQTPIKMKPNIEPP |
| | | ENVEWSGAEASMFRVLIGTYYDNFCAIARLIGTKTCRQVYEFRVKES |
| | | SIIAPVPTEDVDTPPRKKKRKHRLWAAHCRKIQLKKDGSSNHVYNYQ |
| | | PCDHPRQPCDSSCPCVIAQNFCEKFCQCSSECQNRFPGCRCKAQCNT |
| | | KQCPCYLAVRECDPDLCLTCGAADHWDSKNVSCKNCSIQRGSKKHL |
| | | LLAPSDVAGWGIFIKDPVQKNEFISEYCGEIISQDEADRRGKVYDKY |
| | | MCSFLFNLNNDFVVDATRKGNKIRFANHSVNPNCYAKVMMVNGDH |
| | | RIGIFAKRAIQTGEELFFDYRYSQADALKYVGIEREMEIP |
| KRAB/KOX1 | 18 | MDAKSLTAWSRTLVTFKDVFVDFTREEWKLLDTAQQIVYRNVMLE |
| (CRISPRi) | | NYKNLVSLGYQLTKPDVILRLEKGEEPWLV |
| ZIM3 | 19 | NNSQGRVTFEDVTVNFTQGEWQRLNPEQRNLYRDVMLENYSNLVS |
| (CRISPRi) | | VGQGETTKPDVILRLEQGKEPWLEEEEVLGSGRAEKNGDIGGQIWKP |
| | | KDVKESL |
| TrmD | 20 | MWIGIISLFPEMFRAITDYGVTGRAVKNGLLSIQSWSPRDFTHDRHRT |
| (methyltransferase | | VDDRPYGGGPGMLMMVQPLRDAIHAAKAAAGEGAKVIYLSPQGRK |
| from E. coli) | | LDQAGVSELATNQKLILVCGRYEGIDERVIQTEIDEEWSIGDYVLSGG |
| | | ELPAMTLIDSVSRFIPGVLGHEASATEDSFAEGLLDCPHYTRPEVLEG |
| | | MEVPPVLLSGNHAEIRRWRLKQSLGRTWLRRPELLENLALTEEQARL |
| | | LAEFKTEHAQQQHKHDGMA |
| TrmD | 21 | MWIGVISLFPEMFKAITEFGVTGRAVKHNLLKVECWNPRDFTFDKHK |
| (methyltransferase | | TVDDRPYGGGPGMLMMVQPLRDAIHTAKAAAGEGAKVIYLSPQGR |
| from H. influenzae) | | KLDQGGVTELAQNQKLILVCGRYEGIDERLIQTEIDEEWSIGDYVLTG |
| | | GELPAMTLIDAVARFIPGVLGKQASAEEDSFADGLLDCPHYTRPEVL |
| | | EGLTVPPVLMSGHHEEIRKWRLKQSLQRTWLRRPELLEGLALTDEQR |
| | | KLLKEAQAEHNS |
| TrmT5 | 22 | MVLWILWRPFGFSGRFLKLESHSITESKSLIPVAWTSLTQMLLEAPGIF |
| (methyltransferase | | LLGQRKRFSTMPETETHERETELFSPPSDVRGMTKLDRTAFKKTVNIP |
| from H. sapiens) | | VLKVRKEIVSKLMRSLKRAALQRPGIRRVIEDPEDKESRLIMLDPYKI |
| | | FTHDSFEKAELSVLEQLNVSPQISKYNLELTYEHFKSEEILRAVLPEGQ |
| | | DVTSGFSRIGHIAHLNLRDHQLSFKHLIGQVMIDKNPGITSAVNKINNI |
| | | DNMYRNFQMEVLSGEQNMMTKVRENNYTYEFDFSKVYWNPRLSTE |
| | | HSRITELLKPGDVLFDVFAGVGPFAIPVAKKNCTVFANDLNPESHKW |
| | | LLYNCKLNKVDQKVKVFNLDGKDFLQGPVKEELMQLLGLSKERKPS |
| | | VHVVMNLPAKAIEFLSAFKWLLDGQPCSSEFLPIVHCYSFSKDANPA |
| | | EDVRQRAGAVLGISLEACSSVHLVRNVAPNKEMLCITFQIPASVLYK |
| | | NQTRNPENHEDPPLKRQRTAEAFSDEKTQIVSNT |
| Trm5 | 23 | MPLCLKINKKHGEQTRRILIENNLLNKDYKITSEGNYLYLPIKDVDED |
| (methyltransferase | | ILKSILNIEFELVDKELEEKKIIKKPSFREIISKKYRKEIDEGLISLSYDVV |
| from M. jannaschii) | | GDLVILQISDEVDEKIRKEIGELAYKLIPCKGVFRRKSEVKGEFRVREL |
| | | EHLAGENRTLTIHKENGYRLWVDIAKVYFSPRLGGERARIMKKVSLN |
| | | DVVVDMFAGVGPFSIACKNAKKIYAIDINPHAIELLKKNIKLNKLEHK |
| | | IIPILSDVREVDVKGNRVIMNLPKFAHKFIDKALDIVEEGGVIHYYTIG |
| | | KDFDKAIKLFEKKCDCEVLEKRIVKSYAPREYILALDFKINKK |
| Trm5 (Trm5a | 24 | MSGVKVRREDAKKVLELLKSVGILDGKRKAIRDEKYVIFPVTDTNIA |
| methyltransferase | | KSLGLEVVDVELPMRPERQIYKNLEDLLPREIFKKLGRLDIVGDIAIVS |
| from P. abyssi) | | IPDEILSEREVIVSAIRKLYPKVKVIARRGFHSGLYRIRELEVIWGENRL |
| | | HTIHKENGVLIKVDLSKVFFNPRMKGERYRIAQLVNDGERILVPFAG |
| | | VIPYPLVIARFKNVEVYAVEINEFAVKLAEENLELNRDRLKGKIKIIHG |
| | | DVFEVLPNLPNFDRVVSPTPKGVDALSLTLSKAEKFLHYYDFVHESEI |
| | | ERFRERVLEECRRQGKECRVSVRKVSDYKPHVYKVCADVEILS |
| Trm5 (Trm5b | 25 | MTLAVKVPLKEGEIVRRRLIELGALDNTYKIKREGNFLLIPVKFPVKG |
| methyltransferase | | FEVVEAELEQVSRRPNSYREIVNVPQELRRFLPTSFDIIGNIAIIEIPEEL |
| from P. abyssi) | | KGYAKEIGRAIVEVHKNVKAVYMKGSKIEGEYRTRELIHIAGENITET |
| | | IHRENGIRLKLDVAKVYFSPRLATERMRVFKMAQEGEVVFDMFAGV |
| | | GPFSILLAKKAELVFACDINPWAIKYLEENIKLNKVNNVVPILGDSREI |
| | | EVKADRIIMNLPKYAHEFLEHAISCINDGGVIHYYGFGPEGDPYGWH |
| | | LERIRELANKFGVKVEVLGKRVIRNYAPRQYNIAIDFRVSF |
| Trm5 (Trm5c | 26 | MWSLMHPYLPATVRKWTNLNLSNHSFSLNTMKTKRLSEFFPGVRSY |
| methyltransferase | | YIVGEIAIITPKRVNVDYNLVAEKIMQAHPKIKAVYLKKKVKGELRT |
| from S. | | NELEFLSGERISSTIYKENGVLFYVDINKVYVNPSLSGDRLKNLELVE |
| solfataricus) | | EGSTVLDAFTGYGAIALNIAHKKRVYVVAGDINIDGLYMLKKSLSLN |
| | | KIKGEMIDIVQYDAHHLPFRDKVFKLSFGDNPTLIIDFKEELCRVSEN |
| | | VVFYILCESEDKANSSLGRTSWIKINDYSKNLFIFKGLVRC |
| Trm5 (Trm5a | 27 | MFDESKFDVNLKLWALRIPRELCKSASRILNGYMLNMPRIKPITEDPT |
| methyltransferase | | CEKTRLVILSESVKNADLSEIPEEKLNQLKKLSELEVVPHSVTLGYSY |
| from A. | | WSADHLLKQILPDGLDIPSSFETIGHIAHLNLHDELLPFKDVIAKVIYD |
| thaliana) | | KNYPRIKTIVNKVGTISNEFRVPKFEVLAGENGMETEVKQYGARFKL |
| | | DYGLVYWNSRLEHEHMRLSSLFKPGETVCDMFAGIGPFAIPAAQKG |
| | | CFVYANDLNPDSVRYLKINAKFNKVDDLICVHNMDARKFFSHLMAV |
| | | STCEDNLQSVADNDKTKEAAVSRGGETNSSGEEIRESNASINEPLGAN |
| | | KKPSGTTKTENGVGKDCKSIEGHANKRLRQTLLPIAKPWEHIDHVIM |
| | | NLPASALQFLDSFSNVIQKKYWKGPLPLIHCYCFIRASETTEFIIAEAET |
| | | ALKFHIEDPVFHKVRDVAPNKAMFCLSFRLPEACLKQEE |
| Trm5 | 28 | MKIALPVFQKFNRLISSCKMSGVFPYNPPVNRQMRELDRSFFITKIPM |
| (methyltransferase | | CAVKFPEPKNISVFSKNFKNCILRVPRIPHVVKLNSSKPKDELTSVQN |
| from S. | | KKLKTADGNNTPVTKGVLLHESIHSVEDAYGKLPEDALAFLKENSAE |
| cerevisiae) | | IVPHEYVLDYDFWKAEEILRAVLPEQFLEEVPTGFTITGHIAHLNLRTE |
| | | FKPFDSLIGQVILDKNNKIECVVDKVSSIATQFRTFPMKVIAGKSDSLV |
| | | VEQKESNCTFKFDFSKVYWNSRLHTEHERLVKQYFQPGQVVCDVFA |
| | | GVGPFAVPAGKKDVIVLANDLNPESYKYLKENIALNKVAKTVKSFN |
| | | MDGADFIRQSPQLLQQWIQDEEGGKITIPLPLKKRHRSQQHNDQQPP |
| | | QPRTKELIIPSHISHYVMNLPDSAISFLGNFRGIFAAHTKGATDTIQMP |
| | | WVHVHCFEKYPPGDQVTEDELHARVHARIIAALKVTADDLPLNAVS |
| | | LHLVRKVAPTKPMYCASFQLPANV |
| TrmT5 | 29 | MRIVWKLFGFSRRLLQVEWCHPSESILLFTLVPRLRKAPSVFLLGQRQ |
| (methyltransferase | | GLSTMPEIEASVRDSELFSPPSDVRGMRELDRTAFKKTVSIPVLKARK |
| from M. | | EVVNRLMRALRRVALQRPGIKRVIEDPKDEDSRLIMLDPYRMLTADS |
| musculus) | | FDKAELGVLKELDVSPQLSQYNLELTYENFKSEEILKAVLPEGQDVTS |
| | | GFSRVGHIAHLNLRDHQLPFKHLIGQVMVDKNPGITSAVNKTSNIDN |
| | | TYRNFQMEVLCGEENMLTKVRENNYTYEFDFSKVYWNPRLSTEHGR |
| | | ITELLNPGDVLFDVFAGVGPFAIPAARKNCTVFANDLNPESHKWLLH |
| | | NCKLNKVDQKVKVFNMDGKDFIQGPVREELMLRLGLSAEAKPSVHI |
| | | VMNLPAKAIEFLSVFRSLLDGQPCSTELLPTVHCYCFSKDSDPAKDVR |
| | | QQAEAVLGVSLETSSSVHLVRNVAPNKEMLCITFQIPTATLYRNQSLS |
| | | LQNDQEPPLKRQKTGDPFSGEPQIASDS |
| Trm10 | 30 | MSNDEINQNEEKVKRTPPLPPVPEGMSKKQWKKMCKRQRWEENKA |
| (methyltransferase | | KYNAERRVKKKRLRHERSAKIQEYIDRGEEVPQELIREPRINVNQTDS |
| from S. | | GIEIILDCSFDELMNDKEIVSLSNQVTRAYSANRRANHFAEIKVAPFD |
| cerevisiae) | | KRLKQRFETTLKNTNYENWNHFKFLPDDKIMFGDEHISKDKIVYLTA |
| | | DTEEKLEKLEPGMRYIVGGIVDKNRYKELCLKKAQKMGIPTRRLPID |
| | | EYINLEGRRVLTTTHVVQLMLKYFDDHNWKNAFESVLPPRKLDAEA |
| | | KSASSSPAPKDT |
| Trm10 | 31 | MENKDALDIGKDDTNTSEADVSKNETQEQPVLSKSALKRLKRQQEW |
| (methyltransferase | | DAGREKRAEMRREKKRLRKEERKRKIEAGEVVKSQKKRIRLGKVVP |
| from S. pombe) | | SSIRIVLDCAFDDLMNDKEINSLCQQVTRCHSANRTALHPVELFATNF |
| | | GGRLKTRQDFVLKGQQNNWKRYNPTTKSYLEEFESQKEKLVYLSAD |
| | | SDNTITELDEDKIYIIGAIVDKNRYKNLCQNKASEQGIKTAKLPIDEYI |
| | | KITDRKILTVNQVFEILSLWLEYRDWEKAFMEVIPKRKGILLKSDESF |
| | | DVSEDTRSQSNQSDSELEKEN |
| TrmT10 | 32 | MSSEMLPAFIETSNVDKKQGINEDQEESQKPRLGEGCEPISKRQMKK |
| (TrmT10A | | LIKQKQWEEQRELRKQKRKEKRKRKKLERQCQMEPNSDGHDRKRV |
| methyltransferase | | RRDVVHSTLRLIIDCSFDHLMVLKDIKKLHKQIQRCYAENRRALHPV |
| from H. sapiens) | | QFYLTSHGGQLKKNMDENDKGWVNWKDIHIKPEHYSELIKKEDLIY |
| | | LTSDSPNILKELDESKAYVIGGLVDHNHHKGLTYKQASDYGINHAQL |
| | | PLGNFVKMNSRKVLAVNHVFEIILEYLETRDWQEAFFTILPQRKGAV |
| | | PTDKACESASHDNQSVRMEEGGSDSDSSEEEYSRNELDSPHEEKQDK |
| | | ENHTESTVNSLPH |
| TRrmT10 | 33 | MDWKLEGSTQKVESPVLQGQEGILEETGEDGLPEGFQLLQIDAEGEC |
| (TRrmT10B | | QEGEILATGSTAWCSKNVQRKQRHWEKIVAAKKSKRKQEKERRKA |
| methyltransferase | | NRAENPGICPQHSKRFLRALTKDKLLEAKHSGPRLCIDLSMTHYMSK |
| from H. sapiens) | | KELSRLAGQIRRLYGSNKKADRPFWICLTGFTTDSPLYEECVRMNDG |
| | | FSSYLLDITEEDCFSLFPLETLVYLTPDSEHALEDVDLNKVYILGGLVD |
| | | ESIQKKVTFQKAREYSVKTARLPIQEYMVRNQNGKNYHSEILAINQV |
| | | FDILSTYLETHNWPEALKKGVSSGKGYILRNSVE |
| TrmT10 | 34 | MAAFLKMSVSVNFFRPFTRFLVPFTLHRKRNNLTILQRYMSSKIPAVT |
| (TrmT10C | | YPKNESTPPSEELELDKWKTTMKSSVQEECVSTISSSKDEDPLAATRE |
| methyltransferase | | FIEMWRLLGREVPEHITEEELKTLMECVSNTAKKKYLKYLYTKEKVK |
| from H. sapiens) | | KARQIKKEMKAAAREEAKNIKLLETTEEDKQKNFLFLRLWDRNMDI |
| | | AMGWKGAQAMQFGQPLVFDMAYENYMKRKELQNTVSQLLESEGW |
| | | NRRNVDPFHIYFCNLKIDGALHRELVKRYQEKWDKLLLTSTEKSHVD |
| | | LFPKDSIIYLTADSPNVMTTFRHDKVYVIGSFVDKSMQPGTSLAKAK |
| | | RLNLATECLPLDKYLQWEIGNKNLTLDQMIRILLCLKNNGNWQEAL |
| | | QFVPKRKHTGFLEISQHSQEFINRLKKAKT |
| Tn5 transposase | 35 | VGTMITSALHRAADWAKSVFSSAALGDPRRTARLVNVAAQLAKYSG |
| (DNA | | KSITISSEGSKAMQEGAYRFIRNPNVSAEAIRKAGAMQTVKLAQEFPE |
| transposition; | | LLAIEDTTSLSYRHQVAEKLGKLGSIQDKSRGWWVHSVLLLEATTFR |
| targeted gene | | TVGLLHQEWWMRPDDPADADEKESGKWLAAAATSRLRMGSMMSN |
| insertion) | | VIAVCDREADIHAYLQDKLAHNERFVVRSKHPRKDVESGLYLYDHL |
| | | KNQPELGGYQISIAQKGVVDKRGKRKNRPARKASLSLRSGRITLKQG |
| | | NITLNAVLAEEINPPKGETPLKWLLLTSEPVESLAQALRVIDIYTHRW |
| | | RIEEFHKAWKTGAGAERQRMEEPDNLERMVSILSFVAVRLLQLRESF |
| | | TPPQALRAQGLLKEAEHVESQSAETVLTPDECQLLGYLDKGKRKRK |
| | | EKAGSLQWAYMAIARLGGFMDSKRTGIASWGALWEGWEALQSKLD |
| | | GFLAAKDLMAQGIKI |
| SB100X | 36 | MGKSKEISQDLRKRIVDLHKSGSSLGAISKRLAVPRSSVQTIVRKYKH |
| (DNA | | HGTTQPSYRSGRRRVLSPRDERTLVRKVQINPRTTAKDLVKMLEETG |
| transposition; | | TKVSISTVKRVLYRHNLKGHSARKKPLLQNRHKKARLRFATAHGDK |
| targeted gene | | DRTFWRNVLWSDETKIELFGHNDHRYVWRKKGEACKPKNTIPTVKH |
| insertion) | | GGGSIMLWGCFAAGGTGALHKIDGIMDAVQYVDILKQHLKTSVRKL |
| | | KLGRKWVFQHDNDPKHTSKVVAKWLKDNKVKVLEWPSQSPDLNPI |
| | | ENLWAELKKRVRARRPTNLTQLHQLCQEEWAKIHPNYCGKLVEGYP |
| | | KRLTQVKQFKGNATKY |
| Phage encoded | 37 | MPIAPEFLSLAYPGQEFPAYLYGRASRDPKRKGRSVQSQLDEGRATC |
| serine | | LDAGWPIAGEFKDVDRSASAYARRTRDEFEEMIAGIQAGECRILVAF |
| integrases/ | | EASRYYRDLEAYVRLRRVCREAGVLLCYNGQVYDLSKSADRKATA |
| recombinase 2 | | QDAVNAEGEADDIRERNLRTTRLNAKRGGAHGPVPDGYKRRYDPDS |
| (DNA | | GDLVDQIPHPDRAGLITEIFRRAAAAEPLAAICRDLNERGETTHRGKA |
| transposition; | | WQRHHLHAILRNPAYIGHRRHLGVDTGKGMWAPICDDEDFAETFQA |
| targeted gene | | VQEILSLPGRQLSPGPEAQHLQTGIALCGEHPDEPPLRSVTVRGRTNY |
| insertion) | | NCSTRYDVAMREDRMDAFVEESVITWLASDEAVAAFEDNTDDERTR |
| | | KARIRLKVLEEQLEAAQKQARTLRPDGMGMLLSIDSLAGLEAELTPQ |
| | | IDKARQESRSLHVPALLRDLLGKPRADVDRAWNEALTLPQRRMILR |
| | | MVVTIRLFKAGSRGVRAIEPGRITLSYVGEPGFKPVGGNRAKQ |
| Phage encoded | 38 | MAVGIYIRVSTQEQASEGHSIESQKKKLASYCEIQGWDDYRFYIEEGI |
| serine integrase/ | | SGKNTNRPKLKLLMEHIEKGKINILLVYRLDRLTRSVIDLHKLLNFLQ |
| recombinase 13 | | EHGCAFKSATETYDTTTANGRMSMGIVSLLAQWETENMSERIKLNL |
| (DNA | | EHKVLVEGERVGAIPYGFDLSDDEKLVKNEKSAILLDMVERVENGW |
| transposition; | | SVNRIVNYLNLTNNDRNWSPNGVLRLLRNPALYGATRWNDKIAENT |
| targeted gene | | HEGIISKERFNRLQQILADRSIHHRRDVKGTYIFQGVLRCPVCDQTLS |
| insertion) | | VNRFIKKRKDGTEYCGVLYRCQPCIKQNKYNLAIGEARFLKALNEY |
| | | MSTVEFQTVEDEVIPKKSEREMLESQLQQIARKREKYQKAWASDLM |
| | | SDDEFEKLMVETRETYDECKQKLESCEDPIKIDETYLKEIVYMFHQTF |
| | | NDLESEKQKEFISKFIRTIRYTVKEQQPIRPDKSKTGKGKQKVIITEVEF |
| | | YQ |
| Human WT | 39 | MGIQGLLQFIKEASEPIHVRKYKGQVVAVDTYCWLHKGAIACAEKL |
| Exonuclease 1a | | AKGEPTDRYVGFCMKFVNMLLSHGIKPILVFDGCTLPSKKEVERSRR |
| (DNA | | ERRQANLLKGKQLLREGKVSEARECFTRSINITHAMAHKVIKAARSQ |
| transposition; | | GVDCLVAPYEADAQLAYLNKAGIVQAIITEDSDLLAFGCKKVILKMD |
| targeted gene | | QFGNGLEIDQARLGMCRQLGDVFTEEKFRYMCILSGCDYLSSLRGIG |
| insertion) | | LAKACKVLRLANNPDIVKVIKKIGHYLKMNITVPEDYINGFIRANNTF |
| | | LYQLVFDPIKRKLIPLNAYEDDVDPETLSYAGQYVDDSIALQIALGNK |
| | | DINTFEQIDDYNPDTAMPAHSRSRSWDDKTCQKSANVSSIWHRNYSP |
| | | RPESGTVSDAPQLKENPSTVGVERVISTKGLNLPRKSSIVKRPRSAELS |
| | | EDDLLSQYSLSFTKKTKKNSSEGNKSLSFSEVFVPDLVNGPTNKKSVS |
| | | TPPRTRNKFATFLQRKNEESGAVVVPGTRSRFFCSSDSTDCVSNKVSI |
| | | QPLDETAVTDKENNLHESEYGDQEGKRLVDTDVARNSSDDIPNNHIP |
| | | GDHIPDKATVFTDEESYSFKSSKFTRTISPPTLGTLRSCFSWSGGLGDF |
| | | SRTPSPSPSTALQQFRRKSDSPTSLPENNMSDVSQLKSEESSDDESHPL |
| | | REGACSSQSQESGEFSLQSSNASKLSQCSSKDSDSEESDCNIKLLDSQS |
| | | DQTSKLCLSHFSKKDTPLRNKVPGLYKSSSADSLSTTKIKPLGPARAS |
| | | GLSKKPASIQKRKHHNAENKPGLQIKLNELWKNFGFKKFRD |
| MCP domain | 220 | MASNFTQFVLVDNGGTGDVTVAPSNFANGVAEWISSNSRSQAYKVT |
| | | CSVRQSSAQKRKYTIKVEVPKVATQTVGGVELPVAAWRSYLNMELT |
| | | IPIFATNSDCELIVKAMQGLLKDGNPIPSAIAANSGIY |
| sbcB | 221 | MMNDGKQQSTFLFHDYETFGTHPALDRPAQFAAIRTDSEFNVIGEPE |
| exonuclease | | VFYCKPADDYLPQPGAVLITGITPQEARAKGENEAAFAARIHSLFTVP |
| | | KTCILGYNNVREDDEVTRNIFYRNFYDPYAWSWQHDNSRWDLLDV |
| | | MRACYALRPEGINWPENDDGLPSFRLEHLTKANGIEHSNAHDAMAD |
| | | VYATIAMAKLVKTRQPRLFDYLFTHRNKHKLMALIDVPQMKPLVHV |
| | | SGMFGAWRGNTSWVAPLAWHPENRNAVIMVDLAGDISPLLELDSDT |
| | | LRERLYTAKTDLGDNAAVPVKLVHINKCPVLAQANTLRPEDADRLGI |
| | | NRQHCLDNLKILRENPQVREKVVAIFAEAEPFTPSDNVDAQLYNGFF |
| | | SDADRAAMKIVLETEPRNLPALDITFVDKRIEKLLFNYRARNFPGTLD |
| | | YAEQQRWLEHRRQVFTPEFLQGYADELQMLVQQYADDKEKVALLK |
| | | ALWQYAEEIV |
| TadA_CBE5 | 373 | SEVEFSHEYWMRHALTLAKRARDERAGPVGAVLVLNNRVIGEGWN |
| | | RAIGLHDPTAHAEIMALRQGGLVMQNHRLIDATLYVTFEPCVMCAG |
| | | AMINSRIGRVVFGVRNSKRGAAGSLMNVLNYPGMNHRVEITEGILA |
| | | DECAALLCDFYRMPRHVFNSQKKAQSSIN |
| TadA_CBE6 | 374 | SEVEFSHEYWMRHALTLAKRARDEGEAPVGAVLVLNNRVIGEGWN |
| | | RRIGLHDPTAHAEIMALRQGGLVMQNSRLIDATLYVTFEPCVMCAG |
| | | AMINSRIGRVVFGVRNSKRGAAGSLMNVLNYPGMNHRVEITEGILA |
| | | DECAALLCDFYRMPRQVFNAQKKAQSSIN |
| TadA-CBE (1) | 388 | SEVEFSHEYWMRHALTLAKRARDEGAGPVGAVLVLNNRVIGEGWN |
| | | RAIGLHDPTAHAEIMALRQGGLVMQNYRLFDATLYVTFEPCVMCAG |
| | | AMINSRIGRVVFGVRNSKRGAAGSLMNVLNYPGMNHRVEITEGILA |
| | | DECAALLCDFYRIPRQVENSQKKAQSSIN |
| TadA-CBE (2) | 389 | SEVEFSHEYWMRHALTLAKRARDERKAPVGAVLVLNNRVIGEGWN |
| | | RAIGLHDPTAHAEIIALRQGGLVMQNYRLIDATLYVTFEPCVMCAGA |
| | | MINSRIGRVVFGVRNSKRGAAGSLMNVLNYPGMNHRVEITEGILADE |
| | | CAALLCDFYRMPRQVFNAQKKAQSSIN |
| TadA-CBE (3) | 390 | SEVEFSHEYWMRHALTLAKRARDERSVPVGAVLVLNNRVIGEGWN |
| | | RAIGLHDPTAHAEIMALRQGGLVMQNYRLYDATLYTTFEPCVMCAG |
| | | AMIHSRIGRVVFGVRNAKTGAAGSLMDVLHHPGMNHRVEITEGILA |
| | | DECAALLCRFFRMPRRVFNAQKKAQSSTD |
| endo V | 391 | DLASLRAQQIELASSVIREDRLDKDPPDLIAGADVGFEQGGEVTRAA |
| | | MVLLKYPSLELVEYKVARIATTMPYIPGFLSFREYPALLAAWEMLSQ |
| | | KPDLVFVDGHGISHPRRLGVASHFGLLVDVPTIGVAKKRLCGKFEPL |
| | | SSEPGALAPLMDKGEQLAWVWRSKARCNPLFIATGHRVSVDSALAW |
| | | VQRCMKGYRLPEPTRWADAVASERPAFVRYTANQP |
| TDG | 392 | EAENAGSYSLQQAQAFYTFPFQQLMAEAPNMAVVNEQQMPEEVPAP |
| | | APAQEPVQEAPKGRKRKPRTTEPKQPVEPKKPVESKKSGKSAKPKEK |
| | | QEKITDTFKVKRKVDRENGVSEAELLTKTLPDILTFNLDIVIIGINPGL |
| | | MAAYKGHHYPGPGNHFWKCLFMSGLSEVQLNHMDDHTLPGKYGIG |
| | | FTNMVERTTPGSKDLSSKEFREGGRILVQKLQKYQPRIAVFNGKCIYE |
| | | IFSKEVFGVKVKNLEFGLQPHKIPDTETLCYVMPSSSARCAQFPRAQD |
| | | KVHYYIKLKDLRDQLKGIERNMDVQEVQYTFDLQLAQEDAKKMAV |
| | | KEEKYDPGYEAAYGGAYGENPCSSEPCGFSSNGLIESVELRGESAFSG |
| | | IPNGQWMTQSFTDQIPSFSNHCGTQEQEEESHA |
| NTHL1 | 393 | CSPQESGMTALSARMLTRSRSLGPGAGPRGCREEPGPLRRREAAAEA |
| | | RKSHSPVKRPRKAQRLRVAYEGSDSEKGEGAEPLKVPVWEPQDWQ |
| | | QQLVNIRAMRNKKDAPVDHLGTEHCYDSSAPPKVRRYQVLLSLMLS |
| | | SQTKDQVTAGAIQRLRARGLAVDSILQTDDATLGKLIYPVGFWRSKV |
| | | KYIKQTSAILQQHYGGDIPASVAELVALPGVGPKMAHLAMAVAWGT |
| | | VSGIAVDTHVHRIANRLRWTKKATKSPEETRAALEEWLPRELWHEIN |
| | | GLLVGFGQQTCLPVHPRCHACLNQALCPAAQGL |

TABLE 3 provides illustrative sequences of exemplary heterologous peptide modifications of effector protein(s) that are useful in the compositions, systems and methods described herein.

TABLE 3
SEQUENCES OF EXEMPLARY HETEROLOGOUS PEPTIDE FOR
MODIFICATIONS OF EFFECTOR PROTEIN(S)

| SEQ ID | | |
|---|---|---|
| NO: | Description | Sequence |
| 40 | NLS | KR(K/R)R |
| 41 | NLS | (P/R)XXKR(\hat{ }D/E )(K/R) |
| 42 | NLS | KRX(W/F/Y)XXAF |
| 43 | NLS | (R/P)XXKR(K/R)(\hat{ }D/E ) |
| 44 | NLS | LGKR(K/R)(W/F/Y) |
| 45 | NLS | KRX₁₀K(K/R)(K/R) |
| 46 | EEP | GLFXALLXLLXSLWXLLLXA |
| 47 | NLS | K(K/R)RK |
| 48 | NLS | KRX₁₁K(K/R)(K/R) |
| 49 | NLS | KRX₁₂K(K/R)(K/R) |
| 50 | NLS | KRX₁₀K(K/R)X(K/R) |
| 51 | NLS | KRX₁₁K(K/R)X(K/R) |
| 52 | NLS | KRX₁₂K(K/R)X(K/R) |
| 53 | NLS | APKKKRKVGIHGVPAA |
| 54 | NLS | MAPKKKRKV |
| 55 | NLS | KRPAATKKAGQAKKKK |
| 56 | NLS | KRPAATKKAGQAKKKKEF |
| 57 | NLS | PKKKRKV |
| 58 | NLS | PAAKRVKLD |
| 59 | NLS | PKKKRKVGIHGVPAA |
| 219 | NLS | MAPKKKRKVGIHGVPAA |

*wherein X is any naturally occurring amino acid; and \hat{ }D/E is any naturally occurring amino acid except Asp or Glu

TABLE 4 provides illustrative PAM sequences that are useful in the compositions, systems and methods described herein.

TABLE 4
EXEMPLARY PAM SEQUENCES

| SEQ ID NO: | PAM Sequence* (5'->3') |
|---|---|
| 60 | TNTG |
| 61 | TATG |
| 62 | TCTG |
| 63 | TGTG |
| 64 | TTTG |
| 65 | TNTR |
| 66 | NNTN |
| 67 | ANTR |
| 68 | CNTR |
| 69 | GNTR |
| 70 | TNAR |
| 71 | TNCR |
| 72 | TNGR |
| 73 | TNTC |
| 74 | TNTT |
| 75 | VNTY |
| 76 | TNVY |
| 401 | NTTR |
| 402 | NKCG |
| 403 | NNTR |

*wherein each N is independently any one of A, G, C, or T; R
is adenine or guanine, and K is guanine or thymine

TABLE 5 provides illustrative repeat sequences for use in guide nucleic acids that are useful in the compositions, systems and methods described herein.

TABLE 5
EXEMPLARY REPEAT SEQUENCES FOR USE IN GUIDE NUCLEIC ACIDS

| Effector Protein | Repeat Sequence (5' -> 3'), | Repeat Sequence |
|---|---|---|
| SEQ ID NO: | shown as RNA | SEQ ID NO: |
| 1 | AAGGAUGCCAAAC | 77 |
| 1 | UGGUACAUCCAAC | 78 |
| 1 | GUGGAUAUCCAAC | 79 |
| 1 | AGCAGAAUACAAC | 80 |
| 1 | GUGAAUAUCCAAC | 81 |
| 1 | AGGUAUAUCCAAC | 82 |
| 1 | AGCAAUAUACAAC | 83 |
| 1 | GUUUGAGAACCUUAUGAAA | 84 |
| | UUACAAGGAUGCCAAAC | |
| 1 | CUUUUGGAAGUAUAUAAAU | 396 |
| | UUACAUAGGUACAAAAC | |
| 1 | AUAGGUACAAAAC | 397 |

TABLE 6 provides illustrative intermediary sequences for use in guide nucleic acids that are useful in the compositions, systems and methods described herein.

TABLE 6
EXEMPLARY INTERMEDIARY SEQUENCES FOR USE IN GUIDE NUCLEIC
ACIDS

| Effector | | |
|---|---|---|
| Protein | | Intermediary |
| SEQ ID | | Sequence |
| NO. | Intermediary Sequence (5' -> 3'), shown as RNA | SEQ ID NO: |
| 1 | ACAGCUUAUUUGGAAGCUGAAAUGUGAGGUU | 85 |
| | UAUAACACUCACAAGAAUCCU | |
| 1 | UGGGGCAGUUGGUUGCCCUUAGCCUGAGGCA | 86 |
| | UUUAUUGCACUCGGGAAGUACCAUUUCUCA | |
| 1 | UGGGGCAGUUGGUUGCCCUUAGCCUGAGGCA | 87 |
| | UUUAUUGCACUCGGGAAGUACCA | |
| 1 | AGGGCGUGUUGGAGCGCCUUAGUUUGAGGUA | 88 |
| | UCAAGCACUCAAAAAAUCUAC | |
| 1 | ACGGGUGGUUGUACACCCGAAGAGUGAGGUC | 89 |
| | UUAACGGGCACUCGCUAAUCUGAU | |
| 1 | UGGGCGCGUUGGAGCGCCUUGGUUCGAGGUU | 90 |
| | CCCUGCACUCGAAAAAUUCAC | |
| 1 | UGGGCGUGUUGGAACGCCUUAGUUUGAGGUU | 91 |
| | UCAAGCACUCAAAAAAUUCAC | |
| 1 | CGGGGUGGUUGGACACCCUUAAAUUGAGGUU | 92 |
| | CAUCGCACUCGAUAAAUACCA | |
| 1 | GGGGCUGGUUGUACAGCCUGAAGUUGAGGGA | 93 |
| | UGAUUCCACUCGACAAAUUGCU | |
| 1 | CGGGCUGGUUGGACAGCCUUAAACUGAGGUU | 94 |
| | UAACGCACUCGGUAAAUACCC | |
| 1 | GACAGCUUAUUUGGAAGCUGAAAUGUGAGGU | 95 |
| | UUAUAACACUCACAAGAAUCCU | |
| 1 | UGGCUGAUUUUGCAGCCAUAAGGUGAGGAAA | 398 |
| | AUUCACUCACCAAAGCCUAUGU | |

TABLE 7 provides illustrative linkers for use in guide nucleic acids that are useful in the compositions, systems and methods described herein.

TABLE 7
EXEMPLARY LINKERS FOR USE IN GUIDE NUCLEIC ACIDS

| Linker Sequence (5' -> 3'), | LINKERS SEQ |
|---|---|
| shown as RNA | ID NO: |
| GAAA | 96 |

TABLE 7.1 provides illustrative linkers for polypeptide that are useful in the compositions, systems and methods described herein.

TABLE 7.1
EXEMPLARY LINKERS FOR USE AS POLYPEPTIDE LINKER

| Linker Sequence (5′ → 3′), | LINKERS SEQ |
|---|---|
| shown as RNA | ID NO: |
| GS | 97 |
| GSGGS | 98 |
| GGSGGS | 99 |
| GGGS | 100 |
| GGSG | 101 |
| GGSGG | 102 |
| GSGSG | 103 |
| GSGGG | 104 |
| GGGSG | 105 |
| GSSSG | 106 |

TABLE 8 provides illustrative handle sequences for use in guide nucleic acids that are useful in the compositions, systems and methods described herein.

TABLE 8
EXEMPLARY HANDLE SEQUENCES FOR USE IN GUIDE NUCLEIC ACIDS

| Effector | | Handle |
|---|---|---|
| Protein | | Sequence |
| SEQ ID | | SEQ ID |
| NO. | Handle Sequence (5′ → 3′), shown as RNA * | NO: |
| 1 | ACAGCUUAUUUGGAAGCUGAAAUGUGAGGUUUAUAACACUCA | 107 |
| | CAAGAAUCCUGAAAAAGGAUGCCAAAC | |
| 1 | UGGGGCAGUUGGUUGCCCUUAGCCUGAGGCAUUUAUUGCAC | 108 |
| | UCGGGAAGUACCAUUUCUCAGAAAUGGUACAUCCAAC | |
| 1 | UGGGGCAGUUGGUUGCCCUUAGCCUGAGGCAUUUAUUGCAC | 109 |
| | UCGGGAAGUACCAGAAAUGGUACAUCCAAC | |
| 1 | AGGGCGUGUUGGAGCGCCUUAGUUUGAGGUAUCAAGCACUC | 110 |
| | AAAAAAUCUACGAAAGUGGAUAUCCAAC | |
| 1 | ACGGGUGGUUGUACACCCGAAGAGUGAGGUCUUAACGGGCA | 111 |
| | CUCGCUAAUCUGAUGAAAAGCAGAAUACAAC | |
| 1 | UGGGCGCGUUGGAGCGCCUUGGUUCGAGGUUCCCUGCACU | 112 |
| | CGAAAAAUUCACGAAAGUGAAUAUCCAAC | |
| 1 | UGGGCGUGUUGGAACGCCUUAGUUUGAGGUUUCAAGCACUC | 113 |
| | AAAAAAUUCACGAAAGUGGAUAUCCAAC | |
| 1 | CGGGGUGGUUGGACACCCUUAAAUUGAGGUUCAUCGCACUC | 114 |
| | GAUAAAUACCAGAAAAGGUAUAUCCAAC | |
| 1 | GGGGCUGGUUGUACAGCCUGAAGUUGAGGGAUGAUUCCACU | 115 |
| | CGACAAAUUGCUGAAAAGCAAUAUACAAC | |
| 1 | CGGGCUGGUUGGACAGCCUUAAACUGAGGUUUAACGCACUC | 116 |
| | GGUAAAUACCCGAAAAGGUAUAUCCAAC | |
| 1 | GACAGCUUAUUUGGAAGCUGAAAUGUGAGGUUUAUAACACUC | 117 |
| | ACAAGAAUCCUGAAAAAGGAUGCCAAAC | |
| 1 | acaugaggaucacccauguacagcuuauuuggaagcugaaaugugagguuuauaacacuc | 118 |
| | acaagaauccugaaaaaggaugccaaac | |
| 1 | gcacaugaggaucacccaugugcacagcuuauuuggaagcugaaaugugagguuuauaac | 119 |
| | acucacaagaauccugaaaaaggaugccaaac | |
| 1 | gcagcacaugaggaucacccaugugcugcacagcuuauuuggaagcugaaaugugagguu | 120 |
| | uauaacacucacaagaauccugaaaaaggaugccaaac | |
| 1 | cgcuauacgcacaugaggaucacccaugugcguauagcgacagcuuauuuggaagcugaa | 121 |
| | augugagguuuauaacacucacaagaauccugaaaaaggaugccaaac | |
| 1 | gcacaugaggaucacccaugugcaaacagcuuauuuggaagcugaaaugugagguuuaua | 122 |
| | acacucacaagaauccugaaaaaggaugccaaac | |
| 1 | gcacaugaggaucacccaugugcaaaaaacagcuuauuuggaagcugaaaugugagguuu | 123 |
| | auaacacucacaagaauccugaaaaaggaugccaaac | |
| 1 | gcacaugaggaucacccaugugcaaaaaaaaaaacagcuuauuuggaagcugaaauguga | 124 |
| | gguuuauaacacucacaagaauccugaaaaaggaugccaaac | |
| 1 | acaugaggaucacccauguaaacagcuuauuuggaagcugaaaugugagguuuauaacac | 125 |
| | ucacaagaauccugaaaaaggaugccaaac | |
| 1 | acaugaggaucacccauguaaaaaacagcuuauuuggaagcugaaaugugagguuuauaa | 126 |
| | cacucacaagaauccugaaaaaggaugccaaac | |
| 1 | acaugaggaucacccauguaaaaaaaaaaacagcuuauuuggaagcugaaaugugagguu | 127 |
| | uauaacacucacaagaauccugaaaaaggaugccaaac | |
| 1 | acagcuuauuuggaagcugaaaugugagguuacaugaggaucacccauguaacacucaca | 128 |
| | agaauccugaaaaaggaugccaaac | |
| 1 | acagcuuauuuggaagcugaaaugugagguuuaacaugaggaucacccauguauaacacu | 129 |
| | cacaagaauccugaaaaaggaugccaaac | |
| 1 | acagcuuauuuggaagcugaaaugugagguugcacaugaggaucacccaugugcaacacu | 130 |
| | cacaagaauccugaaaaaggaugccaaac | |
| 1 | acagcuuauuuggaagcugaaaugugagguugcagcacaugaggaucacccaugugcugc | 131 |
| | aacacucacaagaauccugaaaaaggaugccaaac | |
| 1 | acagcuuauuuggaagcugaaaugugagguucgcuauacgcacaugaggaucacccaugu | 132 |
| | gcguauagcgaacacucacaagaauccugaaaaaggaugccaaac | |
| 1 | acagcuuauuuggaagcugaaaugugagguuuagcacaugaggaucacccaugugcauaa | 133 |
| | cacucacaagaauccugaaaaaggaugccaaac | |
| 1 | acagcuuauuuggaagcugaaaugugagguuuagcagcacaugaggaucacccaugugcu | 134 |
| | gcauaacacucacaagaauccugaaaaaggaugccaaac | |
| 1 | acagcuuauuuggaagcugaaaugugagguuuacgcuauacgcacaugaggaucacccau | 135 |
| | gugcguauagcgauaacacucacaagaauccugaaaaaggaugccaaac | |
| 1 | acagcuuauuuggaagcugaaaugugagguguagcacaugaggaucacccaugugcauca | 136 |
| | cacucacaagaauccugaaaaaggaugccaaac | |
| 1 | acagcuuauuuggaagcugaaaugugaggugaagcacaugaggaucacccaugugcaaca | 137 |
| | cacucacaagaauccugaaaaaggaugccaaac | |
| 1 | acagcuuauuuggaagcugaaaugugagguuuauaacacucacaagaauccuuacaugag | 138 |
| | gaucacccauguaaggaugccaaac | |
| 1 | acagcuuauuuggaagcugaaaugugagguuuauaacacucacaagaauccuaaacauga | 139 |
| | ggaucacccauguaaaggaugccaaac | |
| 1 | acagcuuauuuggaagcugaaaugugagguuuauaacacucacaagaauccuugcacaug | 140 |
| | aggaucacccaugugcaaggaugccaaac | |
| 1 | acagcuuauuuggaagcugaaaugugagguuuauaacacucacaagaauccuugcagcac | 141 |
| | augaggaucacccaugugcugcaaggaugccaaac | |
| 1 | acagcuuauuuggaagcugaaaugugagguuuauaacacucacaagaauccuucgcuaua | 142 |
| | cgcacaugaggaucacccaugugcguauagcgaaggaugccaaac | |
| 1 | acagcuuauuuggaagcugaaaugugagguuuauaacacucacaagaauccugcacauga | 143 |
| | ggaucacccaugugcaaggaugccaaac | |
| 1 | acagcuuauuuggaagcugaaaugugagguuuauaacacucacaagaauccugcagcaca | 144 |
| | ugaggaucacccaugugcugcaaggaugccaaac | |
| 1 | acagcuuauuuggaagcugaaaugugagguuuauaacacucacaagaauccuaagcagca | 145 |
| | caugaggaucacccaugugcugcaaaggaugccaaac | |
| 1 | acagcuuauuuggaagcugaaaugugagguuuauaacacucacaagaauccuaacgcuau | 146 |
| | acgcacaugaggaucacccaugugcguauagcgaaaggaugccaaac | |
| 1 | acagcuuauuuggaagcugaaaugugagguuuauaacacucacaagaauccuuguaaaca | 147 |
| | ugaggaucacccauguuuacaaggaugccaaac | |
| 1 | acagcuuauuuggaagcugaaaugugagguuuauaacacucacaagaauccuuguaaaaa | 148 |
| | caugaggaucacccauguaauuacaaggaugccaaac | |
| 1 | acagcuuauuuggaagcugaaaugugagguuuauaacacucacaagaauccuuguacaug | 149 |
| | aggaucacccauguacaaggaugccaaac | |
| 1 | acagcuuauuuggaagcugaaaugugagguuuauaacacucacaagaauccuuguaaaca | 150 |
| | ugaggaucacccauguaaacaaggaugccaaac | |
| 1 | ACAGCUUAUUUGGAAGCUGAAAUGUGAGGUUUAUAACACUCA | 399 |
| | CAAGAAUCCUAAGGAUGCCAAAC | |
| 1 | UGGCUGAUUUUGCAGCCAUAAGGUGAGGAAAAUUCACUCACC | 400 |
| | AAAGCCUAUGUAUAGGUACAAAAC | |

* wherein italic represents intermediary sequences, no-formatting represents repeat sequences, bold represents a linker, and small letter without edit represents a handle sequence with aptamer sequence

TABLE 9 provides illustrative target nucleic acids that are useful in the compositions, systems and methods described herein.

TABLE 9
EXEMPLARY TARGET NUCLEIC ACIDS
Exemp1ary targets

AAVS1, ABCA4, ABCB11, ABCC8, ABCD1, ABCG5, ABCG8, ACAD9, ACADM, ACADVL, ACAT1,
ACTA1, ACOX1, ACSF3, ADA, ADAMTS2, ADGRG1, AGA, AGL, AGPS, AGXT, AHI1, AIRE, ALDH3A2,
ALDOB, ALG6, ALK, ALKBH5, ALMS1, ALPL, AMRC9, AMT, ANAPC10, ANAPC11, ANGPTL3,
ANGPTL4, APC, Apo(a), APOCIII, APOEε4, APOL1, APP, AQP2, AR, ARFRP1, ARG1, ARH, ARL13B,
ARL6, ARSA, ARSB, ASL, ASNS, ASPA, ASS1, ATM, ATP6V1B1, ATP7A, ATP7B, ATRX, ATXN1, ATXN10,
ATXN2, ATXN3, ATXN7, ATXN8OS, AXIN1, AXIN2, B2M, BACE-1, BAK1, BAP1, BARD1, BAX2, BBS1,
BBS10, BBS12, BBS2, BCKDHA, BCKDHB, BCL2L2, BCS1L, BEST1, BLM, BMPR1A, BRAF,
BRAFV600E, BRCA1, BRCA2, BRIP1, BSND, C9orf72, CA4, CACNA1A, CAH1, CAPN3, CASR, CBS,
CCNB1 CC2D2A, CCR5, CD1, CD2, CD3, CD3D, CD3Z, CD4, CD5, CD6, CD7, CD8A, CD8B, CD9,
CD14, CD18, CD19, CD21, CD22, CD23, CD27, CD28, CD30, CD33, CD34, CD36, CD38, CD40, CD40L,
CD44, CD46, CD47, CD48, CD52, CD55, CD57, CD58, CD59, CD68, CD69, CD72, CD73, CD74,
CD79A, CD80, CD81, CD83, CD84, CD86, CD90, CD93, CD96, CD99, CD100, CD123, CD160, CD163,
CD164, CD164L2, CD166, CD200, CD204, CD207, CD209, CD226, CD244, CD247, CD274, CD276,
CD300, CD320, CDC73, CDHI, CDH23, CDK11, CDK4, CDKN1A, CDKNIB, CDKN1C, CDKN2A,
CDKN2B, CEBPA, CELA3B, CEP290, CERKL, CFB, CFTR, CHCHD10, CHEK2, CHM, CHRNE, CIDEB,
CIITA, CLN3, CLN5, CLN6, CLN8, CLRN1, CLTA, CMT1A, CNBP, CNGB1, CNGB3, COL1A1, COL1A2,
COL27A1, COL4A3, COL4A4, COL4A5, COL6A1, COL6A2, COL6A3, COL7A1, CPS1, CPT1A, CPT2,
CRB1, CREBBP, CRX, CRYAA, CTNNA1, CTNNB1, CTNND2, CTNS, CTSK, CXCL12, CYBA, CYBB,
CYP11B1, CYP11B2, CYP17A1, CYP19A1, CYP21A2, CYP27A1, DBT, DCC, DCLRE1C, DERL2,
DFNA36, DFNB31, DGAT2, DHCR7, DHDDS, DICER1, DIS3L2, DLD, DMD, DMPK, DNAH5, DNAI1,
DNAI2, DNM2, DNMT1, DPC4, DYSF, EDA, EDN3, EDNRB, EGFR, EIF2B5, EMC2, EMC3, EMD,
EMX1, EN1, EPCAM, ERCC6, ERCC8, ESCO2, ETFA, ETFDH, ETHEI, EVC, EVC2, EYS, F5, F9, FXI,
FAH, FAM161A, FANCA, FANCB, FANCC, FANCD1, FANCD2, FANCE, FANCF, FANCG, FANCI,
FANCJ, FANCL, FANCM, FANCN, FANCP, FANCS, FBN1, FGF14, FGFR2, FGFR3, FGA, FGB, FGG,
FH, FHL1, FIX, FKRP, FKTN, FLCN, FMR1, FOXP3, FSCN2, FSHD1, FUS, FUT8, FVIII, FXII, FXN,
G6PC, GAA, GALC, GALK1, GALT, GAMT, GATA2, GATA-4, GBA, GBE1, GCDH, GCGR, GDNF, GFAP,
GFM1, GHR, GJB1, GJB2, GLA, GLB1, GLDC, GLE1, GNE, GNPTAB, GNPTG, GNS, GPAM, GPC3,
GPR98, GREM1, GRHPR, GRIN2B, H2AFX, H2AX, HADHA, HAX1, HBA1, HBA2, HBB, HBV cccDNA,
HER2, HEXA, HEXB, HFE, HGSNAT, HLCS, HMGCL, HAO1, HOGA1, HOXB13, HPRPF3, HPRT1,
HPS1, HPS3, HRAS, HRD1, HSD3B2, HSD17B4, HSD17B13, HTT, HUS1, HYAL1, HYLS1, IDS, IDUA,
IFITM5, IKBKAP, IL2RG, IL7R, IMPDH1, INPP5E, IRF4, ITGB2, ITPR1, IVD, JAG1, JAK1, JAK3,
KCNC3, KCND3, KCNJ11, KLKB1, KLHL7, KRAS, LAMA1, LAMA2, LAMA3, LAMB3, LAMC2, LCA5,
LDHA, LDLR, LDLRAP1, LHX3, LIFR, LIPA, LMNA, LMOD3, LOR, LOXHD1, LPA, LPL, LRAT, LRP6,
LRPPRC, LRRK2, MADR2, MAN2B1, MAPT, MARC1, MAX, MCM6, MCOLN1, MECP2, MED17, MEFV,
MEN1, MERTK, MESP2, MET, METex14, MFN2, MFSD8, MIA3, MITF, MKL2, MKS1, MLC1, MLH1,
MLH3, MMAA, MMAB, MMACHC, MMADHC, MMD, MPI, MPL, MPV17, MSH2, MSH3, MSH6,
MTHFD1L, MTHFR, MTM1, MTRR, MTTP, MUT, MUTYH, MYC, MYH7, MYO7A, NAGLU, NAGS, NBN,
NDRG1, NDUFAF5, NDUFS6, NEB, NF1, NF2, NKX2-5, NOG, NOTCH1, NOTCH2, NPC1, NPC2,
NPHP1, NPHS1, NPHS2, NRAS, NR2E3, NTHL1, NTRK, NTRKI, OAT, OCT4, OFD1, OPA3, OTC, PAH,
PALB2, PAQR8, PAX3, PC, PCCA, PCCB, PCDH15, PCSK9, PD1, PDCDI, PDE6B, PDGFRA, PDHA1,
PDHB, PEX1, PEX10, PEX12, PEX13, PEX14, PEX16, PEX19, PEX2, PEX26, PEX3, PEX5, PEX6, PEX7,
PFKM, PHGDH, PHOX2B, PKD1, PKD2, PKHDI, PKK, PLEKHG4, PMM2, PMP22, PMS1, PMS2,
PNPLA3, POLD1, POLE, POMGNT1, POT1, POU5F1, PPM1A, PPP2R2B, PPT1, PRCD, PRKAG2,
PRKAR1A, PRKCG, PRNP, PROM1, PROP1, PRPF31, PRPF8, PRPH2, PRPS1, PSAP, PSD3, PSD95,
PSEN1, PSEN2, PSRC1, PTCH1, PTEN, PTS, PUS1, PYGM, RAB23, RAD50, RAD51C, RAD51D, RAG1,
RAG2, RAPSN, RARS2, RB1, RDH12, RECQL4, RET, RHO, RICTOR, RMRP, ROS1, RP1, RP2, RPE65,
RPGR, RPGRIP1L, RPL32P3, RS1, RTCA, RTEL1, RUNX1, SACS, SAMHD1, SCN1A, SCN2A, SDHA,
SDHAF2, SDHB, SDHC, SDHD, SEL1L, SEPSECS, SERPINA1, SERPINC1, SERPING1, SGCA, SGCB,
SGCG, SGSH, SIRT1, SLC12A3, SLC12A6, SLC17A5, SLC22A5, SLC25A13, SLC25A15, SLC26A2,
SLC26A4, SLC35A3, SLC35B4, SLC37A4, SLC39A4, SLC4A11, SLC6A8, SLC7A7, SMAD3, SMAD4,
SMARCA4, SMARCAL1, SMARCB1, SMARCE1, SMN1, SMPD1, SNAI2, SNCA, SNRNP200, SOD1,
SOX10, SPARA7, SPTBN2, STAR, STAT3, STK11, SUFU, SUMF1, SYNE1, SYNE2, SYS1, TARDBP, TAT,
TBK1, TBP, TCIRG1, TCTN3, TECPR2, TERC, TERT, TFR2, TGFBR2, TGM1, TH, TLE3, TMEM127,
TMEM138, TMEM216, TMEM43, TMEM67, TMPRSS6, TNNI2, TNNT1, TNNT3, TOP1, TOPORS, TP53,
TPM2, TPM3, TPP1, TRAC, TRMU, TSC1, TSC2, TSFM, TSPAN14, TTBK2, TTC8, TTPA, TTR, TULP1,
TYMP, UBE2G2, UBE2J1, UBE3A, USH1C, USHIG, USH2A, VEGF, VHL, VPS13A, VPS13B, VPS35,
VPS45, VRK1, VSX2, VWF, WAS, WDR19, WDR48, WNT10A, WRN, WS2B, WS2C, WT1, XPA, XPC, XPF,
XRCC3, YAP1, ZAC1, ZEB1, ZFYVE26, and ZNF423

TABLE 10 provides illustrative diseases and syndromes for compositions, systems and methods described herein.

TABLE 10
DISEASES AND SYNDROMES
Exemplary Diseases and Syndromes

11-hydroxylase deficiency; 17,20-desmolase deficiency; 17-hydroxylase deficiency; 3-hydroxyisobutyrate
aciduria; 3-hydroxysteroid dehydrogenase deficiency; 46,XY gonadal dysgenesis; AAA syndrome; ABCA3
deficiency; ABCC8-associated hyperinsulinism; aceruloplasminemia; acromegaly; achondrogenesis type 2;
acral peeling skin syndrome; acrodermatitis enteropathica; adrenocortical micronodular hyperplasia;
adrenoleukodystrophies; adrenomyeloneuropathies; Aicardi-Goutieres syndrome; Alagille disease (also
called Alagille Syndrome); Alexander Disease; Alpers syndrome; alpha-1 antitrypsin deficiency (AATD);
alpha-mannosidosis; Alstrom syndrome; Alzheimer′s disease; amebic dysentery; amelogenesis imperfecta;
amish type microcephaly; amyotrophic lateral sclerosis (ALS); anaplastic large cell lymphoma; anauxetic
dysplasia; androgen insensitivity syndrome; angiopathic thrombosis; antiphospholipid syndrome; Antley-
Bixler syndrome; APECED; Apert syndrome; aplasia of lacrimal and salivary glands; arginase-1 deficiency;
argininosuccinic aciduria; argininemia; arrhythmogenic right ventricular dysplasia; Arts syndrome;
ARVD2; arylsulfatase deficiency type metachromatic leukodystrophy; ataxia telangiectasia; atherosclerotic
cardiovascular disease; autoimmune lymphoproliferative syndrome; autoimmune polyglandular syndrome
type 1; autosomal dominant anhidrotic ectodermal dysplasia; autosomal dominant deafness; autosomal
dominant polycystic kidney disease; autosomal recessive microtia; autosomal recessive renal glucosuria;
autosomal visceral heterotaxy; babesiosis; balantidial dysentery; Bardet-Biedl syndrome; Bartter syndrome;
basal cell nevus syndrome; Batten disease; benign recurrent intrahepatic cholestasis; beta-mannosidosis; β-
thalassemia; Bethlem myopathy; Blackfan-Diamond anemia; bleeding disorder (coagulation);
blepharophimosis; Byler disease; C syndrome; CADASIL; calcific aortic stenosis; calcification of joints and
arteries; carbamoyl phosphate synthetase I deficiency; cardiofaciocutaneous syndrome; Carney triad;
carnitine palmitoyltransferase deficiencies; cartilage-hair hypoplasia; cblC type of combined methylmalonic
aciduria; CD18 deficiency; CD3Z-associated primary T-cell immunodeficiency; CD40L deficiency;
CDAGS syndrome; CDG1A; CDG1B; CDG1M; CDG2C; CEDNIK syndrome; central core disease;
centronuclear myopathy; cerebral capillary malformation; cerebrooculofacioskeletal syndrome type 4;
cerebrooculogacioskeletal syndrome; cerebrotendinous xanthomatosis; Chaga′s Disease; Charcot Marie
Tooth Disesase; cherubism; CHILD syndrome; chronic granulomatous disease; chronic recurrent multifocal
osteomyelitis; cirrhosis; citrin deficiency; citrullinemia type I; citrullinemia type II; classic
hemochromatosis; CNPPB syndrome; cobalamin C disease; Cockayne syndrome; coenzyme Q10
deficiency; Coffin-Lowry syndrome; Cohen syndrome; combined deficiency of coagulation factors V;
common variable immune deficiency 3; complement hyperactivation; complete androgen insentivity; cone
rod dystrophies; conformational diseases; congenital adrenal hyperplasia; congenital bile adid synthesis
defect type 1; congenital bile adid synthesis defect type 2; congenital defect in bile acid synthesis type;
congenital erythropoietic porphyria; congenital generalized osteosclerosis; congenital hyperplasia (CAH);
congenital muscular dystrophy 1A (MDC1A); Cornelia de Lange syndrome; coronary heart disease; Cousin
syndrome; Cowden disease; COX deficiency; Cri du chat syndrome; Crigler-Najjar disease; Crigler-Najjar
syndrome type 1; Crisponi syndrome; Crouzon syndrome; Currarino syndrome; Curth-Macklin type
ichthyosis hystrix; cutis laxa; cystic fibrosis; cystinosis; d-2-hydroxyglutaric aciduria; DDP syndrome;
Dejerine-Sottas disease; Denys-Drash syndrome; Dercum disease; desmin cardiomyopathy; desmin
myopathy; DGUOK-associated mitochondrial DNA depletion; diabetes Type I; diabetes Type II; disorders
of glutamate metabolism; distal spinal muscular atrophy type 5; DNA repair diseases; dominant optic
atrophy; Doyne honeycomb retinal dystrophy; Dravet Syndrome; Duchenne muscular dystrophy;
dyskeratosis congenita; Ehlers-Danlos syndrome type 4; Ehlers-Danlos syndromes; Elejalde disease; Ellis-
van Creveld disease; Emery-Dreifuss muscular dystrophies; encephalomyopathic mtDNA depletion
syndrome; encephalitis; enzymatic diseases; EPCAM-associated congenital tufting enteropathy;
epidermolysis bullosa with pyloric atresia; epilepsy; fabry disease; facioscapulohumeral muscular
dystrophy; Factor V Leiden thrombophilia; Faisalabad histiocytosis; familial atypical mycobacteriosis;
familial capillary malformation-arteriovenous; Familial Creutzfeld-Jakob disease; familial esophageal
achalasia; familial glomuvenous malformation; familial hemophagocytic lymphohistiocytosis; familial
mediterranean fever; familial megacalyces; familial schwannomatosis; familial spina bifida; familial splenic
asplenia/hypoplasia; familial thrombotic thrombocytopeniarpura; Fanconi disease (Fanconi anemia);
Feingold syndrome; FENIB; fibrodysplasia ossificans progressiva; FKTN; Fragile X syndrome; Francois-
Neetens fleck corneal dystrophy; Frasier syndrome; Friedreich′s ataxia; FTDP-17; Fuchs corneal dystrophy;
fucosidosis; G6PD deficiency; galactosialidosis; Galloway syndrome; Gardner syndrome; Gaucher disease;
Gitelman syndrome; GLUT1 deficiency; GM2- Gangliosidoses (e.g., Tay Sachs Disease, Sandhoff Disease)
glycogen storage disease type 1b; glycogen storage disease type 2; glycogen storage disease type 3; glycogen
storage disease type 4; glycogen storage disease type 9a; glycogen storage diseases; GM1-gangliosidosis;
Greenberg syndrome; Greig cephalopolysyndactyly syndrome; hair genetic diseases; hairy cell leukemia;
HANAC syndrome; harlequin type ichtyosis congenita; HDR syndrome; hearing loss; hemochromatosis
type 3; hemochromatosis type 4; hemolytic anemia; hemolytic uremic syndrome; hemophilia A; hemophilia
B; hereditary angioedema type 3; hereditary angioedemas; hereditary hemorrhagic telangiectasia; hereditary
hypofibrinogenemia; hereditary intraosseous vascular malformation; hereditary leiomyomatosis and renal
cell cancer; hereditary neuralgic amyotrophy; hereditary sensory and autonomic neuropathy type;
Hermansky-Pudlak disease; HHH syndrome; HHT2; hidrotic ectodermal dysplasia type 1; hidrotic
ectodermal dysplasias; histiocytic sarcoma; HNF4A-associated hyperinsulinism; HNPCC; homozygous
familial hypercholesterolemia; human immunodeficiency with microcephaly; Human monkeypox (MPX);
human papilloma virus (HPV) infection; Huntington′s disease; hyper-IgD syndrome; hyperinsulinism-
hyperammonemia syndrome; hypercholesterolemia; hypertrophy of the retinal pigment epithelium;
hypochondrogenesis; hypohidrotic ectodermal dysplasia; ICF syndrome; idiopathic congenital intestinal
pseudo-obstruction; immunodeficiency 13; immunodeficiency 17; immunodeficiency 25;
immunodeficiency with hyper-IgM type 1; immunodeficiency with hyper-IgM type 3; immunodeficiency
with hyper-IgM type 4; immunodeficiency with hyper-IgM type 5; immunoglobulin alpha deficiency; inborn
errors of thyroid metabolism; infantile myofibromatosis; infantile visceral myopathy; infantile X-linked
spinal muscular atrophy; intrahepatic cholestasis of pregnancy; IPEX syndrome; IRAK4 deficiency; isolated
congenital asplenia; Jeune syndrome; Johanson-Blizzard syndrome; Joubert syndrome; JP-HHT syndrome;
juvenile hemochromatosis; juvenile hyalin fibromatosis; juvenile nephronophthisis; Kabuki mask
syndrome; Kallmann syndromes; Kartagener syndrome; KCNJ11-associated hyperinsulinism; Kearns-Sayre
syndrome; Kostmann disease; Kozlowski type of spondylometaphyseal dysplasia; Krabbe disease; LADD
syndrome; late infantile-onset neuronal ceroid lipofuscinosis; LCK deficiency; LDHCP syndrome; Leber
Congenital Amaurosis Teyp 10; Legius syndrome; Leigh syndrome; lethal congenital contracture syndrome
2; lethal congenital contracture syndromes; lethal contractural syndrome type 3; lethal neonatal CPT
deficiency type 2; lethal osteosclerotic bone dysplasia; leukocyte adhesion deficiency; Li Fraumeni
syndrome; LIG4 syndrome; limb girdle muscular dystrophies (LGMD1B, LGMD2A, LGMD2B);
lipodystrophy; lissencephaly type 1; lissencephaly type 3; Loeys-Dietz syndrome; low phospholipid-
associated cholelithiasis; Lynch Syndrome; lysinuric protein intolerance; a lysosomal storage disease (e.g.,
Hunter syndrome, Hurler syndrome); macular dystrophy; Maffucci syndrome; Majeed syndrome; mannose-
binding protein deficiency; mantle cell lymphoma; Marfan disease; Marshall syndrome; MASA syndrome;
mastocytosis; MCAD deficiency; McCune-Albright syndrome; MCKD2; Meckel syndrome; MECP2
Duplication Syndrome; Meesmann corneal dystrophy; megacystis-microcolon-intestinal hypoperistalsis;
megaloblastic anemia type 1; MEHMO; MELAS; Melnick-Needles syndrome; MEN2s; meningitis; Menkes
disease; metachromatic leukodystrophies; methymalonic acidemia due to transcobalamin receptor defect;
methylmalonic acidurias; methylvalonic aciduria; microcoria-congenital nephrosis syndrome; microvillous
atrophy; migraine; mitochondrial neurogastrointestinal encephalomyopathy; monilethrix; monosomy X;
mosaic trisomy 9 syndrome; Mowat-Wilson syndrome; mucolipidosis type 2; mucolipidosis type Ma;
mucolipidosis type IV; mucopolysaccharidoses; mucopolysaccharidosis type 3A; mucopolysaccharidosis
type 3C; mucopolysaccharidosis type 4B; multiminicore disease; multiple acyl-CoA dehydrogenation
deficiency; multiple cutaneous and mucosal venous malformations; multiple endocrine neoplasia type 1;
multiple sulfatase deficiency; mycosis fungoides; myotonic dystrophy; NAIC; nail-patella syndrome;
nemaline myopathies; neonatal diabetes mellitus; neonatal surfactant deficiency; nephronophtisis; Netherton
disease; neurofibromatoses; neurofibromatosis type 1; Niemann-Pick disease type A; Niemann-Pick disease
type B; Niemann-Pick disease type C; NKX2E; non-alcoholic fatty liver disease (NAFLD); non-alcoholic
steatohepatitis (NASH); Noonan syndrome; North American Indian childhood cirrhosis; NROB1
duplication-associated DSD; ocular genetic diseases; oculo-auricular syndrome; OLEDAID;
oligomeganephronia; oligomeganephronic renal hypolasia; Ollier disease; Opitz-Kaveggia syndrome;
ornithine transcarbamylase deficiency (OTCD); orofaciodigital syndrome type 1; orofaciodigital syndrome
type 2; osseous Paget disease; osteogenesis imperfecta; otopalatodigital syndrome type 2; OXPHOS
diseases; palmoplantar hyperkeratosis; panlobar nephroblastomatosis; Parkes-Weber syndrome; Parkinson′s
disease; partial deletion of 21q22.2-q22.3; Pearson syndrome; Pelizaeus-Merzbacher disease; Pendred
syndrome; pentalogy of Cantrell; peroxisomal acyl-CoA-oxidase deficiency; Peutz-Jeghers syndrome;
Pfeiffer syndrome; Pierson syndrome; pigmented nodular adrenocortical disease; pipecolic acidemia; Pitt-
Hopkins syndrome; plasmalogens deficiency; platelet glycoprotein IV deficiency; pleuropulmonary
blastoma and cystic nephroma; polycystic kidney disease; polycystic ovarian disease; polycystic
lipomembranous osteodysplasia; Pompe disease; including infantile onset Pompe disease (IOPD) and late
onset Pompe disease (LOPD); porphyrias; PRKAG2 cardiac syndrome; premature ovarian failure; primary
erythermalgia; primary hemochromatoses; primary hyperoxaluria; progressive familial intrahepatic
cholestasis; propionic acidemia; protein-losing enteropathy; pyruvate decarboxylase deficiency;
RAPADILINO syndrome; renal cystinosis; retinitis pigmentosa; Rett Syndrome; rhabdoid tumor
predisposition syndrome; Rieger syndrome; ring chromosome 4; Roberts syndrome; Robinow-Sorauf
syndrome; Rothmund-Thomson syndrome; severe combined immunodeficiency disorder (SCID); Saethre-
Chotzen syndrome; Sandhoff disease; SC phocomelia syndrome; SCAS; Schinzel phocomelia syndrome;
severe hypertriglyceridemia; short rib-polydactyly syndrome type 1; short rib-polydactyly syndrome type 4;
short-rib polydactyly syndrome type 2; short-rib polydactyly syndrome type 3; Shwachman disease;
Shwachman-Diamond disease; sickle cell anemia; Silver-Russell syndrome; Simpson-Golabi-Behmel
syndrome; Smith-Lemli-Opitz syndrome; SPG7-associated hereditary spastic paraplegia; spherocytosis;
spinocerebellar ataxia; spinal muscular atrophy; split-hand/foot malformation with long bone deficiencies;
spondylocostal dysostosis; sporadic amyotrophic lateral sclerosis; sporadic visceral myopathy with inclusion
bodies; storage diseases; Stargardt macular dystrophy; STRA6-associated syndrome; stroke; Tay-Sachs
disease; thanatophoric dysplasia; thrombophilia due to antithrombin III deficiency; thyroid metabolism
diseases; Tourette syndrome; transcarbamylase deficiency; transthyretin-associated amyloidosis; trisomy
13; trisomy 22; trisomy 2p syndrome; tuberous sclerosis; tufting enteropathy; ullrich congenital muscular
dystrophy (UCMD); urea cycle diseases; Usher Syndrome; Van Den Ende-Gupta syndrome; Van der Woude
syndrome; variegated mosaic aneuploidy syndrome; VLCAD deficiency; von Hippel-Lindau disease; von
Willebrand disease; Waardenburg syndrome; WAGR syndrome; Walker-Warburg syndrome; Werner
syndrome; Wilson′s disease; Wiskott-Aldrich Syndrome; Wolcott-Rallison syndrome; Wolfram syndrome;
X-linked agammaglobulinemia; X-linked chronic idiopathic intestinal pseudo-obstruction; X-linked cleft
palate with ankyloglossia; X-linked dominant chondrodysplasia punctata; X-linked ectodermal dysplasia;
X-linked Emery-Dreifuss muscular dystrophy; X-linked lissencephaly; X-linked lymphoproliferative
disease; X-linked visceral heterotaxy; xanthinuria type 1; xanthinuria type 2; xeroderma pigmentosum;
XPV; and Zellweger disease.

### EXAMPLES

The following examples are included for illustrative purposes only and are not intended to limit the scope of the invention.

### Example 1: Polypeptide for Gene Editing Gene in Eukaryotic Cells

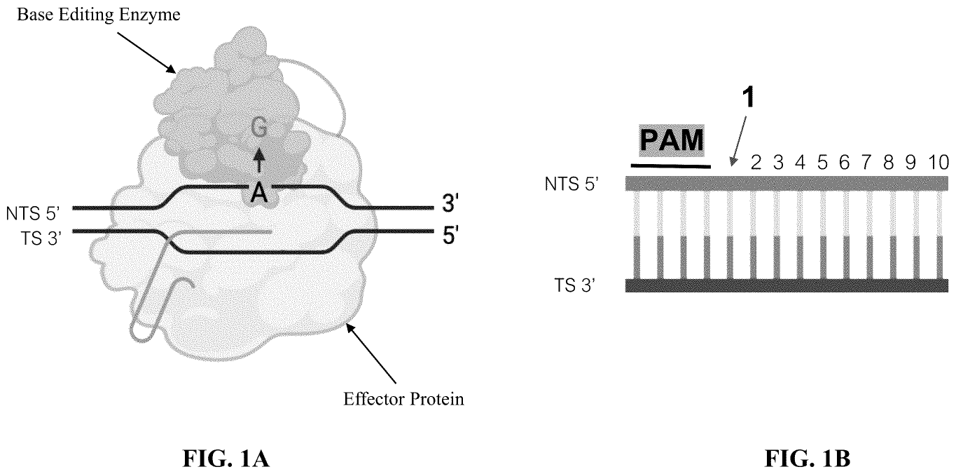

Eukaryotic cells are transfected with nucleic acids encoding a polypeptide (e.g., an effector protein, an effector partner, a fusion protein, or a combination thereof) and a guide nucleic acid for modifying the target nucleic acid. FIGS. 1A-1B show schematics of activities for the polypeptide. The nucleic acid encoding the polypeptide can be delivered as a plasmid or an mRNA. The polypeptide comprises an amino acid sequence that has at least 80% identity to any one of the sequences recited in TABLE 1, TABLE 2 or a combination thereof. The nucleic acid encoding the guide nucleic acid can be delivered as a plasmid or a guide RNA. The guide RNA comprises a handle sequence and a spacer sequence. The handle sequence can be at least 80% identical to any one of the sequences recited in TABLE 5, TABLE 6 and TABLE 8. The spacer sequence can target any one of the genes listed in TABLE 9.

The eukaryotic cells can be transfected by electroporation, lipofection, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof. After about 48 hours to about 96 hours of post-transfection, the eukaryotic cells are screened to assess base substitution and/or percentage of indel generation. DNA or RNA is isolated from the transfected eukaryotic cells and indels are detected by next generation sequencing (NGS) of PCR amplicons at the targeted loci. Indel percentage is calculated as the fraction of sequencing reads containing insertions or deletions relative to an unedited reference sequence.

### Example 2: AAV Vectors for Gene Editing

An AAV vector is constructed to contain a transgene between its ITRs, the transgene providing or encoding, in a 5′ to 3′ direction, a first promoter, a guide nucleic acid, additional promoter(s) and polypeptide(s) as illustrated in FIGS. 2A-2C, are packaged into an AAV vector. The effector protein has an amino acid sequence that has at least 80% identity to a sequence recited in TABLE 1. The effector partner has an amino acid sequence that has at least 80% identity to a sequence recited in TABLE 2. The guide nucleic acid comprises a sequence that has at least 90% identity to any one of sequences recited in TABLE 5, TABLE 6 and TABLE 8. As illustrated in FIGS. 2A-2C, the effector protein can be expressed either ubiquitously or tissue-specifically based on the second promotor the AAV vector is engineered to have. Any one of the additional promoters may be a tissue specific promoter. The AAV vector is expressed with supporting plasmids to produce an adeno-associated virus (AAV).

### Example 3: PAM Screening for Effector Protein CasM.265466

Effector protein CasM.265466 and guide RNA combinations represented in TABLE 11 were screened by in vitro enrichment (IVE) for PAM recognition. TABLE 11 show the components of each effector protein-guide RNA complex assayed for PAM recognition. The amino acid sequence of CasM.265466 is shown in TABLE 1 herein. The nucleotide sequences of the guide components are shown in TABLE 11 herein. For example, as shown in TABLE 11, an effector protein comprising an amino acid sequence of SEQ ID NO: 1 complexed with a guide comprising a crRNA of SEQ ID NO: 151 and a tracrRNA of SEQ ID NO: 154 was screened for PAM recognition.

TABLE 11
EXEMPLARY COMPOSITIONS USED FOR PAM SCREENING

| No. | Composition | Nucleotide Sequence |
|---|---|---|
| 1 | Effector | CasM.265466 (SEQ ID NO: 1) |
| | Protein | |
| | crRNA | GUUUGAGAACCUUAUGAAAUUACAAGG |
| | | AUGCCAAACUAUUAAAUACUCGUAUUG |
| | | CU (SEQ ID NO: 151) |
| | tracrRNA | ACAGCUUAUUUGGAAGCUGAAAUGUGA |
| | | GGUUUAUAACACUCACAAGAAUCCU |
| | | (SEQ ID NO: 154) |
| 2 | Effector | CasM.265466 (SEQ ID NO: 1) |
| | Protein | |
| | crRNA | GUUUGAGAACCUUAUGAAAUUACAAGG |
| | | AUGCCAAACUAUUAAAUACUCGUAUUG |
| | | CU (SEQ ID NO: 152) |
| | tracrRNA | UAUAUUUGAUAAAAAUAUACAGCUUAU |
| | | UUGGAAGCUGAAAUGUGAGGUUUAUAA |
| | | CACUCACAAGAAUCC |
| | | (SEQ ID NO: 155) |
| 3 | Effector | CasM.265466 (SEQ ID NO: 1) |
| | Protein | |
| | sgRNA | ACAGCUUAUUUGGAAGCUGAAAUGUGA |
| | | GGUUUAUAACACUCACAAGAAUCCUGA |
| | | AAAAGGAUGCCAAACUAUUAAAUACUC |
| | | GUAUUGCU (SEQ ID NO: 153) |

Briefly, effector proteins were complexed with corresponding guide RNAs for 15 minutes at 37° C. The complexes were added to an IVE reaction mix. PAM screening reactions used 10 μl of RNP in 100 μl reactions with 1,000 ng of a 5′ PAM library in 1× Cutsmart buffer and were carried out for 15 minutes at 25° C., 45 minutes at 37° C. and 15 minutes at 45° C. Reactions were terminated with 1 μl of proteinase K and 5 μl of 500 mM EDTA for 30 minutes at 37° C. Next generation sequencing was performed on cut sequences to identify enriched PAM sequence for CasM.265466. Cis cleavage by each complex was confirmed by gel electrophoresis. The most enriched PAM was represented by the nucleotide sequence 5′-TNTR-3′ (SEQ ID NO: 65), wherein N is any nucleotide and R is adenine or guanine. Similar experiments were performed, and additional PAMs that were found to be enriched included NTTR (SEQ ID NO: 401), NKCG (SEQ ID NO: 402), and NNTR (SEQ ID NO: 403), wherein N is any nucleotide, R is adenine or guanine, and K is guanine or thymine.

### Example 4: Additional PAM Screening for CasM.265466

Prior in vitro screening as described in Example 3 for effector protein CasM.265466 (SEQ ID NO: 1) PAM recognition demonstrated that the most enriched PAM sequence for CasM.265466 (SEQ ID NO: 1) was a TNTR (SEQ ID NO: 65) PAM sequence, but also indicated that the effector protein may tolerate a more flexible PAM sequences beyond TNTR (SEQ ID NO: 65) without significantly compromising nuclease activity. Effector protein and flexible PAM group combinations as recited in TABLE 12 were screened to confirm that chromosomal DNA may be efficiently targeted in mammalian cells (HEK293T) using a more flexible PAM sequence.

Single and double point mutations were made along TNTR (SEQ ID NO: 65).

TABLE 12
PAM SEQUENCES

| SEQ ID NO: | PAM Group* |
|---|---|
| 66 | NNTN |
| 67 | ANTR |
| 68 | CNTR |
| 69 | GNTR |
| 70 | TNAR |
| 71 | TNCR |
| 72 | TNGR |
| 73 | TNTC |
| 74 | TNTT |
| 75 | VNTY |
| 76 | TNVY |

*wherein each N is any nucleotide, each R is A or G, and each V is A, C or G.

At least six spacers that previously showed >3% indel rate were selected for each PAM group identified in TABLE 12.

Single guide nucleic acids (sgRNA) comprising a handle sequence of SEQ ID NO: 107 and a spacer sequence comprising 20 nucleotides was used.

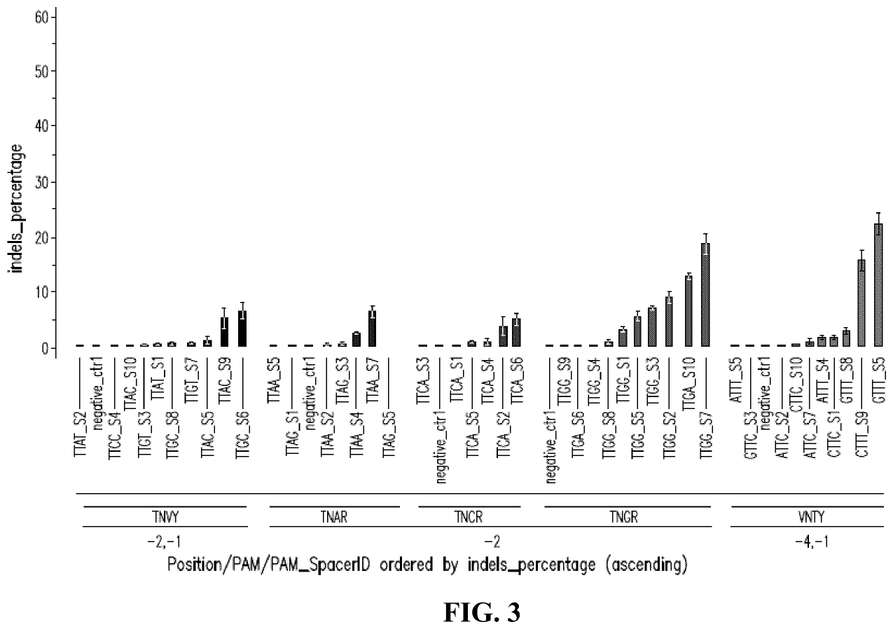

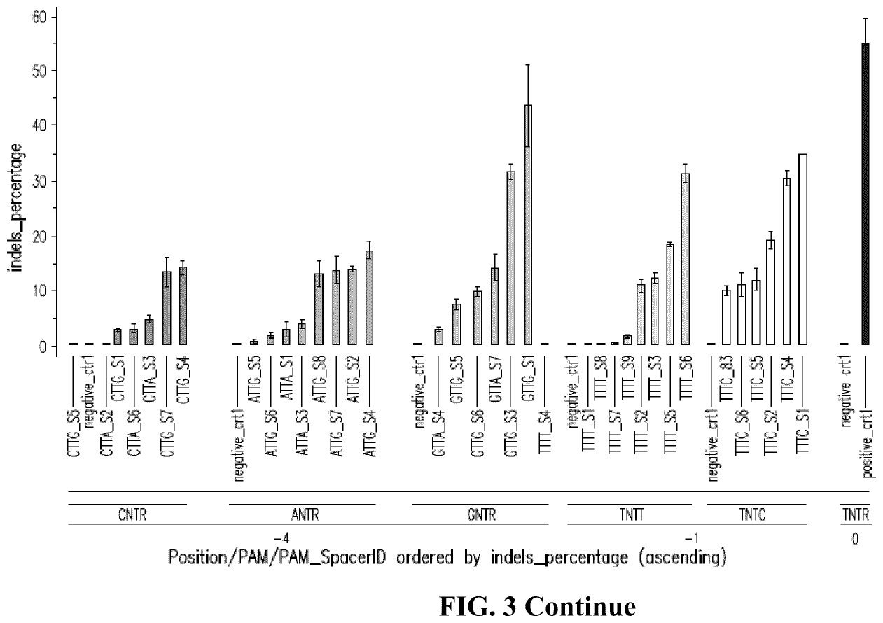

Plasmids encoding CasM.265466 effector protein (SEQ ID NO: 1) and plasmids encoding the sgRNAs were delivered via lipofection to HEK293T cells and permitted to grow to allow for indel formation. Cells were lysed and indels were detected by next generation sequencing. Indel percentage was calculated and plotted as shown in FIG. 3.

While the top performing complexes were found to produce up to or greater than 30% indel, the data also demonstrates that single and double point mutations at −4 and −1 were the most permissive for allowing nuclease activity. Furthermore, the CasM.265466 effector protein (SEQ ID NO: 1) complexed with two different sgRNAs having different spacer sequences generated 20% indel at targeted sequences adjacent to an NNTN (SEQ ID NO: 66) PAM. Therefore, these results further confirm the results of Example 3 and demonstrate that the CasM.265466 effector protein (SEQ ID NO: 1) recognizes a flexible NNTN (SEQ ID NO: 66) PAM sequence.

### Example 5. Arginine Mutation Scanning of CasM.265466 to Identify Charge Substitution Rules of Effector Protein Activity

CasM.265466 arginine mutants were tested for their ability to produce indels in HEK293T cells. A total of 368 arginine mutants were tested. Briefly, a first plasmid encoding a CasM.265466 arginine mutant and a second plasmid encoding a single guide RNA were delivered by lipofection to HEK293T cells. The sgRNA comprised a handle sequence of SEQ ID NO: 107 and a spacer sequence of UCUUCGCCCAGAGCAUCCCA (SEQ ID NO: 156). The sgRNA comprised a spacer sequence that was designed to hybridize to a target sequence adjacent to a PAM of TNTR (SEQ ID NO: 65) (e.g., TTTG (SEQ ID NO: 64)). For lipofections, 15 ng of the nuclease mutant and 150 ng of the guide RNA encoding plasmid were delivered to ~30,000 HEK293T cells in 200 μl using TransIT-293 lipofection reagent. Lipofected cells were grown for ~72 hrs at 37° C. to allow for indel formation. Indels were detected by next generation sequencing of PCR amplicons at the targeted loci and indel percentage was calculated as the fraction of sequencing reads containing insertions or deletions relative to an unedited reference sequence. Sequencing libraries with less than 20% of reads aligning to the reference nucleotide sequence were excluded from the analysis for quality control purposes. Wildtype CasM.265466 was included as positive control and reference for the mutants.

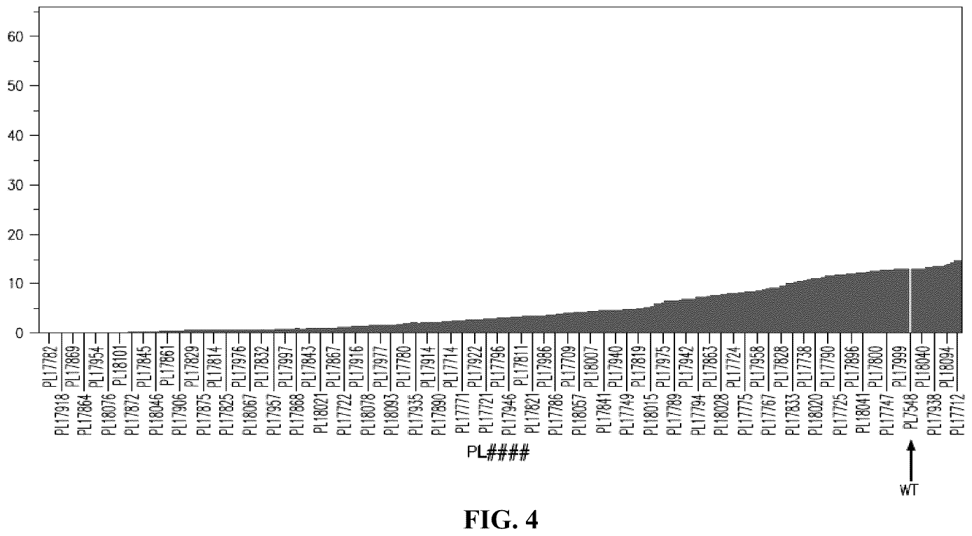

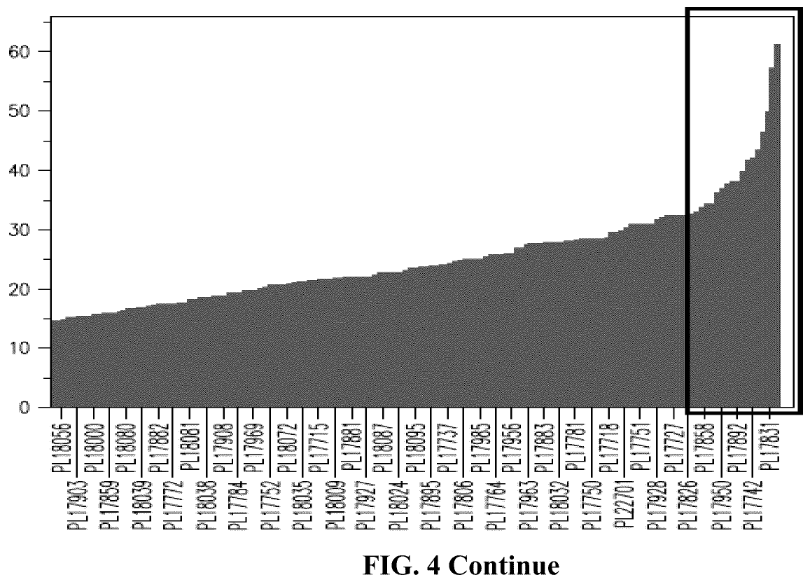

The mean indel percentage for each of the arginine mutant is shown in FIG. 4. An analysis of FIG. 4 indicates that positive charge of arginine may strengthen the interaction between the effector protein and the negatively charged DNA backbone. Top 10 arginine mutants that showed increase in indel potency includes I80R, T84R, K105R, G210R, C202R, A218R, D220R, E225R, C246R, and Q360R.

### Example 6. CasM.265466 Arginine Mutants and Their Potency for Indel Generation

The top ten nuclease mutants, each comprising different CasM.265466 arginine mutant, as identified in Example 5 were tested for their ability to produce indels in HEK293T cells over a variety of doses. Briefly, a first plasmid encoding a CasM.265466 mutant and a second plasmid encoding a single guide RNA (sgRNA) were delivered by lipofection to HEK293T cells. The nucleotide sequence of the sgRNAs comprised a handle sequence of SEQ ID NO: 107 and a spacer sequence of SEQ ID NO: 156. The sgRNA spacer was designed to hybridize to a target sequence adjacent to a PAM of TNTR (SEQ ID NO: 65) (e.g., TTTG (SEQ ID NO: 64)). For lipofections, the CasM.265466 mutant and sgRNA were delivered to ~30,000 HEK293T cells in 200 μl using TransIT-293 lipofection reagent. Each of the ten nuclease mutants were tested at a dose ranging from 1.17 ng to 150 ng. The sgRNA encoding plasmid was used at a concentration of 150 ng. Lipofected cells were grown for ~72 hrs at 37° C. to allow for indel formation. Indels were detected by next generation sequencing of PCR amplicons at the targeted loci and indel percentage was calculated as the fraction of sequencing reads containing insertions or deletions relative to an unedited reference nucleotide sequence. Sequencing libraries with less than 20% of reads aligning to the reference nucleotide sequence were excluded from the analysis for quality control purposes. Wildtype CasM.265466 was included as positive control and reference for the mutants.

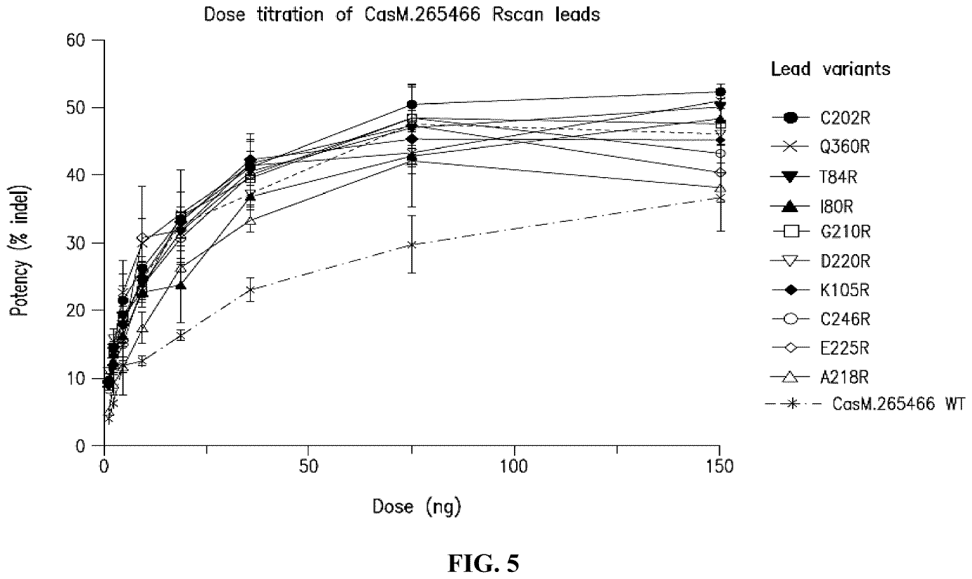

The mean indel percentage and standard deviation based on three replicates is reported in FIG. 5. An analysis of FIG. 5 indicates that arginine substitution can increase potency of the effector protein in the generation of indels.

### Example 7: Editing Efficiency and Integration Rates of CasM.265466 Effector Protein Variants in HEK293T Cells

CasM.265466 effector protein (SEQ ID NO: 1) and CasM.265466 effector protein variants having either a D220R or a E225R substitution was tested for their editing efficiency and ability to integrate double-stranded oligodeoxynucleotide (dsODN) in HEK293T cells as compared to Cas9, empty plasmids and non-targeting (NT) controls.

Briefly, 1 μg of Cas9 and an equivalent amount of the CasM.265466 effector protein or variants thereof (0.63 μg) were complexed with guide nucleic acids (0.37 μg) targeting MLH1, and delivered by nucleofection to HEK293T cells (2.3E+05 cells). Likewise, the controls, empty plasmid, Cas9 and NT controls, were complexed with 2, 1, and 0 μg of guide nucleic acids respectively, and also delivered as described. Guide sequence for CasM.265466 WT and D220R variant comprises a handle sequence of SEQ ID NO: 107 and a spacer sequence comprising a nucleotide sequence of AGUCUCCAGGAAGAAAUUAA (SEQ ID NO: 157). Editing efficiency and integration were assessed by NGS.

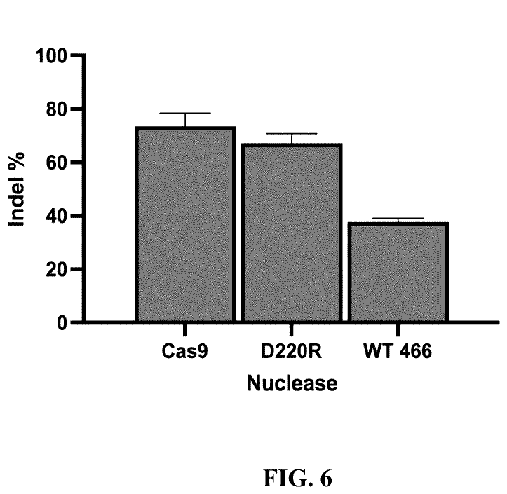

The results confirm the findings from Example 5, namely that the variant effector proteins continue to have an increased editing efficiency as compared to the CasM.265466 effector protein, and had an editing efficiency comparable to Cas9 (FIG. 6). The editing efficiency of the E225R variant was comparable to the editing efficiency of the D220R variant. The D220R and E225R variants were also capable of integrating the dsODN.

### Example 8: Engineered Variants of CasM.265466

A potency assay was performed to evaluate the activity of CasM.265466 protein and engineered variants thereof. The variants were identical to CasM.265466 protein with the exception of the following amino acid substitutions: D220R and A306K. HEK293T cells were transfected with plasmids encoding these proteins and a single guide nucleic acid targeting MLH1. Wildtype CasM.265466 and Cas9 were included as controls. The nucleotide sequence of the sgRNA used with CasM.265466 and variants thereof comprised a handle sequence of SEQ ID NO: 107 and a spacer sequence of SEQ ID NO: 157.

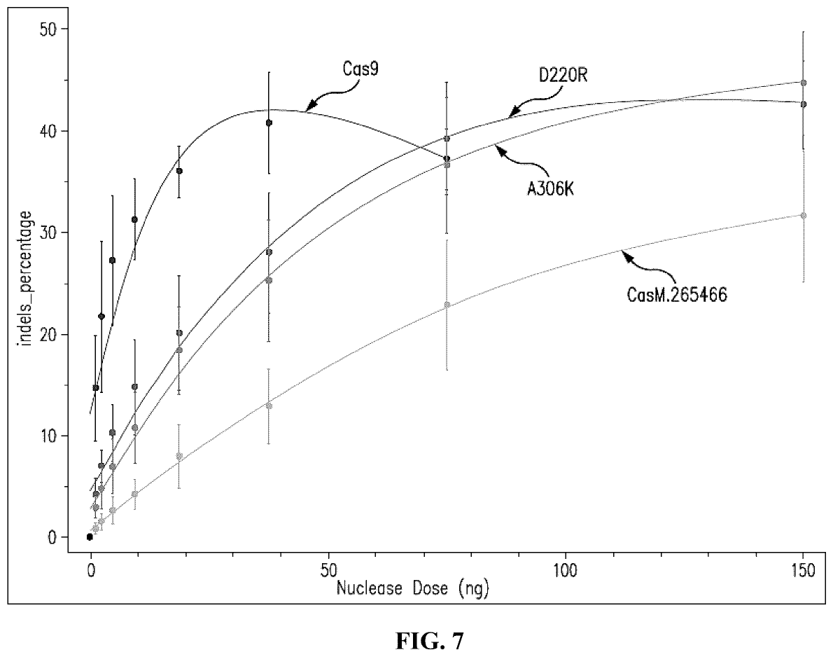

The percentage of target nucleic acids exhibiting nucleotide insertion(s)/deletion(s) (% indel), indicative of nuclease activity, was assessed with next generation sequencing (NGS). As shown in FIG. 7, D220R and A306K were able to generate higher indel percentage relative to the corresponding wildtype CasM.265466 protein.

### Example 9: Rational Engineering of CasM. 265466 Variants

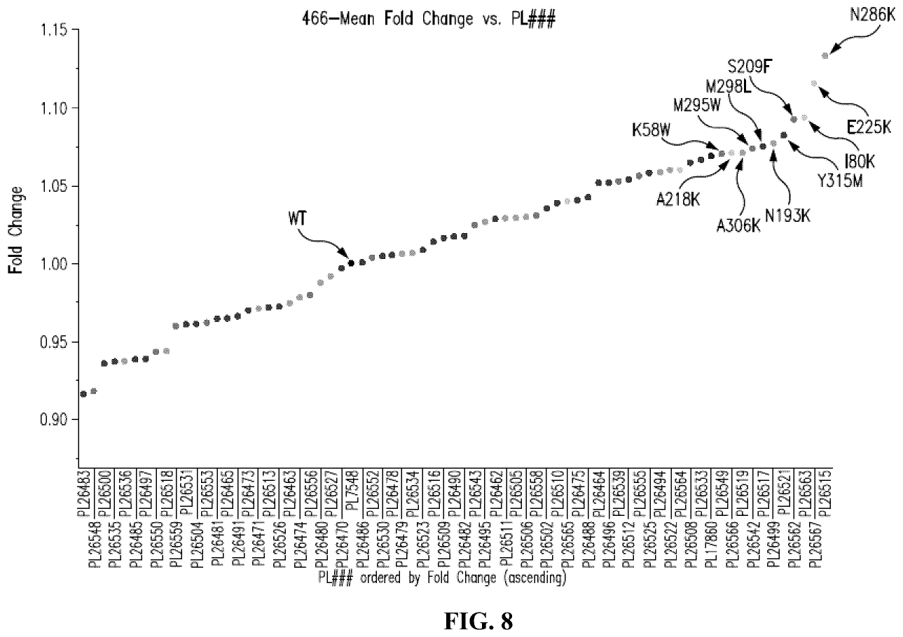

CasM.265466 protein and engineered variants thereof were tested for their ability to produce indels in a mammalian cell line (e.g., HEK293T cells). Briefly, a plasmid encoding the effector proteins and a guide RNA were delivered by lipofection to the mammalian cells. This was performed with guide RNA comprising a handle sequence of SEQ ID NO: 107 and a spacer sequence of SEQ ID NO: 157, wherein the guide RNA was targeting loci adjacent to a PAM of TNTR (SEQ ID NO: 65). Indels in the loci were detected by next generation sequencing of PCR amplicons at the targeted loci and % indel was calculated as the fraction of sequencing reads containing insertions or deletions relative to an unedited reference nucleotide sequence. FIG. 8 shows the activity of the engineered variants relative to that of wildtype as fold change. TABLE 13 lists CasM.265466 variants that demonstrated increased indel production relative to wildtype CasM.265466 (starting with highest fold change at graph to the left).

TABLE 13
Indel Producing Variants of SEQ ID NO: 1

| | Amino Acid Substitution |
|---|---|
| PL##### | Relative to SEQ ID NO: 1 |
| PL26515 | N286K |
| PL26567 | E225K |
| PL26563 | I80K |
| PL26562 | S209F |
| PL26521 | Y315M |
| PL26499 | N193K |
| PL26517 | M298L |
| PL26542 | M295W |
| PL26519 | A306K |
| PL26566 | A218K |
| PL26549 | K58W |

### Example 10: Cis Cleavage Assay for CasM.265466 Variants

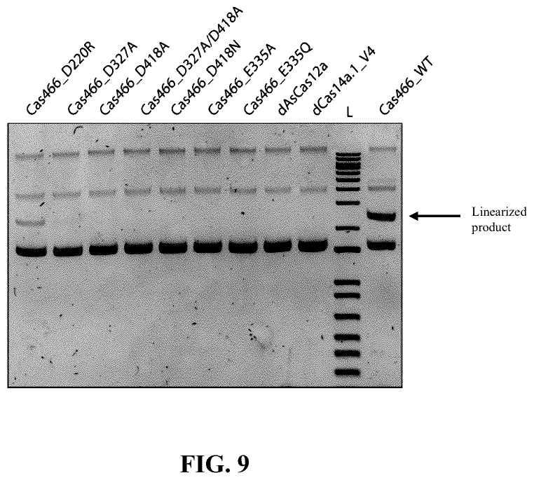

This experiment was performed to determine if variants of CasM.265466 can perform cis cleavage. Briefly, effector protein, CasM.265466, was mutated to generate nuclease dead variants using the following substitutions: D237A, D237A & D418A, D418A, D418N, E335A, and E335Q. The variants were then complexed with sgRNA (SEQ ID NO: 153) for 20 minutes at room temperature. The complexes were added to an IVE reaction mix. Cis cleavage assay was carried out with 5 μl of RNP for at least 30 minutes at 37° C. for identifying catalytically dead variants. A plasmid containing TTTG PAM was used as target nucleotide. Wildtype CasM.265466 (Cas466_WT); CasM.265466 variant D220R (Cas466_D220R); dAsCas12a and Cas14a1_D326A/D510A (dCas14a.1) were included as controls. Cis cleavage by each complex was assessed by gel electrophoresis. The results are shown in FIG. 9. An absence of a linearized product indicates a lack of cleavage activity.

### Example 11: Binding Affinity of CasM.265466 Variants

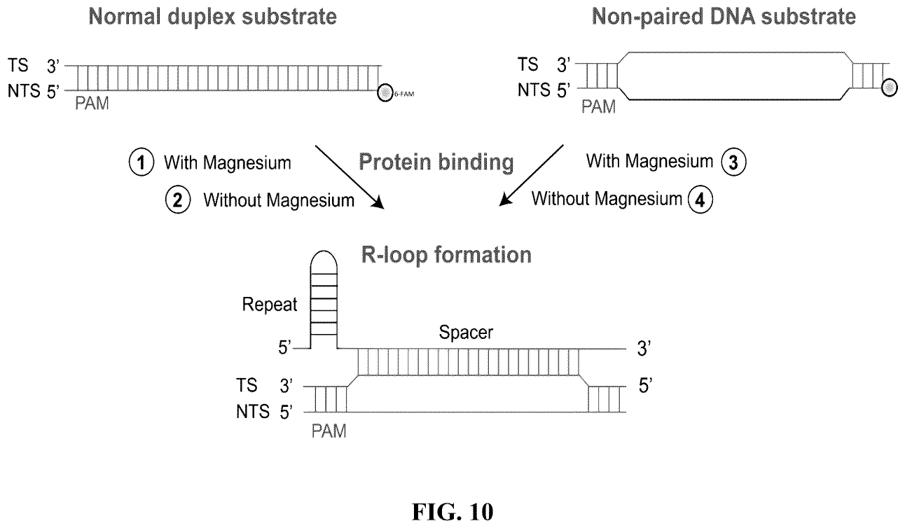

Fluorescence polarization assays were performed to assess the DNA binding affinity of the RNP complexes comprising CasM.265466 variants and sgRNA (SEQ ID NO: 153) generated in Example 10. Reactions were carried out in both the absence and presence of magnesium, the latter to liken the reaction to cellular conditions. Two different DNA substrates were tested with and without magnesium: double stranded duplex DNA (“normal duplex substrate”) and non-paired DNA substrate. These conditions are represented in FIG. 10. Without being bound by theory, non-paired DNA substrate may help mimic an R loop making it easier for an RNP to bind linear DNA. The assay generates different kinds of emissions depending on whether or not a RNP: DNA complex is formed. In the absence of a complex, emissions are depolarized emissions. In the presence of a complex, there are polarized emissions.

The RNP complexes were serially diluted ranging from 500 nM-0.5 nM. Reactions were carried out with the fluorescence polarization assay buffer (20 mM HEPES pH 7.5, 0.2 mg/mL BSA, 1 mM TCEP, 100 mM NaCl, with or without 5 mM Magnesium acetate/EDTA). A 60 bp linear DNA labelled with 6-FAM at the 3′ end was used at a concentration of 1 nM for all experiments. DNA only wells and buffer only wells served as controls. The reactions were carried out in black 384-well flat bottom plates and incubated at 37° C. for 30 minutes and read using the Biotek-Synergy H2 plate reader using the fluorescence polarization filter. The reads were adjusted to extended gain and the well height was calibrated before each run. The graphs were interpreted on PRISM using a combination of their K_{D} values and the maximum polarization value that was recorded at saturating concentrations—referred to as the plateau values.

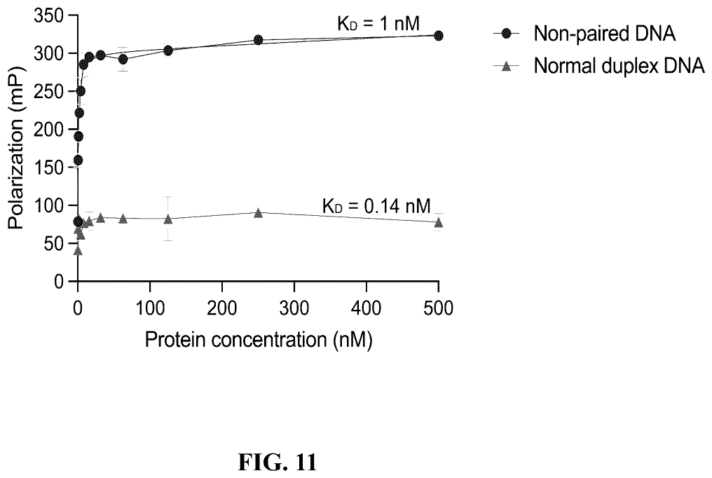

FIG. 11 shows results of the fluorescence polarization assay for the RNP comprising wildtype CasM.265466 with non-paired DNA and normal duplex DNA. Apparent K_{D} value of the RNP comprising wildtype CasM.265466 for non-paired DNA and normal duplex DNA were calculated and are also shown on FIG. 11.

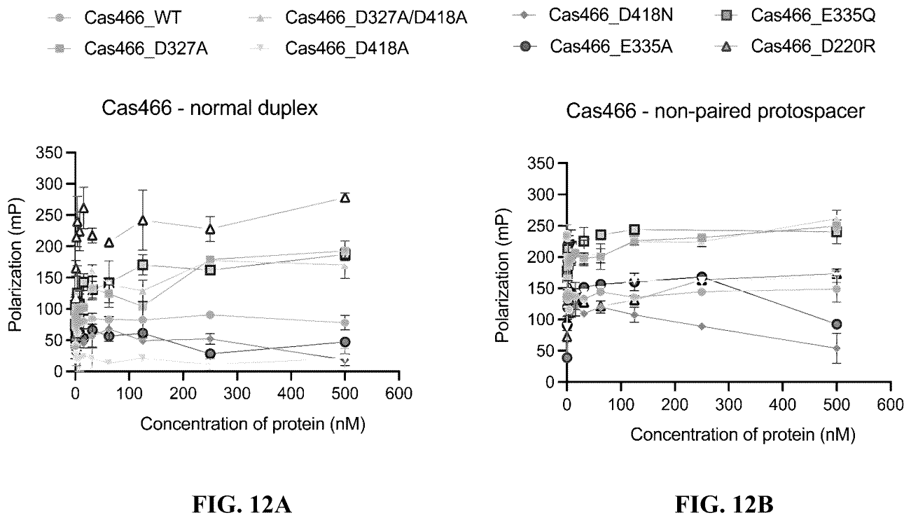

FIGS. 12A-12B shows results of fluorescence polarization assay for the RNPs comprising variants of CasM.265466, relative to corresponding wildtype effector protein. K_{D} values (nM) for the RNPs comprising variants of CasM.265466 were calculated based on results shown in FIGS. 12A-12B. Calculated K_{D} values indicated that all variants bind normal duplex substrate and non-paired DNA substrate. In this experiment, the RNP comprising wildtype CasM.265466 demonstrated a higher preference for non-paired DNA substrate than normal duplex substrate.

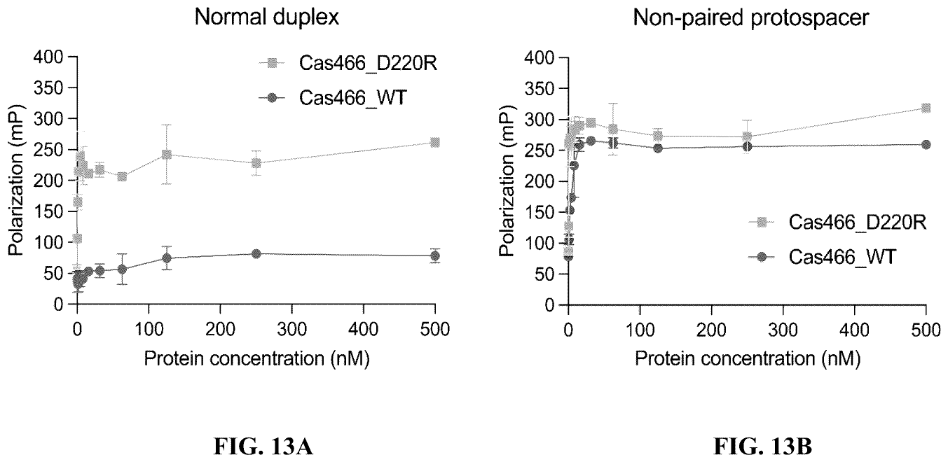

FIGS. 13A-13B show results for the RNPs comprising D220R and the wildtype effector protein only. Results indicate that the RNP comprising D220R can bind to both DNA substrates better than the RNP comprising wildtype CasM.265466.

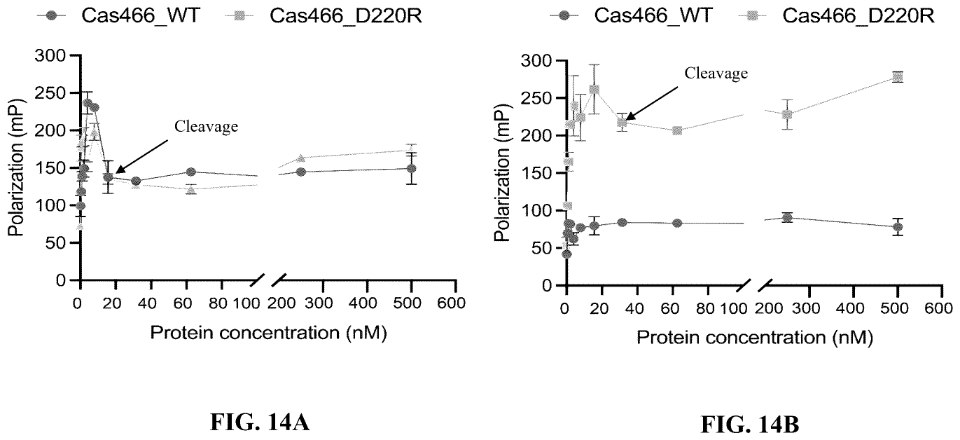

FIGS. 14A-14B show cis cleavage for catalytically active variant D220R relative to the wildtype effector protein. An analysis of FIGS. 14A-14B show a sharp drop in signal, which can be due to cis cleavage. The results from the fluorescence polarization assay for the RNP comprising D220R variant are consistent with the observations in Example 8. Moreover, the RNPs comprising wildtype CasM.265466 and D220R variant performed cis cleavage in the presence of magnesium.

### Example 12: Base Editing Activity of CasM.265466 Effector Protein with E335Q Substitution Fused to an Adenine Base Editing Enzyme

Four nucleic acid vectors encoding engineered polypeptides were constructed for determining base editing activity. The engineered polypeptides tested that were tested are summarized in TABLE 14. This experiment used a sgRNA comprising a handle sequence of SEQ ID NO: 107. For positive control, nSpyCas9 BE control was used.

TABLE 14
Engineered Polypeptides for Base edition

| Name | Description of Engineered Polypeptides |
|---|---|
| ABE8e_466-D220R- | C-terminus of ABE8e (SEQ ID NO: 2) fused to N-terminus of |
| E335Q | CasM.265466 (SEQ ID NO: 1) having D220R and E335Q substitutions |
| 466-D220R- | N-terminus of ABE8e (SEQ ID NO: 2) fused to C-terminus of |
| E335Q_ABE8e | CasM.265466 (SEQ ID NO: 1) CasM.265466 with D220R and E335Q |
| ABE8e_466-E335Q | C-terminus of ABE8e (SEQ ID NO: 2) fused to N-terminus of |
| | CasM.265466 (SEQ ID NO: 1) CasM.265466 with E335Q |
| 466-E335Q_ABE8e | N-terminus of ABE8e (SEQ ID NO:2) fused to C-terminus of |
| | CasM.265466 (SEQ ID NO: 1) CasM.265466 with E335Q |

The base editing activity of each engineered polypeptide was tested in eukaryotic mammalian (HEK293T) cells. Cells were transfected with the nucleic acid vectors and guide RNAs. After sufficient incubation, DNA was extracted from the transfected cells. Target sequences were PCR amplified and sequenced by NGS and MiSeq. The presence of base modifications was analyzed from sequencing data. For determining an editing window, positions are fixed counting from 5′ of the non-target strand, wherein position 1 is the first nucleotide of the non-target strand after PAM sequence. All four guide nucleic acids comprised a 20 nucleotide spacer sequence. Results were recorded as a change in % base call relative to the negative control.

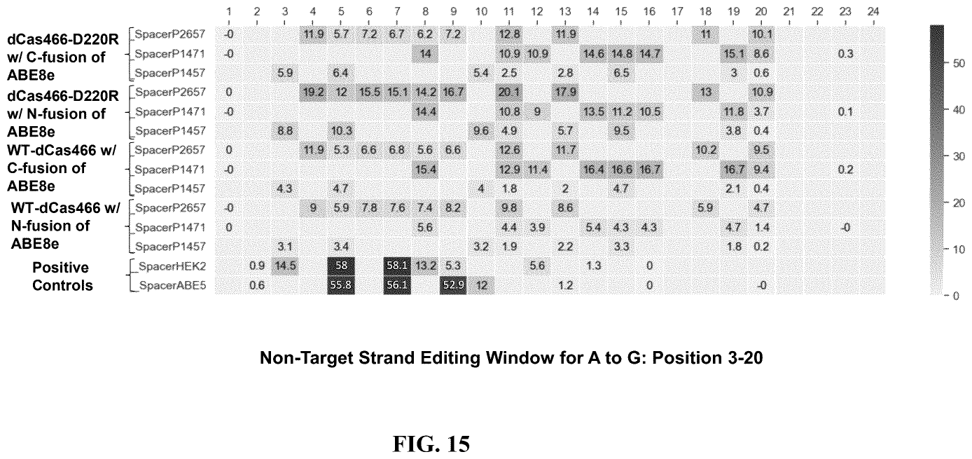

FIG. 15 shows the editing windows on the non-target strand for the tested polypeptides as a heat map color distribution. All engineered polypeptides were capable of base editing. dCas466-D220R w N-fusion of ABE8e provided as much as 20% adenine to guanine editing on the non-target strand. The majority of editing occurred in an editing window of positions 3-20.

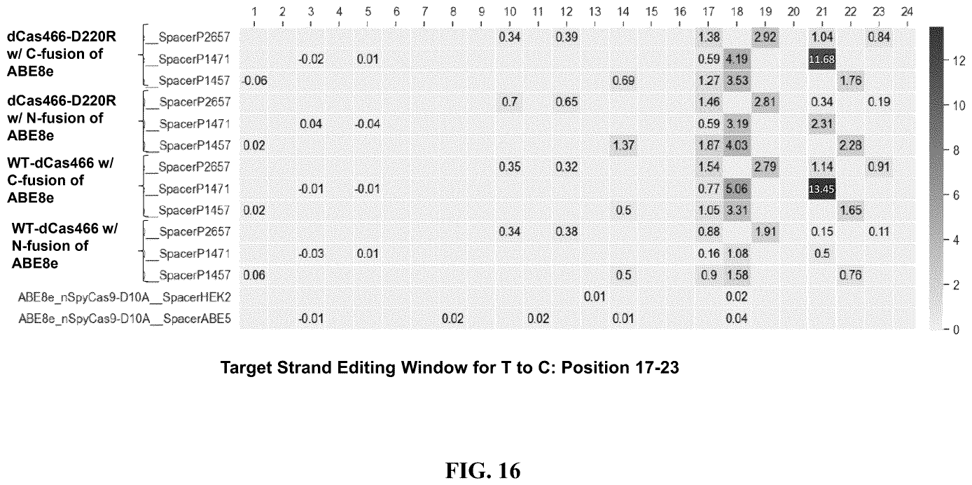

FIG. 16 shows the editing windows on the target strand for the tested polypeptides as a heat map color distribution. All engineered polypeptides were capable of base editing. dCas466 fused to the N-terminus of ABE8e provided as much as 13% adenine to guanine editing on the target strand. The majority of editing occurred in an editing window of positions 17-23.

### Example 13: Base Editing Activity of Engineered Proteins Fused to an Adenine Base Editing Enzyme

Six nucleic acid vectors encoding engineered polypeptides were constructed for determining base editing activity. The engineered polypeptides tested that were tested are summarized in TABLE 15. This experiment used sgRNAs comprising a handle sequence of SEQ ID NO: 107. For positive control, nSpyCas9 BE control was used.

TABLE 15
Engineered Polypeptides for Base edition

| Name | Description of Engineered Polypeptides |
|---|---|
| a265466-D220R- | N-terminus of ABE8e (SEQ ID NO: 2) fused to C-terminus of |
| D237A_ABE8e | CasM.265466 (SEQ ID NO: 1) having D220R and D237A substitutions |
| ABE8e_a265466- | C-terminus of ABE8e (SEQ ID NO: 2) fused to N-terminus of |
| D220R-D237A | CasM.265466 (SEQ ID NO: 1) having D220R and D237A substitutions |
| a265466-D220R- | N-terminus of ABE8e (SEQ ID NO: 2) fused to C-terminus of |
| D237N_ABE8e | CasM.265466 (SEQ ID NO: 1) having D220R and D237N substitutions |
| ABE8e_a265466- | C-terminus of ABE8e (SEQ ID NO: 2) fused to N-terminus of |
| D220R-D237N | CasM.265466 (SEQ ID NO: 1) having D220R and D237N substitutions |
| a265466-D220R- | N-terminus of ABE8e (SEQ ID NO: 2) fused to C-terminus of |
| E335Q_ABE8e | CasM.265466 (SEQ ID NO: 1) having D220R and E335Q substitutions |
| ABE8e_a265466- | C-terminus of ABE8e (SEQ ID NO: 2) fused to N-terminus of |
| D220R-E335Q | CasM.265466 (SEQ ID NO: 1) having D220R and E335Q substitutions |

For each polypeptide, the base editing activity was tested in eukaryotic mammalian (HEK293T) cells. Cells were transfected with the nucleic acid vectors and guide RNAs. After sufficient incubation, DNA was extracted from the transfected cells. Target sequences were PCR amplified and sequenced by NGS and MiSeq. The presence of base modifications was analyzed from sequencing data. For determining editing window, positions are fixed counting from 5′ of the non-target strand, wherein position 1 is the first nucleotide of the non-target strand after PAM sequence. It should be noted that editing window determined based on the four guide nucleic acids having 20 nucleotide long spacer.

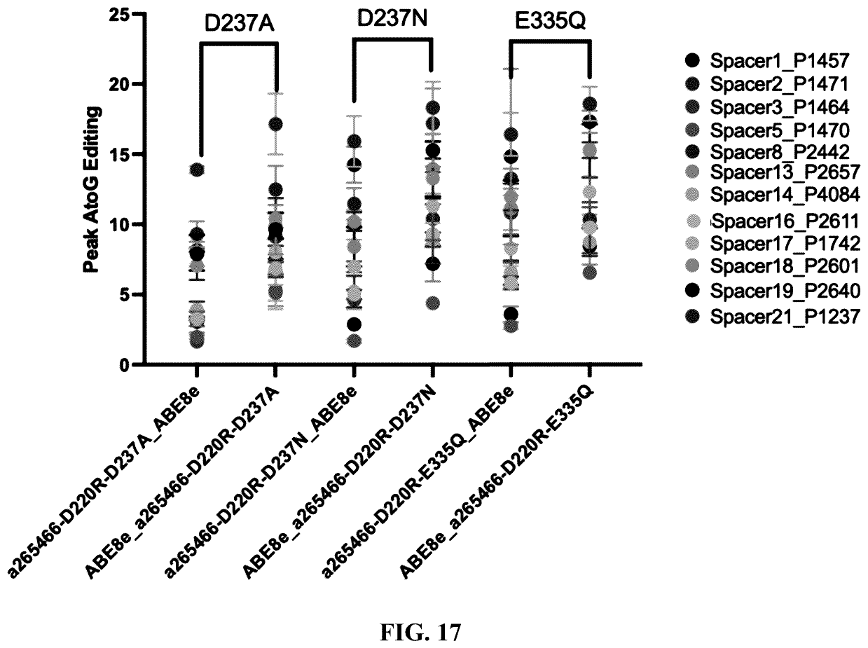

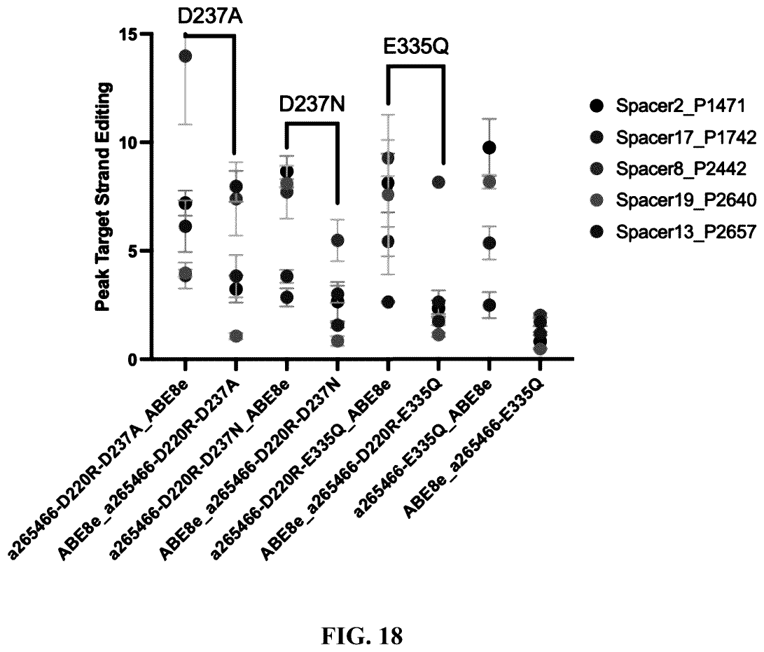

Results were recorded as a change in % base call relative to the negative control. FIG. 17 summarizes result of the change in % base in the non-target strand. Contrarily, FIG. 18 summarizes result of the change in % base in the target strand.

### Example 14: Base Editing Activity of Cytosine Base Editor System

Six nucleic acid vectors encoding engineered polypeptides are constructed for determining base editing activity. The engineered polypeptides tested that are tested are summarized in TABLE 16. Twenty-two guide nucleic acids from Example 13 are tested in combination with each engineered polypeptide comprising a cytosine base editing enzyme. The cytosine base editing enzyme can be any one of APOBEC1, APOBEC2, APOBEC3C, APOBEC3D, APOBEC3F, APOBEC3G, APOBEC3H, APOBEC4, APOBEC3A. For positive control, nSpyCas9 BE control is used.

TABLE 16
Engineered Polypeptides for Base edition

| Name | Description of Engineered Polypeptides |
|---|---|
| a265466-D220R- | N-terminus of CBE fused to C-terminus of CasM.265466 |
| D237A_CBE | (SEQ ID NO: 1) having D220R and D237A substitutions |
| CBE_a265466-D220R- | C-terminus of CBE fused to N-terminus of CasM.265466 |
| D237A | (SEQ ID NO: 1) having D220R and D237A substitutions |
| a265466-D220R- | N-terminus of CBE fused to C-terminus of CasM.265466 |
| D237N_CBE | (SEQ ID NO: 1) having D220R and D237N substitutions |
| CBE_a265466-D220R- | C-terminus of CBE fused to N-terminus of CasM.265466 |
| D237N | (SEQ ID NO: 1) having D220R and D237N substitutions |
| a265466-D220R- | N-terminus of CBE fused to C-terminus of CasM.265466 |
| E335Q_CBE | (SEQ ID NO: 1) having D220R and E335Q substitutions |
| CBE_a265466-E335Q- | C-terminus of CBE fused to N-terminus of CasM.265466 |
| D237N | (SEQ ID NO: 1) having D220R and E335Q substitutions |

For each polypeptide, the base editing activity is tested in eukaryotic mammalian (HEK293T) cells in presence of a uracil glycosylase inhibitor. Cells are transfected with the nucleic acid vectors and guide RNAs. After sufficient incubation, DNA is extracted from the transfected cells. Target sequences are PCR amplified and sequenced by NGS and MiSeq. The presence of base modifications is analyzed from sequencing data. For determining editing window, positions are fixed counting from 5′ of the non-target strand, wherein position 1 is the first nucleotide of the non-target strand after PAM sequence.

### Example 15: Indel Activity of CasM.265466 Fusion Protein

This experiment tested the ability of CasM.265466 to function with a guide nucleic acid (ACAGCUUAUUUGGAAGCUGAAAUGUGAGGUUUAUAACACUCACAAGAAUCCUGAAAA AGGAUGCCAAACACCAAAAAAUAUACGCUAUA (SEQ ID NO: 204)) modified to have an MS2 aptamer sequence (ACAUGAGGAUCACCCAUGU (SEQ ID NO: 218)) inserted into the sgRNA sequence. The MS2 aptamer sequence is located 5′ or 3′ to the spacer sequence. Exonuclease sbcB was fused to an MCP domain that is capable of binding the MS2 aptamer. TABLE 17 lists exemplary modified sgRNA sequences that were tested.

TABLE 17
Aptamer modified sgRNA sequences

| | SEQ | | Mean | Std Dev |
|---|---|---|---|---|
| | ID | | (% | (% |
| Description | NO | sgRNA Sequence | indels) | indels) |
| 5-prime-- | 158 | ACAUGAGGAUCACCCAUGUACAGCUUAU | 48.37 | 2.32 |
| MS2- | | UUGGAAGCUGAAAUGUGAGGUUUAUAA | | |
| nolinker-- | | CACUCACAAGAAUCCUGAAAAAGGAUGC | | |
| vbbPL7548 | | CAAACACCAAAAAAUAUACGCUAUA | | |
| 5-prime-- | 159 | GCACAUGAGGAUCACCCAUGUGCACAGC | 45.82 | 0.36 |
| MS2-2 bp- | | UUAUUUGGAAGCUGAAAUGUGAGGUUU | | |
| linker-- | | AUAACACUCACAAGAAUCCUGAAAAAG | | |
| vbbPL7548 | | GAUGCCAAACACCAAAAAAUAUACGCUA | | |
| | | UA | | |
| 5-prime-- | 160 | GCAGCACAUGAGGAUCACCCAUGUGCUG | 41.85 | 3.93 |
| MS2-5 bp- | | CACAGCUUAUUUGGAAGCUGAAAUGUG | | |
| linker-- | | AGGUUUAUAACACUCACAAGAAUCCUG | | |
| vbbPL7548 | | AAAAAGGAUGCCAAACACCAAAAAAUA | | |
| | | UACGCUAUA | | |
| 5-prime-- | 161 | CGCUAUACGCACAUGAGGAUCACCCAUG | 36.92 | 2.86 |
| MS2-10 bp- | | UGCGUAUAGCGACAGCUUAUUUGGAAG | | |
| linker-- | | CUGAAAUGUGAGGUUUAUAACACUCAC | | |
| vbbPL7548 | | AAGAAUCCUGAAAAAGGAUGCCAAACA | | |
| | | CCAAAAAAUAUACGCUAUA | | |
| 5-prime-- | 162 | GCACAUGAGGAUCACCCAUGUGCAAACA | 48.06 | 5.21 |
| MS2-2 bp- | | GCUUAUUUGGAAGCUGAAAUGUGAGGU | | |
| 2 nt-linker-- | | UUAUAACACUCACAAGAAUCCUGAAAA | | |
| vbbPL7548 | | AGGAUGCCAAACACCAAAAAAUAUACGC | | |
| | | UAUA | | |
| 5-prime-- | 163 | GCACAUGAGGAUCACCCAUGUGCAAAAA | 43.22 | 1.66 |
| MS2-2 bp- | | ACAGCUUAUUUGGAAGCUGAAAUGUGA | | |
| 5 nt-linker-- | | GGUUUAUAACACUCACAAGAAUCCUGA | | |
| vbbPL7548 | | AAAAGGAUGCCAAACACCAAAAAAUAU | | |
| | | ACGCUAUA | | |
| 5-prime-- | 164 | GCACAUGAGGAUCACCCAUGUGCAAAAA | 42.42 | 1.80 |
| MS2-2 bp- | | AAAAAACAGCUUAUUUGGAAGCUGAAA | | |
| 10 nt-linker-- | | UGUGAGGUUUAUAACACUCACAAGAAU | | |
| vbbPL7548 | | CCUGAAAAAGGAUGCCAAACACCAAAAA | | |
| | | AUAUACGCUAUA | | |
| 5-prime-- | 165 | ACAUGAGGAUCACCCAUGUAAACAGCUU | 47.83 | 2.92 |
| MS2-2 nt- | | AUUUGGAAGCUGAAAUGUGAGGUUUAU | | |
| linker-- | | AACACUCACAAGAAUCCUGAAAAAGGA | | |
| vbbPL7548 | | UGCCAAACACCAAAAAAUAUACGCUAUA | | |
| 5-prime-- | 166 | ACAUGAGGAUCACCCAUGUAAAAAACA | 40.99 | 1.05 |
| MS2-5 nt- | | GCUUAUUUGGAAGCUGAAAUGUGAGGU | | |
| linker- | | UUAUAACACUCACAAGAAUCCUGAAAA | | |
| vbbPL7548 | | AGGAUGCCAAACACCAAAAAAUAUACGC | | |
| | | UAUA | | |
| 5-prime-- | 167 | ACAUGAGGAUCACCCAUGUAAAAAAAA | 42.61 | 3.13 |
| MS2-10 nt- | | AAACAGCUUAUUUGGAAGCUGAAAUGU | | |
| linker-- | | GAGGUUUAUAACACUCACAAGAAUCCU | | |
| vbbPL7548 | | GAAAAAGGAUGCCAAACACCAAAAAAU | | |
| | | AUACGCUAUA | | |
| Stem2--MS2- | 168 | ACAGCUUAUUUGGAAGCUGAAAUGUGA | 31.48 | 2.40 |
| nolinker- | | GGUUACAUGAGGAUCACCCAUGUAACAC | | |
| nobubble-- | | UCACAAGAAUCCUGAAAAAGGAUGCCA | | |
| vbbPL7548 | | AACACCAAAAAAUAUACGCUAUA | | |
| Stem2--MS2- | 169 | ACAGCUUAUUUGGAAGCUGAAAUGUGA | 33.55 | 1.80 |
| nolinker- | | GGUUUAACAUGAGGAUCACCCAUGUAU | | |
| Midbubble-- | | AACACUCACAAGAAUCCUGAAAAAGGA | | |
| vbbPL7548 | | UGCCAAACACCAAAAAAUAUACGCUAUA | | |
| Stem2--MS2- | 170 | ACAGCUUAUUUGGAAGCUGAAAUGUGA | 30.11 | 1.94 |
| 2 bp-linker- | | GGUUGCACAUGAGGAUCACCCAUGUGCA | | |
| nobubble-- | | ACACUCACAAGAAUCCUGAAAAAGGAU | | |
| vbbPL7548 | | GCCAAACACCAAAAAAUAUACGCUAUA | | |
| Stem2--MS2- | 171 | ACAGCUUAUUUGGAAGCUGAAAUGUGA | 25.13 | 1.58 |
| 5 bp-linker- | | GGUUGCAGCACAUGAGGAUCACCCAUGU | | |
| nobubble-- | | GCUGCAACACUCACAAGAAUCCUGAAAA | | |
| vbbPL7548 | | AGGAUGCCAAACACCAAAAAAUAUACGC | | |
| | | UAUA | | |
| Stem2--MS2- | 172 | ACAGCUUAUUUGGAAGCUGAAAUGUGA | 20.58 | 1.17 |
| 10 bp-linker- | | GGUUCGCUAUACGCACAUGAGGAUCACC | | |
| nobubble-- | | CAUGUGCGUAUAGCGAACACUCACAAGA | | |
| vbbPL7548 | | AUCCUGAAAAAGGAUGCCAAACACCAAA | | |
| | | AAAUAUACGCUAUA | | |
| Stem2--MS2- | 173 | ACAGCUUAUUUGGAAGCUGAAAUGUGA | 32.94 | 2.70 |
| 2 bp-linker- | | GGUUUAGCACAUGAGGAUCACCCAUGU | | |
| MidBubble-- | | GCAUAACACUCACAAGAAUCCUGAAAAA | | |
| vbbPL7548 | | GGAUGCCAAACACCAAAAAAUAUACGCU | | |
| | | AUA | | |
| Stem2--MS2- | 174 | ACAGCUUAUUUGGAAGCUGAAAUGUGA | 30.15 | 1.21 |
| 5 bp-linker- | | GGUUUAGCAGCACAUGAGGAUCACCCAU | | |
| MidBubble-- | | GUGCUGCAUAACACUCACAAGAAUCCUG | | |
| vbbPL7548 | | AAAAAGGAUGCCAAACACCAAAAAAUA | | |
| | | UACGCUAUA | | |
| Stem2--MS2- | 175 | ACAGCUUAUUUGGAAGCUGAAAUGUGA | 25.79 | 2.00 |
| 10 bp-linker- | | GGUUUACGCUAUACGCACAUGAGGAUC | | |
| MidBubble-- | | ACCCAUGUGCGUAUAGCGAUAACACUCA | | |
| vbbPL7548 | | CAAGAAUCCUGAAAAAGGAUGCCAAAC | | |
| | | ACCAAAAAAUAUACGCUAUA | | |
| Stem2--MS2- | 176 | ACAGCUUAUUUGGAAGCUGAAAUGUGA | 34.59 | 4.69 |
| 2 bp-linker- | | GGUGUAGCACAUGAGGAUCACCCAUGU | | |
| MidBubble- | | GCAUCACACUCACAAGAAUCCUGAAAAA | | |
| T31G-A36C-- | | GGAUGCCAAACACCAAAAAAUAUACGCU | | |
| vbbPL7548 | | AUA | | |
| Stem2--MS2- | 177 | ACAGCUUAUUUGGAAGCUGAAAUGUGA | 29.25 | 5.52 |
| 2 bp-linker- | | GGUGAAGCACAUGAGGAUCACCCAUGU | | |
| MidBubble- | | GCAACACACUCACAAGAAUCCUGAAAAA | | |
| T31G-A36C- | | GGAUGCCAAACACCAAAAAAUAUACGCU | | |
| T32A-T34A-- | | AUA | | |
| vbbPL7548 | | | | |
| Stem3--MS2- | 178 | ACAGCUUAUUUGGAAGCUGAAAUGUGA | 36.56 | 1.55 |
| nolinker- | | GGUUUAUAACACUCACAAGAAUCCUUAC | | |
| withT-- | | AUGAGGAUCACCCAUGUAAGGAUGCCA | | |
| vbbPL7548 | | AACACCAAAAAAUAUACGCUAUA | | |
| Stem3--MS2- | 179 | ACAGCUUAUUUGGAAGCUGAAAUGUGA | 37.02 | 3.01 |
| nolinker- | | GGUUUAUAACACUCACAAGAAUCCUAA | | |
| Midbubble-- | | ACAUGAGGAUCACCCAUGUAAAGGAUG | | |
| vbbPL7548 | | CCAAACACCAAAAAAUAUACGCUAUA | | |
| Stem3--MS2- | 180 | ACAGCUUAUUUGGAAGCUGAAAUGUGA | 32.80 | 2.22 |
| 2 bp-linker- | | GGUUUAUAACACUCACAAGAAUCCUUGC | | |
| nobubble- | | ACAUGAGGAUCACCCAUGUGCAAGGAU | | |
| withT-- | | GCCAAACACCAAAAAAUAUACGCUAUA | | |
| vbbPL7548 | | | | |
| Stem3--MS2- | 181 | ACAGCUUAUUUGGAAGCUGAAAUGUGA | 32.06 | 1.95 |
| 5 bp-linker- | | GGUUUAUAACACUCACAAGAAUCCUUGC | | |
| nobubble- | | AGCACAUGAGGAUCACCCAUGUGCUGCA | | |
| withT-- | | AGGAUGCCAAACACCAAAAAAUAUACGC | | |
| vbbPL7548 | | UAUA | | |
| Stem3--MS2- | 182 | ACAGCUUAUUUGGAAGCUGAAAUGUGA | 29.01 | 2.92 |
| 10 bp-linker- | | GGUUUAUAACACUCACAAGAAUCCUUCG | | |
| nobubble- | | CUAUACGCACAUGAGGAUCACCCAUGUG | | |
| withT-- | | CGUAUAGCGAAGGAUGCCAAACACCAAA | | |
| vbbPL7548 | | AAAUAUACGCUAUA | | |
| Stem3--MS2- | 183 | ACAGCUUAUUUGGAAGCUGAAAUGUGA | 35.86 | 1.32 |
| 2 bp-linker- | | GGUUUAUAACACUCACAAGAAUCCUGCA | | |
| nobubble- | | CAUGAGGAUCACCCAUGUGCAAGGAUGC | | |
| woT-- | | CAAACACCAAAAAAUAUACGCUAUA | | |
| vbbPL7548 | | | | |
| Stem3--MS2- | 184 | ACAGCUUAUUUGGAAGCUGAAAUGUGA | 34.51 | 1.37 |
| 5 bp-linker- | | GGUUUAUAACACUCACAAGAAUCCUGCA | | |
| nobubble- | | GCACAUGAGGAUCACCCAUGUGCUGCAA | | |
| woT-- | | GGAUGCCAAACACCAAAAAAUAUACGCU | | |
| vbbPL7548 | | AUA | | |
| Stem3--MS2- | 185 | ACAGCUUAUUUGGAAGCUGAAAUGUGA | 32.95 | 3.49 |
| 5 bp-linker- | | GGUUUAUAACACUCACAAGAAUCCUAA | | |
| MidBubble-- | | GCAGCACAUGAGGAUCACCCAUGUGCUG | | |
| vbbPL7548 | | CAAAGGAUGCCAAACACCAAAAAAUAU | | |
| | | ACGCUAUA | | |
| Stem3--MS2- | 186 | ACAGCUUAUUUGGAAGCUGAAAUGUGA | 25.25 | 2.09 |
| 10 bp-linker- | | GGUUUAUAACACUCACAAGAAUCCUAAC | | |
| MidBubble-- | | GCUAUACGCACAUGAGGAUCACCCAUGU | | |
| vbbPL7548 | | GCGUAUAGCGAAAGGAUGCCAAACACCA | | |
| | | AAAAAUAUACGCUAUA | | |
| Stem3-- | 187 | ACAGCUUAUUUGGAAGCUGAAAUGUGA | 25.69 | 2.60 |
| 17 ntRpt- | | GGUUUAUAACACUCACAAGAAUCCUUG | | |
| MS2- | | UAAACAUGAGGAUCACCCAUGUUUACA | | |
| nobubble-- | | AGGAUGCCAAACACCAAAAAAUAUACGC | | |
| vbbPL7548 | | UAUA | | |
| Stem3-- | 188 | ACAGCUUAUUUGGAAGCUGAAAUGUGA | 34.02 | 1.74 |
| 17 ntRpt- | | GGUUUAUAACACUCACAAGAAUCCUUG | | |
| MS2- | | UAAAAACAUGAGGAUCACCCAUGUAAU | | |
| Midbubble-- | | UACAAGGAUGCCAAACACCAAAAAAUA | | |
| vbbPL7548 | | UACGCUAUA | | |
| Stem3-- | 189 | ACAGCUUAUUUGGAAGCUGAAAUGUGA | 35.78 | 0.92 |
| 15 ntRpt- | | GGUUUAUAACACUCACAAGAAUCCUUG | | |
| MS2- | | UACAUGAGGAUCACCCAUGUACAAGGA | | |
| nobubble-- | | UGCCAAACACCAAAAAAUAUACGCUAUA | | |
| vbbPL7548 | | | | |
| Stem3-- | 190 | ACAGCUUAUUUGGAAGCUGAAAUGUGA | 36.38 | 4.09 |
| 15 ntRpt- | | GGUUUAUAACACUCACAAGAAUCCUUG | | |
| MS2- | | UAAACAUGAGGAUCACCCAUGUAAACA | | |
| Midbubble-- | | AGGAUGCCAAACACCAAAAAAUAUACGC | | |
| vbbPL7548 | | UAUA | | |
| 3prime-- | 191 | ACAGCUUAUUUGGAAGCUGAAAUGUGA | 35.67 | 3.25 |
| MS2- | | GGUUUAUAACACUCACAAGAAUCCUGA | | |
| nolinker-- | | AAAAGGAUGCCAAACACCAAAAAAUAU | | |
| vbbPL7548 | | ACGCUAUAACAUGAGGAUCACCCAUGU | | |
| 3prime-- | 192 | ACAGCUUAUUUGGAAGCUGAAAUGUGA | 16.21 | 1.57 |
| MS2-3 nt- | | GGUUUAUAACACUCACAAGAAUCCUGA | | |
| 2 bp-linker-- | | AAAAGGAUGCCAAACACCAAAAAAUAU | | |
| vbbPL7548 | | ACGCUAUAAAAGCACAUGAGGAUCACCC | | |
| | | AUGUGC | | |
| 3prime-- | 193 | ACAGCUUAUUUGGAAGCUGAAAUGUGA | 34.21 | 3.31 |
| MS2-6 nt- | | GGUUUAUAACACUCACAAGAAUCCUGA | | |
| 2 bp-linker-- | | AAAAGGAUGCCAAACACCAAAAAAUAU | | |
| vbbPL7548 | | ACGCUAUAAAAAAAGCACAUGAGGAUC | | |
| | | ACCCAUGUGC | | |
| 3prime-- | 194 | ACAGCUUAUUUGGAAGCUGAAAUGUGA | 40.87 | 2.94 |
| MS2-10 nt- | | GGUUUAUAACACUCACAAGAAUCCUGA | | |
| 2 bp-linker-- | | AAAAGGAUGCCAAACACCAAAAAAUAU | | |
| vbbPL7548 | | ACGCUAUAAAAAAAAAAAGCACAUGAG | | |
| | | GAUCACCCAUGUGC | | |
| 3prime-- | 195 | ACAGCUUAUUUGGAAGCUGAAAUGUGA | 28.39 | 1.77 |
| MS2-5 nt- | | GGUUUAUAACACUCACAAGAAUCCUGA | | |
| 5 bp-linker-- | | AAAAGGAUGCCAAACACCAAAAAAUAU | | |
| vbbPL7548 | | ACGCUAUAAAAAAGCAGCACAUGAGGA | | |
| | | UCACCCAUGUGCUGC | | |
| Stem0--MS2- | 196 | UAUAUUUACAUGAGGAUCACCCAUGUA | 46.94 | 1.30 |
| nolinker- | | AAUAUACAGCUUAUUUGGAAGCUGAAA | | |
| nobubble-- | | UGUGAGGUUUAUAACACUCACAAGAAU | | |
| vbbPL7548 | | CCUGAAAAAGGAUGCCAAACACCAAAAA | | |
| | | AUAUACGCUAUA | | |
| Stem0--MS2- | 197 | UAUAUUUGAACAUGAGGAUCACCCAUG | 34.76 | 2.31 |
| nolinker- | | UAAAAAUAUACAGCUUAUUUGGAAGCU | | |
| Midbubble-- | | GAAAUGUGAGGUUUAUAACACUCACAA | | |
| vbbPL7548 | | GAAUCCUGAAAAAGGAUGCCAAACACCA | | |
| | | AAAAAUAUACGCUAUA | | |
| Stem0--MS2- | 198 | UAUAUUUGCACAUGAGGAUCACCCAUG | 34.27 | 1.71 |
| 2 bp-linker- | | UGCAAAUAUACAGCUUAUUUGGAAGCU | | |
| nobubble-- | | GAAAUGUGAGGUUUAUAACACUCACAA | | |
| vbbPL7548 | | GAAUCCUGAAAAAGGAUGCCAAACACCA | | |
| | | AAAAAUAUACGCUAUA | | |
| Stem0--MS2- | 199 | UAUAUUUGCAGCACAUGAGGAUCACCCA | 33.91 | 2.95 |
| 5 bp-linker- | | UGUGCUGCAAAUAUACAGCUUAUUUGG | | |
| nobubble-- | | AAGCUGAAAUGUGAGGUUUAUAACACU | | |
| vbbPL7548 | | CACAAGAAUCCUGAAAAAGGAUGCCAA | | |
| | | ACACCAAAAAAUAUACGCUAUA | | |
| Stem0--MS2- | 200 | UAUAUUUCGCUAUACGCACAUGAGGAU | 34.02 | 3.36 |
| 10 bp-linker- | | CACCCAUGUGCGUAUAGCGAAAUAUACA | | |
| nobubble-- | | GCUUAUUUGGAAGCUGAAAUGUGAGGU | | |
| vbbPL7548 | | UUAUAACACUCACAAGAAUCCUGAAAA | | |
| | | AGGAUGCCAAACACCAAAAAAUAUACGC | | |
| | | UAUA | | |
| Stem0--MS2- | 201 | UAUAUUUGAGCACAUGAGGAUCACCCA | 35.85 | 2.44 |
| 2 bp-linker- | | UGUGCAAAAAUAUACAGCUUAUUUGGA | | |
| MidBubble-- | | AGCUGAAAUGUGAGGUUUAUAACACUC | | |
| vbbPL7548 | | ACAAGAAUCCUGAAAAAGGAUGCCAAA | | |
| | | CACCAAAAAAUAUACGCUAUA | | |
| Stem0--MS2- | 202 | UAUAUUUGAGCAGCACAUGAGGAUCAC | 26.81 | 1.96 |
| 5 bp-linker- | | CCAUGUGCUGCAAAAAUAUACAGCUUA | | |
| MidBubble-- | | UUUGGAAGCUGAAAUGUGAGGUUUAUA | | |
| vbbPL7548 | | ACACUCACAAGAAUCCUGAAAAAGGAU | | |
| | | GCCAAACACCAAAAAAUAUACGCUAUA | | |
| Stem0--MS2- | 203 | UAUAUUUGACGCUAUACGCACAUGAGG | 33.19 | 1.98 |
| 10 bp-linker- | | AUCACCCAUGUGCGUAUAGCGAAAAAU | | |
| MidBubble-- | | AUACAGCUUAUUUGGAAGCUGAAAUGU | | |
| vbbPL7548 | | GAGGUUUAUAACACUCACAAGAAUCCU | | |
| | | GAAAAAGGAUGCCAAACACCAAAAAAU | | |
| | | AUACGCUAUA | | |
| NegControl-- | 204 | ACAGCUUAUUUGGAAGCUGAAAUGUGA | 47.16 | 2.75 |
| Original- | | GGUUUAUAACACUCACAAGAAUCCUGA | | |
| sgRNA- | | AAAAGGAUGCCAAACACCAAAAAAUAU | | |
| control-- | | ACGCUAUA | | |
| vbbPL7548 | | | | |
| NegControl-- | 205 | ACAGCUUAUUUGGAAGCUGAAAUGUGA | 44.99 | 2.61 |
| Original- | | GGUUUAUAACACUCACAAGAAUCCUUG | | |
| sgRNAwithT- | | AAAAAGGAUGCCAAACACCAAAAAAUA | | |
| control-- | | UACGCUAUA | | |
| vbbPL7548 | | | | |
| NegControl-- | 206 | UAUAUUUGAUAAAAAUAUACAGCUUAU | 32.03 | 2.53 |
| Stem0- | | UUGGAAGCUGAAAUGUGAGGUUUAUAA | | |
| sgRNA- | | CACUCACAAGAAUCCUGAAAAAGGAUGC | | |
| control-- | | CAAACACCAAAAAAUAUACGCUAUA | | |
| vbbPL7548 | | | | |
| NegControl-- | 207 | UAUAUUUGAUAAAAAUAUACAGCUUAU | 31.59 | 2.62 |
| Stem0- | | UUGGAAGCUGAAAUGUGAGGUUUAUAA | | |
| sgRNAwithT- | | CACUCACAAGAAUCCUUGAAAAAGGAU | | |
| control-- | | GCCAAACACCAAAAAAUAUACGCUAUA | | |
| vbbPL7548 | | | | |
| NegControl-- | 208 | GCGCGAGGAUCACCCGCGCACAGCUUAU | 47.03 | 2.63 |
| 5-prime- | | UUGGAAGCUGAAAUGUGAGGUUUAUAA | | |
| RandomStem-- | | CACUCACAAGAAUCCUGAAAAAGGAUGC | | |
| vbbPL7548 | | CAAACACCAAAAAAUAUACGCUAUA | | |
| NegControl-- | 209 | ACAGCUUAUUUGGAAGCUGAAAUGUGA | 26.26 | 1.28 |
| Stem2- | | GGUUUAGCGCGAGGAUCACCCGCGCAUA | | |
| RandomStem-- | | ACACUCACAAGAAUCCUGAAAAAGGAU | | |
| vbbPL7548 | | GCCAAACACCAAAAAAUAUACGCUAUA | | |
| NegControl-- | 210 | ACAGCUUAUUUGGAAGCUGAAAUGUGA | 34.94 | 1.89 |
| Stem3- | | GGUUUAUAACACUCACAAGAAUCCUUGC | | |
| RandomStem-- | | GCGAGGAUCACCCGCGCAAGGAUGCCAA | | |
| vbbPL7548 | | ACACCAAAAAAUAUACGCUAUA | | |
| NegControl-- | 211 | ACAGCUUAUUUGGAAGCUGAAAUGUGA | 22.38 | 1.47 |
| 3prime- | | GGUUUAUAACACUCACAAGAAUCCUGA | | |
| RandomStem-- | | AAAAGGAUGCCAAACACCAAAAAAUAU | | |
| vbbPL7548 | | ACGCUAUAAAAGCGCGAGGAUCACCCGC | | |
| | | GC | | |
| NegControl-- | 212 | UAUAUUUGAGCGCGAGGAUCACCCGCGC | 37.61 | 1.52 |
| Stem0- | | AAAAAUAUACAGCUUAUUUGGAAGCUG | | |
| RandomStem-- | | AAAUGUGAGGUUUAUAACACUCACAAG | | |
| vbbPL7548 | | AAUCCUGAAAAAGGAUGCCAAACACCAA | | |
| | | AAAAUAUACGCUAUA | | |
| PosControl-- | 213 | GGACAUGAGGAUCACCCAUGUCCGAACA | 49.63 | 2.76 |
| Archive- | | GCUUAUUUGGAAGCUGAAAUGUGAGGU | | |
| 5primeMS2-- | | UUAUAACACUCACAAGAAUCCUGAAAA | | |
| vbbPL7548 | | AGGAUGCCAAACACCAAAAAAUAUACGC | | |
| | | UAUA | | |
| PosControl-- | 214 | ACAGCUUAUUUGGAAGCUGAAAUGUGA | 29.47 | 2.17 |
| Archive- | | GGUUUGGACAUGAGGAUCACCCAUGUCC | | |
| stem2MS2-- | | AAACACUCACAAGAAUCCUGAAAAAGG | | |
| vbbPL7548 | | AUGCCAAACACCAAAAAAUAUACGCUAU | | |
| | | A | | |
| PosControl-- | 215 | ACAGCUUAUUUGGAAGCUGAAAUGUGA | 36.06 | 0.71 |
| Archive- | | GGUUUAUAACACUCACAAGAAUCCUGG | | |
| stem3MS2-- | | ACAUGAGGAUCACCCAUGUCCAAGGAUG | | |
| vbbPL7548 | | CCAAACACCAAAAAAUAUACGCUAUA | | |
| PosControl-- | 216 | ACAGCUUAUUUGGAAGCUGAAAUGUGA | 24.59 | 1.99 |
| Archive- | | GGUUUAUAACACUCACAAGAAUCCUGA | | |
| 3primeMS2-- | | AAAAGGAUGCCAAACACCAAAAAAUAU | | |
| vbbPL7548 | | ACGCUAUAGGACAUGAGGAUCACCCAUG | | |
| | | UCC | | |

Observance of indels indicated that CasM.265466 was able to function with the modified guide nucleic acid in the presence of an MCP-sbcB fusion protein. Interestingly, fusion of exonuclease sbcB to CasM.265466 significantly decreased indel window size in HEK293T cells.

The result of this experiment indicates that a guide nucleic acid with an aptamer can be useful for recruiting other proteins besides exonucleases, e.g., proteins for precision editing such as deaminases and reverse transcriptases.

### Example 16. Modulation of In Vitro Cis Cleavage Activity of Effector Proteins by Varying Length of a Spacer Sequence

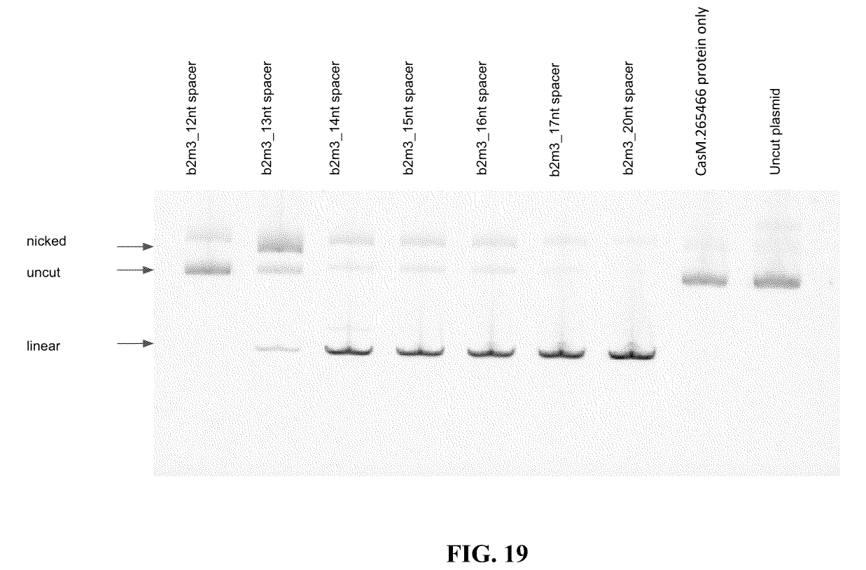

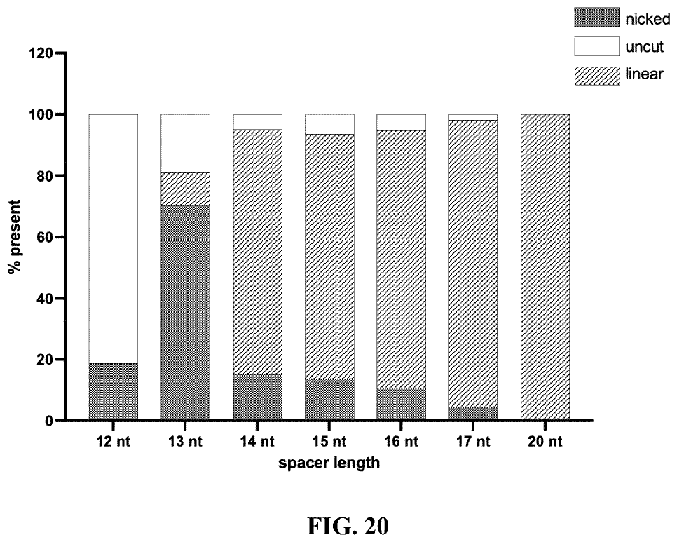

An in vitro screening was carried out to determine the effect of the length of a spacer sequence of a guide nucleic acid on an effector protein's cis cleavage activity (e.g., nickase activity). Specifically, an isolated CasM.265466 WT effector protein (SEQ ID NO: 1) was incubated with a target nucleic acid (e.g., a supercoiled double stranded plasmid) and guide nucleic acids (e.g., sgRNA) having different lengths of spacer sequences. The guide nucleic acids were engineered to target a sequence within B2M gene (e.g., B2M3 target sequence). Specifically, the guide nucleic acids with the spacer sequences of 12, 13, 14, 15, 16, 17 and 20 nucleotides were tested for a cis cleavage assay. Briefly, 50 nm of the effector protein, 50 nM of the guide nucleic acid, the target nucleic acid, and a buffer (e.g., 200 mM HEPES pH 7.5 at 37° C., 3 mM Mg(OAc)₂, 1 mM TCEP, and 0.2 mg/mL BSA, or other suitable 10× cleavage buffer) were incubated at 37° C. In some experiments, incubated compositions further comprised a stop mix (e.g., proteinase K+EDTA, or formamide dye). Cis cleavage activity (e.g., nickase activity) of the effector protein was confirmed by gel electrophoresis. FIG. 19 show the results of the cis cleavage activity. FIG. 20 shows quantitative analysis of FIG. 19 data.

An analysis of FIGS. 19 and 20 indicates that the catalytic activity of the effector proteins can be modified by changing the length of the spacer sequence of the guide nucleic acid. For example, a spacer sequence of 12 and 13 nucleotides long appears to have preference for nicking the double stranded target nucleic acid over generation of a double strand break.

### Example 17: Determining Analog Incubation Conditions for Assessing Translocation Rates Across Three Different Target Nucleic Acids (B2M, TRAC, CIITA) after Multiplex Editing with D220R Variant of CasM.265466

Described herein is the method for measuring and evaluating D220R variant of CasM.265466 translocations (chromosomal rearrangements) rates in comparison to Cas9. The guide nucleic acids were engineered to target B2M gene, TRAC gene, or CIITA gene. Briefly, T cells were thawed and activated with CD3/CD28 dyna beads for 72 hours. Followingly, 3e5 activated T cells were electroporated with mRNA encoding effector protein (5 μg) and guide nucleic acids (500 μM) for editing single gene or all three genes simultaneously. The electroporated T cells were cultured for 3 days in a 48 well plate. The electroporated cells were then evaluated for editing efficiency by FACS and NGS analysis, translocations/chromosomal rearrangement assessment was determined by dGH assay, and cell viability was assessed by counting trypan blue stained T cells by Countess cell counter. For dGH assay, the electroporated cells were treated with an analog 3 days post-electroporation for 17 hours. Followingly, the cells were treated with colcemid for 4 hours. Alternatively, the electroporated cells were also treated with an analog 3 days post-electroporation for 14 hours followed by colcemid treatment for 4 hours (the results are not shown).

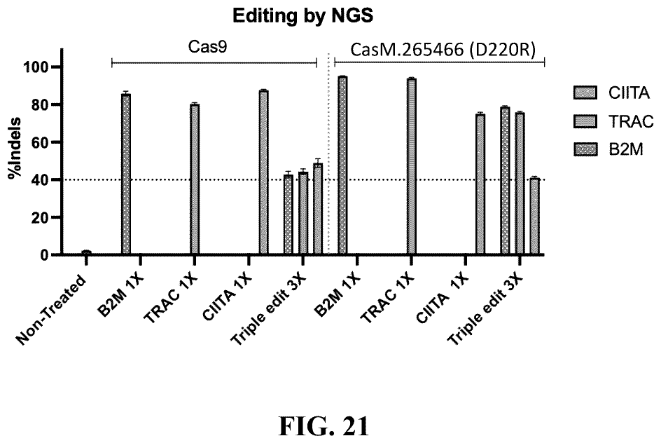

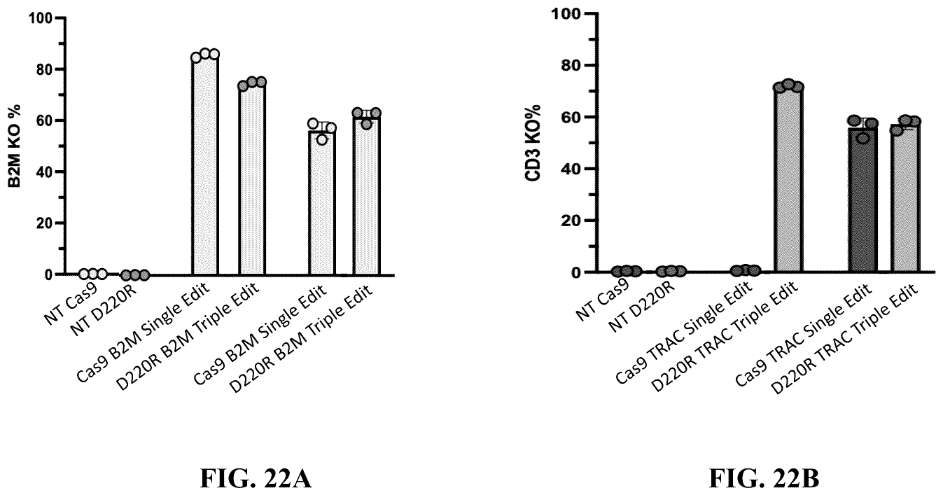

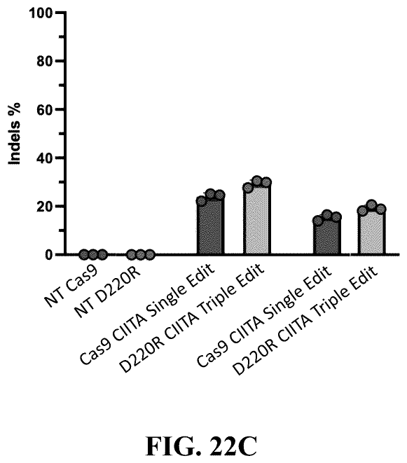

FIG. 21 shows results of NGS study. Similarly, FIGS. 22A-22C shows results of FACS. An analysis of FIG. 21 and FIGS. 22A-22C indicated that editing efficiency for Cas9 and D220R variant of CasM.265466 effector protein was similar for both single gene editing and simultaneous triple gene editing. The analysis further indicates that fewer T cells were observed to be consistently triple gene edited by Cas9 effector protein relative to D220R variant of CasM.265466 effector protein.

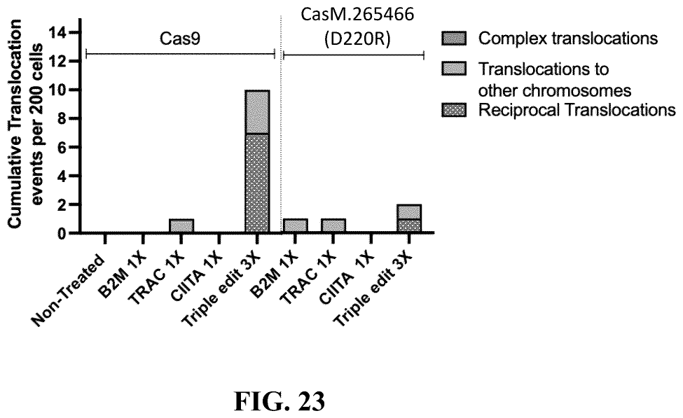

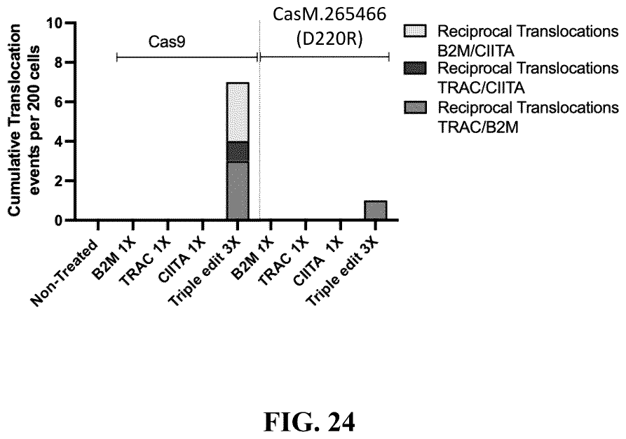

FIG. 23 shows results of translocation rates based on dGH assay. Similarly, FIG. 24 shows results of reciprocal translocations rates based on dGH assay. An analysis of FIG. 23 and FIG. 24 indicated that primary human T cells that were triple edited with D220R variant of CasM.265466 showed fewer translocations relative to Cas9 edited T cells.

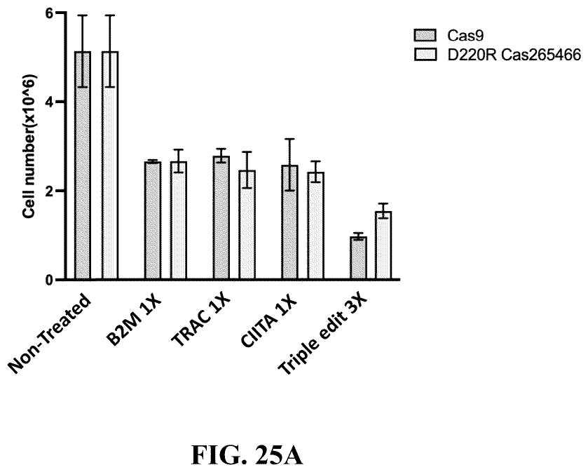

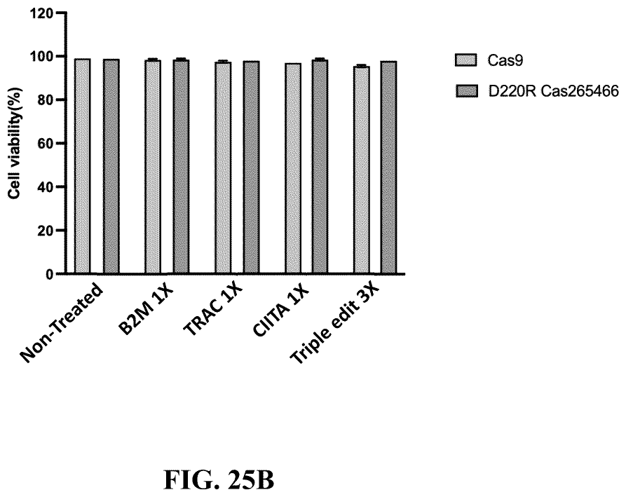

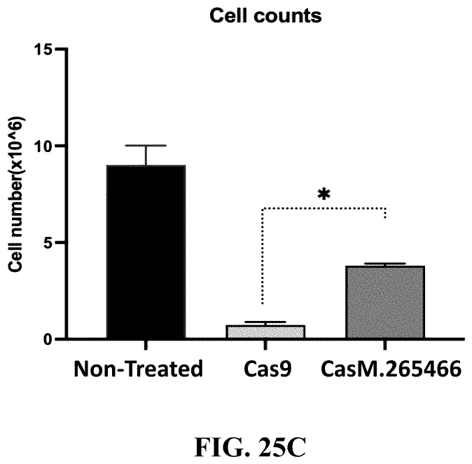

FIG. 25A and FIG. 25C show results of cell counts and FIG. 25B shows results of cell viability.

### Example 18: Optimizing Dose of mRNA Encoding Effector Protein for Assessing Translocation Rates Across Three Different Target Nucleic Acids (B2M, TRAC, CIITA) after Multiplex Editing with D220R Variant of CasM.265466

Described herein is a method for measuring and evaluating D220R variant of CasM.265466 translocations (chromosomal rearrangements) rates in comparison to Cas9. The guide nucleic acids were engineered to target B2M gene, TRAC gene, or CIITA gene. Briefly, T cells were thawed and activated with CD3/CD28 dyna beads for 72 hours. Followingly, 3e5 activated T cells were electroporated with mRNA encoding effector protein (10 μg) and guide nucleic acids (500 μM) for editing single gene or all three genes simultaneously. The electroporated T cells were cultured for 3 days in a 48 well plate. The electroporated cells were then evaluated for editing efficiency by NGS analysis. For dGH assay, the electroporated cells were treated with an analog 3 days post-electroporation for 17 hours. Followingly, the cells were treated with colcemid for 3 hours.

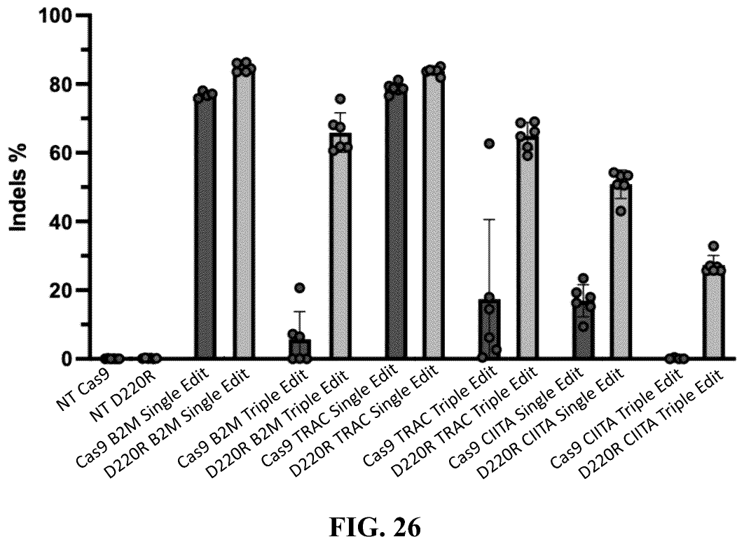

FIG. 26 shows results of an NGS study. An analysis of FIG. 26 indicated that D220R variant of CasM.265466 had higher or similar editing activity relative to Cas9. Additionally, D220R variant of CasM.265466 consistently showed superior editing activity for triple edit relative to Cas9. For single edit, D220R variant of CasM.265466 showed similar, if not higher, editing activity relative to Cas9.

### Example 19: Dose Titration of mRNA Encoding Effector Protein for Assessing Translocation Rates Across Three Different Target Nucleic Acids (B2M, TRAC, CIITA) after Multiplex Editing with D220R Variant of CasM.265466

Described herein is a method for measuring and evaluating D220R variant of CasM.265466 translocations (chromosomal rearrangements) rates in comparison to Cas9. The guide nucleic acids were engineered to target B2M gene, TRAC gene, or CIITA gene. Briefly, T cells were thawed and activated with CD3/CD28 dyna beads for 72 hours. Following activation, 3e5 activated T cells were electroporated with mRNA encoding effector protein (6 μg or 9 μg) and guide nucleic acids (500 μM) for editing single gene or all three genes simultaneously. The electroporated T cells were cultured for 3 days in a 48 well plate. The electroporated cells were then evaluated for editing efficiency by FACS and NGS analysis, translocations/chromosomal rearrangement assessment was determined by dGH assay, and cell viability was assessed by counting trypan blue stained T cells by Countess cell counter. For dGH assay, the electroporated cells were treated with an analog 3 days post-electroporation for 17 hours. Then the cells were treated with colcemid for 3 hours.

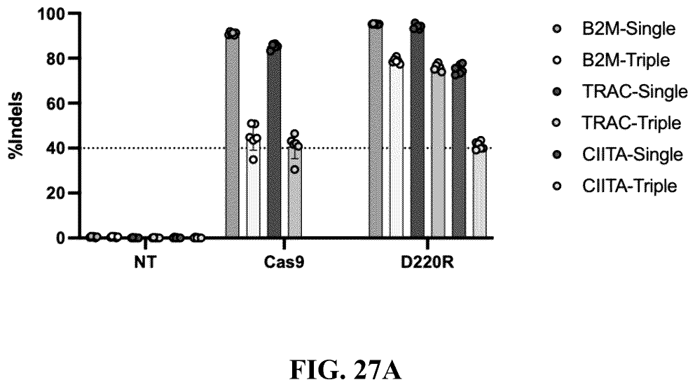

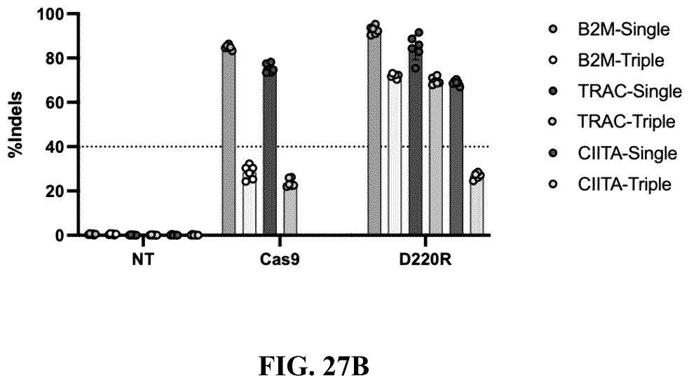

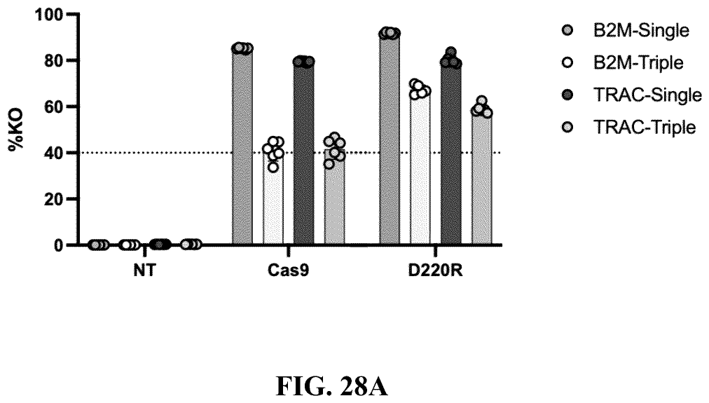

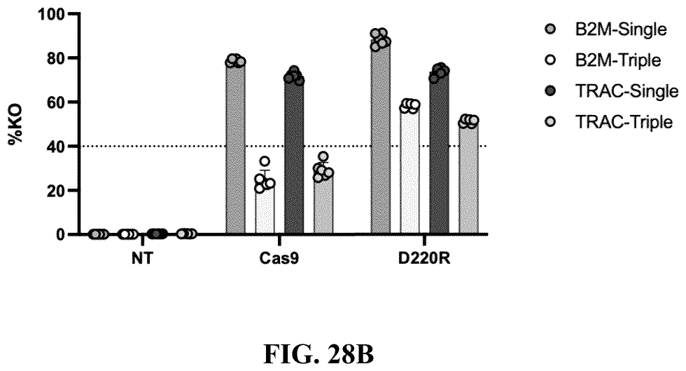

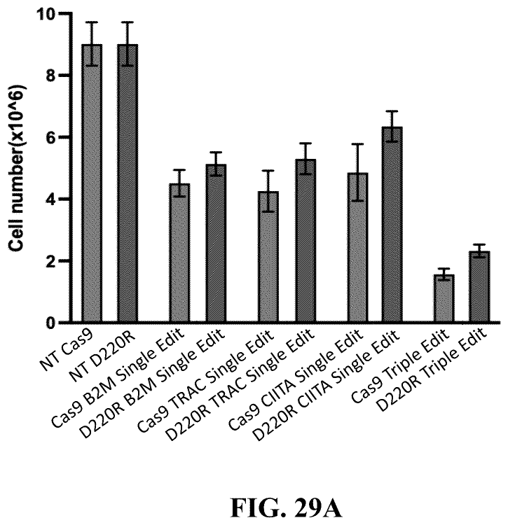

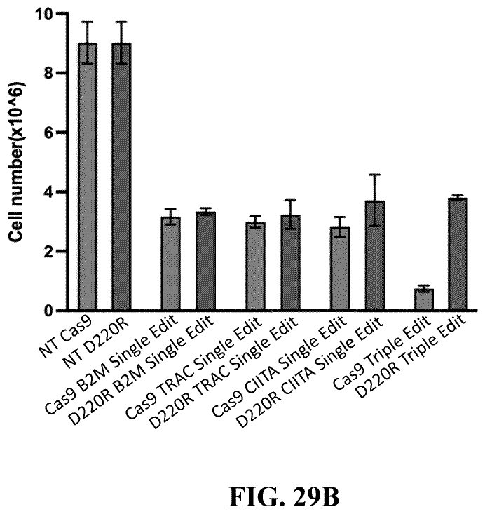

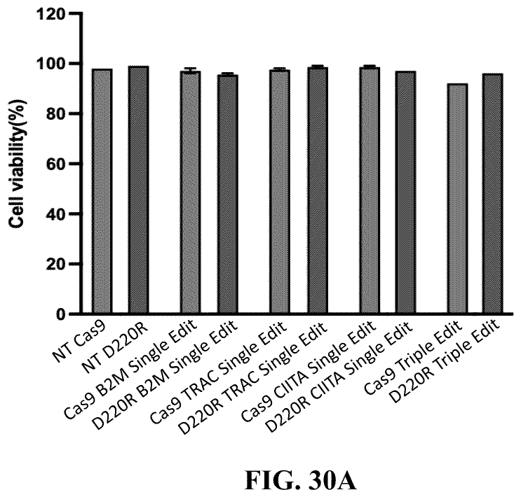

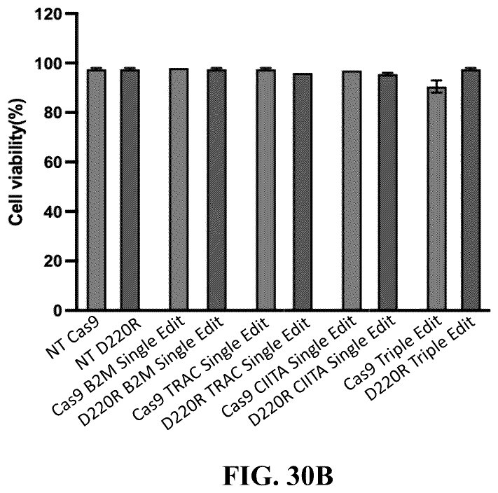

FIG. 27A and FIG. 27B show results of an NGS study at 6 μg or 9 μg, respectively. Similarly, FIG. 28A and FIG. 28B show results of FACS study at 6 μg or 9 μg, respectively. FIG. 29A and FIG. 29B show results of cell counts at 6 μg or 9 μg, respectively. FIG. 30A and FIG. 30B show results of cell viability at 6 μg or 9 μg, respectively. An analysis of FIGS. 27A-27B and 28A-28B indicates that D220R variant of CasM.265466 is capable of generating % indel that is similar to or higher than Cas9. An analysis of FIGS. 29A-29B indicates that D220R variant of CasM.265466 does not negatively affect cell growth relative to Cas9, and in some cases, D220R variant of CasM.265466 showed higher cell growth relative to Cas9. Similarly, an analysis of FIGS. 30A-30B indicates that D220R variant of CasM.265466 does not negatively affect cell viability relative to Cas9.

### Example 20: In Vivo Editing of Pcsk9 Gene Using CasM.265466 Effector Protein System

Described herein is the method of editing target nucleic acid in vivo by CasM.265466 effector protein system relative to Cas9 effector protein system. Vectors were engineered to express effector protein and guide nucleic acids. Briefly, three effector proteins (SaCas9, CasM.265466, or CasM.265466 D220R) and guide RNA (targeting mouse Pcsk9) were cloned, packaged into AAV8 particles, and delivered to 8-week old C57B/6 mice by intravenous injection. The target nucleic acid was contained within the Pcsk9 gene. The guide nucleic acid comprised a handle sequence of ACAGCUUAUUUGGAAGCUGAAAUGUGAGGUUUAUAACACUCACAAGAAUCCUGAAAA AGGAUGCCAAAC (SEQ ID NO: 107). After two and four weeks, livers and serum were harvested for NGS and ELISA respectively. FIG. 31 shows results of an NGS study. An analysis of FIG. 31 indicates that D220R variant of CasM.265466 generates more % indel relative to corresponding wildtype effector protein.

### Example 21: Determining Off-Target Activity of CasM.265466 Effector Protein System

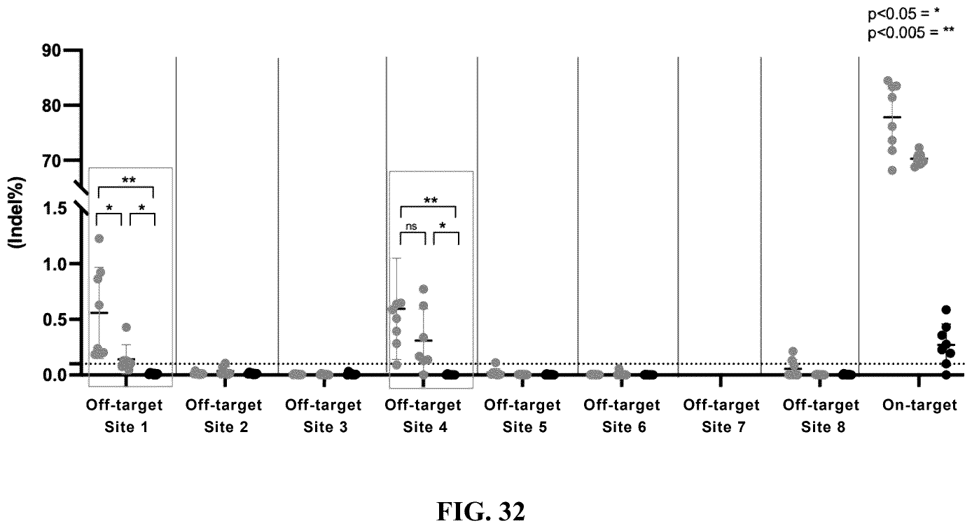

Described herein is the off-target activity of CasM.265466 effector protein system. Briefly, 350,000 activated T cells were electroporated using Neon system with mRNA encoding either wildtype CasM.265466 or D220R variant thereof, and a guide nucleic acid targeting TRAC gene. These groups of treated T cells lysates were then probed against primers designed for the 8 off target sites identified in silico for this guide using amplicon-sequencing. FIG. 32 shows results of NGS study conducted for determining off-target activity. An analysis of FIG. 32 indicates that both CasM.265466 effector protein and D220R variant thereof have very low off-target activity.

### Example 22: Indel Activity of CasM.265466 Variants

Fifty-one nucleic acid vectors encoding engineered polypeptide variants were constructed for assessing the indel activity of CasM.265466 variants. The engineered polypeptides that were tested are summarized in TABLE 18. This experiment used sgRNAs comprising a handle sequence of SEQ ID NO: 107 and a spacer sequence. The spacer sequence was complementary to a target sequence of MLH1. For positive control, WT CasM.265466 effector protein was used.

TABLE 18
Engineered Polypeptides for Base Editing

| Single | | | | | | Variants with |
|---|---|---|---|---|---|---|
| substitution | | | | | | three |
| variant | Variants with two substitutions | | | | | substitutions |
| D220R | T84R and | T84R and | C202R and | S209F and | D220R and | A75F, D220R |
| | D171K | E362D | S209F | D220R | E362D | and Q360R |
| A306K | T84R and | T84R and | C202R and | S209F and | D220R and | N193K, D220R |
| | C202K | A393E | S209Y | Q360R | A393R | and Q360R |
| | T84R and | D171K and | C202R and | S209Y and | E225R and | S209F, D220R |
| | S209Y | C202R | E225K | D220R | K250N | and Q360R |
| | T84R and | D171K and | C202R and | S209Y and | E225R and | D220R, K250N |
| | E225K | D220R | K250N | Q360R | A306K | and Q360R |
| | T84R and | D171K and | C202R and | D220R and | N286K and | D220R, A306K |
| | K250N | E225R | Y315M | K250N | Q360R | and Q360R |
| | T84R and | D171K and | C202R and | D220R and | A306K and | D220R, Q360R |
| | N286K | Q360R | E362D | N286K | Q360R | and E362D |
| | T84R and | N193K and | C202R and | D220R and | Y315M | D220R, Q360R |
| | A306K | D220R | A393E | A306K | and Q360R | and A393E |
| | T84R and | C202R and | S209F and | D220R and | Q360R and | |
| | Y315M | D220R | E225R | Y315M | A393E | |

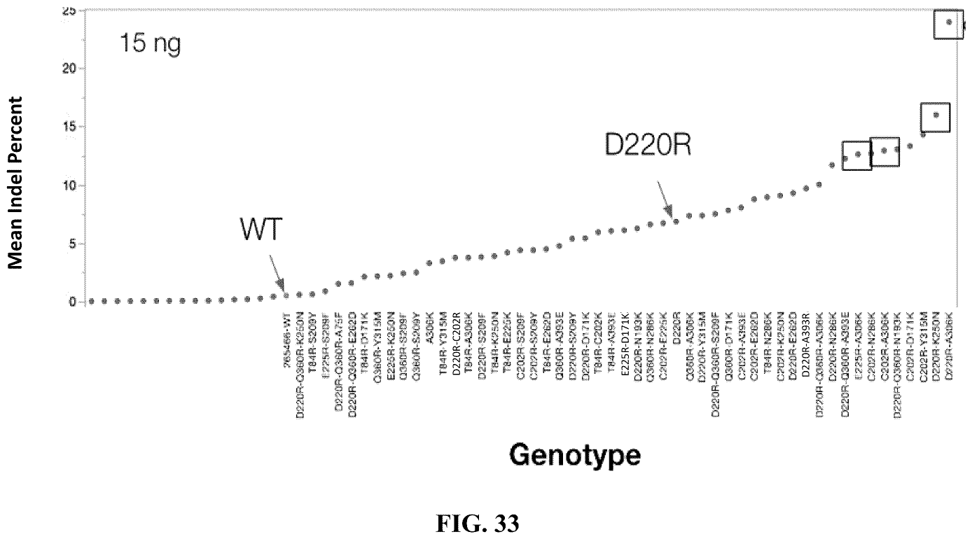

For each polypeptide, indel activity was determined in eukaryotic mammalian cells. Briefly, cells were transfected with the nucleic acid vectors and guide RNAs. The cells were harvested 72 hrs later and % indel was analyzed via NGS. FIG. 33 shows results. The boxed data points from left to right correspond to E225R/A306K, C202R/A306K, D220R/K250N, and D220R/A306K. Notably, variants E225R/A306K, C202R/A306K and D220R/K250N demonstrated about 2.5 fold greater indel activity than D220R variant and about 10-15 fold greater indel activity than WT CasM.265466. D220R/A306K demonstrated greater than 4-fold improvement over D220R variant and greater than 20-fold improvement over WT CasM.265466.

### Example 23: Determination of Effect of Linker Length Variation on Base Editing Activity of Engineered Proteins Fused to an Adenine Base Editing Enzyme

Twenty-six nucleic acid vectors encoding engineered polypeptides were constructed for determining the effect of variation in linker length on base editing activity on a target nucleic acid, wherein the target nucleic acid comprised a target strand and a non-target strand. The engineered polypeptides that were tested are summarized in TABLE 19. Briefly, the adenine base editing enzyme ABE8e (SEQ ID NO: 2) was fused to either the C-terminus or N-terminus of an effector protein variant (CasM.265466 (SEQ ID NO: 1) having D220R and E335Q substitutions) directly (0aa) or by a GS-rich linker of two amino acids (2aa), three amino acids (3aa), five amino acids (5aa), ten amino acids (10aa), twenty amino acids (20aa) or forty amino acids (40aa). The experiment used two sgRNAs: (a) a first sgRNA (PL28330) comprised a handle sequence of SEQ ID NO: 107 and a spacer sequence of SEQ ID NO: 157; and (b) a second sgRNA (PL28341) comprised a handle sequence of SEQ ID NO: 107 and a spacer sequence of SEQ ID NO: 219.

TABLE 19
Engineered Polypeptides for Base edition

| Name | Linkers Tested | Description of Engineered Polypeptides |
|---|---|---|
| C-terminus | 0aa, 2aa, 5aa, 10aa, 20aa, | N-terminus of ABE8e (SEQ ID NO: 2) fused to C- |
| | 40aa GS-rich linkers | terminus of CasM.265466 (SEQ ID NO: 1) having |
| | | D220R and E335Q substitutions |
| N-terminus | 0aa, 2aa, 3aa, 5aa, 10aa, | C-terminus of ABE8e (SEQ ID NO: 2) fused to N- |
| | 20aa, 40aa GS-rich | terminus of CasM.265466 (SEQ ID NO: 1) having |
| | linkers | D220R and E335Q substitutions |

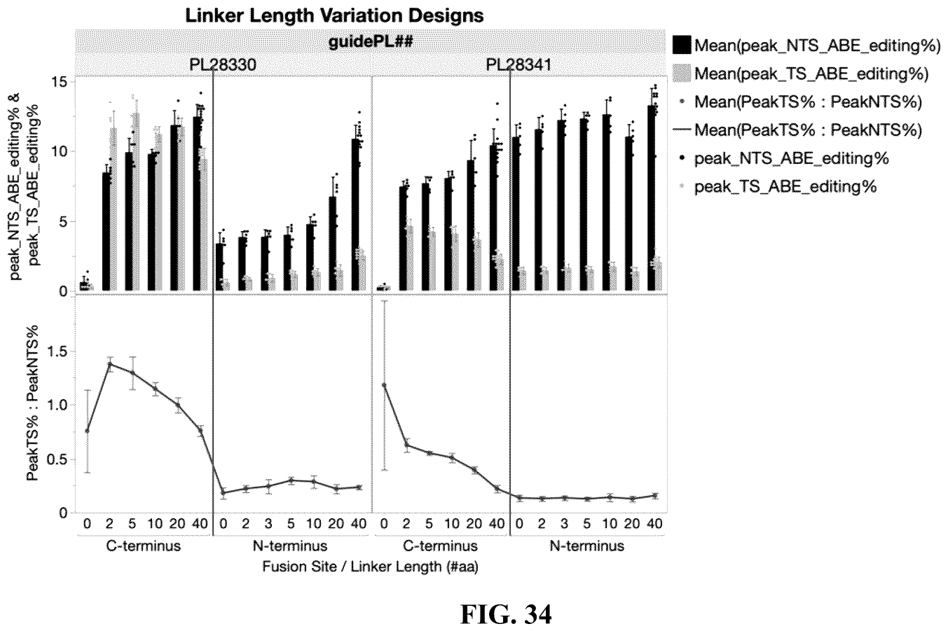

For each polypeptide, % editing of the target nucleic acid was determined in eukaryotic mammalian cells in presence of a uracil glycosylase inhibitor. Cells are transfected with the nucleic acid vectors and guide RNAs. After sufficient incubation, DNA was extracted from the transfected cells. Target sequences were PCR amplified and sequenced by NGS and MiSeq. The presence of base modifications was analyzed from sequencing data (FIG. 34). An analysis of FIG. 34 indicates that a length of linker effects preference for editing of the target strand relative to editing of the non-target strand of the engineered polypeptide, wherein the engineered polypeptide comprises C-terminus of the effector protein variant fused to the adenine base editing enzyme.

### Example 24: Determination of Effect of Linker Length Variation on Nicking Activity of Engineered Proteins

Four nucleic acid vectors encoding engineered polypeptides were constructed for determining the effect of variation in length of a spacer sequence of a guide RNA on nicking activity on a target nucleic acid, wherein the target nucleic acid comprises a target strand and a non-target strand. The engineered polypeptides that were tested included a first effector protein variant (CasM.265466 (SEQ ID NO: 1) having a D220R substitution (ActiveD220R)) and a second effector protein variant (CasM.265466 (SEQ ID NO: 1) having D220R and E335Q substitutions (dCasD220R)), each fused to an adenine base editing enzyme ABE8e (SEQ ID NO: 2) at either C-terminus or N-terminus. The experiment used sgRNAs comprising a handle sequence of SEQ ID NO: 107 and the spacer sequence of 12 nucleotides, 14 nucleotides, 18 nucleotides, or 20 nucleotides.

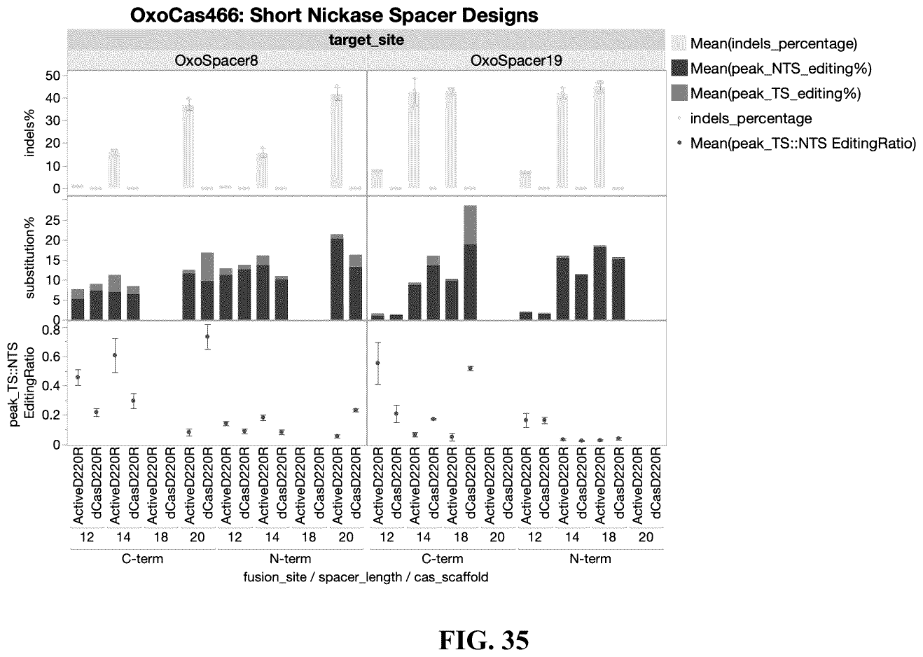

For each guide RNAs, indel activity was determined in eukaryotic mammalian cells. Cells are transfected with the nucleic acid vectors and guide RNAs. After sufficient incubation, DNA was extracted from the transfected cells. Target sequences were PCR amplified and sequenced by NGS and MiSeq. The presence of nicking was determined from NGS data (FIG. 35). An analysis of FIG. 35 indicates that reducing a spacer length to 12-14 nucleotides may improve preference for nicking of the target strand relative to nicking of the non-target strand. Additionally, the analysis indicates that the guide RNA having a spacer length of 12-14 nucleotides may reduce editing efficiency relative to the guide RNA having a spacer length of 18-20 nucleotides.

### Example 25: Determining Base Editing Activity of Cytosine Base Editor System

Two nucleic acid vectors (PL31754 and PL31755), each encoding engineered polypeptides, were constructed for determining cytosine base editing activity. The engineered polypeptides comprised a Cytosine Deaminase fused to the C-terminus of an effector protein variant (CasM.265466 (SEQ ID NO: 1) having D220R and E335Q substitutions) by a XTEN40 linker. The vectors PL31754 and PL31755, comprised Cytosine Deaminase comprising an amino acid sequence of SEQ ID NO: 373 or SEQ ID NO: 374, respectively. The engineered polypeptide further comprised a uracil glycosylase inhibitor (2×UGI) linked to C-terminus of the Cytosine Deaminase by a linker. Sixteen different guide RNAs were used.

For each engineered polypeptide, the base editing activity was tested in eukaryotic mammalian (HEK293T) cells. Cells were transfected with the nucleic acid vectors and guide RNAs. After sufficient incubation, DNA was extracted from the transfected cells. Target sequences were PCR amplified and sequenced by NGS and MiSeq. The presence of base modifications was analyzed from the sequencing data. For determining editing window, positions were fixed counting from 5′ of the non-target strand, wherein position 1 is the first nucleotide of the non-target strand after PAM sequence.

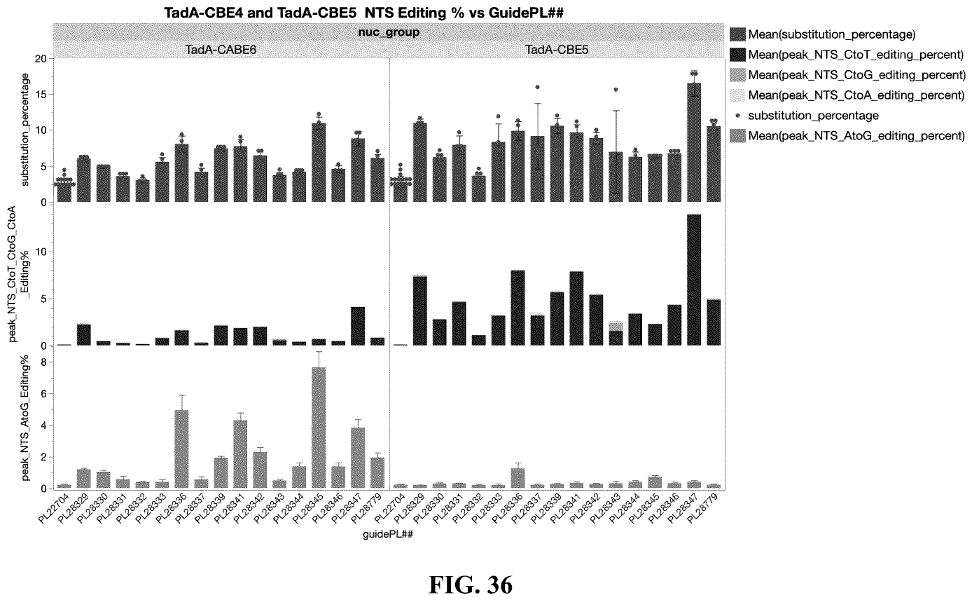

Representative base editing data for two of the five tested cytosine deaminase are shown in FIG. 36, which includes: (1) % substitution generated by each guide nucleic acid; (2) % editing generated by each guide nucleic acid from C to T, C to G and C to A; and (3) % editing generated by each guide nucleic acid from A to G. An analysis of FIG. 36 indicates that cytosine base editing activity of cytosine deaminase can be used for editing of a target nucleic acid (e.g., introduction of stop codon into a coding sequence of a gene).

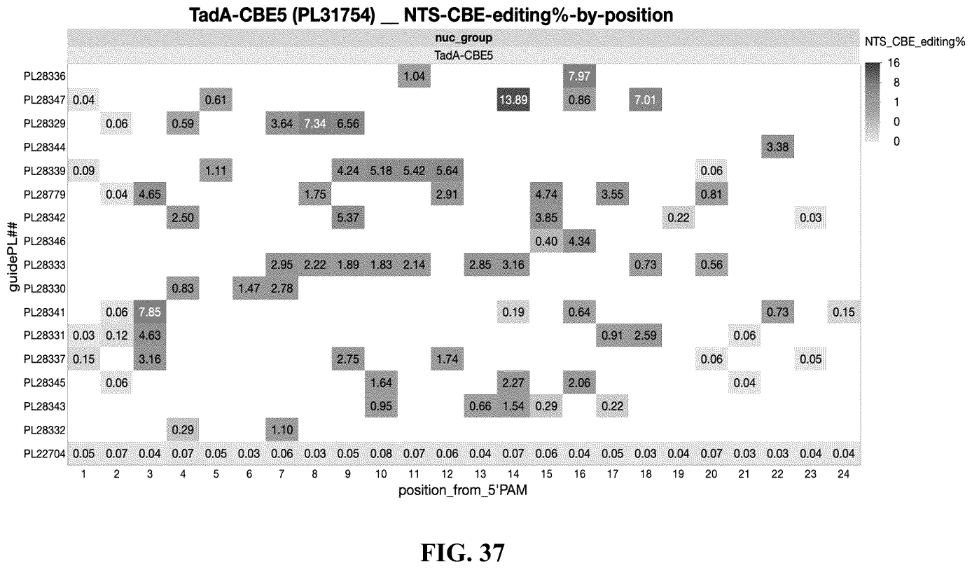

FIG. 37 shows the editing windows on the non-target strand for the engineered polypeptide delivered by PL31754 vector as a heat map color distribution. All engineered polypeptides were capable of base editing. The majority of editing occurred in an editing window of positions 3-19.

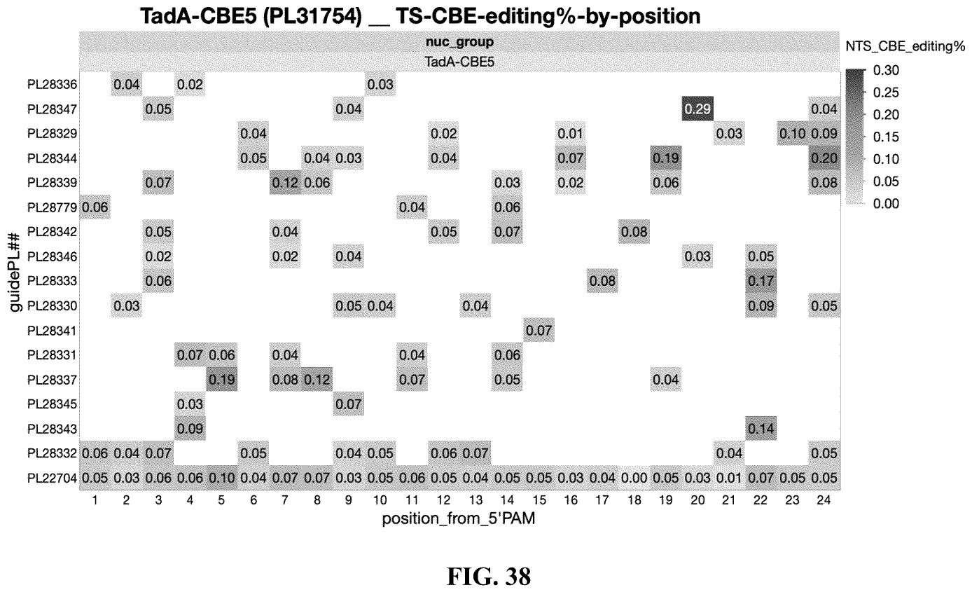

FIG. 38 shows the editing windows on the target strand for the engineered polypeptide delivered by PL31754 vector as a heat map color distribution. All engineered polypeptides were capable of base editing. The majority of editing occurred in an editing window of positions 3-19.

### Example 26: Lipid Nanoparticle Formulations

In order to assess lipofection of mRNAs encoding various effector proteins, lipid nanoparticles (LNPs) are formulated using an mRNA having a nucleotide sequence encoding an effector protein variant (CasM.265466 (SEQ ID NO: 1) having D220R substitution), and two different guide RNAs. Each guide RNA comprises a handle sequence of ACAGCUUAUUUGGAAGCUGAAAUGUGAGGUUUAUAACACUCACAAGAAUCCUGAAAA AGGAUGCCAAAC (SEQ ID NO: 107) and a spacer sequence selected from GGGUUUUGUCCUCACUCUGA (SEQ ID NO: 222) and UAGAACCUUGAUGACAUAGC (SEQ ID NO: 223). Cas9 mRNA and R8217 Axolab guide RNA are used as a positive control. The guide RNAs each target human PCSK9. The mRNA and guide RNAs are mixed and diluted to various concentrations, as shown in TABLE 20 below, at a pH of about 3 to about 5 in acetate buffer or citrate buffer. Ionizable lipids, phospholipids, cholesterol and PEG lipids are dissolved in ethanol at various molar ratios. The lipid mixtures and RNA mixtures are further mixed in a microfluidic device at the various ionizable/RNA weight to weight ratios. The formulations are then further dialyzed against a Tris-saline buffer or phosphate buffered saline (PBS) at a pH of about 7.4 and concentrated to a desired concentration by Amicon filtration. The isolated LNPs are characterized to determine the encapsulation efficiency (EE), polydispersity index (PDI) and average particle size, as shown in TABLE 20 below.

TABLE 20
LNP Formulations

| | | Ionizable | Phospho- | Chol- | PEG |
|---|---|---|---|---|---|
| | | lipid | lipid | esterol | lipid |
| | | molar | molar | molar | molar |
| No. | Ionizable lipid | ratio | ratio | ratio | ratio |
| 1 | DLin-MC3-DMA (4-(dimethylamino)- | 50 | 10 DSPC | 38.5 | 1.5 |
| | butanoic acid, (10Z,13Z)-1-(9Z,12Z)-9,12- | | | | |
| | octadecadien-1-yl-10,13-nonadecadien-1-yl | | | | |
| | ester) (CAS NO. 1224606-06-7) | | | | |
| 2 | DLin-KC2-DMA (N,N-dimethyl-2,2-di- | 50 | 10 DSPC | 38.5 | 1.5 |
| | (9Z,12Z)-9,12-octadecadien-1-yl-1,3- | | | | |
| | dioxolane-4-ethanamine) (CAS NO. | | | | |
| | 1190197-97-7) | | | | |
| 3 | SM-102 (L710-1) (8-[(2-hydroxyethyl)[6- | 50 | 10 DSPC | 38.5 | 1.5 |
| | oxo-6-(undecyloxy)hexyl]amino]-octanoic | | | | |
| | acid, 1-octylnonyl ester) (CAS NO. | | | | |
| | 2089251-47-6) | | | | |
| 4 | Lipid5 (L710-2) (2-hexyl-decanoic acid, | 50 | 10 DSPC | 38.5 | 1.5 |
| | 1,1′-[[(4-hydroxybutyl)imino]di-6,1- | | | | |
| | hexanediyl] ester) (CAS NO. 2089251- | | | | |
| | 33-0) | | | | |
| 5 | LP01 (3-[4,4-Bis(octyloxy)-1-oxobutoxy]-2- | 45 | 9 DSPC | 44 | 2 |
| | [[[[3-(diethylamino)propoxy]carbonyl]oxy] | | | | |
| | methyl]propyl(9Z,12Z)-9,12-octadecadienoate) | | | | |
| | (CAS NO. 1799316-64-5) | | | | |
| 6 | ALC-0315 (2-hexyl-decanoic acid, 1,1′- | 46.3 | 9.4 DSPC | 42.7 | 1.6 |
| | [[(4-hydroxybutyl)imino]di-6,1-hexanediyl] | | | | ALC- |
| | ester) (CAS NO. 2036272-55-4) | | | | 0159 |
| | | | | | (amide |
| | | | | | PEG) |
| 7 | cKK-E12 (3,6-bis({4-[bis(2- | 35 | 16 DOPE | 46.5 | 2.5 |
| | hydroxydodecyl)amino]butyl} piperazine- | | | | |
| | 2,5-dione) (CAS NO. 1432494-65-9) | | | | |
| 8 | OF-02 (3,6-bis[4-[bis[(9Z,12Z)-2-hydroxy- | 35 | 16 DOPE | 46.5 | 2.5 |
| | 9,12-octadecadien-1-yl]amino]butyl]-2,5- | | | | |
| | piperazinedione) (CAS NO. 1883431-67-1) | | | | |
| 9 | TT3 (N1,N3,N5-tris(3- | 35 | 16 DOPE | 46.5 | 2.5 |
| | (didodecylamino)propyl)benzene-1,3,5- | | | | |
| | tricarboxamide) (CAS NO. 1821214-50-9) | | | | |
| 10 | FTT5 (Hexa(octan-4-yl) 9,9′,9″,9′″,9″″,9″″′- | 35 | 16 DOPE | 46.5 | 2.5 |
| | (((benzenetricarbonyltris(azanediyl))tris(pro | | | | |
| | pane-3,1-diyl)) | | | | |
| | tris(azanetriyl))hexanonanoate) (CAS NO. | | | | |
| | 2328129-27-5) | | | | |
| 11 | C12-200 (1,1′-[2-[4-[2-[[2-[bis(2- | 35 | 16 DOPE | 46.5 | 2.5 |
| | hydroxydodecyl)amino]ethyl](2- | | | | |
| | hydroxydodecyl)amino]ethyl]-1- | | | | |
| | piperazinyl]ethyl]imino]bis-2-dodecanol) | | | | |
| | (CAS NO. 1220890-25-4) | | | | |
| 12 | 5A2-SC8 (4,7,10,13,16- | 35 | 16 DOPE | 46.5 | 2.5 |
| | Pentaazanonadecanedioic acid, 4,10,16- | | | | |
| | tris [3-[2-[2-methyl-3-(octylthio)-1-oxoprop | | | | |
| | oxy]ethoxy]-3-oxopropyl]-, 1,19-bis [2-[2- | | | | |
| | methyl-3-(octylthio)-1-oxopropoxy]ethyl] | | | | |
| | ester) (CAS NO. 1857341-90-2) | | | | |
| 13 | 306-O12B (tetrakis(2- | 50 | 10 DOPC | 38.5 | 1.5 |
| | (octyldisulfaneyl)ethyl) 3,3′,3″,3′″- | | | | |
| | (((methylazanediyl)bis(propane-3,1- | | | | |
| | diyl))bis(azanetriyl))tetrapropionate) (CAS | | | | |
| | NO. 2566523-06-4) | | | | |
| 14 | ssPalmO-Phe (Bis[2-(4-{2-[4-(cis-9- | 52.5 | 7.5 DOPC | 40 | 3 |
| | octadecenoyloxy)phenylacetoxy]ethyl} piper | | | | |
| | idinyl)ethyl] disulfide) (CAS NO. 2377474- | | | | |
| | 67-2) | | | | |
| 15 | OF-C4-Deg-Lin (9,12-octadecadienoic acid, | 35 | 16 DOPE | 46.5 | 2.5 |
| | (9Z,12Z)-1,1′,1″,1′′′-[(3,6-dioxo-2,5- | | | | |
| | piperazinediyl)bis(4,1-butanediylnitrilodi- | | | | |
| | 4,1-butanediyl)] ester) (CAS NO. 1853203- | | | | |
| | 01-6) | | | | |
| 16 | 306Oi10 (tetrakis(8-methylnonyl) | 35 | 16 DOPE | 46.5 | 2.5 |
| | 3,3′,3″,3″′-(((methylazanediyl)bis(propane- | | | | |
| | 3,1-diyl))bis(azanetriyl))tetrapropionate) | | | | |
| | (CAS NO. 2322290-93-5) | | | | |
| 17 | BAMEA-O16B (bis(2- | 35 | 16 DOPE | 46.5 | 2.5 |
| | (dodecyldisulfaneyl)ethyl) 3,3′-((3-methyl- | | | | |
| | 9-oxo-10-oxa-13,14-dithia-3,6- | | | | |
| | diazahexacosyl)azanediyl)dipropionate) | | | | |
| | (CAS NO. 2490668-30-7) | | | | |
| 18 | nonyl 8-((7,7-bis(octyloxy)heptyl)(2- | 30 | 10 DSPC | 58.5 | 1.5 |
| | hydroxyethyl)amino)octanoate (CAS NO. | | | | |
| | 2415092-81-6) | | | | |
| 19 | ALC-A9 (1,19-Bis(2-butyloctyl) 10-[[3- | 46.3 | 9.4 DSPC | 45.7 | 1.6 |
| | (dimethylamino) propyl](1- | | | | ALC- |
| | oxononyl)amino]nonadecanedioate) (CAS | | | | 0159 |
| | NO. 2036272-50-9) | | | | |
| 20 | Lipid 2,2 (8,8) 4C CH3 (heptadecan-9-yl | 50 | 7 DSPC | 40 | 3 |
| | (Z)-N-(((3- | | | | |
| | (dimethylamino)propyl)thio)carbonyl)-N- | | | | |
| | (2-(non-2-en-1-yloxy)-2- | | | | |
| | oxoethyl)glycinate) (CAS NO. 2230647-28- | | | | |
| | 4) | | | | |
| 21 | ATX-001 (di((Z)-non-2-en-1-yl) 8,8′- | 50 | 7 DSPC | 40 | 3 |
| | ((2-((2- | | | | |
| | (dimethylamino)ethyl)thio)acetyl)azanediyl) | | | | |
| | dioctanoate) (CAS NO. 1777792-33-2) | | | | |
| 22 | ATX-002 (di((Z)-non-2-en-1-yl) 8,8′-((((2- | 50 | 7 DSPC | 40 | 3 |
| | (dimethylamino)ethyl)thio)carbonyl) | | | | |
| | azanediyl)dioctanoate) (CAS NO. 1777792-34-3) | | | | |
| 23 | CL1 (Pentanoic acid, 5-(dimethylamino)-, | 54.6 | 10.9 DSPC | 32.8 | 1.6 |
| | (6Z)-1,2-di-(4Z)-4-decen-1-yl-6-dodecen- | | | | |
| | 1-yl ester) (CAS NO. 1450888-71-7) | | | | |
| 24 | LP01 (3-[4,4-Bis(octyloxy)-1-oxobutoxy]- | 40 | 17 DOPE | 42 | 1 |
| | 2-[[[[3- | | | | |
| | (diethylamino)propoxy]carbonyl]oxy]methyl] | | | | |
| | propyl (9Z,12Z)-9,12-octadecadienoate) | | | | |
| | (CAS NO. 1799316-64-5) | | | | |
| 26 | CKK-E12-Cas9 | 35 | 16 DOPE | 46.5 | 2.5 |
| 27 | CKK-E12-Cas9 frozen | 35 | 16 DOPE | 46.5 | 2.5 |

LNPs prepared as described above are administered to male C57BL/6J mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively.

### Example 27: Determination of a Fusion Protein Mediated In Vivo Base Editing Activity in ANGPTL3

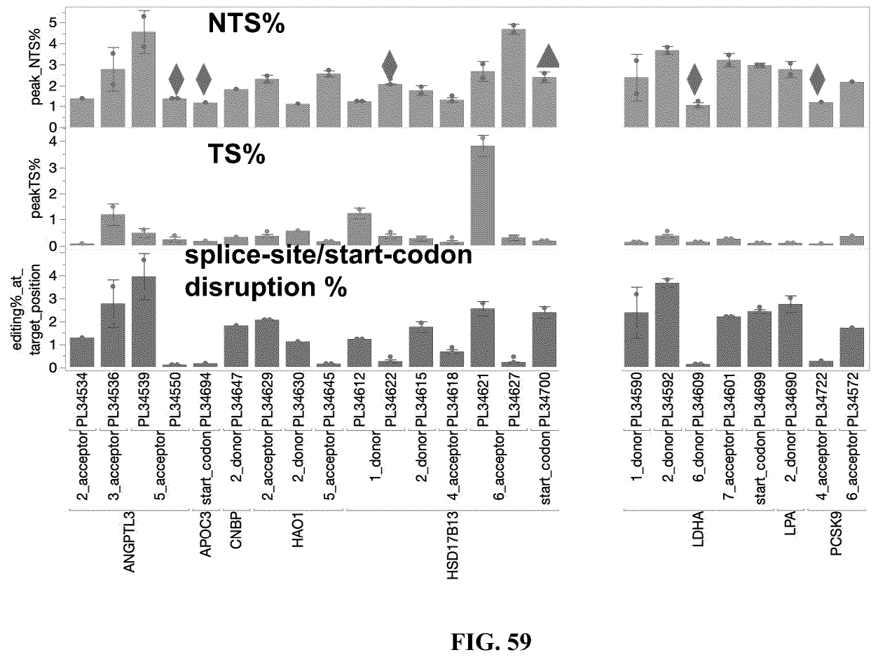

Systems comprising a fusion protein and a guide nucleic acid were tested for their ability to modify ANGPTL3 in a human cell. Briefly, HEK293T cells were transfected with one or more plasmids encoding the fusion protein and the guide nucleic acid. After sufficient incubation, cells were harvested, and base editing was analyzed by NGS. The fusion protein comprised ABE8e (SEQ ID NO: 2) fused to CasM.265466 (SEQ ID NO: 1) having D220R and E335Q substitutions. The N-terminus of ABE8e was fused to the C-terminus of CasM.265466. The guide nucleic acids were engineered such that a guide nucleic acid-effector protein complex targets and edits exon 1, exon 2, exon 3, exon 4, exon 5, exon 6 or exon 7 as provided in TABLE 21. Specifically, the guide nucleic acids were engineered to comprise a handle sequence of SEQ ID NO: 107 and a spacer sequence selected from TABLE 21. TABLE 21 also provides specific PAM sequences that are recognized by the RNP complex of the fusion protein and guide nucleic acid. The spacer sequences of SEQ ID NOs: 224-245 were engineered to cause splice site disruption. The spacer sequences of SEQ ID NOs: 246-248 were engineered to cause start codon disruption. Data for exemplary guide nucleic acids are provided in FIG. 59. Plasmid numbers, PL34534, PL34536, PL34539, and PL34550 described in FIG. 59 correspond to guide nucleic acids comprising spacer sequences according to SEQ ID NOS: 226, 228, 231, and 242, respectively.

TABLE 21
Guide RNAs for Editing of ANGPTL3

| | | Spacer | | PAM |
|---|---|---|---|---|
| Handle | | SEQ | | Sequence |
| SEQ ID | | ID | Target Exon of | recognized |
| NO: | Spacer Sequence | NO: | ANGPTL3 | by RNP |
| 107 | CUUACUUUAAGUGAAGUUAC | 224 | exon #1 (NM_014495.4) | TCTA |
| 107 | UUUUCUACUUACUUUAAGUG | 225 | exon #1 (NM_014495.4) | TTTA |
| 107 | UCCAGACUUUUGUAGAAAAA | 226 | exon #2 (NM_014495.4) | TATA |
| 107 | AAAUACUGACUUACCUGAUU | 227 | exon #2 (NM_014495.4) | ATTA |
| 107 | UCAGCUCAGAAGGACUAGUA | 228 | exon #3 (NM_014495.4) | TTTA |
| 107 | UCUUACCAUCAUGUUUUACA | 229 | exon #3 (NM_014495.4) | AGTG |
| 107 | UUGAUUCUAGGCAUUCCUGC | 230 | exon #4 (NM_014495.4) | TTTA |
| 107 | UUCAGGUAGUCCAUGGACAU | 231 | exon #5 (NM_014495.4) | TATA |
| 107 | GUCCCCUUACCAUCAAGCCU | 232 | exon #5 (NM_014495.4) | TGTA |
| 107 | AAACUUUUCUUUUCAGGAGA | 233 | exon #6 (NM_014495.4) | TTTA |
| 107 | UCAGAAAAAGAUACCUGAAU | 234 | exon #6 (NM_014495.4) | GGTA |
| 107 | UCUCCUUUAGGAGGCUGGUG | 235 | exon #7 (NM_014495.4) | TCTA |
| 107 | UCUUGUUUUUCUACAAAAGU | 236 | exon #2 (NM_014495.4) | ATTA |
| 107 | AAAGAAAUAGAAAAUCAGGU | 237 | exon #2 (NM_014495.4) | AATA |
| 107 | AAUACUAGUCCUUCUGAGCU | 238 | exon #3 (NM_014495.4) | CTTG |
| 107 | AGAAAUGUAAAACAUGAUGG | 239 | exon #3 (NM_014495.4) | AATA |
| 107 | CAUUCAGCAGGAAUGCCUAG | 240 | exon #4 (NM_014495.4) | GGTA |
| 107 | GUGGUACAUUCAGCAGGAAU | 241 | exon #4 (NM_014495.4) | AATG |
| 107 | AAUUAAUGUCCAUGGACUAC | 242 | exon #5 (NM_014495.4) | GTTG |
| 107 | GUUUUGGGAGGCUUGAUGGU | 243 | exon #5 (NM_014495.4) | TATG |
| 107 | GGCCCAACCAAAAUUCUCCU | 244 | exon #6 (NM_014495.4) | TCTA |
| 107 | UCCAGAGGGUUAUUCAGGUA | 245 | exon #6 (NM_014495.4) | ACTG |
| 107 | AAAAUGUUCACAAUUAAGCU | 246 | exon #1 (NM_014495.4) | GATA |
| 107 | AAAUUGAAAAUCAAGAUAAA | 247 | exon #1 (NM_014495.4) | CTTG |
| 107 | AACAUUUUUAUCUUGAUUUU | 248 | exon #1 (NM_014495.4) | TGTG |

Systems that provide base editing in HEK293T cells are tested in vivo. Briefly, LNPs comprising the mRNA encoding the effector protein and guide RNAs are prepared according to the method described in Example 26. LNPs are then administered to humanized mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively. The presence of base modifications is analyzed from sequencing data.

### Example 28: Determination of a Fusion Protein Mediated In Vivo Base Editing Activity in APOC3

Systems comprising a fusion protein and a guide nucleic acid were tested for their ability to modify APOC3 in a human cell. Briefly, HEK293T cells were transfected with one or more plasmids encoding the fusion protein and the guide nucleic acid. After sufficient incubation, cells were harvested, and base editing was analyzed by NGS. The fusion protein comprised ABE8e (SEQ ID NO: 2) fused CasM.265466 (SEQ ID NO: 1) having D220R and E335Q substitutions. The N-terminus of ABE8e was fused to the C-terminus of CasM.265466. The guide nucleic acids were engineered such that a guide nucleic acid-effector protein complex targets and edits exon 1, exon 2, exon 3, or exon 4 as provided in TABLE 22. Specifically, the guide nucleic acids were engineered to comprise a handle sequence of SEQ ID NO: 107 and a spacer sequence selected from TABLE 22. TABLE 22 also provides specific PAM sequences that are recognized by the RNP complex of the fusion protein and guide nucleic acid. The spacer sequences of SEQ ID NOs: 246-257 were engineered to cause splice site disruption. The spacer sequences of SEQ ID NOs: 258-259 were engineered to cause start codon disruption. Data for exemplary guide nucleic acids are provided in FIG. 59. Plasmid numbers PL34694 described in FIG. 59 corresponds to guide nucleic acid comprising spacer sequence according to SEQ ID NO: 258.

TABLE 22
Guide RNAs for Editing of APOC3

| | | Spacer | | PAM |
|---|---|---|---|---|
| Handle | | SEQ | | Sequence |
| SEQ ID | | ID | | recognized |
| NO: | Spacer Sequence | NO: | Target Exon of APOC3 | by RNP |
| 107 | CUUACGGGCAGAGGCCAGGA | 249 | exon #2 (NM_000040.3) | AGTG |
| 107 | CUCUUUCCUCAGGAGCUUCA | 250 | exon #3 (NM_000040.3) | CCTG |
| 107 | AUUUAGGGGCUGGGUGACCG | 251 | exon #4 (NM_000040.3) | ACTG |
| 107 | ACUGAUUUAGGGGCUGGGUG | 252 | exon #4 (NM_000040.3) | CCTG |
| 107 | CUUCCCCUGACUGAUUUAGG | 253 | exon #4 (NM_000040.3) | GTTG |
| 107 | GAGGCAGCUGCUCCAGGUAA | 254 | exon #1 (NM_000040.3) | CCTA |
| 107 | CAUGGCACCUCUGUUCCUGC | 255 | exon #2 (NM_000040.3) | GCTG |
| 107 | GCGCUCCUGGCCUCUGCCCG | 256 | exon #2 (NM_000040.3) | CCTG |
| 107 | AAGCCAUCGGUCACCCAGCC | 257 | exon #4 (NM_000040.3) | ACTG |
| 107 | CAGGAACAGAGGUGCCAUGC | 258 | exon #2 (NM_000040.3) | CTTG |
| 107 | CCCGGGGCUGCAUGGCACCU | 259 | exon #2 (NM_000040.3) | AGTA |

Systems that provide base editing in HEK293T cells are tested in vivo. Briefly, LNPs comprising the mRNA encoding the effector protein and guide RNAs are prepared according to the method described in Example 26. LNPs are then administered to humanized mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively. The presence of base modifications is analyzed from sequencing data.

### Example 29: Determination of a Fusion Protein Mediated In Vivo Base Editing Activity in PCSK9

Systems comprising a fusion protein and a guide nucleic acid were tested for their ability to modify PCSK9 in a human cell. Briefly, HEK293T cells were transfected with one or more plasmids encoding the fusion protein and the guide nucleic acid. After sufficient incubation, cells were harvested, and base editing was analyzed by NGS. The fusion protein comprised ABE8e (SEQ ID NO: 2) fused to CasM.265466 (SEQ ID NO: 1) having D220R and E335Q substitutions. The N-terminus of ABE8e was fused to the C-terminus of CasM.265466. The guide nucleic acids were engineered such that a guide nucleic acid-effector protein complex targets and edits exon 1, exon 2, exon 3, exon 4, exon 5, exon 6, exon 7, exon 8, exon 9, exon 10, exon 11 or exon 12 as provided in TABLE 23. Specifically, the guide nucleic acids were engineered to comprise a handle sequence of SEQ ID NO: 107 and a spacer sequence selected from TABLE 23. TABLE 23 also provides specific PAM sequences that are recognized by the RNP complex of the fusion protein and guide nucleic acid. The spacer sequences of SEQ ID NOs: 260-285 were engineered to cause splice site disruption. The spacer sequences of SEQ ID NOs: 286-287 were engineered to cause start codon disruption. Data for exemplary guide nucleic acids are provided in FIG. 59. Plasmid numbers PL34722 and PL34572 described in FIG. 59 correspond to guide nucleic acid comprising spacer sequence according to SEQ ID NO: 265 and SEQ ID NO: 269, respectively.

TABLE 23
Guide RNAs for Editing PCSK9

| | | Spacer | | PAM |
|---|---|---|---|---|
| Handle | | SEQ | | Sequence |
| SEQ ID | | ID | | recognized |
| NO: | Spacer Sequence | NO: | Target Exon of PCSK9 | by RNP |
| 107 | CACCCGCACCUUGGCGCAGC | 260 | exon #1 (NM_174936.4) | CCTA |
| 107 | GGGCCAGGAUCCGUGGAGGU | 261 | exon #2 (NM_174936.4) | CATG |
| 107 | GCUCACCAGCUCCAGCAGGU | 262 | exon #2 (NM_174936.4) | GGTG |
| 107 | GCUUCUGCAGGCCUUGAAGU | 263 | exon #3 (NM_174936.4) | TCTG |
| 107 | GGGGUCUUACCGGGGGGCUG | 264 | exon #3 (NM_174936.4) | GATG |
| 107 | GAAAGACGGAGGCAGCCUGG | 265 | exon #4 (NM_174936.4) | CTTG |
| 107 | CUUACCUGUCUGUGGAAGCG | 266 | exon #4 (NM_174936.4) | CGTG |
| 107 | UUCGUCGAGCAGGCCAGCAA | 267 | exon #5 (NM_174936.4) | TGTG |
| 107 | GGGCCAUCACUUACCUAUGA | 268 | exon #5 (NM_174936.4) | TCTG |
| 107 | UUCCUCCCAGGCCUGGAGUU | 269 | exon #6 (NM_174936.4) | TTTG |
| 107 | AUGACCUGGAAAGGUGAGGA | 270 | exon #7 (NM_174936.4) | TGTG |
| 107 | CACCAGGCAUUGCAGCCAUG | 271 | exon #8 (NM_174936.4) | CCTG |
| 107 | CUUACCUGCCCCAUGGGUGC | 272 | exon #8 (NM_174936.4) | CCTG |
| 107 | CAGUCACCUCCAUGCGCUCG | 273 | exon #9 (NM_174936.4) | GGTA |
| 107 | ACUCUAAGGCCCAAGGGGGC | 274 | exon #10 (NM_174936.4) | TTTG |
| 107 | CCCCAGGCUGCAGCUCCCAC | 275 | exon #11 (NM_174936.4) | TCTG |
| 107 | GCAGGUGACCGUGGCCUGCG | 276 | exon #12 (NM_174936.4) | TCTG |
| 107 | CCUCGCCGCGGCACAGGUGG | 277 | exon #1 (NR_110451.2) | TCTG |
| 107 | CCAGGCAACCUCCACGGAUC | 278 | exon #2 (NM_174936.4) | GGTG |
| 107 | GCGACCUGCUGGAGCUGGUG | 279 | exon #2 (NM_174936.4) | AGTG |
| 107 | AGUGGCGACCUGCUGGAGCU | 280 | exon #2 (NM_174936.4) | GATG |
| 107 | ACUGUCACACUUGCUGGCCU | 281 | exon #5 (NM_174936.4) | CATG |
| 107 | CUCCCCAGCCUCAGCUCCCG | 282 | exon #6 (NM_174936.4) | TCTA |
| 107 | GCCCCAACUGUGAUGACCUG | 283 | exon #7 (NM_174936.4) | GGTG |
| 107 | CCCCCCAGCACCCAUGGGGC | 284 | exon #8 (NM_174936.4) | CCTG |
| 107 | CAAAACAGCUGCCAACCUGC | 285 | exon #9 (NM_174936.4) | CCTG |
| 107 | CCCAUGAGGGCCAGGGGAGA | 286 | exon #1 (NM_174936.4) | GGTG |
| 107 | GCCCUCAUGGGCACCGUCAG | 287 | exon #1 (NM_174936.4) | CCTG |

Systems that provide base editing in HEK293T cells are tested in vivo. Briefly, LNPs comprising the mRNA encoding the effector protein and guide RNAs are prepared according to the method described in Example 26. LNPs are then administered to humanized mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively. The presence of base modifications is analyzed from sequencing data.

### Example 30: Determination of a Fusion Protein Mediated In Vivo Base Editing Activity in LDHA

Systems comprising a fusion protein and a guide nucleic acid were tested for their ability to modify LDHA in a human cell. Briefly, HEK293T cells were transfected with one or more plasmids encoding the fusion protein and the guide nucleic acid. After sufficient incubation, cells were harvested, and base editing was analyzed by NGS. The fusion protein comprises an amino acid sequence N-terminus of ABE8e (SEQ ID NO: 2) fused to C-terminus of CasM.265466 (SEQ ID NO: 1) having D220R and E335Q substitutions. The guide nucleic acids were engineered such that a guide nucleic acid-effector protein complex targets and edits exon 1, exon 2, exon 3, exon 4, exon 5, exon 6 or exon 7 as provided in TABLE 24. Specifically, the guide nucleic acids were engineered to comprise a handle sequence of SEQ ID NO: 107 and a spacer sequence selected from TABLE 24. TABLE 24 also provides specific PAM sequences that are recognized by the RNP complex of the fusion protein and guide nucleic acid. The spacer sequences of SEQ ID NOs: 288-310 were engineered to cause splice site disruption. The spacer sequences of SEQ ID NO: 311 was engineered to cause start codon disruption. Data for exemplary guide nucleic acids are provided in FIG. 59. Plasmid numbers PL34590, PL34592, PL34609, PL34601, and PL34699 described in FIG. 59 corresponds to guide nucleic acid comprising spacer sequences according to SEQ ID NOS: 289, 291, 308, 300, and 311, respectively.

TABLE 24
Guide RNAs for Editing LDHA

| | | Spacer | | PAM |
|---|---|---|---|---|
| Handle | | SEQ | | Sequence |
| SEQ ID | | ID | | recognized |
| NO: | Spacer Sequence | NO: | Target Exon of LDHA | by RNP |
| 107 | CCGUACCGGGAAUGCACGUC | 288 | exon #1 (NM_001165415.2) | CCTA |
| 107 | AAGAGUGAAGGCCCAUACCU | 289 | exon #1 (NM_001165414.2) | TGTG |
| 107 | UUUUUCCUCCUAUAGAUUCC | 290 | exon #2 (NM_001165415.2) | AATG |
| 107 | GUAGACUCUCACUUACCUUC | 291 | exon #2 (NM_001165415.2) | TGTG |
| 107 | AUGUCUUUUAGGACUUGGCA | 292 | exon #3 (NM_001165415.2) | TTTG |
| 107 | AACUUGUUGAAAUCAACCUU | 293 | exon #3 (NM_001165415.2) | TATA |
| 107 | AAAUCAACCUUUGCCAGAGA | 294 | exon #3 (NM_001165415.2) | GTTG |
| 107 | UUCCCCUUUUCUCUAGACUA | 295 | exon #4 (NM_001165415.2) | ATTA |
| 107 | UUUUCUCCUUUUUCAUAGUG | 296 | exon #5 (NM_001165415.2) | TTTA |
| 107 | CUUACCACUGGAAUCUCCAU | 297 | exon #5 (NM_001165415.2) | TATG |
| 107 | CUAUUUCUUUUAGUGCCUGU | 298 | exon #6 (NM_001165415.2) | TGTA |
| 107 | UGAACCUCUUUCCACUGUUC | 299 | exon #6 (NM_001165415.2) | CTTG |
| 107 | AAGAAGAGUGCAGAUACACU | 300 | exon #7 (NM_001165415.2) | TTTG |
| 107 | GACCUACCUUAAUCAUGGUG | 301 | exon #7 (NM_001165414.2) | CATA |
| 107 | UUCCUUUUCCACGCUAAGGU | 302 | exon #1 (NM_001165414.2) | GATA |
| 107 | GACUUGGAACCAAAAGGAAU | 303 | exon #2 (NM_001165415.2) | ATTG |
| 107 | CCAUCAGUAUCUUAAUGAAG | 304 | exon #2 (NM_001165415.2) | TGTG |
| 107 | UGCCAUCAGUAUCUUAAUGA | 305 | exon #2 (NM_001165415.2) | CCTG |
| 107 | GAGUUUGCAGUUACAUUAUA | 306 | exon #4 (NM_001165415.2) | CTTG |
| 107 | GGGAACAUGGAGAUUCCAGU | 307 | exon #5 (NM_001165415.2) | CTTG |
| 107 | AGGAACAGUGGAAAGAGGUU | 308 | exon #6 (NM_001165415.2) | GATA |
| 107 | AGUUUGAUCACCUCAUAAGC | 309 | exon #7 (NM_001165414.2) | TTTG |
| 107 | GAUCCCCCAAAGUGUAUCUG | 310 | exon #7 (NM_001165415.2) | TTTG |
| 107 | GAGUUGCCAUAUUGGACUUG | 311 | exon #2 (NM_001165415.2) | TTTA |

Systems that provide base editing in HEK293T cells are tested in vivo. Briefly, LNPs comprising the mRNA encoding the effector protein and guide RNAs are prepared according to the method described in Example 26. LNPs are then administered to humanized mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively. The presence of base modifications is analyzed from sequencing data.

### Example 31: Determination of a Fusion Protein Mediated In Vivo Base Editing Activity in HSD17B13

Systems comprising a fusion protein and a guide nucleic acid were tested for their ability to modify HSD17B13 in a human cell. Briefly, HEK293T cells were transfected with one or more plasmids encoding the fusion protein and the guide nucleic acid. After sufficient incubation, cells were harvested, and base editing was analyzed by NGS. The fusion protein comprised ABE8e (SEQ ID NO: 2) fused to CasM.265466 (SEQ ID NO: 1) having D220R and E335Q substitutions. The N-terminus of ABE8e was fused to the C-terminus of CasM.265466. The guide nucleic acids were engineered such that a guide nucleic acid-effector protein complex targets and edits exon 1, exon 2, exon 3, exon 4, exon 5 or exon 6 as provided in TABLE 25. Specifically, the guide nucleic acids were engineered to comprise a handle sequence of SEQ ID NO: 107 and a spacer sequence selected from TABLE 25. TABLE 25 also provides specific PAM sequences that are recognized by the RNP complex of the fusion protein and guide RNA. The spacer sequences of SEQ ID NOs: 312-327 were engineered to cause splice site disruption. The spacer sequences of SEQ ID NO: 328 was engineered to cause start codon disruption. Data for exemplary guide nucleic acids are provided in FIG. 59. Plasmid numbers PL34612, PL34622, PL34615, PL34618, PL34621, PL34627, and PL34700 described in FIG. 59 corresponds to guide nucleic acids comprising spacer sequences according to SEQ ID NOs: 312, 322, 315, 318, 321, 327, and 328, respectively.

TABLE 25
Guide RNAs for Editing HSD17B13

| | | Spacer | | PAM |
|---|---|---|---|---|
| Handle | | SEQ | | Sequence |
| SEQ ID | | ID | Target Exon of | recognized |
| NO: | Spacer Sequence | NO: | HSD17B13 | by RNP |
| 107 | CAUUACCUUAUUAAUAUCCC | 312 | exon #1 (NM_178135.5) | TATA |
| 107 | UUUCUUCCUAAUUACAGCGC | 313 | exon #2 (NM_178135.5) | TTTG |
| 107 | AUUACAGCGCGGUGUGGAGG | 314 | exon #2 (NM_178135.5) | CCTA |
| 107 | AACCUGCAGUCUCACCUGAU | 315 | exon #2 (NM_178135.5) | TGTG |
| 107 | UUCUUUCAAUUUCAAAAGGU | 316 | exon #3 (NM_178135.5) | AGTG |
| 107 | ACUCACACUCACCCAAAAAU | 317 | exon #3 (NM_178135.5) | TCTG |
| 107 | CAAUGAUCCUUAAGAUCACA | 318 | exon #4 (NM_178135.5) | TCTG |
| 107 | AUACUUACCAAUAUGGGAUG | 319 | exon #4 (NM_178135.5) | TGTG |
| 107 | ACCUUACCUUGUGCUUGGAU | 320 | exon #5 (NM_178135.5) | TTTG |
| 107 | AUCACAGAUUAUGGCCUGUA | 321 | exon #6 (NM_178135.5) | TTTA |
| 107 | GUUCUGUGGGAUAUUAAUAA | 322 | exon #1 (NM_178135.5) | ATTG |
| 107 | CAGUUUCCUCCACACCGCGC | 323 | exon #2 (NM_178135.5) | GCTG |
| 107 | UCGCUCUCUAAAUCAGGUGA | 324 | exon #2 (NM_178135.5) | TCTA |
| 107 | CAUCACCCACUUCUUUCUUC | 325 | exon #3 (NM_178135.5) | GTTA |
| 107 | GAAGAAGUGCUUUUGUGAUC | 326 | exon #4 (NM_178135.5) | GATG |
| 107 | UCUCCAAUACAGGCCAUAAU | 327 | exon #6 (NM_178135.5) | TCTG |
| 107 | UAGGAUGAUGUUCAUGGCUU | 328 | exon #1 (NM_178135.5) | TTTC |

Systems that provide base editing in HEK293T cells are tested in vivo. Briefly, LNPs comprising the mRNA encoding the effector protein and guide RNAs are prepared according to the method described in Example 26. LNPs are then administered to humanized mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively. The presence of base modifications is analyzed from sequencing data.

### Example 32: Determination of a Fusion Protein Mediated In Vivo Base Editing Activity in HA01

Systems comprising a fusion protein and a guide nucleic acid were tested for their ability to modify HAO1 in a human cell. Briefly, HEK293T cells were transfected with one or more plasmids encoding the fusion protein and the guide nucleic acid. After sufficient incubation, cells were harvested, and base editing was analyzed by NGS. The fusion protein comprised ABE8e (SEQ ID NO: 2) fused to CasM.265466 (SEQ ID NO: 1) having D220R and E335Q substitutions. The N-terminus of ABE8e was fused to the C-terminus of CasM.265466. The guides were engineered such that a guide nucleic acid-effector protein complex targets and edits exon 1, exon 2, exon 3, exon 4, exon 5, exon 6 or exon 7 as provided in TABLE 26. Specifically, the guide nucleic acids were engineered to comprise a handle sequence of SEQ ID NO: 107 and a spacer sequence selected from TABLE 26. TABLE 26 also provides specific PAM sequences that are recognized by the RNP complex of the fusion protein and guide nucleic acid. The spacer sequences of SEQ ID NOs: 329-347 were engineered to cause splice site disruption. The spacer sequences of SEQ ID NOs: 348-349 were engineered to cause start codon disruption. Data for exemplary guide nucleic acids are provided in FIG. 59. Plasmid numbers PL34629, PL34630, and PL34645 described in FIG. 59 correspond to guide nucleic acids comprising spacer sequences according to SEQ ID NOs: 330, 331, and 346, respectively.

TABLE 26
Guide RNAs Editing HAO1

| | | Spacer | | PAM |
|---|---|---|---|---|
| Handle | | SEQ | | Sequence |
| SEQ ID | | ID | | recognized |
| NO: | Spacer Sequence | NO: | Target Exon of HAO1 | by RNP |
| 107 | AAAAAUAAAUUUUCUUACCU | 329 | exon #1 (NM_017545.3) | TTTA |
| 107 | AUUCUAGAUGGAAGCUGUAU | 330 | exon #2 (NM_017545.3) | TTTA |
| 107 | GUGACAAUCUUCCUCCUACC | 331 | exon #2 (NM_017545.3) | TGTG |
| 107 | AUCAUCCCCUUUCUUUCUCA | 332 | exon #3 (NM_017545.3) | CTTG |
| 107 | GUUACCUGAGUUGUGGCGGC | 333 | exon #3 (NM_017545.3) | CATG |
| 107 | CAUUUCUUAUUUUAGGAUGA | 334 | exon #4 (NM_017545.3) | TATG |
| 107 | AACGAACCUCUCAAAAUGCC | 335 | exon #4 (NM_017545.3) | AATA |
| 107 | CUCUCUCCAGGUGAUGAUGC | 336 | exon #5 (NM_017545.3) | TTTA |
| 107 | CCAAAACUCACAGUGGCUGG | 337 | exon #5 (NM_017545.3) | TCTG |
| 107 | AACUUUUCUUUCCCCAGAUU | 338 | exon #6 (NM_017545.3) | ATTG |
| 107 | UCCAGUUACCUGGAAAGCUA | 339 | exon #6 (NM_017545.3) | TTTG |
| 107 | UCUUUUCACUUGGUUAGGGG | 340 | exon #7 (NM_017545.3) | TGTA |
| 107 | UUGCAGCAUUUUCCAGGUAA | 341 | exon #1 (NM_017545.3) | AATA |
| 107 | AUAAUAUUGCAGCAUUUUCC | 342 | exon #1 (NM_017545.3) | GCTG |
| 107 | CCCGUUCCCAGGGACUGACA | 343 | exon #3 (NM_017545.3) | CATG |
| 107 | GUUUCAAAAUUUUUCAUCCU | 344 | exon #4 (NM_017545.3) | ACTG |
| 107 | CAAAGGGCAUUUUGAGAGGU | 345 | exon #4 (NM_017545.3) | GTTG |
| 107 | ACAGCCUCCCUGGCAUCAUC | 346 | exon #5 (NM_017545.3) | TTTA |
| 107 | GGGCUUAGCUUUCCAGGUAA | 347 | exon #6 (NM_017545.3) | TTTG |
| 107 | GGAUAGCAAUAACCUGUGAA | 348 | exon #1 (NM_017545.3) | TCTG |
| 107 | AAAAUGCUCCCCCGGCUAAU | 349 | exon #1 (NM_017545.3) | TGTG |

Systems that provide base editing in HEK293T cells are tested in vivo. Briefly, LNPs comprising the mRNA encoding the effector protein and guide RNAs are prepared according to the method described in Example 26. LNPs are then administered to humanized mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively. The presence of base modifications is analyzed from sequencing data.

### Example 33: Determination of a Fusion Protein Mediated In Vivo Base Editing Activity in CNBP

Systems comprising a fusion protein and a guide nucleic acid were tested for their ability to modify CNBP in a human cell. Briefly, HEK293T cells were transfected with one or more plasmids encoding the fusion protein and the guide nucleic acid. After sufficient incubation, cells were harvested, and base editing was analyzed by NGS. The fusion protein comprised ABE8e (SEQ ID NO: 2) fused to CasM.265466 (SEQ ID NO: 1) having D220R and E335Q substitutions. The N-terminus of ABE8e was fused to the C-terminus of CasM.265466. The guides were engineered such that a guide nucleic acid-effector protein complex targets and edits exon 1, exon 2, exon 3, exon 4 or exon 5 as provided in TABLE 27. Specifically, the guide nucleic acids were engineered to comprise a handle sequence of SEQ ID NO: 107 and a spacer sequence selected from TABLE 27. TABLE 27 also provides specific PAM sequences that were recognized by the RNP complex of the fusion protein and guide nucleic acid. The spacer sequences of SEQ ID NOs: 350-365 were engineered to cause splice site disruption. Data for exemplary guide nucleic acids are provided in FIG. 59. Plasmid numbers PL34647 described in FIG. 59 corresponds to guide nucleic acid comprising spacer sequence according to SEQ ID NO: 350.

TABLE 27
Guide RNAs for Editing CNBP

| | | Spacer | | PAM |
|---|---|---|---|---|
| Handle | | SEQ | | Sequence |
| SEQ ID | | ID | | recognized |
| NO: | Spacer Sequence | NO: | Target Exon of CNBP | by RNP |
| 107 | UUCGACAAAAUACCUCUAUC | 350 | exon #2 (NM_003418.5) | TCTA |
| 107 | UCCGAGGUAAAACCACCUCU | 351 | exon #2 (NM_001127196.2) | TCTA |
| 107 | CUCAAAACAGGUUUCCAGUU | 352 | exon #3 (NM_003418.5) | TTTG |
| 107 | CUUACCAUCCUCCUGAAGAU | 353 | exon #3 (NM_003418.5) | AATA |
| 107 | UUGAAGCCUGCUAUAACUGC | 354 | exon #4 (NM_003418.5) | ATTG |
| 107 | ACACCUUACCUAUAGCACUU | 355 | exon #4 (NM_003418.5) | TCTG |
| 107 | AUUUCUUUUUUAGGUGUGGU | 356 | exon #5 (NM_003418.5) | TTTA |
| 107 | CGGGCCCGCUCCGACGCGGA | 357 | exon #1 (NM_003418.5) | GCTG |
| 107 | GUUUUACCUCGGAUAGAGGU | 358 | exon #2 (NM_003418.5) | GGTG |
| 107 | AGAAGCCGUGGCAGAGGUGG | 359 | exon #2 (NM_001127196.2) | AATG |
| 107 | GAAUGAGAAGCCGUGGCAGA | 360 | exon #2 (NM_001127196.2) | CGTG |
| 107 | UGAUCUUCAGGAGGAUGGUA | 361 | exon #3 (NM_003418.5) | ATTG |
| 107 | CCGCAGUUAUAGCAGGCUUC | 362 | exon #4 (NM_003418.5) | TCTA |
| 107 | CACCAAAGUGAAGUGCUAUA | 363 | exon #4 (NM_003418.5) | ACTG |
| 107 | ACCAGUUUCACCACACCUAA | 364 | exon #5 (NM_003418.5) | CATG |
| 107 | CAUGACCAGUUUCACCACAC | 365 | exon #5 (NM_003418.5) | GCTA |

Systems that provide base editing in HEK293T cells are tested in vivo. Briefly, LNPs comprising the mRNA encoding the effector protein and guide RNAs are prepared according to the method described in Example 26. LNPs are then administered to humanized mice in a 6 day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively. The presence of base modifications is analyzed from sequencing data.

### Example 34: Determination of a Fusion Protein Mediated In Vivo Base Editing Activity in LPA

Systems comprising a fusion protein and a guide RNA were tested for their ability to modify LPA in a human cell. Briefly, HEK293T cells were transfected with one or more plasmids encoding the fusion protein and the guide nucleic acid. After sufficient incubation, cells were harvested, and base editing was analyzed by NGS. The fusion protein comprised ABE8e (SEQ ID NO: 2) fused to CasM.265466 (SEQ ID NO: 1) having D220R and E335Q substitutions. The N-terminus of ABE8e was fused to the C-terminus of CasM.265466. The guide nucleic acids were engineered such that a guide nucleic acid-effector protein complex targets and edits exon 2 as provided in TABLE 28. Specifically, the guide nucleic acids were engineered to comprise a handle sequence of SEQ ID NO: 107 and a spacer sequence selected from TABLE 28. TABLE 28 also provides specific PAM sequences that are recognized by the RNP complex of the fusion protein and guide nucleic acid. The spacer sequences of SEQ ID NOs: 366-368 were engineered to cause splice site disruption. Data for exemplary guide nucleic acids are provided in FIG. 59. Plasmid numbers PL34690 described in FIG. 59 corresponds to guide nucleic acid comprising spacer sequence according to SEQ ID NO: 367.

TABLE 28
Guide RNAs for Editing LPA

| | | Spacer | | PAM |
|---|---|---|---|---|
| Handle | | SEQ | | Sequence |
| SEQ ID | | ID | | recognized |
| NO: | Spacer Sequence | NO: | Target Exon of LPA | by RNP |
| 107 | AUUUUAGCAGCACCUGAGCA | 366 | exon #2 (NM_005577.4) | TTTA |
| 107 | AGAUUAAUGACAUACGCAUU | 367 | exon #2 (NM_005577.4) | TGTA |
| 107 | GCUUUGCUCAGGUGCUGCUA | 368 | exon #2 (NM_005577.4) | CATG |

Systems that provide base editing in HEK293T cells are tested in vivo. Briefly, LNPs comprising the mRNA encoding the effector protein and guide RNAs are prepared according to the method described in Example 26. LNPs are then administered to humanized mice in a 6-day study (n=5) to test for mRNA delivery to animals in vivo. LNPs are dosed intravenously via the tail vein at doses of 0.5 mg/kg and 2 mg/kg. After 6 days, livers and serum are harvested for NGS and ELISA analysis, respectively. The presence of base modifications is analyzed from sequencing data.

### Example 35: In Vivo Testing of LNP Formulations

Different LNPs and formulations of the present disclosure are tested for in vivo editing in mice with mRNA encoding a variant of CasM.265466 with a D220R amino acid substitution (D220R effector protein variant) and associated mouse Pcsk9 guide nucleic acid. The D220R effector protein variant is modified to comprise NLS sequences at N-terminus (SEQ ID NO: 219) and C-terminus (SEQ ID NO: 55). The mRNA encoding the D220R effector variant is represented by the polynucleotide sequence recited in TABLE 29. One of skill in the art understands that alternative NLSs and UTRs could be used in this experiment.

TABLE 29
mRNA Sequence Encoding Fusion Protein
SEQ ID NO: 371

AGGAGAGAAAAGAAGAGUAAGAAGAAAUAUAAGAGCCACCauggc
cccaaagaagaagcggaaggucgguauccacggagucccagcagc
cAUGUCCGUCUUAACCCGAAAGGUGCAACUGAUACCUGUGGGGGA
CAAGGAGGAGAGGGACCGAGUGUACAAGUACCUCCGUGAUGGCAU
AGAGGCCCAAAACAGGGCUAUGAACCUCUACAUGUCUGGCCUAUA
CUUCGCCGCCAUAAACGAGGCAUCCAAGGAGGACAGAAAGGAGCU
GAACCAACUCUACAGCAGAAUAGCCACCUCCAGCAAGGGCUCUGC
CUACACUACUGACAUUGAAUUCCCUACAGGUCUGGCCAGCACCUC
UACCCUCAGCAUGGCCGUGAGGCAAGACUUUACUAAGAGCUUAAA
AGACGGCUUGAUGUACGGGCGUGUGUCCCUACCCACCUAUCGAAA
GGACAACCCUCUGUUCGUGGACGUCAGAUUCGUUGCCUUACGGGG
GACGAAGCAAAAGUACAACGGCUUGUACCAUGAGUACAAGUCCCA
CACCGAGUUUCUGGACAACCUGUACAGCUCUGACCUGAAGGUCUA
CAUCAAGUUUGCCAACGACAUUACGUUUCAGGUCAUCUUCGGGAA
CCCACGCAAAAGCUCCGCUCUUAGGUCAGAGUUCCAGAAUAUCUU
CGAGGAGUACUACAAGGUGUGUCAGUCGUCCAUUCAGUUCUCUGG
GACCAAAAUCAUCCUGAACAUGGCGAUGAGAAUCCCAGAUAAGGA
GAUCGAGUUGGAUGAAGAUGUGUGCGUCGGAGUUGAUCUGGGCAU
UGCUAUUCCCGCGGUGUGCGCUCUUAACAAAAAUAGAUACUCACG
UGUCAGCAUUGGUUCAAAAGAGGACUUUCUGAGGGUGCGCACUAA
AAUCAGGAAUCAACGCAAACGGCUGCAGACAAACCUGAAAUCGUC
AAACGGUGGACACGGACGGAAGAAGAAGAUGAAGCCCAUGGAUCG
GUUUAGAGAUUAUGAAGCGAAUUGGGUGCAGAAUUAUAACCACUA
UGUUAGUCGGCAGGUAGUUGAUUUUGCAGUUAAAAAUAAGGCCAA
AUAUAUUAAUCUCGAAAAUCUCGAAGGCAUCCGCGAUGAUGUAAA
GAAUGAAUGGCUUCUUAGUAACUGGAGUUAUUAUCAGUUGCAGCA
GUAUAUUACAUAUAAAGCUAAAACAUAUGGGAUCGAAGUAAGAAA
AAUUAAUCCGUAUCAUACAUCACAGCGCUGUAGUUGCUGCGGAUA
UGAAGAUGCUGGAAAUAGACCCAAAAAGGAAAAAGGACAGGCAUA
UUUCAAAUGUUUAAAGUGUGGUGAGGAGAUGAAUGCAGAUUUUAA
UGCUGCACGGAACAUCGCAAUGUCUACAGAAUUUCAGAGUGGCAA
GAAAACUAAGAAACAGAAAAAGGAACAGCACGAAAAUAAAaaaag
gccggcggccacgaaaaaggccggccaggcaaaaaagaaaaagug
aUUAAUUAAGCUGCCUUCUGCGGGGCUUGCCUUCUGGCCAUGCCC
UUCUUCUCUCCCUUGCACCUGUACCUCUUGGUCUUUGAAUAAAGC
CUGAGUAGGAAGaaaaaaaaaaaaaaaaaaaaaaaaaaaaaaaaa
aaaaaaaaaaaaaaaaaaaaaaaaaaaaaaaaaaaaaaaaaaaaa
aaaaaaaaaaaaaaaaaaaaaaaaaau
*Bold and Upper Case = 5′ UTR; Lower Case = a nucleotide sequence encoding N-terminal NLS; Upper Case = nucleotide sequence encoding fusion protein; Bold and Lower Case = a nucleotide sequence encoding C-terminal NLS; Upper Case and Italicized = 3′ UTR; and Lower Case and Italic = polyA tail.

The LNPs are administered to mice along with an associated mouse guide nucleic acid targeting Pcsk9 gene (mA*mU*mA*GAUUGCUCCUUACGAGGAGACGAGCAACGGCGGAAmG*mG*mU (SEQ ID NO: 372)) intravenously through tail vein at 2 mg/kg dose. Blood samples and/or tissue samples (e.g., liver samples) are harvested at various time points after administration and editing of Pcsk9 (measured as % indel) is analyzed by NGS.

### Example 36: Determining Optimal Length of a Spacer Sequence in Guide Nucleic Acid with a CasM.265466 Effector Protein Variant

D220R variant of CasM.265466 (SEQ ID NO: 1) and three gRNAs, R10250, R10252 and R10260, were tested for their ability to integrate a luciferase reporter into a target nucleic acid in primary human hepatocytes (PHH). Each gRNA comprised a handle sequence (SEQ ID NO: 107), and a spacer sequence having a length ranging from 16 nucleotides to 27 nucleotides. Briefly, PHH cells were transfected at 1:1 mRNA to gRNA ratio using Lipofectamine MessengerMax and co-transduced with AAVDJ-nLuc reporter at 1E4 MOI. After 72 hours, indels were quantified by NGS and luciferase reporter integration was quantified by luciferase assay.

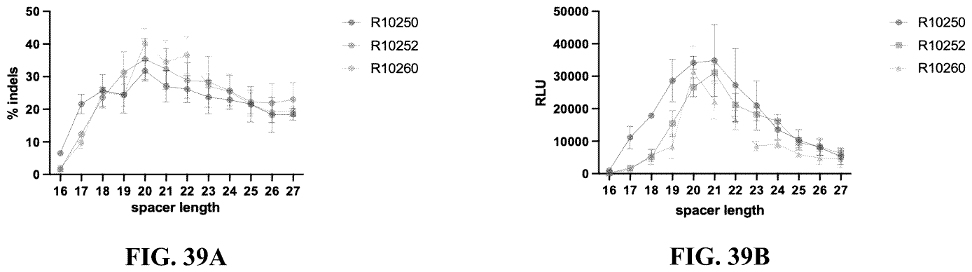

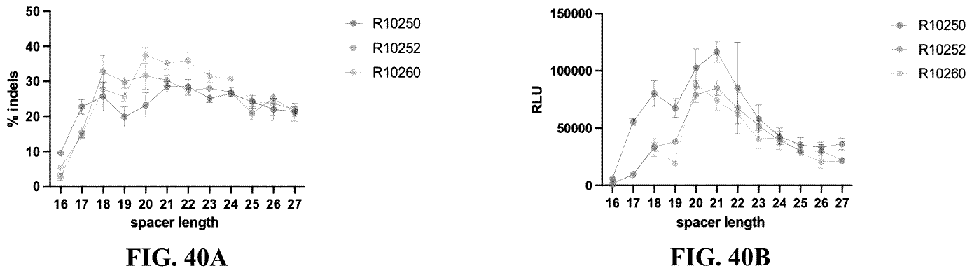

FIG. 39A shows results of the NGS assay. FIG. 39B shows results of the luciferase assay. The experiment was repeated in PHH derived from a different donor. FIGS. 40A and 40B show results of the NGS assay and the luciferase assay, respectively, from the PHH derived from a different donor. An analysis of FIGS. 39A-39B and FIGS. 40A-40B indicates that the combination of D220R variant of CasM.265466 (SEQ ID NO: 1) and gRNAs show high activity when the spacer has a length of 20-21 nucleotides.

### Example 37: CasM.265466 System Mediated Editing of Target Nucleic Acid Using Guides Having Unbiased Tiling of Modifications

A set of chemical modifications was designed to identify compatible modifications for a guide nucleic acid in combination with D220R variant of CasM.265466 (SEQ ID NO: 1). The guide comprised a handle sequence having a length of 69 nucleotides (SEQ ID NO: 107), and a spacer sequence having a length of 20 nucleotides. The modifications included one or more 2-O-Me sugar modifications, and one or more PS backbone modifications. The system was then evaluated in Hep3B cells.

Briefly, Hep3B cells were transfected with 200 ng of mRNA encoding D220R variant of CasM.265466 (SEQ ID NO: 1), and a gRNA harboring the indicated 2-O-Me sugar modification or PS backbone modifications at an indicated position within the handle sequence. The Hep3B cells were transfected by MessengerMax using the mRNA encoding D220R variant of CasM.265466 (SEQ ID NO: 1), and the gRNA at a ratio of 1:1. After 48 hours of the transfection, indels were quantified by NGS.

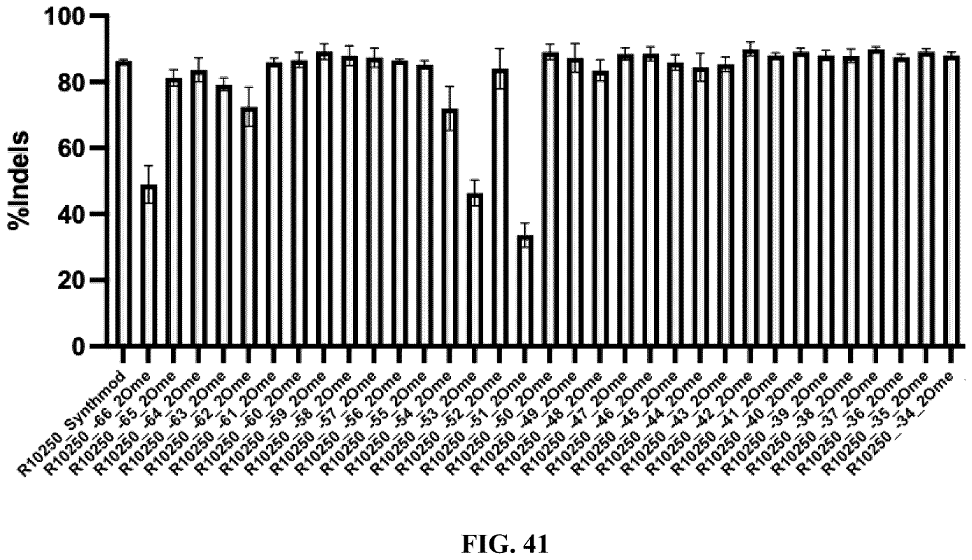

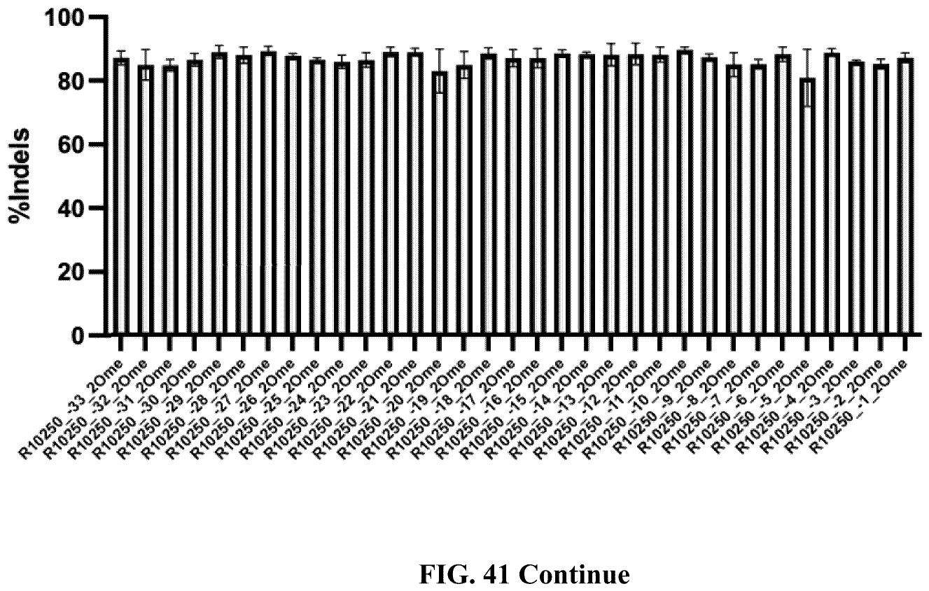

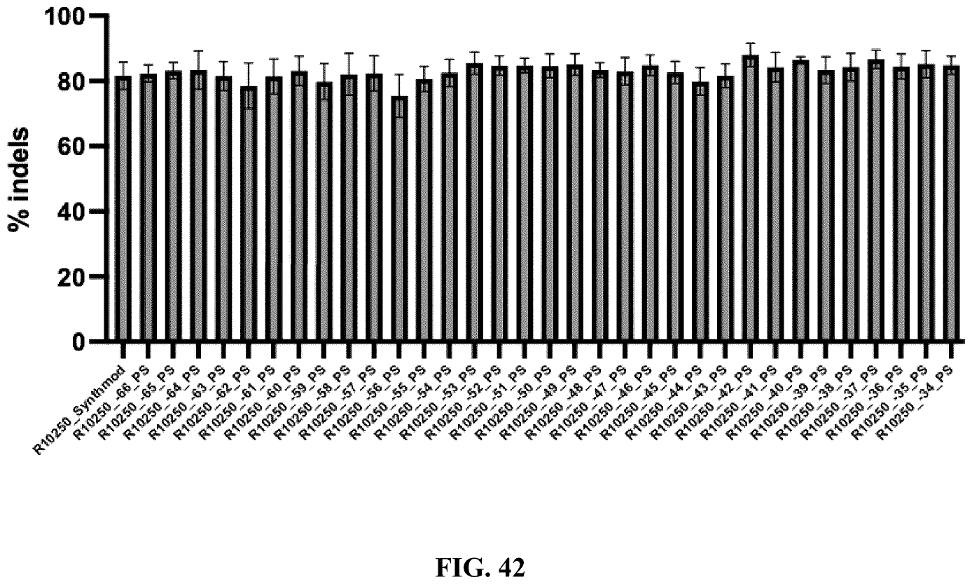

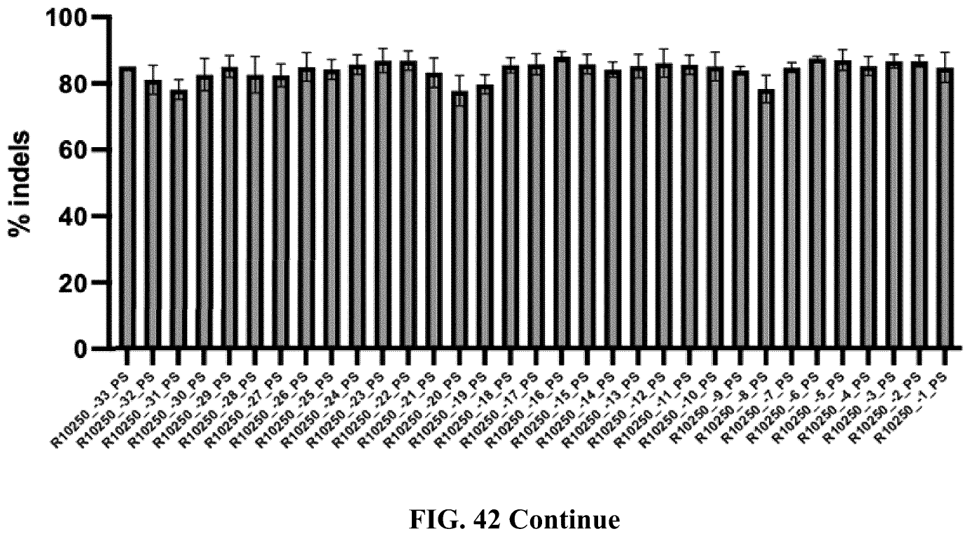

FIG. 41 shows results of NGS assay for guides having 2-O-Me modifications. FIG. 42 shows results of NGS assay for guides having PS backbone modifications. An analysis of FIG. 41 indicates that at least some 2-O-Me modifications affect activity of the combination of the effector protein and the guide nucleic acid. Conversely, an analysis of FIG. 42 indicates PS backbone modifications are tolerated at all tested positions.

### Example 38: CasM.265466 System Mediated Editing of Target Nucleic Acid Using Guides Having Combinatorial Modifications

A set of chemical modifications was designed to identify compatible modifications for a guide nucleic acid in combination with D220R variant of CasM.265466 (SEQ ID NO: 1). The guide comprised a handle sequence having a length of 69 nucleotides (SEQ ID NO: 107), and a spacer sequence having a length of 20 nucleotides. The modifications included combinatorial modifications, 2-O-Me sugar modifications, PS backbone modifications and U-A >G-C covariations, that were rationally designed based on predicted structure of the guide nucleic acid. FIG. 43 shows various guide nucleic acid modifications that were tested. The system was then evaluated in Hep3B cells.

Briefly, Hep3B cells were transfected with 100 ng of mRNA encoding D220R variant of CasM.265466 (SEQ ID NO: 1), and a gRNA harboring the indicated modifications at an indicated position within the spacer sequence at 0, 6, or 12 hours prior to transfection. The Hep3B cells were transfected by MessengerMax using at a ratio of 1:1 for the mRNA encoding D220R variant of CasM.265466 (SEQ ID NO: 1), and the gRNA. After 48 hours of the transfection, indels were quantified by NGS.

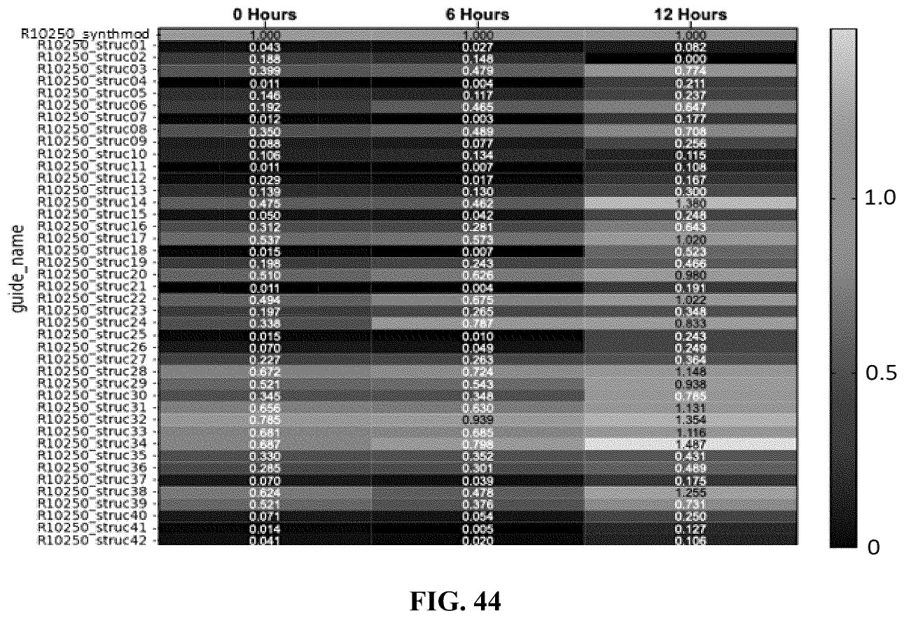

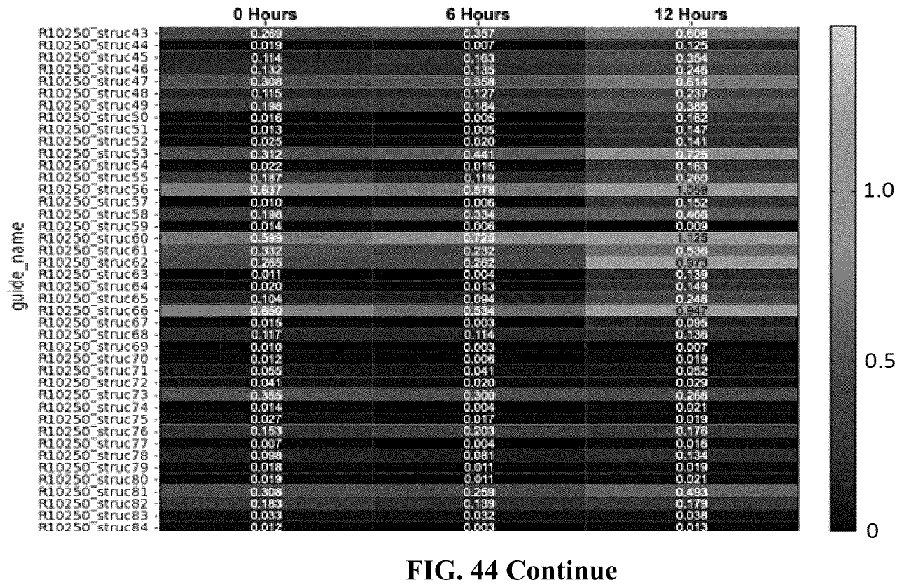

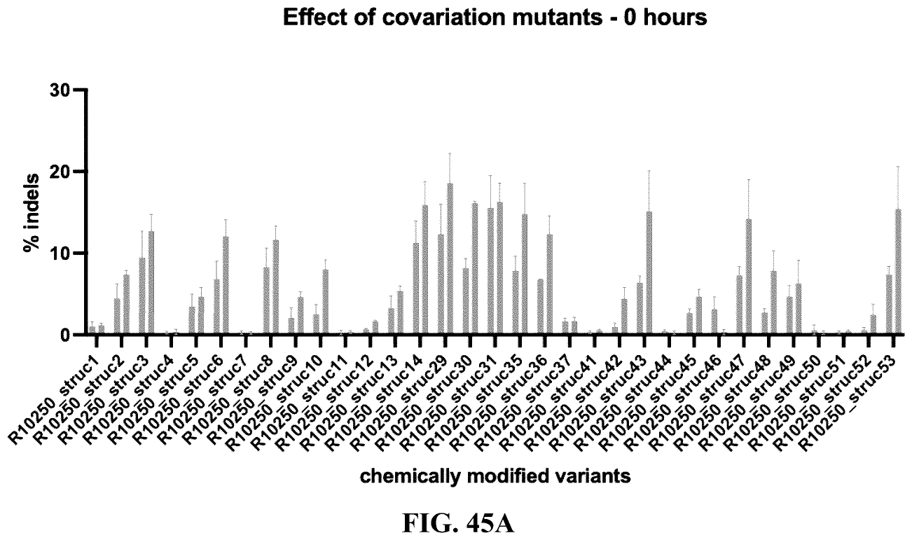

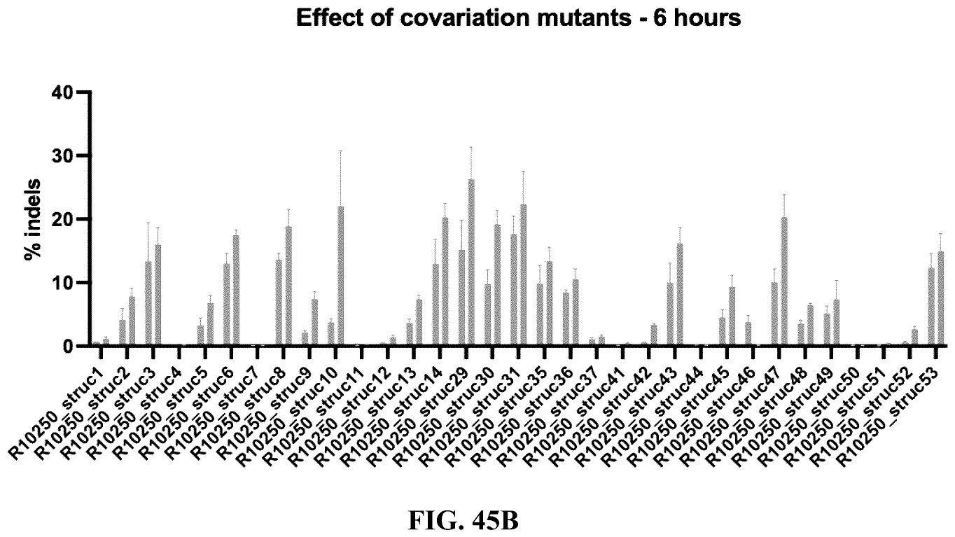

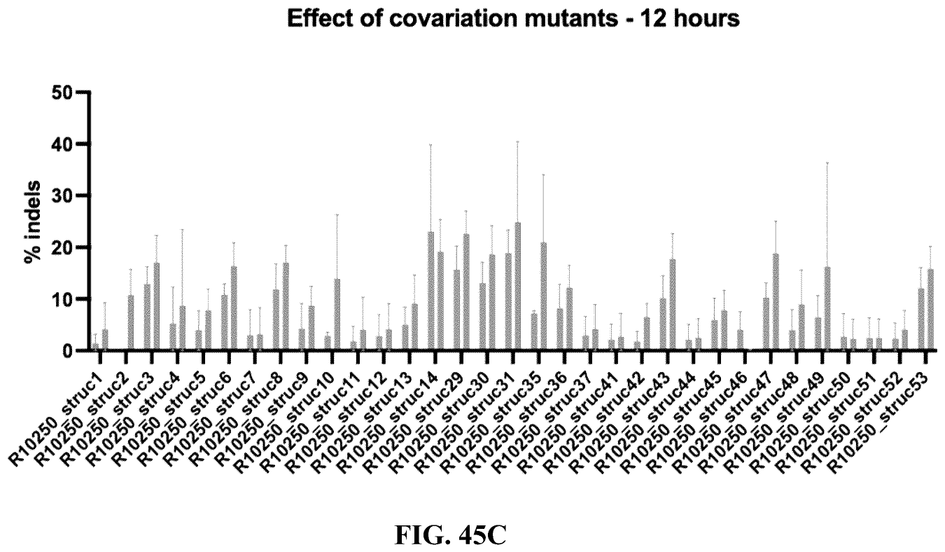

FIG. 44 shows results of NGS assay for guides having various combinatorial modifications. FIGS. 45A-45C summarizes results of FIG. 44 with a focus on the effect of U-A >G-C covariations. An analysis of FIGS. 44 and 45A-45C indicates that at least some GC covariation modifications affect activity of the combination of the effector protein and the guide nucleic acid.

### Example 39: CasM.265466 Variants Biased for Target Strand Editing

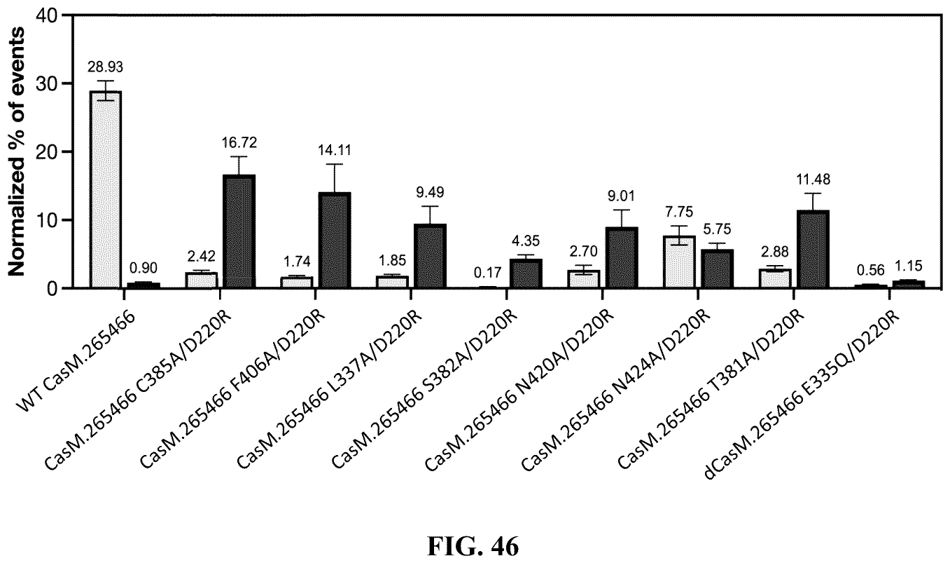

Variants of CasM.265466 were tested for their ability to nick a target nucleic acid and introduce base edits. Briefly, HEK293T cells were transfected with a plasmid encoding a variant of CasM.265466 fused to a deaminase protein and a plasmid encoding a guide RNA targeting a human gene. Amino acid sequences of CasM.265466 variants are summarized in TABLE 1.1 (SEQ ID NOS: 375-381). The nucleotide sequence of an exemplary guide RNA that was used in these assays comprised a handle sequence of SEQ ID NO: 107 and a spacer sequence of SEQ ID NO: 157. Editing of a target-strand (TS) (the strand bound by the guide RNA), and a non-target strand (NTS) (the strand not bound by the guide RNA), was determine. A ratio of a TS editing over an NTS editing was reported as the nickase score. The desirable NTS nick activity by the engineered CasM.265466 would increase accumulation of base editing on the TS increasing the ratio of TS/NTS editing and thus, the nickase score. Wildtype (WT) CasM.265466 (SEQ ID NO: 1) was used as a control for nuclease activity and dCasM.265466 (D220R/E335Q) was used as a negative control. Results are shown in FIG. 46. For each variant, nuclease indels (left bar) and nickase score (right bar) were analyzed. The desirable NTS nick activity by the engineered CasM.265466 would increase accumulation of base editing on the TS increasing the ratio of TS/NTS editing and thus, the nickase score.

### Example 40: CasM.265466 Variants Biased for Non-Target Strand Editing

Fusion protein systems comprising variants of CasM.265466 fused to a deaminase protein were tested for their ability to selectively edit a target-strand (TS) (the strand bound by the guide RNA) of a target nucleic acid relative to a non-target strand (NTS) (the strand not bound by the guide RNA) of the target nucleic acid. Briefly, cells were transfected with a plasmid encoding the fusion protein and one of eight guide RNAs. The variant of CasM.265466 included CasM.265466 S382A/D220R (SEQ ID NO: 379), CasM.265466 F406A/D220R (SEQ ID NO: 377), CasM.265466 L337A/D220R (SEQ ID NO: 381), CasM.265466 C385A/D220R (SEQ ID NO: 378), CasM.265466 T381A/D220R (SEQ ID NO: 380), CasM.265466 N420A/D220R (SEQ ID NO: 375), CasM.265466 N336A/D220R (SEQ ID NO: 382) and CasM.265466 Y379A/D220R (SEQ ID NO: 383). Fusion protein systems comprising dCasM.265466 D220R/E335Q and CasM.265466 D220R (SEQ ID NO: 385) effector proteins were used as controls. Nucleotide sequence of the guide RNA that was used in these assays comprised a handle sequence of SEQ ID NO: 107 and a spacer sequence of SEQ ID NO: 157, 228, 269, 289, 291, 312, 321 or 386 as described in TABLE 30.

TABLE 30
Guide RNAs for Gene Editing

| | | Handle | Spacer | |
|---|---|---|---|---|
| | Target | SEQ ID | SEQ ID | Spacer |
| Plasmid | Gene | NO: | NO: | Sequence |
| PL28330 | MLH1 | 107 | 157 | AGUCUCCAGG |
| | | | | AAGAAAUUAA |
| PL28336 | B2M | 107 | 386 | AGUGUAUAAG |
| | | | | CAUAUCAAUA |
| PL34536 | ANGPTL3 | 107 | 228 | UCAGCUCAGA |
| | | | | AGGACUAGUA |
| PL34572 | PCSK9 | 107 | 269 | UUCCUCCCAG |
| | | | | GCCUGGAGUU |
| PL34590 | LDHA | 107 | 289 | AAGAGUGAAG |
| | | | | GCCCAUACCU |
| PL34592 | LDHA | 107 | 291 | GUAGACUCUC |
| | | | | ACUUACCUUC |
| PL34612 | HSD17B13 | 107 | 312 | CAUUACCUUA |
| | | | | UUAAUAUCCC |
| PL34621 | HSD17B13 | 107 | 321 | AUCACAGAUU |
| | | | | AUGGCCUGUA |

NGS analysis was performed to determine % indel generated by each system. Editing of the TS and the NTS was also determined based on the NGS analysis. A nickase score was calculated using the following formula:

$$Nickase score = \frac{( \% T S - \% NTS )}{( \% TS + \% NTS )} \times ( 1 - indel ) \qquad Formula 1$$

wherein, % indels are normalized to % indel observed for CasM.265466 D220R for each guide RNA. The equation confines the value of the nickase score to −1≤x>+1, wherein a score of +1 indicates 0% indel and complete bias towards TS editing, a score of −1 indicates 0% indels and complete bias towards NTS editing, and a score of 0 indicates either 100% indels or no bias towards wither strand editing.

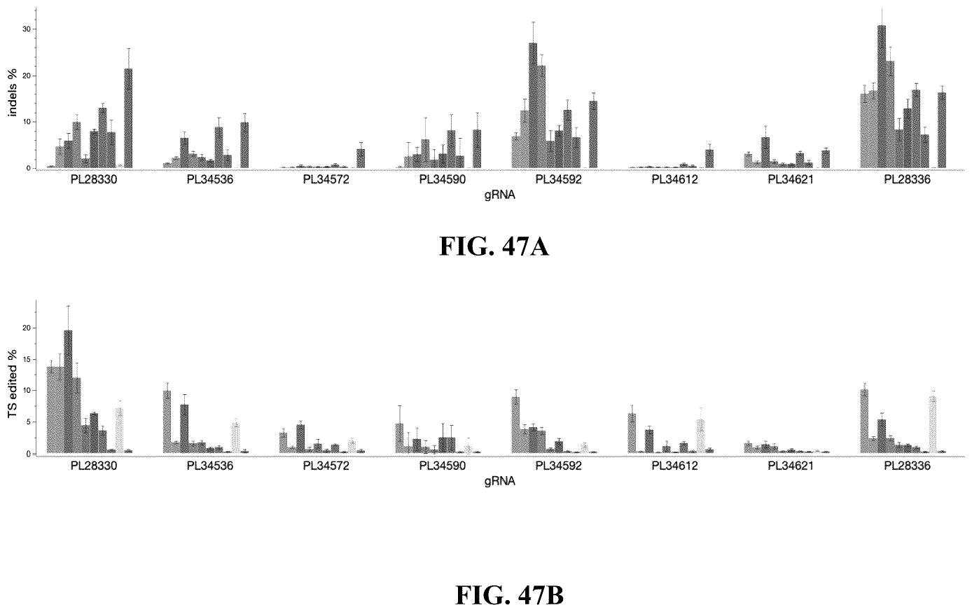

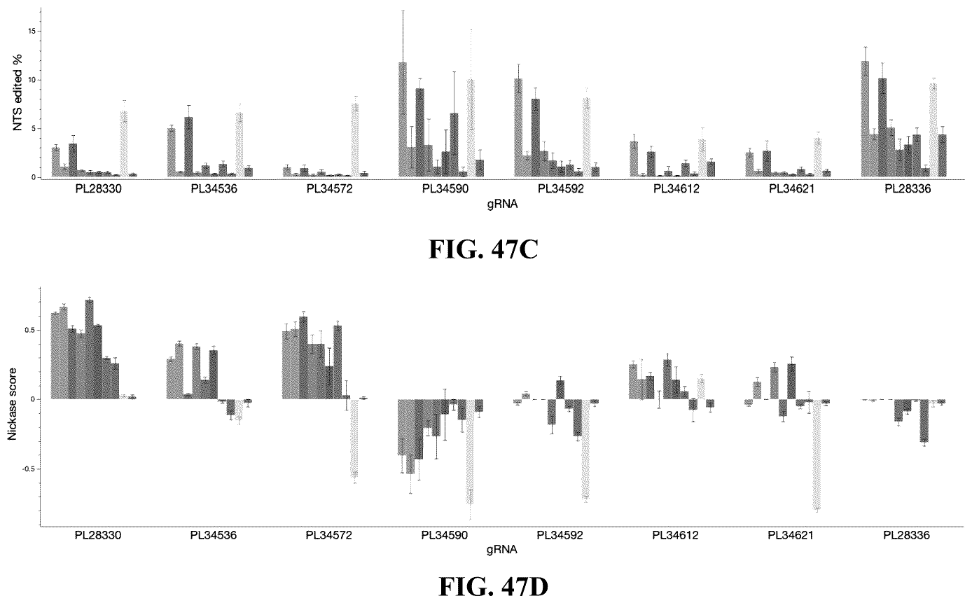

Results of % indels generated, % TS edited, and % NTS edited by each effector protein systems are shown in FIGS. 47A-47D. An analysis of FIGS. 47A-47D indicates that, amongst the tested effector protein systems, the systems comprising at least one of CasM.265466 S382A/D220R (SEQ ID NO: 379), CasM.265466 F406A/D220R (SEQ ID NO: 377), and CasM.265466 L337A/D220R (SEQ ID NO: 381) had higher nickase activity relative to the other tested systems.

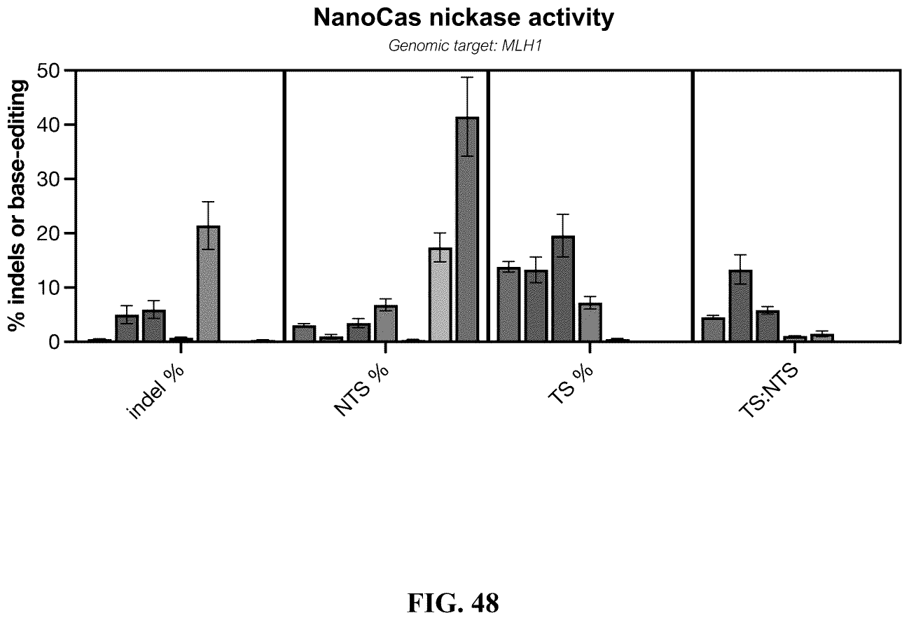

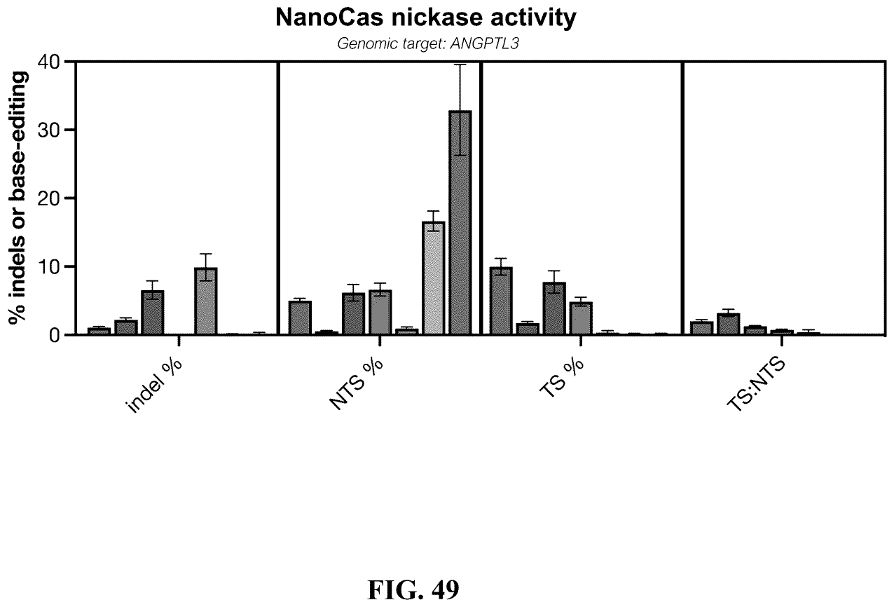

The experiment was repeated for three systems, CasM.265466 S382A/D220R (SEQ ID NO: 379), CasM.265466 F406A/D220R (SEQ ID NO: 377), and CasM.265466 L337A/D220R (SEQ ID NO: 381), each targeting MLH1 or ANGPTL3. The experiment comprised four control effector protein systems, dCasM.265466 D220R/E335Q, CasM.265466 D220R, dCas9 and nCas9. Results of the % indels generated, % TS edited, % NTS edited, and TS: NTS ratio observed for each effector protein system are shown in FIGS. 48 and 49. An analysis of FIGS. 48 and 49 indicates that the system comprising CasM.265466 S382A/D220R (SEQ ID NO: 379) showed relatively higher desired activity as it showed near baseline levels of indel, lower NTS % and higher TS % compared to dCasM.265466 D220R/E335Q.

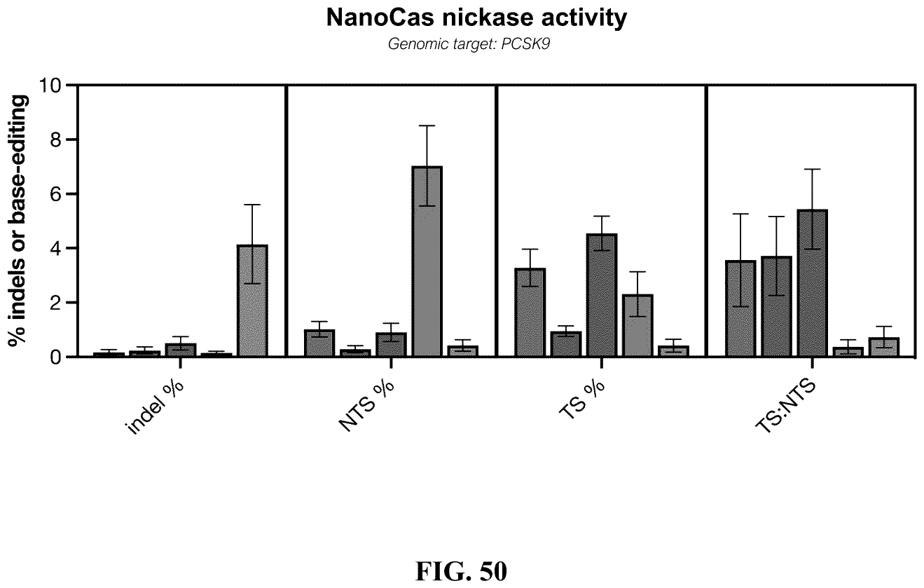

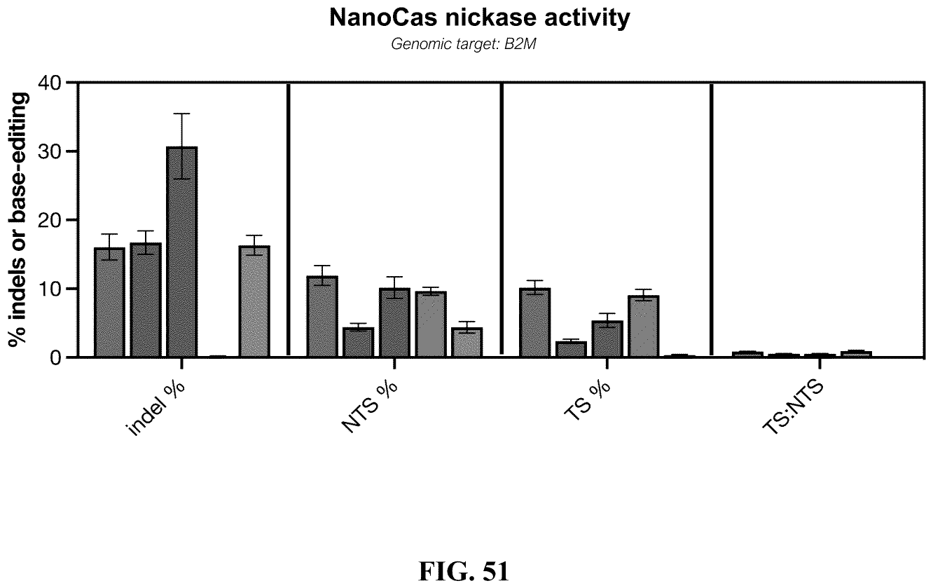

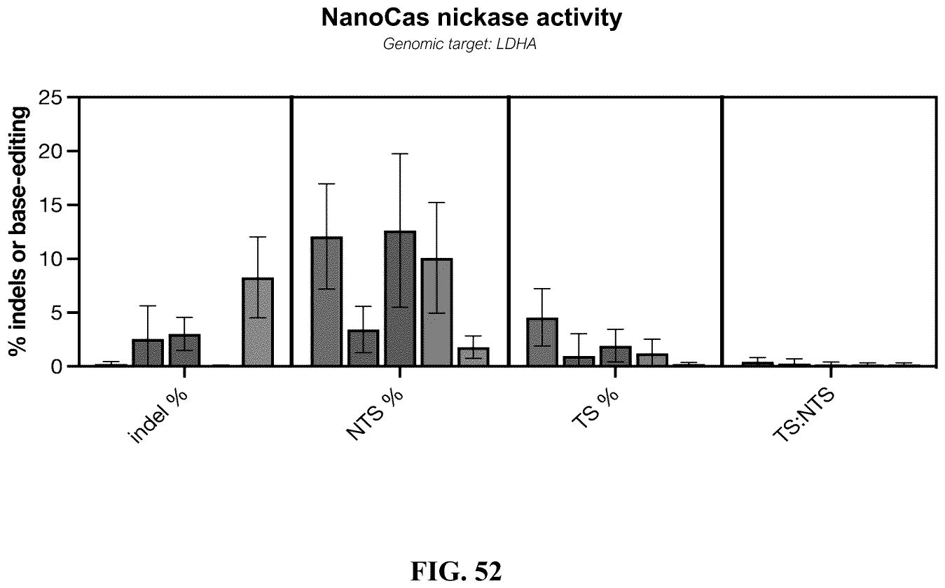

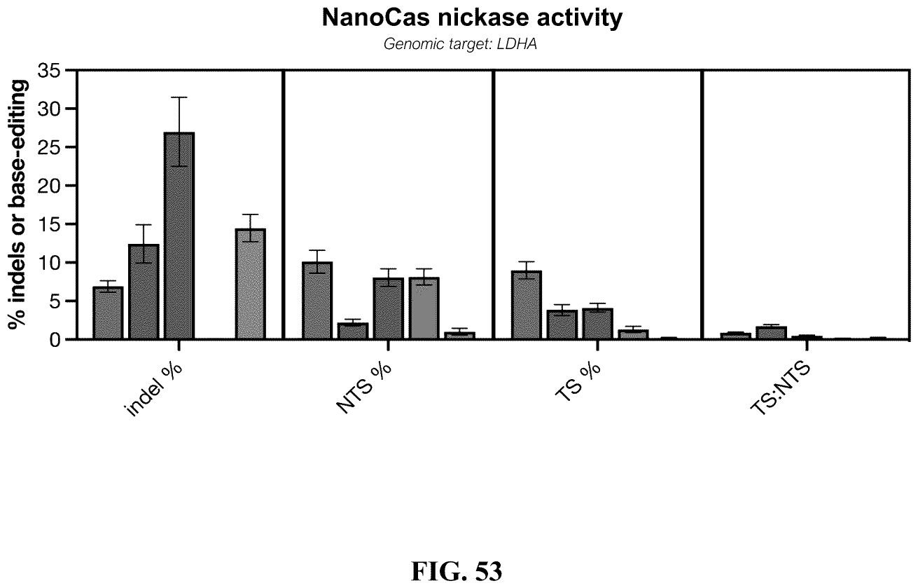

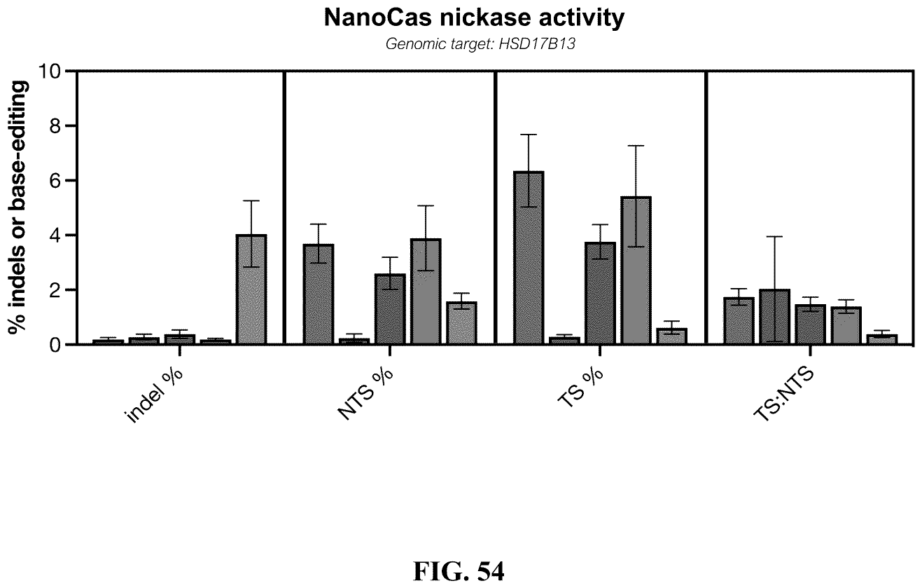

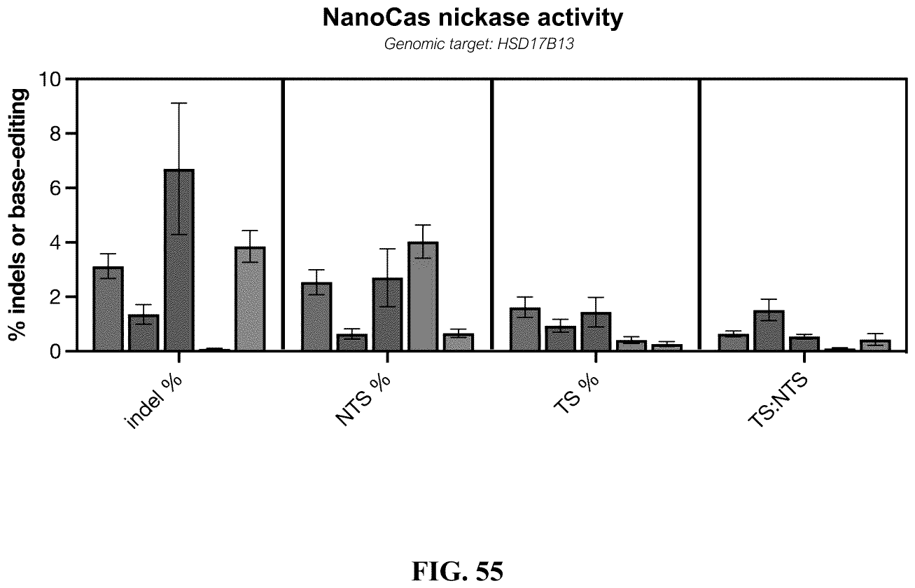

Similarly, the experiment was repeated for three systems, CasM.265466 S382A/D220R (SEQ ID NO: 379), CasM.265466 F406A/D220R (SEQ ID NO: 377), and CasM.265466 L337A/D220R (SEQ ID NO: 381), each targeting PCSK9, B2M, LDHA, or HSD17B13. However, the experiment comprises two control effector protein systems, dCasM.265466 D220R/E335Q and CasM.265466 D220R. Results of the % indels generated, % TS edited, % NTS edited, and TS: NTS ratio observed for each effector protein system are shown in FIGS. 50-55. An analysis of FIG. 50 indicates that the system comprising CasM.265466 S382A/D220R (SEQ ID NO: 379) showed relatively higher desired activity as it showed near baseline levels of indel, lower NTS % and higher TS % compared to dCasM.265466 D220R/E335Q. An analysis of FIG. 51 indicates that all systems showed similar % indel relative to CasM.265466 D220R indicating that, at least in some cases, nickase activity is target nucleic acid and/or guide RNA dependent. An analysis of FIGS. 52-55 indicates that while the system comprising CasM.265466 S382A/D220R (SEQ ID NO: 379) showed relatively higher desired activity as it showed near baseline levels of indel, lower NTS % and higher TS % compared to dCasM.265466 D220R/E335Q, nickase activity was guide RNA dependent and performance varied even within the same genomic target.

### Example 41: Effects of Guide RNA Concentration on Nickase Score of CasM.265466 Variant Systems

Effects of guide RNA concentration on % indel and nickase score for fusion protein systems of Example 40 comprising variants of CasM.265466 fused to a deaminase protein were tested. Briefly, cells were transfected with a plasmid encoding fusion proteins of CasM.265466, CasM.265466 S382A/D220R (SEQ ID NO: 379), CasM.265466 F406A/D220R (SEQ ID NO: 377), CasM.265466 L337A/D220R (SEQ ID NO: 381), CasM.265466 C385A/D220R (SEQ ID NO: 378), CasM.265466 T381A/D220R (SEQ ID NO: 380), CasM.265466 N420A/D220R (SEQ ID NO: 375), CasM.265466 N336A/D220R (SEQ ID NO: 382), CasM.265466 Y379A/D220R (SEQ ID NO: 383), CasM.265466 N424A/D220R (SEQ ID NO: 384), dCasM.265466 D220R/E335Q or CasM.265466 D220R (SEQ ID NO: 385), fused to a deaminase protein, and one of eight guide RNAs. Nucleotide sequence of the guide RNA that was used in these assays comprised a handle sequence of SEQ ID NO: 107 and a spacer sequence of SEQ ID NO: 157, 228, 269, 289, 291, 312, 321 or 386 as described in TABLE 30. NGS analysis was performed to determine % indel generated by each system. Editing of a target-strand (TS) (the strand bound by the guide RNA) of a target nucleic acid, and a non-target strand (NTS) (the strand not bound by the guide RNA) of the target nucleic acid was determined based on the NGS analysis. Nickase score was calculated using formula 1 as described in Example 40.

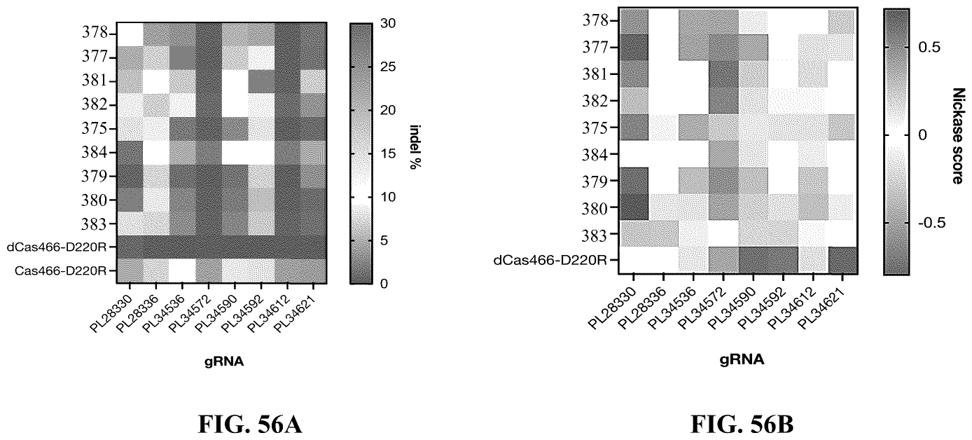

Results of % indels generated, and nickase score for each effector protein systems are shown in FIGS. 56A-56B. An analysis of FIGS. 56A-56B indicates that amongst the tested fusion protein systems, the systems comprising CasM.265466 S382A/D220R (SEQ ID NO: 379), CasM.265466 F406A/D220R (SEQ ID NO: 377), and CasM.265466 L337A/D220R (SEQ ID NO: 381) showed high nickase activity relative to the other tested systems.

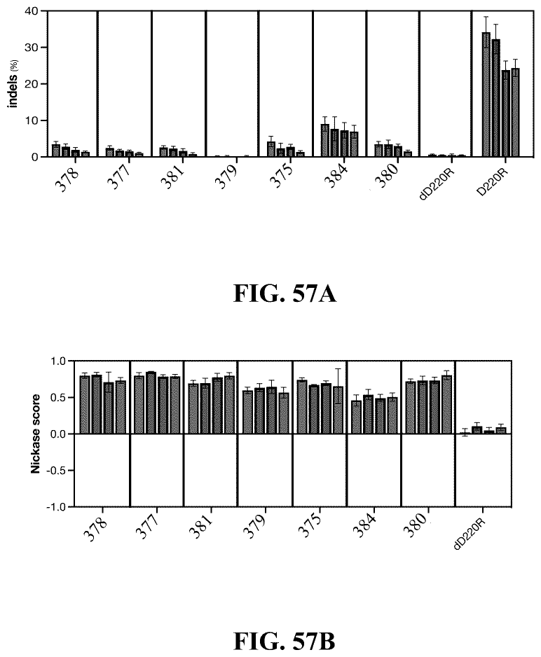

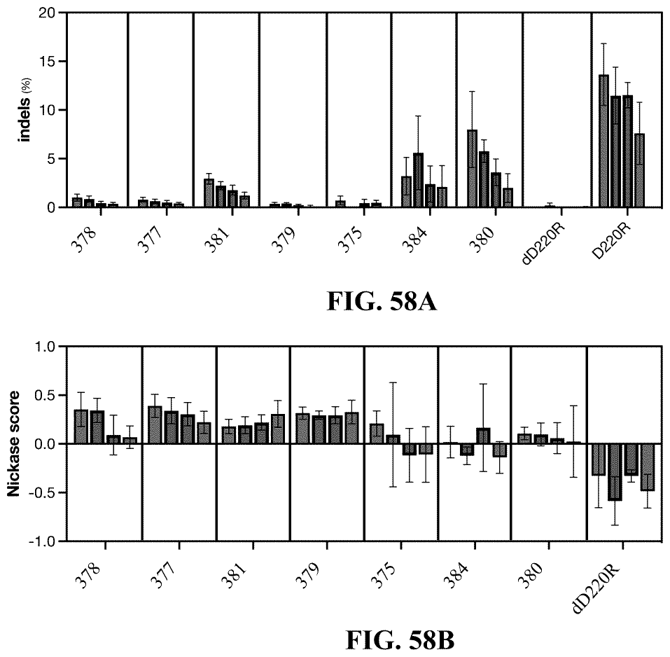

Next, gene editing by RNA encoding the fusion proteins and one of eight guide RNAs were tested. In this experiment, concentration of the RNA encoding the fusion protein was kept constant at 150 ng, whereas a range of concentration of guide RNAs, from 25 ng to 150 ng, were tested. The tested fusion protein systems included effector proteins having amino acid sequences of SEQ ID NO: 375, 377-381, or 384. CasM.265466 D220R (SEQ ID NO: 385) was used as a positive control for % indel. dCasM.265466 D220R/E335Q was used as a negative control for nickase score and % indel. Two guide RNAs, PL28330 or PL34536, were tested. Results of % indels generated, and nickase score for each fusion protein systems with PL28330 are shown in FIGS. 57A-57B. An analysis of FIGS. 57A-57B indicates that all variants showed improved nickase score and lover indel levels. Results of % indels generated, and nickase score for each fusion protein systems with PL34536 are shown in FIGS. 58A-58B. An analysis of FIGS. 58A-58B indicates that amongst the tested fusion protein systems, the systems comprising CasM.265466 S382A/D220R (SEQ ID NO: 379), CasM.265466 F406A/D220R (SEQ ID NO: 377), and CasM.265466 L337A/D220R (SEQ ID NO: 381) showed high nickase activity and low indel levels relative to the other tested systems.

### Example 42. Fusion of an Endonuclease or Glycosylase to a CasM.265466 Deaminase Fusions

A fusion protein comprising CasM.265466 (D220R, E335Q) and ABE8e having 2 NLS at each terminus with a 50 amino acid linker between CasM.265466 and ABE8e was fused to different endonucleases (EndoV) and glysosylases (NTHL1 and TDG) as described in TABLE 31. EndoV is represented by SEQ ID NO: 391, TDG is represented by SEQ ID NO: 392 and NTHL1 is represented by SEQ ID NO: 393.

TABLE 31

| Description (N term to C term) | Protein PL## |
|---|---|
| EndoV-CasM.265466 (D220R, E335Q)-ABE8e | PL35525 |
| TDG-CasM.265466 (D220R, E335Q)-ABE8e | PL35526 |
| NTHL1-CasM.265466 (D220R, E335Q)-ABE8e | PL35527 |
| CasM.265466 (D220R, E335Q)-ABE8e control | PL28806 |

The protein fusions in TABLE 32 were tested at four different targets. The guide scaffold sequence used was 5′-ACAGCUUAUUUGGAAGCUGAAAUGUGAGGUUUAUAACACUCACAAGAAUCCU GAAAAAGGAUGCCAAAC-3′ (SEQ ID NO: 107), the 3′ end of which was linked to the 5′ end of a spacer shown in TABLE 32.

TABLE 32

| Plasmid | Target | Spacer | |
|---|---|---|---|
| ID | gene | SEQ ID NO: | Spacer sequence |
| PL28330 | MLH1 | 157 | AGUCUCCAGGAAGAAAUUAA |
| PL28336 | B2M | 386 | AGUGUAUAAGCAUAUCAAUA |
| PL28341 | DMD | 394 | ACCAAAAAAUAUACGCUAUA |
| PL28779 | Fut8 | 395 | GCCUUAACAAGCUGCUCUUC |

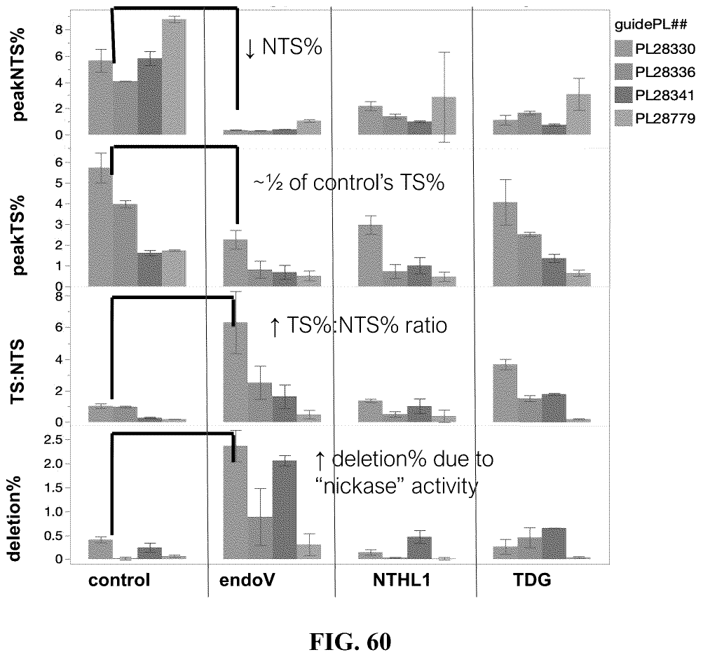

Briefly, plasmid transfections in HEK293T cells were carried out as follows. Equal amounts of nuclease expressing plasmid and guide expressing plasmids were delivered by lipofection to HEK293T cells. Transfection reagent was diluted and mixed with the plasmid DNA at the ratio of 2:1 lipid: DNA. Lipid: DNA mixture was incubated for 15 minutes at room temperature before adding it to cells. Cells were incubated for 3 days before being lysed and subjected to PCR amplification. Indels and substitution percentages were detected by next generation sequencing of PCR amplicons at the targeted loci and indel percentage was calculated as the fraction of sequencing reads containing insertions or deletions or substitutions relative to an unedited reference sequence. Results are shown in FIG. 60.

### Example 43. CasM.265466 Precise Deletion and Insertion Achieved with dsDNA Donor Alone

Effects of NHEJ inhibitor and DNA repair protein on CasM.265466 generated indels were evaluated. The results showed that NHEJ inhibitor (e.g., UbvG08) can reduce CasM.265466 generated indels by about 50%. Similarly, addition of DNA repair protein (Rad51) consistently reduced CasM.265466 generated indels (85-97%). Rad51 also increased substitutions which indicates HR copy of SNPs present among 3N in HEK293T. Although nuclease based HDR approaches may be challenging (i.e ssODN donor delivery issues), these results suggested that CasM.265466 breaks could work well with a double-strand donor, and possibly suitable for in vivo editing.

In order to evaluate CasM.265466 mediated HDR insertion and deletion, HEK293T cells were transfected with CasM.265466, a guide nucleic acid, and a dsDNA HDR donor designed to provide (1) a 5-nucleotide insertion or (2) a 15-nucleotide deletion, if incorporated. Cells were subsequently treated with UbvG03, UbvG03+Rad51, or UbvG03+Rad52. Target gene editing was analyzed by NGS 48 hours post transfection.

Results showed CasM.265466 could incorporate a dsDNA donor providing a 15-nucleotide deletion without any co-factors. NHEJi (UbvG08 peptide) increased the occurrence of the 15-nucleotide deletion by 60% while reducing byproducts by ~55%. Similarly, CasM.265466 incorporated the dsDNA donor providing a 5-nucleotide insertion without any other co-factors. Additionally, it was also observed that NHEJi increased the occurrence of the 5-nucleotide insertion by more than 100%. While Rad51 and Rad52 did not provide a noticeable increase in precise editing, they substantially reduced (~50%) the amount of observed byproduct relative to cells treated with dsDNA HDR donor alone.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1-76. (canceled)

77. A composition for modifying a nucleobase of a target nucleic acid, the system comprising:
a. an effector protein, or a nucleic acid encoding the effector protein, wherein the effector protein comprises an amino acid sequence that is at least 90% identical to SEQ ID NO: 1;
b. a base editing enzyme, or a nucleic acid encoding the base editing enzyme; and
c. a guide RNA or a nucleic acid that encodes the guide RNA, wherein the guide RNA comprises a first region that interacts with the effector protein, and a second region comprising a spacer sequence that hybridizes to a target sequence in the target nucleic acid.

78. The composition of claim 77, wherein the base editing enzyme comprises a deaminase.

79. The composition of claim 78, wherein the deaminase is selected from an adenine deaminase and a cytosine deaminase.

80. The composition of claim 77, wherein the base editing enzyme comprises an amino acid sequence that is at least 90% identical to a sequence selected from SEQ ID NOS: 2-9.

81. The composition of claim 77, wherein the effector protein is connected to the base editing enzyme, optionally via a linker.

82. The composition of claim 77, wherein the effector protein comprises nickase activity.

83. The composition of claim 77, wherein the effector protein comprises at least one amino acid substitution relative to SEQ ID NO: 1, wherein the at least one amino acid substitution is selected from D237A, D418A, D418N, E335A, and E335Q, and a combination thereof.

84. The composition of claim 77, wherein the effector protein comprises at least one amino acid substitution relative to SEQ ID NO: 1, wherein the at least one amino acid substitution is selected from I80R, T84R, K105R, G210R, C202R, A218R, D220R, E225R, C246R, and Q360R, and a combination thereof.

85. The composition of claim 77, wherein the effector protein comprises at least one amino acid substitution relative to SEQ ID NO: 1, wherein the at least one amino acid substitution is selected from K58W, 180K, N193K, S209F, A218K, E225K, N286K, M295W, M298L, A306K, and Y315M, and a combination thereof.

86. The composition of claim 77, wherein the effector protein comprises at least one amino acid substitution relative to SEQ ID NO: 1, wherein the at least one amino acid substitution is selected from L337A, T381A, S382A, C385A, F406A, N420A, and N424A, and a combination thereof.

87. The composition of claim 77, wherein the effector protein comprises at least one amino acid substitution relative to SEQ ID NO: 1, wherein the at least one amino acid substitution is selected from A75F, T84R, K250N, D171K, N193K, C202K, C202R, S209F, S209Y, D220R, E225K, E225R, K250N, N286K, A306K, Y315M, Q360R, E362D, A393R and A393E, and a combination thereof.

88. The composition of claim 77, wherein the effector protein comprises at least one amino acid substitution relative to SEQ ID NO: 1, wherein the at least one amino acid substitution is selected from:
a) D220R and D237A;
b) D220R and D237N; and
c) D220R and E335Q.

89. The composition of claim 77, wherein guide RNA comprises a nucleotide sequence that is at least 80% identical to a sequence selected from TABLE 5, TABLE 6, and TABLE 8.

90. The composition of claim 77, wherein the guide RNA comprises an aptamer.

91. The composition of claim 90, comprising an additional polypeptide or nucleic acid encoding the same, wherein the additional polypeptide comprises an aptamer binding protein.

92. The composition of claim 91, wherein the additional polypeptide comprises an exonuclease.

93. A genetically modified cell comprising or modified by the composition of claim 77.

94. A lipid nanoparticle comprising the composition of claim 77.

95. An adeno associated viral vector comprising the composition of claim 77, wherein the composition comprises the nucleic acid encoding the effector protein, the nucleic acid encoding the base editing enzyme, and the nucleic acid encoding the guide RNA.

96. A method of modifying one or more nucleobases of a target nucleic acid comprising contacting the target nucleic acid with the composition of claim 77.


SEQUENCE LISTING (403 sequences)

SEQ ID NO: 1 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 2 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 3 - protein, 364 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 4 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 5 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 6 - protein, 198 aa, synthetic construct
EASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 7 - protein, 228 aa, synthetic construct
SSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLK

SEQ ID NO: 8 - protein, 224 aa, synthetic construct
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 9 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 10 - protein, 854 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSPKKKRKVEASGGGSGGGSGGGSGEAAPCDCDGGTQKEKGPYYTHLGAGPSVAAVRELMETRFGQKGKAIRIEKIVFTGKEGKSSQGCPVAKWVIRRSGPEEKLICLVRERVDHHCSTAVIVVLILLWEGIPRLMADRLYKELTENLRSYSGHPTDRRCTLNKKRTCTCQGIDPKTCGASFSFGCSWSMYFNGCKFGRSENPRKFRLAPNYPLHNYYKRITGMSSEGSDVKTGWIIPDRKTLISREEKQLEKNLQELATVLAPLYKQMAPVAYQNQVEYEEVAGDCRLGNEEGRPFSGVTCCMDFCAHSHKDIHNMHNGSTVVCTLIRADGRDTNCPEDEQLHVLPLYRLADTDEFGSVEGMKAKIKSGAIQVNGPTRKRRLRFTEPVPRCGKRAKMKQNHNKSGSHNTKSFSSASSTSHLVKDESTDFCPLQASSAETSTCTYSKTASGGFAETSSILHCTMPSGAHSGANAAAGECTGTVQPAEVAAHPHQSLPTADSPVHAEPLTSPSEQLTSNQSNQQLPLLSNSQKLASCQVEDERHPEADEPQHPEDDNLPQLDEFWSDSEEIYADPSFGGVAIAPIHGSVLIECARKELHATTSLRSPKRGVPFRVSLVFYQHKSLNKPNHGFDINKIKCKCKKVTKKKPADRECPDVSPEANLSHQIPSRVASTLTRDNVVTVSPYSLTHVAGPYNRWVAAADYKDDDDK

SEQ ID NO: 11 - protein, 1006 aa, synthetic construct
QSQPGHNQMLRPIKTEPVSKPSSYRYPLSPPQENMSSRIKQEISSPSRDNGQPKSIIETMEQHLKQFQLKSLCDYKALTLKSQKHVKVPTDIQAAESENHARAAEPQATKSTDCSVLDDVSESDTPGEQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETKKKQSPPRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKHSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTVYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 12 - protein, 617 aa, synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 13 - protein, 523 aa, synthetic construct
DALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 14 - protein, 322 aa, synthetic construct
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 15 - protein, 380 aa, synthetic construct
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFST

SEQ ID NO: 16 - protein, 50 aa, synthetic construct
DALDDFDLDMLGSDALDDFDLDMLGSDALDDFDLDMLGSDALDDFDLDML

SEQ ID NO: 17 - protein, 745 aa, synthetic construct
GQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 18 - protein, 75 aa, synthetic construct
MDAKSLTAWSRTLVTFKDVFVDFTREEWKLLDTAQQIVYRNVMLENYKNLVSLGYQLTKPDVILRLEKGEEPWLV

SEQ ID NO: 19 - protein, 99 aa, synthetic construct
NNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 20 - protein, 255 aa, Escherichia coli
MWIGIISLFPEMFRAITDYGVTGRAVKNGLLSIQSWSPRDFTHDRHRTVDDRPYGGGPGMLMMVQPLRDAIHAAKAAAGEGAKVIYLSPQGRKLDQAGVSELATNQKLILVCGRYEGIDERVIQTEIDEEWSIGDYVLSGGELPAMTLIDSVSRFIPGVLGHEASATEDSFAEGLLDCPHYTRPEVLEGMEVPPVLLSGNHAEIRRWRLKQSLGRTWLRRPELLENLALTEEQARLLAEFKTEHAQQQHKHDGMA

SEQ ID NO: 21 - protein, 246 aa, Haemophilus influenzae
MWIGVISLFPEMFKAITEFGVTGRAVKHNLLKVECWNPRDFTFDKHKTVDDRPYGGGPGMLMMVQPLRDAIHTAKAAAGEGAKVIYLSPQGRKLDQGGVTELAQNQKLILVCGRYEGIDERLIQTEIDEEWSIGDYVLTGGELPAMTLIDAVARFIPGVLGKQASAEEDSFADGLLDCPHYTRPEVLEGLTVPPVLMSGHHEEIRKWRLKQSLQRTWLRRPELLEGLALTDEQRKLLKEAQAEHNS

SEQ ID NO: 22 - protein, 509 aa, Homo sapiens
MVLWILWRPFGFSGRFLKLESHSITESKSLIPVAWTSLTQMLLEAPGIFLLGQRKRFSTMPETETHERETELFSPPSDVRGMTKLDRTAFKKTVNIPVLKVRKEIVSKLMRSLKRAALQRPGIRRVIEDPEDKESRLIMLDPYKIFTHDSFEKAELSVLEQLNVSPQISKYNLELTYEHFKSEEILRAVLPEGQDVTSGFSRIGHIAHLNLRDHQLSFKHLIGQVMIDKNPGITSAVNKINNIDNMYRNFQMEVLSGEQNMMTKVRENNYTYEFDFSKVYWNPRLSTEHSRITELLKPGDVLFDVFAGVGPFAIPVAKKNCTVFANDLNPESHKWLLYNCKLNKVDQKVKVFNLDGKDFLQGPVKEELMQLLGLSKERKPSVHVVMNLPAKAIEFLSAFKWLLDGQPCSSEFLPIVHCYSFSKDANPAEDVRQRAGAVLGISLEACSSVHLVRNVAPNKEMLCITFQIPASVLYKNQTRNPENHEDPPLKRQRTAEAFSDEKTQIVSNT

SEQ ID NO: 23 - protein, 336 aa, Methanocaldococcus jannaschii
MPLCLKINKKHGEQTRRILIENNLLNKDYKITSEGNYLYLPIKDVDEDILKSILNIEFELVDKELEEKKIIKKPSFREIISKKYRKEIDEGLISLSYDVVGDLVILQISDEVDEKIRKEIGELAYKLIPCKGVFRRKSEVKGEFRVRELEHLAGENRTLTIHKENGYRLWVDIAKVYFSPRLGGERARIMKKVSLNDVVVDMFAGVGPFSIACKNAKKIYAIDINPHAIELLKKNIKLNKLEHKIIPILSDVREVDVKGNRVIMNLPKFAHKFIDKALDIVEEGGVIHYYTIGKDFDKAIKLFEKKCDCEVLEKRIVKSYAPREYILALDFKINKK

SEQ ID NO: 24 - protein, 333 aa, Pyrococcus abyssi
MSGVKVRREDAKKVLELLKSVGILDGKRKAIRDEKYVIFPVTDTNIAKSLGLEVVDVELPMRPERQIYKNLEDLLPREIFKKLGRLDIVGDIAIVSIPDEILSEREVIVSAIRKLYPKVKVIARRGFHSGLYRIRELEVIWGENRLHTIHKENGVLIKVDLSKVFFNPRMKGERYRIAQLVNDGERILVPFAGVIPYPLVIARFKNVEVYAVEINEFAVKLAEENLELNRDRLKGKIKIIHGDVFEVLPNLPNFDRVVSPTPKGVDALSLTLSKAEKFLHYYDFVHESEIERFRERVLEECRRQGKECRVSVRKVSDYKPHVYKVCADVEILS

SEQ ID NO: 25 - protein, 330 aa, Pyrococcus abyssi
MTLAVKVPLKEGEIVRRRLIELGALDNTYKIKREGNFLLIPVKFPVKGFEVVEAELEQVSRRPNSYREIVNVPQELRRFLPTSFDIIGNIAIIEIPEELKGYAKEIGRAIVEVHKNVKAVYMKGSKIEGEYRTRELIHIAGENITETIHRENGIRLKLDVAKVYFSPRLATERMRVFKMAQEGEVVFDMFAGVGPFSILLAKKAELVFACDINPWAIKYLEENIKLNKVNNVVPILGDSREIEVKADRIIMNLPKYAHEFLEHAISCINDGGVIHYYGFGPEGDPYGWHLERIRELANKFGVKVEVLGKRVIRNYAPRQYNIAIDFRVSF

SEQ ID NO: 26 - protein, 277 aa, Sulfolobus solfataricus
MWSLMHPYLPATVRKWTNLNLSNHSFSLNTMKTKRLSEFFPGVRSYYIVGEIAIITPKRVNVDYNLVAEKIMQAHPKIKAVYLKKKVKGELRTNELEFLSGERISSTIYKENGVLFYVDINKVYVNPSLSGDRLKNLELVEEGSTVLDAFTGYGAIALNIAHKKRVYVVAGDINIDGLYMLKKSLSLNKIKGEMIDIVQYDAHHLPFRDKVFKLSFGDNPTLIIDFKEELCRVSENVVFYILCESEDKANSSLGRTSWIKINDYSKNLFIFKGLVRC

SEQ ID NO: 27 - protein, 468 aa, Arabidopsis thaliana
MFDESKFDVNLKLWALRIPRELCKSASRILNGYMLNMPRIKPITEDPTCEKTRLVILSESVKNADLSEIPEEKLNQLKKLSELEVVPHSVTLGYSYWSADHLLKQILPDGLDIPSSFETIGHIAHLNLHDELLPFKDVIAKVIYDKNYPRIKTIVNKVGTISNEFRVPKFEVLAGENGMETEVKQYGARFKLDYGLVYWNSRLEHEHMRLSSLFKPGETVCDMFAGIGPFAIPAAQKGCFVYANDLNPDSVRYLKINAKFNKVDDLICVHNMDARKFFSHLMAVSTCEDNLQSVADNDKTKEAAVSRGGETNSSGEEIRESNASINEPLGANKKPSGTTKTENGVGKDCKSIEGHANKRLRQTLLPIAKPWEHIDHVIMNLPASALQFLDSFSNVIQKKYWKGPLPLIHCYCFIRASETTEFIIAEAETALKFHIEDPVFHKVRDVAPNKAMFCLSFRLPEACLKQEE

SEQ ID NO: 28 - protein, 499 aa, Saccharomyces cerevisiae
MKIALPVFQKFNRLISSCKMSGVFPYNPPVNRQMRELDRSFFITKIPMCAVKFPEPKNISVFSKNFKNCILRVPRIPHVVKLNSSKPKDELTSVQNKKLKTADGNNTPVTKGVLLHESIHSVEDAYGKLPEDALAFLKENSAEIVPHEYVLDYDFWKAEEILRAVLPEQFLEEVPTGFTITGHIAHLNLRTEFKPFDSLIGQVILDKNNKIECVVDKVSSIATQFRTFPMKVIAGKSDSLVVEQKESNCTFKFDFSKVYWNSRLHTEHERLVKQYFQPGQVVCDVFAGVGPFAVPAGKKDVIVLANDLNPESYKYLKENIALNKVAKTVKSFNMDGADFIRQSPQLLQQWIQDEEGGKITIPLPLKKRHRSQQHNDQQPPQPRTKELIIPSHISHYVMNLPDSAISFLGNFRGIFAAHTKGATDTIQMPWVHVHCFEKYPPGDQVTEDELHARVHARIIAALKVTADDLPLNAVSLHLVRKVAPTKPMYCASFQLPANV

SEQ ID NO: 29 - protein, 501 aa, Mus musculus
MRIVWKLFGFSRRLLQVEWCHPSESILLFTLVPRLRKAPSVFLLGQRQGLSTMPEIEASVRDSELFSPPSDVRGMRELDRTAFKKTVSIPVLKARKEVVNRLMRALRRVALQRPGIKRVIEDPKDEDSRLIMLDPYRMLTADSFDKAELGVLKELDVSPQLSQYNLELTYENFKSEEILKAVLPEGQDVTSGFSRVGHIAHLNLRDHQLPFKHLIGQVMVDKNPGITSAVNKTSNIDNTYRNFQMEVLCGEENMLTKVRENNYTYEFDFSKVYWNPRLSTEHGRITELLNPGDVLFDVFAGVGPFAIPAARKNCTVFANDLNPESHKWLLHNCKLNKVDQKVKVFNMDGKDFIQGPVREELMLRLGLSAEAKPSVHIVMNLPAKAIEFLSVFRSLLDGQPCSTELLPTVHCYCFSKDSDPAKDVRQQAEAVLGVSLETSSSVHLVRNVAPNKEMLCITFQIPTATLYRNQSLSLQNDQEPPLKRQKTGDPFSGEPQIASDS

SEQ ID NO: 30 - protein, 293 aa, Saccharomyces cerevisiae
MSNDEINQNEEKVKRTPPLPPVPEGMSKKQWKKMCKRQRWEENKAKYNAERRVKKKRLRHERSAKIQEYIDRGEEVPQELIREPRINVNQTDSGIEIILDCSFDELMNDKEIVSLSNQVTRAYSANRRANHFAEIKVAPFDKRLKQRFETTLKNTNYENWNHFKFLPDDKIMFGDEHISKDKIVYLTADTEEKLEKLEPGMRYIVGGIVDKNRYKELCLKKAQKMGIPTRRLPIDEYINLEGRRVLTTTHVVQLMLKYFDDHNWKNAFESVLPPRKLDAEAKSASSSPAPKDT

SEQ ID NO: 31 - protein, 304 aa, Schizosaccharomyces pombe
MENKDALDIGKDDTNTSEADVSKNETQEQPVLSKSALKRLKRQQEWDAGREKRAEMRREKKRLRKEERKRKIEAGEVVKSQKKRIRLGKVVPSSIRIVLDCAFDDLMNDKEINSLCQQVTRCHSANRTALHPVELFATNFGGRLKTRQDFVLKGQQNNWKRYNPTTKSYLEEFESQKEKLVYLSADSDNTITELDEDKIYIIGAIVDKNRYKNLCQNKASEQGIKTAKLPIDEYIKITDRKILTVNQVFEILSLWLEYRDWEKAFMEVIPKRKGILLKSDESFDVSEDTRSQSNQSDSELEKEN

SEQ ID NO: 32 - protein, 339 aa, Homo sapiens
MSSEMLPAFIETSNVDKKQGINEDQEESQKPRLGEGCEPISKRQMKKLIKQKQWEEQRELRKQKRKEKRKRKKLERQCQMEPNSDGHDRKRVRRDVVHSTLRLIIDCSFDHLMVLKDIKKLHKQIQRCYAENRRALHPVQFYLTSHGGQLKKNMDENDKGWVNWKDIHIKPEHYSELIKKEDLIYLTSDSPNILKELDESKAYVIGGLVDHNHHKGLTYKQASDYGINHAQLPLGNFVKMNSRKVLAVNHVFEIILEYLETRDWQEAFFTILPQRKGAVPTDKACESASHDNQSVRMEEGGSDSDSSEEEYSRNELDSPHEEKQDKENHTESTVNSLPH

SEQ ID NO: 33 - protein, 316 aa, Homo sapiens
MDWKLEGSTQKVESPVLQGQEGILEETGEDGLPEGFQLLQIDAEGECQEGEILATGSTAWCSKNVQRKQRHWEKIVAAKKSKRKQEKERRKANRAENPGICPQHSKRFLRALTKDKLLEAKHSGPRLCIDLSMTHYMSKKELSRLAGQIRRLYGSNKKADRPFWICLTGFTTDSPLYEECVRMNDGFSSYLLDITEEDCFSLFPLETLVYLTPDSEHALEDVDLNKVYILGGLVDESIQKKVTFQKAREYSVKTARLPIQEYMVRNQNGKNYHSEILAINQVFDILSTYLETHNWPEALKKGVSSGKGYILRNSVE

SEQ ID NO: 34 - protein, 403 aa, Homo sapiens
MAAFLKMSVSVNFFRPFTRFLVPFTLHRKRNNLTILQRYMSSKIPAVTYPKNESTPPSEELELDKWKTTMKSSVQEECVSTISSSKDEDPLAATREFIEMWRLLGREVPEHITEEELKTLMECVSNTAKKKYLKYLYTKEKVKKARQIKKEMKAAAREEAKNIKLLETTEEDKQKNFLFLRLWDRNMDIAMGWKGAQAMQFGQPLVFDMAYENYMKRKELQNTVSQLLESEGWNRRNVDPFHIYFCNLKIDGALHRELVKRYQEKWDKLLLTSTEKSHVDLFPKDSIIYLTADSPNVMTTFRHDKVYVIGSFVDKSMQPGTSLAKAKRLNLATECLPLDKYLQWEIGNKNLTLDQMIRILLCLKNNGNWQEALQFVPKRKHTGFLEISQHSQEFINRLKKAKT

SEQ ID NO: 35 - protein, 479 aa, synthetic construct
VGTMITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEKLGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIAQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 36 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 37 - protein, 509 aa, synthetic construct
MPIAPEFLSLAYPGQEFPAYLYGRASRDPKRKGRSVQSQLDEGRATCLDAGWPIAGEFKDVDRSASAYARRTRDEFEEMIAGIQAGECRILVAFEASRYYRDLEAYVRLRRVCREAGVLLCYNGQVYDLSKSADRKATAQDAVNAEGEADDIRERNLRTTRLNAKRGGAHGPVPDGYKRRYDPDSGDLVDQIPHPDRAGLITEIFRRAAAAEPLAAICRDLNERGETTHRGKAWQRHHLHAILRNPAYIGHRRHLGVDTGKGMWAPICDDEDFAETFQAVQEILSLPGRQLSPGPEAQHLQTGIALCGEHPDEPPLRSVTVRGRTNYNCSTRYDVAMREDRMDAFVEESVITWLASDEAVAAFEDNTDDERTRKARIRLKVLEEQLEAAQKQARTLRPDGMGMLLSIDSLAGLEAELTPQIDKARQESRSLHVPALLRDLLGKPRADVDRAWNEALTLPQRRMILRMVVTIRLFKAGSRGVRAIEPGRITLSYVGEPGFKPVGGNRAKQ

SEQ ID NO: 38 - protein, 474 aa, synthetic construct
MAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQ

SEQ ID NO: 39 - protein, 805 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSRSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFKSSKFTRTISPPTLGTLRSCFSWSGGLGDFSRTPSPSPSTALQQFRRKSDSPTSLPENNMSDVSQLKSEESSDDESHPLREGACSSQSQESGEFSLQSSNASKLSQCSSKDSDSEESDCNIKLLDSQSDQTSKLCLSHFSKKDTPLRNKVPGLYKSSSADSLSTTKIKPLGPARASGLSKKPASIQKRKHHNAENKPGLQIKLNELWKNFGFKKFRD

SEQ ID NO: 40-41 - skipped (2 entries)

SEQ ID NO: 42 - protein, 8 aa, unidentified
  VARIANT 3: note = Any amino acid
  VARIANT 4: note = W, F, or Y
  VARIANT 5: note = Any amino acid
  VARIANT 6: note = Any amino acid
KRXXXXAF

SEQ ID NO: 43 - skipped

SEQ ID NO: 44 - protein, 6 aa, unidentified
  VARIANT 5: note = K or R
  VARIANT 6: note = W, F, or Y
LGKRXX

SEQ ID NO: 45 - skipped

SEQ ID NO: 46 - protein, 20 aa, unidentified
  VARIANT 4: note = Any amino acid
  VARIANT 8: note = Any amino acid
  VARIANT 11: note = Any amino acid
  VARIANT 15: note = Any amino acid
  VARIANT 19: note = Any amino acid
GLFXALLXLLXSLWXLLLXA

SEQ ID NO: 47-52 - skipped (6 entries)

SEQ ID NO: 53 - protein, 16 aa, unidentified
APKKKRKVGIHGVPAA

SEQ ID NO: 54 - protein, 9 aa, unidentified
MAPKKKRKV

SEQ ID NO: 55 - protein, 16 aa, unidentified
KRPAATKKAGQAKKKK

SEQ ID NO: 56 - protein, 18 aa, unidentified
KRPAATKKAGQAKKKKEF

SEQ ID NO: 57 - protein, 7 aa, unidentified
PKKKRKV

SEQ ID NO: 58 - protein, 9 aa, unidentified
PAAKRVKLD

SEQ ID NO: 59 - protein, 15 aa, unidentified
PKKKRKVGIHGVPAA

SEQ ID NO: 60-76 - skipped (17 entries)

SEQ ID NO: 77 - RNA, 13 nt, synthetic construct
  source 1..13: mol_type = other RNA
aaggatgccaaac

SEQ ID NO: 78 - RNA, 13 nt, synthetic construct
  source 1..13: mol_type = other RNA
tggtacatccaac

SEQ ID NO: 79 - RNA, 13 nt, synthetic construct
  source 1..13: mol_type = other RNA
gtggatatccaac

SEQ ID NO: 80 - RNA, 13 nt, synthetic construct
  source 1..13: mol_type = other RNA
agcagaatacaac

SEQ ID NO: 81 - RNA, 13 nt, synthetic construct
  source 1..13: mol_type = other RNA
gtgaatatccaac

SEQ ID NO: 82 - RNA, 13 nt, synthetic construct
  source 1..13: mol_type = other RNA
aggtatatccaac

SEQ ID NO: 83 - RNA, 13 nt, synthetic construct
  source 1..13: mol_type = other RNA
agcaatatacaac

SEQ ID NO: 84 - RNA, 36 nt, synthetic construct
  source 1..36: mol_type = other RNA
gtttgagaaccttatgaaattacaaggatgccaaac

SEQ ID NO: 85 - RNA, 52 nt, synthetic construct
  source 1..52: mol_type = other RNA
acagcttatttggaagctgaaatgtgaggtttataacactcacaagaatcct

SEQ ID NO: 86 - RNA, 61 nt, synthetic construct
  source 1..61: mol_type = other RNA
tggggcagttggttgcccttagcctgaggcatttattgcactcgggaagtaccatttctca

SEQ ID NO: 87 - RNA, 54 nt, synthetic construct
  source 1..54: mol_type = other RNA
tggggcagttggttgcccttagcctgaggcatttattgcactcgggaagtacca

SEQ ID NO: 88 - RNA, 52 nt, synthetic construct
  source 1..52: mol_type = other RNA
agggcgtgttggagcgccttagtttgaggtatcaagcactcaaaaaatctac

SEQ ID NO: 89 - RNA, 55 nt, synthetic construct
  source 1..55: mol_type = other RNA
acgggtggttgtacacccgaagagtgaggtcttaacgggcactcgctaatctgat

SEQ ID NO: 90 - RNA, 52 nt, synthetic construct
  source 1..52: mol_type = other RNA
tgggcgcgttggagcgccttggttcgaggttccctgcactcgaaaaattcac

SEQ ID NO: 91 - RNA, 52 nt, synthetic construct
  source 1..52: mol_type = other RNA
tgggcgtgttggaacgccttagtttgaggtttcaagcactcaaaaaattcac

SEQ ID NO: 92 - RNA, 52 nt, synthetic construct
  source 1..52: mol_type = other RNA
cggggtggttggacacccttaaattgaggttcatcgcactcgataaatacca

SEQ ID NO: 93 - RNA, 53 nt, synthetic construct
  source 1..53: mol_type = other RNA
ggggctggttgtacagcctgaagttgagggatgattccactcgacaaattgct

SEQ ID NO: 94 - RNA, 52 nt, synthetic construct
  source 1..52: mol_type = other RNA
cgggctggttggacagccttaaactgaggtttaacgcactcggtaaataccc

SEQ ID NO: 95 - RNA, 53 nt, synthetic construct
  source 1..53: mol_type = other RNA
gacagcttatttggaagctgaaatgtgaggtttataacactcacaagaatcct

SEQ ID NO: 96-97 - skipped (2 entries)

SEQ ID NO: 98 - protein, 5 aa, synthetic construct
GSGGS

SEQ ID NO: 99 - protein, 6 aa, synthetic construct
GGSGGS

SEQ ID NO: 100 - protein, 4 aa, synthetic construct
GGGS

SEQ ID NO: 101 - protein, 4 aa, synthetic construct
GGSG

SEQ ID NO: 102 - protein, 5 aa, synthetic construct
GGSGG

SEQ ID NO: 103 - protein, 5 aa, synthetic construct
GSGSG

SEQ ID NO: 104 - protein, 5 aa, synthetic construct
GSGGG

SEQ ID NO: 105 - protein, 5 aa, synthetic construct
GGGSG

SEQ ID NO: 106 - protein, 5 aa, synthetic construct
GSSSG

SEQ ID NO: 107 - RNA, 69 nt, synthetic construct
  source 1..69: mol_type = other RNA
acagcttatttggaagctgaaatgtgaggtttataacactcacaagaatcctgaaaaaggatgccaaac

SEQ ID NO: 108 - RNA, 78 nt, synthetic construct
  source 1..78: mol_type = other RNA
tggggcagttggttgcccttagcctgaggcatttattgcactcgggaagtaccatttctcagaaatggtacatccaac

SEQ ID NO: 109 - RNA, 71 nt, synthetic construct
  source 1..71: mol_type = other RNA
tggggcagttggttgcccttagcctgaggcatttattgcactcgggaagtaccagaaatggtacatccaac

SEQ ID NO: 110 - RNA, 69 nt, synthetic construct
  source 1..69: mol_type = other RNA
agggcgtgttggagcgccttagtttgaggtatcaagcactcaaaaaatctacgaaagtggatatccaac

SEQ ID NO: 111 - RNA, 72 nt, synthetic construct
  source 1..72: mol_type = other RNA
acgggtggttgtacacccgaagagtgaggtcttaacgggcactcgctaatctgatgaaaagcagaatacaac

SEQ ID NO: 112 - RNA, 69 nt, synthetic construct
  source 1..69: mol_type = other RNA
tgggcgcgttggagcgccttggttcgaggttccctgcactcgaaaaattcacgaaagtgaatatccaac

SEQ ID NO: 113 - RNA, 69 nt, synthetic construct
  source 1..69: mol_type = other RNA
tgggcgtgttggaacgccttagtttgaggtttcaagcactcaaaaaattcacgaaagtggatatccaac

SEQ ID NO: 114 - RNA, 69 nt, synthetic construct
  source 1..69: mol_type = other RNA
cggggtggttggacacccttaaattgaggttcatcgcactcgataaataccagaaaaggtatatccaac

SEQ ID NO: 115 - RNA, 70 nt, synthetic construct
  source 1..70: mol_type = other RNA
ggggctggttgtacagcctgaagttgagggatgattccactcgacaaattgctgaaaagcaatatacaac

SEQ ID NO: 116 - RNA, 69 nt, synthetic construct
  source 1..69: mol_type = other RNA
cgggctggttggacagccttaaactgaggtttaacgcactcggtaaatacccgaaaaggtatatccaac

SEQ ID NO: 117 - RNA, 70 nt, synthetic construct
  source 1..70: mol_type = other RNA
gacagcttatttggaagctgaaatgtgaggtttataacactcacaagaatcctgaaaaaggatgccaaac

SEQ ID NO: 118 - RNA, 88 nt, synthetic construct
  source 1..88: mol_type = other RNA
acatgaggatcacccatgtacagcttatttggaagctgaaatgtgaggtttataacactcacaagaatcctgaaaaaggatgccaaac

SEQ ID NO: 119 - RNA, 92 nt, synthetic construct
  source 1..92: mol_type = other RNA
gcacatgaggatcacccatgtgcacagcttatttggaagctgaaatgtgaggtttataacactcacaagaatcctgaaaaaggatgccaaac

SEQ ID NO: 120 - RNA, 98 nt, synthetic construct
  source 1..98: mol_type = other RNA
gcagcacatgaggatcacccatgtgctgcacagcttatttggaagctgaaatgtgaggtttataacactcacaagaatcctgaaaaaggatgccaaac

SEQ ID NO: 121 - RNA, 108 nt, synthetic construct
  source 1..108: mol_type = other RNA
cgctatacgcacatgaggatcacccatgtgcgtatagcgacagcttatttggaagctgaaatgtgaggtttataacactcacaagaatcctgaaaaaggatgccaaac

SEQ ID NO: 122 - RNA, 94 nt, synthetic construct
  source 1..94: mol_type = other RNA
gcacatgaggatcacccatgtgcaaacagcttatttggaagctgaaatgtgaggtttataacactcacaagaatcctgaaaaaggatgccaaac

SEQ ID NO: 123 - RNA, 97 nt, synthetic construct
  source 1..97: mol_type = other RNA
gcacatgaggatcacccatgtgcaaaaaacagcttatttggaagctgaaatgtgaggtttataacactcacaagaatcctgaaaaaggatgccaaac

SEQ ID NO: 124 - RNA, 102 nt, synthetic construct
  source 1..102: mol_type = other RNA
gcacatgaggatcacccatgtgcaaaaaaaaaaacagcttatttggaagctgaaatgtgaggtttataacactcacaagaatcctgaaaaaggatgccaaac

SEQ ID NO: 125 - RNA, 90 nt, synthetic construct
  source 1..90: mol_type = other RNA
acatgaggatcacccatgtaaacagcttatttggaagctgaaatgtgaggtttataacactcacaagaatcctgaaaaaggatgccaaac

SEQ ID NO: 126 - RNA, 93 nt, synthetic construct
  source 1..93: mol_type = other RNA
acatgaggatcacccatgtaaaaaacagcttatttggaagctgaaatgtgaggtttataacactcacaagaatcctgaaaaaggatgccaaac

SEQ ID NO: 127 - RNA, 98 nt, synthetic construct
  source 1..98: mol_type = other RNA
acatgaggatcacccatgtaaaaaaaaaaacagcttatttggaagctgaaatgtgaggtttataacactcacaagaatcctgaaaaaggatgccaaac

SEQ ID NO: 128 - RNA, 85 nt, synthetic construct
  source 1..85: mol_type = other RNA
acagcttatttggaagctgaaatgtgaggttacatgaggatcacccatgtaacactcacaagaatcctgaaaaaggatgccaaac

SEQ ID NO: 129 - RNA, 89 nt, synthetic construct
  source 1..89: mol_type = other RNA
acagcttatttggaagctgaaatgtgaggtttaacatgaggatcacccatgtataacactcacaagaatcctgaaaaaggatgccaaac

SEQ ID NO: 130 - RNA, 89 nt, synthetic construct
  source 1..89: mol_type = other RNA
acagcttatttggaagctgaaatgtgaggttgcacatgaggatcacccatgtgcaacactcacaagaatcctgaaaaaggatgccaaac

SEQ ID NO: 131 - RNA, 95 nt, synthetic construct
  source 1..95: mol_type = other RNA
acagcttatttggaagctgaaatgtgaggttgcagcacatgaggatcacccatgtgctgcaacactcacaagaatcctgaaaaaggatgccaaac

SEQ ID NO: 132 - RNA, 105 nt, synthetic construct
  source 1..105: mol_type = other RNA
acagcttatttggaagctgaaatgtgaggttcgctatacgcacatgaggatcacccatgtgcgtatagcgaacactcacaagaatcctgaaaaaggatgccaaac

SEQ ID NO: 133 - RNA, 93 nt, synthetic construct
  source 1..93: mol_type = other RNA
acagcttatttggaagctgaaatgtgaggtttagcacatgaggatcacccatgtgcataacactcacaagaatcctgaaaaaggatgccaaac

SEQ ID NO: 134 - RNA, 99 nt, synthetic construct
  source 1..99: mol_type = other RNA
acagcttatttggaagctgaaatgtgaggtttagcagcacatgaggatcacccatgtgctgcataacactcacaagaatcctgaaaaaggatgccaaac

SEQ ID NO: 135 - RNA, 109 nt, synthetic construct
  source 1..109: mol_type = other RNA
acagcttatttggaagctgaaatgtgaggtttacgctatacgcacatgaggatcacccatgtgcgtatagcgataacactcacaagaatcctgaaaaaggatgccaaac

SEQ ID NO: 136 - RNA, 93 nt, synthetic construct
  source 1..93: mol_type = other RNA
acagcttatttggaagctgaaatgtgaggtgtagcacatgaggatcacccatgtgcatcacactcacaagaatcctgaaaaaggatgccaaac

SEQ ID NO: 137 - RNA, 93 nt, synthetic construct
  source 1..93: mol_type = other RNA
acagcttatttggaagctgaaatgtgaggtgaagcacatgaggatcacccatgtgcaacacactcacaagaatcctgaaaaaggatgccaaac

SEQ ID NO: 138 - RNA, 85 nt, synthetic construct
  source 1..85: mol_type = other RNA
acagcttatttggaagctgaaatgtgaggtttataacactcacaagaatccttacatgaggatcacccatgtaaggatgccaaac

SEQ ID NO: 139 - RNA, 87 nt, synthetic construct
  source 1..87: mol_type = other RNA
acagcttatttggaagctgaaatgtgaggtttataacactcacaagaatcctaaacatgaggatcacccatgtaaaggatgccaaac

SEQ ID NO: 140 - RNA, 89 nt, synthetic construct
  source 1..89: mol_type = other RNA
acagcttatttggaagctgaaatgtgaggtttataacactcacaagaatccttgcacatgaggatcacccatgtgcaaggatgccaaac

SEQ ID NO: 141 - RNA, 95 nt, synthetic construct
  source 1..95: mol_type = other RNA
acagcttatttggaagctgaaatgtgaggtttataacactcacaagaatccttgcagcacatgaggatcacccatgtgctgcaaggatgccaaac

SEQ ID NO: 142 - RNA, 105 nt, synthetic construct
  source 1..105: mol_type = other RNA
acagcttatttggaagctgaaatgtgaggtttataacactcacaagaatccttcgctatacgcacatgaggatcacccatgtgcgtatagcgaaggatgccaaac

SEQ ID NO: 143 - RNA, 88 nt, synthetic construct
  source 1..88: mol_type = other RNA
acagcttatttggaagctgaaatgtgaggtttataacactcacaagaatcctgcacatgaggatcacccatgtgcaaggatgccaaac

SEQ ID NO: 144 - RNA, 94 nt, synthetic construct
  source 1..94: mol_type = other RNA
acagcttatttggaagctgaaatgtgaggtttataacactcacaagaatcctgcagcacatgaggatcacccatgtgctgcaaggatgccaaac

SEQ ID NO: 145 - RNA, 97 nt, synthetic construct
  source 1..97: mol_type = other RNA
acagcttatttggaagctgaaatgtgaggtttataacactcacaagaatcctaagcagcacatgaggatcacccatgtgctgcaaaggatgccaaac

SEQ ID NO: 146 - RNA, 107 nt, synthetic construct
  source 1..107: mol_type = other RNA
acagcttatttggaagctgaaatgtgaggtttataacactcacaagaatcctaacgctatacgcacatgaggatcacccatgtgcgtatagcgaaaggatgccaaac

SEQ ID NO: 147 - RNA, 93 nt, synthetic construct
  source 1..93: mol_type = other RNA
acagcttatttggaagctgaaatgtgaggtttataacactcacaagaatccttgtaaacatgaggatcacccatgtttacaaggatgccaaac

SEQ ID NO: 148 - RNA, 97 nt, synthetic construct
  source 1..97: mol_type = other RNA
acagcttatttggaagctgaaatgtgaggtttataacactcacaagaatccttgtaaaaacatgaggatcacccatgtaattacaaggatgccaaac

SEQ ID NO: 149 - RNA, 89 nt, synthetic construct
  source 1..89: mol_type = other RNA
acagcttatttggaagctgaaatgtgaggtttataacactcacaagaatccttgtacatgaggatcacccatgtacaaggatgccaaac

SEQ ID NO: 150 - RNA, 93 nt, synthetic construct
  source 1..93: mol_type = other RNA
acagcttatttggaagctgaaatgtgaggtttataacactcacaagaatccttgtaaacatgaggatcacccatgtaaacaaggatgccaaac

SEQ ID NO: 151 - RNA, 56 nt, synthetic construct
  source 1..56: mol_type = other RNA
gtttgagaaccttatgaaattacaaggatgccaaactattaaatactcgtattgct

SEQ ID NO: 152 - RNA, 56 nt, synthetic construct; identical to SEQ ID NO: 151
  source 1..56: mol_type = other RNA

SEQ ID NO: 153 - RNA, 89 nt, synthetic construct
  source 1..89: mol_type = other RNA
acagcttatttggaagctgaaatgtgaggtttataacactcacaagaatcctgaaaaaggatgccaaactattaaatactcgtattgct

SEQ ID NO: 154 - RNA, 52 nt, synthetic construct; identical to SEQ ID NO: 85
  source 1..52: mol_type = other RNA

SEQ ID NO: 155 - RNA, 69 nt, synthetic construct
  source 1..69: mol_type = other RNA
tatatttgataaaaatatacagcttatttggaagctgaaatgtgaggtttataacactcacaagaatcc

SEQ ID NO: 156 - RNA, 20 nt, synthetic construct
  source 1..20: mol_type = other RNA
tcttcgcccagagcatccca

SEQ ID NO: 157 - RNA, 20 nt, synthetic construct
  source 1..20: mol_type = other RNA
agtctccaggaagaaattaa

SEQ ID NO: 158 - RNA, 108 nt, synthetic construct
  source 1..108: mol_type = other RNA
acatgaggatcacccatgtacagcttatttggaagctgaaatgtgaggtttataacactcacaagaatcctgaaaaaggatgccaaacaccaaaaaatatacgctata

SEQ ID NO: 159 - RNA, 112 nt, synthetic construct
  source 1..112: mol_type = other RNA
gcacatgaggatcacccatgtgcacagcttatttggaagctgaaatgtgaggtttataacactcacaagaatcctgaaaaaggatgccaaacaccaaaaaatatacgctata

SEQ ID NO: 160 - RNA, 118 nt, synthetic construct
  source 1..118: mol_type = other RNA
gcagcacatgaggatcacccatgtgctgcacagcttatttggaagctgaaatgtgaggtttataacactcacaagaatcctgaaaaaggatgccaaacaccaaaaaatatacgctata

SEQ ID NO: 161 - RNA, 128 nt, synthetic construct
  source 1..128: mol_type = other RNA
cgctatacgcacatgaggatcacccatgtgcgtatagcgacagcttatttggaagctgaaatgtgaggtttataacactcacaagaatcctgaaaaaggatgccaaacaccaaaaaatatacgctata

SEQ ID NO: 162 - RNA, 114 nt, synthetic construct
  source 1..114: mol_type = other RNA
gcacatgaggatcacccatgtgcaaacagcttatttggaagctgaaatgtgaggtttataacactcacaagaatcctgaaaaaggatgccaaacaccaaaaaatatacgctata

SEQ ID NO: 163 - RNA, 117 nt, synthetic construct
  source 1..117: mol_type = other RNA
gcacatgaggatcacccatgtgcaaaaaacagcttatttggaagctgaaatgtgaggtttataacactcacaagaatcctgaaaaaggatgccaaacaccaaaaaatatacgctata

SEQ ID NO: 164 - RNA, 122 nt, synthetic construct
  source 1..122: mol_type = other RNA
gcacatgaggatcacccatgtgcaaaaaaaaaaacagcttatttggaagctgaaatgtgaggtttataacactcacaagaatcctgaaaaaggatgccaaacaccaaaaaatatacgctata

SEQ ID NO: 165 - RNA, 110 nt, synthetic construct
  source 1..110: mol_type = other RNA
acatgaggatcacccatgtaaacagcttatttggaagctgaaatgtgaggtttataacactcacaagaatcctgaaaaaggatgccaaacaccaaaaaatatacgctata

SEQ ID NO: 166 - RNA, 113 nt, synthetic construct
  source 1..113: mol_type = other RNA
acatgaggatcacccatgtaaaaaacagcttatttggaagctgaaatgtgaggtttataacactcacaagaatcctgaaaaaggatgccaaacaccaaaaaatatacgctata

SEQ ID NO: 167 - RNA, 118 nt, synthetic construct
  source 1..118: mol_type = other RNA
acatgaggatcacccatgtaaaaaaaaaaacagcttatttggaagctgaaatgtgaggtttataacactcacaagaatcctgaaaaaggatgccaaacaccaaaaaatatacgctata

SEQ ID NO: 168 - RNA, 105 nt, synthetic construct
  source 1..105: mol_type = other RNA
acagcttatttggaagctgaaatgtgaggttacatgaggatcacccatgtaacactcacaagaatcctgaaaaaggatgccaaacaccaaaaaatatacgctata

SEQ ID NO: 169 - RNA, 109 nt, synthetic construct
  source 1..109: mol_type = other RNA
acagcttatttggaagctgaaatgtgaggtttaacatgaggatcacccatgtataacactcacaagaatcctgaaaaaggatgccaaacaccaaaaaatatacgctata

SEQ ID NO: 170 - RNA, 109 nt, synthetic construct
  source 1..109: mol_type = other RNA
acagcttatttggaagctgaaatgtgaggttgcacatgaggatcacccatgtgcaacactcacaagaatcctgaaaaaggatgccaaacaccaaaaaatatacgctata

SEQ ID NO: 171 - RNA, 115 nt, synthetic construct
  source 1..115: mol_type = other RNA
acagcttatttggaagctgaaatgtgaggttgcagcacatgaggatcacccatgtgctgcaacactcacaagaatcctgaaaaaggatgccaaacaccaaaaaatatacgctata

SEQ ID NO: 172 - RNA, 125 nt, synthetic construct
  source 1..125: mol_type = other RNA
acagcttatttggaagctgaaatgtgaggttcgctatacgcacatgaggatcacccatgtgcgtatagcgaacactcacaagaatcctgaaaaaggatgccaaacaccaaaaaatatacgctata

SEQ ID NO: 173 - RNA, 113 nt, synthetic construct
  source 1..113: mol_type = other RNA
acagcttatttggaagctgaaatgtgaggtttagcacatgaggatcacccatgtgcataacactcacaagaatcctgaaaaaggatgccaaacaccaaaaaatatacgctata

SEQ ID NO: 174 - RNA, 119 nt, synthetic construct
  source 1..119: mol_type = other RNA
acagcttatttggaagctgaaatgtgaggtttagcagcacatgaggatcacccatgtgctgcataacactcacaagaatcctgaaaaaggatgccaaacaccaaaaaatatacgctata

SEQ ID NO: 175 - RNA, 129 nt, synthetic construct
  source 1..129: mol_type = other RNA
acagcttatttggaagctgaaatgtgaggtttacgctatacgcacatgaggatcacccatgtgcgtatagcgataacactcacaagaatcctgaaaaaggatgccaaacaccaaaaaatatacgctata

SEQ ID NO: 176 - RNA, 113 nt, synthetic construct
  source 1..113: mol_type = other RNA
acagcttatttggaagctgaaatgtgaggtgtagcacatgaggatcacccatgtgcatcacactcacaagaatcctgaaaaaggatgccaaacaccaaaaaatatacgctata

SEQ ID NO: 177 - RNA, 113 nt, synthetic construct
  source 1..113: mol_type = other RNA
acagcttatttggaagctgaaatgtgaggtgaagcacatgaggatcacccatgtgcaacacactcacaagaatcctgaaaaaggatgccaaacaccaaaaaatatacgctata

SEQ ID NO: 178 - RNA, 105 nt, synthetic construct
  source 1..105: mol_type = other RNA
acagcttatttggaagctgaaatgtgaggtttataacactcacaagaatccttacatgaggatcacccatgtaaggatgccaaacaccaaaaaatatacgctata

SEQ ID NO: 179 - RNA, 107 nt, synthetic construct
  source 1..107: mol_type = other RNA
acagcttatttggaagctgaaatgtgaggtttataacactcacaagaatcctaaacatgaggatcacccatgtaaaggatgccaaacaccaaaaaatatacgctata

SEQ ID NO: 180 - RNA, 109 nt, synthetic construct
  source 1..109: mol_type = other RNA
acagcttatttggaagctgaaatgtgaggtttataacactcacaagaatccttgcacatgaggatcacccatgtgcaaggatgccaaacaccaaaaaatatacgctata

SEQ ID NO: 181 - RNA, 115 nt, synthetic construct
  source 1..115: mol_type = other RNA
acagcttatttggaagctgaaatgtgaggtttataacactcacaagaatccttgcagcacatgaggatcacccatgtgctgcaaggatgccaaacaccaaaaaatatacgctata

SEQ ID NO: 182 - RNA, 125 nt, synthetic construct
  source 1..125: mol_type = other RNA
acagcttatttggaagctgaaatgtgaggtttataacactcacaagaatccttcgctatacgcacatgaggatcacccatgtgcgtatagcgaaggatgccaaacaccaaaaaatatacgctata

SEQ ID NO: 183 - RNA, 108 nt, synthetic construct
  source 1..108: mol_type = other RNA
acagcttatttggaagctgaaatgtgaggtttataacactcacaagaatcctgcacatgaggatcacccatgtgcaaggatgccaaacaccaaaaaatatacgctata

SEQ ID NO: 184 - RNA, 114 nt, synthetic construct
  source 1..114: mol_type = other RNA
acagcttatttggaagctgaaatgtgaggtttataacactcacaagaatcctgcagcacatgaggatcacccatgtgctgcaaggatgccaaacaccaaaaaatatacgctata

SEQ ID NO: 185 - RNA, 117 nt, synthetic construct
  source 1..117: mol_type = other RNA
acagcttatttggaagctgaaatgtgaggtttataacactcacaagaatcctaagcagcacatgaggatcacccatgtgctgcaaaggatgccaaacaccaaaaaatatacgctata

SEQ ID NO: 186 - RNA, 127 nt, synthetic construct
  source 1..127: mol_type = other RNA
acagcttatttggaagctgaaatgtgaggtttataacactcacaagaatcctaacgctatacgcacatgaggatcacccatgtgcgtatagcgaaaggatgccaaacaccaaaaaatatacgctata

SEQ ID NO: 187 - RNA, 113 nt, synthetic construct
  source 1..113: mol_type = other RNA
acagcttatttggaagctgaaatgtgaggtttataacactcacaagaatccttgtaaacatgaggatcacccatgtttacaaggatgccaaacaccaaaaaatatacgctata

SEQ ID NO: 188 - RNA, 117 nt, synthetic construct
  source 1..117: mol_type = other RNA
acagcttatttggaagctgaaatgtgaggtttataacactcacaagaatccttgtaaaaacatgaggatcacccatgtaattacaaggatgccaaacaccaaaaaatatacgctata

SEQ ID NO: 189 - RNA, 109 nt, synthetic construct
  source 1..109: mol_type = other RNA
acagcttatttggaagctgaaatgtgaggtttataacactcacaagaatccttgtacatgaggatcacccatgtacaaggatgccaaacaccaaaaaatatacgctata

SEQ ID NO: 190 - RNA, 113 nt, synthetic construct
  source 1..113: mol_type = other RNA
acagcttatttggaagctgaaatgtgaggtttataacactcacaagaatccttgtaaacatgaggatcacccatgtaaacaaggatgccaaacaccaaaaaatatacgctata

SEQ ID NO: 191 - RNA, 108 nt, synthetic construct
  source 1..108: mol_type = other RNA
acagcttatttggaagctgaaatgtgaggtttataacactcacaagaatcctgaaaaaggatgccaaacaccaaaaaatatacgctataacatgaggatcacccatgt

SEQ ID NO: 192 - RNA, 115 nt, synthetic construct
  source 1..115: mol_type = other RNA
acagcttatttggaagctgaaatgtgaggtttataacactcacaagaatcctgaaaaaggatgccaaacaccaaaaaatatacgctataaaagcacatgaggatcacccatgtgc

SEQ ID NO: 193 - RNA, 118 nt, synthetic construct
  source 1..118: mol_type = other RNA
acagcttatttggaagctgaaatgtgaggtttataacactcacaagaatcctgaaaaaggatgccaaacaccaaaaaatatacgctataaaaaaagcacatgaggatcacccatgtgc

SEQ ID NO: 194 - RNA, 122 nt, synthetic construct
  source 1..122: mol_type = other RNA
acagcttatttggaagctgaaatgtgaggtttataacactcacaagaatcctgaaaaaggatgccaaacaccaaaaaatatacgctataaaaaaaaaaagcacatgaggatcacccatgtgc

SEQ ID NO: 195 - RNA, 123 nt, synthetic construct
  source 1..123: mol_type = other RNA
acagcttatttggaagctgaaatgtgaggtttataacactcacaagaatcctgaaaaaggatgccaaacaccaaaaaatatacgctataaaaaagcagcacatgaggatcacccatgtgctgc

SEQ ID NO: 196 - RNA, 121 nt, synthetic construct
  source 1..121: mol_type = other RNA
tatatttacatgaggatcacccatgtaaatatacagcttatttggaagctgaaatgtgaggtttataacactcacaagaatcctgaaaaaggatgccaaacaccaaaaaatatacgctata

SEQ ID NO: 197 - RNA, 125 nt, synthetic construct
  source 1..125: mol_type = other RNA
tatatttgaacatgaggatcacccatgtaaaaatatacagcttatttggaagctgaaatgtgaggtttataacactcacaagaatcctgaaaaaggatgccaaacaccaaaaaatatacgctata

SEQ ID NO: 198 - RNA, 125 nt, synthetic construct
  source 1..125: mol_type = other RNA
tatatttgcacatgaggatcacccatgtgcaaatatacagcttatttggaagctgaaatgtgaggtttataacactcacaagaatcctgaaaaaggatgccaaacaccaaaaaatatacgctata

SEQ ID NO: 199 - RNA, 131 nt, synthetic construct
  source 1..131: mol_type = other RNA
tatatttgcagcacatgaggatcacccatgtgctgcaaatatacagcttatttggaagctgaaatgtgaggtttataacactcacaagaatcctgaaaaaggatgccaaacaccaaaaaatatacgctata

SEQ ID NO: 200 - RNA, 141 nt, synthetic construct
  source 1..141: mol_type = other RNA
tatatttcgctatacgcacatgaggatcacccatgtgcgtatagcgaaatatacagcttatttggaagctgaaatgtgaggtttataacactcacaagaatcctgaaaaaggatgccaaacaccaaaaaatatacgctata

SEQ ID NO: 201 - RNA, 129 nt, synthetic construct
  source 1..129: mol_type = other RNA
tatatttgagcacatgaggatcacccatgtgcaaaaatatacagcttatttggaagctgaaatgtgaggtttataacactcacaagaatcctgaaaaaggatgccaaacaccaaaaaatatacgctata

SEQ ID NO: 202 - RNA, 135 nt, synthetic construct
  source 1..135: mol_type = other RNA
tatatttgagcagcacatgaggatcacccatgtgctgcaaaaatatacagcttatttggaagctgaaatgtgaggtttataacactcacaagaatcctgaaaaaggatgccaaacaccaaaaaatatacgctata

SEQ ID NO: 203 - RNA, 145 nt, synthetic construct
  source 1..145: mol_type = other RNA
tatatttgacgctatacgcacatgaggatcacccatgtgcgtatagcgaaaaatatacagcttatttggaagctgaaatgtgaggtttataacactcacaagaatcctgaaaaaggatgccaaacaccaaaaaatatacgctata

SEQ ID NO: 204 - RNA, 89 nt, synthetic construct
  source 1..89: mol_type = other RNA
acagcttatttggaagctgaaatgtgaggtttataacactcacaagaatcctgaaaaaggatgccaaacaccaaaaaatatacgctata

SEQ ID NO: 205 - RNA, 90 nt, synthetic construct
  source 1..90: mol_type = other RNA
acagcttatttggaagctgaaatgtgaggtttataacactcacaagaatccttgaaaaaggatgccaaacaccaaaaaatatacgctata

SEQ ID NO: 206 - RNA, 107 nt, synthetic construct
  source 1..107: mol_type = other RNA
tatatttgataaaaatatacagcttatttggaagctgaaatgtgaggtttataacactcacaagaatcctgaaaaaggatgccaaacaccaaaaaatatacgctata

SEQ ID NO: 207 - RNA, 108 nt, synthetic construct
  source 1..108: mol_type = other RNA
tatatttgataaaaatatacagcttatttggaagctgaaatgtgaggtttataacactcacaagaatccttgaaaaaggatgccaaacaccaaaaaatatacgctata

SEQ ID NO: 208 - RNA, 108 nt, synthetic construct
  source 1..108: mol_type = other RNA
gcgcgaggatcacccgcgcacagcttatttggaagctgaaatgtgaggtttataacactcacaagaatcctgaaaaaggatgccaaacaccaaaaaatatacgctata

SEQ ID NO: 209 - RNA, 109 nt, synthetic construct
  source 1..109: mol_type = other RNA
acagcttatttggaagctgaaatgtgaggtttagcgcgaggatcacccgcgcataacactcacaagaatcctgaaaaaggatgccaaacaccaaaaaatatacgctata

SEQ ID NO: 210 - RNA, 105 nt, synthetic construct
  source 1..105: mol_type = other RNA
acagcttatttggaagctgaaatgtgaggtttataacactcacaagaatccttgcgcgaggatcacccgcgcaaggatgccaaacaccaaaaaatatacgctata

SEQ ID NO: 211 - RNA, 111 nt, synthetic construct
  source 1..111: mol_type = other RNA
acagcttatttggaagctgaaatgtgaggtttataacactcacaagaatcctgaaaaaggatgccaaacaccaaaaaatatacgctataaaagcgcgaggatcacccgcgc

SEQ ID NO: 212 - RNA, 125 nt, synthetic construct
  source 1..125: mol_type = other RNA
tatatttgagcgcgaggatcacccgcgcaaaaatatacagcttatttggaagctgaaatgtgaggtttataacactcacaagaatcctgaaaaaggatgccaaacaccaaaaaatatacgctata

SEQ ID NO: 213 - RNA, 114 nt, synthetic construct
  source 1..114: mol_type = other RNA
ggacatgaggatcacccatgtccgaacagcttatttggaagctgaaatgtgaggtttataacactcacaagaatcctgaaaaaggatgccaaacaccaaaaaatatacgctata

SEQ ID NO: 214 - RNA, 111 nt, synthetic construct
  source 1..111: mol_type = other RNA
acagcttatttggaagctgaaatgtgaggtttggacatgaggatcacccatgtccaaacactcacaagaatcctgaaaaaggatgccaaacaccaaaaaatatacgctata

SEQ ID NO: 215 - RNA, 108 nt, synthetic construct
  source 1..108: mol_type = other RNA
acagcttatttggaagctgaaatgtgaggtttataacactcacaagaatcctggacatgaggatcacccatgtccaaggatgccaaacaccaaaaaatatacgctata

SEQ ID NO: 216 - RNA, 112 nt, synthetic construct
  source 1..112: mol_type = other RNA
acagcttatttggaagctgaaatgtgaggtttataacactcacaagaatcctgaaaaaggatgccaaacaccaaaaaatatacgctataggacatgaggatcacccatgtcc

SEQ ID NO: 217 - protein, 26 aa, synthetic construct
GSGGSPAGSPTSTEEGTSESATPGSG

SEQ ID NO: 218 - RNA, 19 nt, synthetic construct
  source 1..19: mol_type = other RNA
acatgaggatcacccatgt

SEQ ID NO: 219 - protein, 17 aa, unidentified
MAPKKKRKVGIHGVPAA

SEQ ID NO: 220 - protein, 130 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 221 - protein, 475 aa, synthetic construct
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIV

SEQ ID NO: 222 - RNA, 20 nt, synthetic construct
  source 1..20: mol_type = other RNA
gggttttgtcctcactctga

SEQ ID NO: 223 - RNA, 20 nt, synthetic construct
  source 1..20: mol_type = other RNA
tagaaccttgatgacatagc

SEQ ID NO: 224 - RNA, 20 nt, synthetic construct
  source 1..20: mol_type = other RNA
cttactttaagtgaagttac

SEQ ID NO: 225 - RNA, 20 nt, synthetic construct
  source 1..20: mol_type = other RNA
ttttctacttactttaagtg

SEQ ID NO: 226 - RNA, 20 nt, synthetic construct
  source 1..20: mol_type = other RNA
tccagacttttgtagaaaaa

SEQ ID NO: 227 - RNA, 20 nt, synthetic construct
  source 1..20: mol_type = other RNA
aaatactgacttacctgatt

SEQ ID NO: 228 - RNA, 20 nt, synthetic construct
  source 1..20: mol_type = other RNA
tcagctcagaaggactagta

SEQ ID NO: 229 - RNA, 20 nt, synthetic construct
  source 1..20: mol_type = other RNA
tcttaccatcatgttttaca

SEQ ID NO: 230 - RNA, 20 nt, synthetic construct
  source 1..20: mol_type = other RNA
ttgattctaggcattcctgc

SEQ ID NO: 231 - RNA, 20 nt, synthetic construct
  source 1..20: mol_type = other RNA
ttcaggtagtccatggacat

SEQ ID NO: 232 - RNA, 20 nt, synthetic construct
  source 1..20: mol_type = other RNA
gtccccttaccatcaagcct

SEQ ID NO: 233 - RNA, 20 nt, synthetic construct
  source 1..20: mol_type = other RNA
aaacttttcttttcaggaga

SEQ ID NO: 234 - RNA, 20 nt, synthetic construct
  source 1..20: mol_type = other RNA
tcagaaaaagatacctgaat

SEQ ID NO: 235 - RNA, 20 nt, synthetic construct
  source 1..20: mol_type = other RNA
tctcctttaggaggctggtg

SEQ ID NO: 236 - RNA, 20 nt, synthetic construct
  source 1..20: mol_type = other RNA
tcttgtttttctacaaaagt

SEQ ID NO: 237 - RNA, 20 nt, synthetic construct
  source 1..20: mol_type = other RNA
aaagaaatagaaaatcaggt

SEQ ID NO: 238 - RNA, 20 nt, synthetic construct
  source 1..20: mol_type = other RNA
aatactagtccttctgagct

SEQ ID NO: 239 - RNA, 20 nt, synthetic construct
  source 1..20: mol_type = other RNA
agaaatgtaaaacatgatgg

SEQ ID NO: 240 - RNA, 20 nt, synthetic construct
  source 1..20: mol_type = other RNA
cattcagcaggaatgcctag

SEQ ID NO: 241 - RNA, 20 nt, synthetic construct
  source 1..20: mol_type = other RNA
gtggtacattcagcaggaat

SEQ ID NO: 242 - RNA, 20 nt, synthetic construct
  source 1..20: mol_type = other RNA
aattaatgtccatggactac

SEQ ID NO: 243 - RNA, 20 nt, synthetic construct
  source 1..20: mol_type = other RNA
gttttgggaggcttgatggt

SEQ ID NO: 244 - RNA, 20 nt, synthetic construct
  source 1..20: mol_type = other RNA
ggcccaaccaaaattctcct

SEQ ID NO: 245 - RNA, 20 nt, synthetic construct
  source 1..20: mol_type = other RNA
tccagagggttattcaggta

SEQ ID NO: 246 - RNA, 20 nt, synthetic construct
  source 1..20: mol_type = other RNA
aaaatgttcacaattaagct

SEQ ID NO: 247 - RNA, 20 nt, synthetic construct
  source 1..20: mol_type = other RNA
aaattgaaaatcaagataaa

SEQ ID NO: 248 - RNA, 20 nt, synthetic construct
  source 1..20: mol_type = other RNA
aacatttttatcttgatttt

SEQ ID NO: 249 - RNA, 20 nt, synthetic construct
  source 1..20: mol_type = other RNA
cttacgggcagaggccagga

SEQ ID NO: 250 - RNA, 20 nt, synthetic construct
  source 1..20: mol_type = other RNA
ctctttcctcaggagcttca

SEQ ID NO: 251 - RNA, 20 nt, synthetic construct
  source 1..20: mol_type = other RNA
atttaggggctgggtgaccg

SEQ ID NO: 252 - RNA, 20 nt, synthetic construct
  source 1..20: mol_type = other RNA
actgatttaggggctgggtg

SEQ ID NO: 253 - RNA, 20 nt, synthetic construct
  source 1..20: mol_type = other RNA
cttcccctgactgatttagg

SEQ ID NO: 254 - RNA, 20 nt, synthetic construct
  source 1..20: mol_type = other RNA
gaggcagctgctccaggtaa

SEQ ID NO: 255 - RNA, 20 nt, synthetic construct
  source 1..20: mol_type = other RNA
catggcacctctgttcctgc

SEQ ID NO: 256 - RNA, 20 nt, synthetic construct
  source 1..20: mol_type = other RNA
gcgctcctggcctctgcccg

SEQ ID NO: 257 - RNA, 20 nt, synthetic construct
  source 1..20: mol_type = other RNA
aagccatcggtcacccagcc

SEQ ID NO: 258 - RNA, 20 nt, synthetic construct
  source 1..20: mol_type = other RNA
caggaacagaggtgccatgc

SEQ ID NO: 259 - RNA, 20 nt, synthetic construct
  source 1..20: mol_type = other RNA
cccggggctgcatggcacct

SEQ ID NO: 260 - RNA, 20 nt, synthetic construct
  source 1..20: mol_type = other RNA
cacccgcaccttggcgcagc

SEQ ID NO: 261 - RNA, 20 nt, synthetic construct
  source 1..20: mol_type = other RNA
gggccaggatccgtggaggt

SEQ ID NO: 262 - RNA, 20 nt, synthetic construct
  source 1..20: mol_type = other RNA
gctcaccagctccagcaggt

SEQ ID NO: 263 - RNA, 20 nt, synthetic construct
  source 1..20: mol_type = other RNA
gcttctgcaggccttgaagt

SEQ ID NO: 264 - RNA, 20 nt, synthetic construct
  source 1..20: mol_type = other RNA
ggggtcttaccggggggctg

SEQ ID NO: 265 - RNA, 20 nt, synthetic construct
  source 1..20: mol_type = other RNA
gaaagacggaggcagcctgg

SEQ ID NO: 266 - RNA, 20 nt, synthetic construct
  source 1..20: mol_type = other RNA
cttacctgtctgtggaagcg

SEQ ID NO: 267 - RNA, 20 nt, synthetic construct
  source 1..20: mol_type = other RNA
ttcgtcgagcaggccagcaa

SEQ ID NO: 268 - RNA, 20 nt, synthetic construct
  source 1..20: mol_type = other RNA
gggccatcacttacctatga

SEQ ID NO: 269 - RNA, 20 nt, synthetic construct
  source 1..20: mol_type = other RNA
ttcctcccaggcctggagtt

SEQ ID NO: 270 - RNA, 20 nt, synthetic construct
  source 1..20: mol_type = other RNA
atgacctggaaaggtgagga

SEQ ID NO: 271 - RNA, 20 nt, synthetic construct
  source 1..20: mol_type = other RNA
caccaggcattgcagccatg

SEQ ID NO: 272 - RNA, 20 nt, synthetic construct
  source 1..20: mol_type = other RNA
cttacctgccccatgggtgc

SEQ ID NO: 273 - RNA, 20 nt, synthetic construct
  source 1..20: mol_type = other RNA
cagtcacctccatgcgctcg

SEQ ID NO: 274 - RNA, 20 nt, synthetic construct
  source 1..20: mol_type = other RNA
actctaaggcccaagggggc

SEQ ID NO: 275 - RNA, 20 nt, synthetic construct
  source 1..20: mol_type = other RNA
ccccaggctgcagctcccac

SEQ ID NO: 276 - RNA, 20 nt, synthetic construct
  source 1..20: mol_type = other RNA
gcaggtgaccgtggcctgcg

SEQ ID NO: 277 - RNA, 20 nt, synthetic construct
  source 1..20: mol_type = other RNA
cctcgccgcggcacaggtgg

SEQ ID NO: 278 - RNA, 20 nt, synthetic construct
  source 1..20: mol_type = other RNA
ccaggcaacctccacggatc

SEQ ID NO: 279 - RNA, 20 nt, synthetic construct
  source 1..20: mol_type = other RNA
gcgacctgctggagctggtg

SEQ ID NO: 280 - RNA, 20 nt, synthetic construct
  source 1..20: mol_type = other RNA
agtggcgacctgctggagct

SEQ ID NO: 281 - RNA, 20 nt, synthetic construct
  source 1..20: mol_type = other RNA
actgtcacacttgctggcct

SEQ ID NO: 282 - RNA, 20 nt, synthetic construct
  source 1..20: mol_type = other RNA
ctccccagcctcagctcccg

SEQ ID NO: 283 - RNA, 20 nt, synthetic construct
  source 1..20: mol_type = other RNA
gccccaactgtgatgacctg

SEQ ID NO: 284 - RNA, 20 nt, synthetic construct
  source 1..20: mol_type = other RNA
ccccccagcacccatggggc

SEQ ID NO: 285 - RNA, 20 nt, synthetic construct
  source 1..20: mol_type = other RNA
caaaacagctgccaacctgc

SEQ ID NO: 286 - RNA, 20 nt, synthetic construct
  source 1..20: mol_type = other RNA
cccatgagggccaggggaga

SEQ ID NO: 287 - RNA, 20 nt, synthetic construct
  source 1..20: mol_type = other RNA
gccctcatgggcaccgtcag

SEQ ID NO: 288 - RNA, 20 nt, synthetic construct
  source 1..20: mol_type = other RNA
ccgtaccgggaatgcacgtc

SEQ ID NO: 289 - RNA, 20 nt, synthetic construct
  source 1..20: mol_type = other RNA
aagagtgaaggcccatacct

SEQ ID NO: 290 - RNA, 20 nt, synthetic construct
  source 1..20: mol_type = other RNA
tttttcctcctatagattcc

SEQ ID NO: 291 - RNA, 20 nt, synthetic construct
  source 1..20: mol_type = other RNA
gtagactctcacttaccttc

SEQ ID NO: 292 - RNA, 20 nt, synthetic construct
  source 1..20: mol_type = other RNA
atgtcttttaggacttggca

SEQ ID NO: 293 - RNA, 20 nt, synthetic construct
  source 1..20: mol_type = other RNA
aacttgttgaaatcaacctt

SEQ ID NO: 294 - RNA, 20 nt, synthetic construct
  source 1..20: mol_type = other RNA
aaatcaacctttgccagaga

SEQ ID NO: 295 - RNA, 20 nt, synthetic construct
  source 1..20: mol_type = other RNA
ttccccttttctctagacta

SEQ ID NO: 296 - RNA, 20 nt, synthetic construct
  source 1..20: mol_type = other RNA
ttttctcctttttcatagtg

SEQ ID NO: 297 - RNA, 20 nt, synthetic construct
  source 1..20: mol_type = other RNA
cttaccactggaatctccat

SEQ ID NO: 298 - RNA, 20 nt, synthetic construct
  source 1..20: mol_type = other RNA
ctatttcttttagtgcctgt

SEQ ID NO: 299 - RNA, 20 nt, synthetic construct
  source 1..20: mol_type = other RNA
tgaacctctttccactgttc

SEQ ID NO: 300 - RNA, 20 nt, synthetic construct
  source 1..20: mol_type = other RNA
aagaagagtgcagatacact

SEQ ID NO: 301 - RNA, 20 nt, synthetic construct
  source 1..20: mol_type = other RNA
gacctaccttaatcatggtg

SEQ ID NO: 302 - RNA, 20 nt, synthetic construct
  source 1..20: mol_type = other RNA
ttccttttccacgctaaggt

SEQ ID NO: 303 - RNA, 20 nt, synthetic construct
  source 1..20: mol_type = other RNA
gacttggaaccaaaaggaat

SEQ ID NO: 304 - RNA, 20 nt, synthetic construct
  source 1..20: mol_type = other RNA
ccatcagtatcttaatgaag

SEQ ID NO: 305 - RNA, 20 nt, synthetic construct
  source 1..20: mol_type = other RNA
tgccatcagtatcttaatga

SEQ ID NO: 306 - RNA, 20 nt, synthetic construct
  source 1..20: mol_type = other RNA
gagtttgcagttacattata

SEQ ID NO: 307 - RNA, 20 nt, synthetic construct
  source 1..20: mol_type = other RNA
gggaacatggagattccagt

SEQ ID NO: 308 - RNA, 20 nt, synthetic construct
  source 1..20: mol_type = other RNA
aggaacagtggaaagaggtt

SEQ ID NO: 309 - RNA, 20 nt, synthetic construct
  source 1..20: mol_type = other RNA
agtttgatcacctcataagc

SEQ ID NO: 310 - RNA, 20 nt, synthetic construct
  source 1..20: mol_type = other RNA
gatcccccaaagtgtatctg

SEQ ID NO: 311 - RNA, 20 nt, synthetic construct
  source 1..20: mol_type = other RNA
gagttgccatattggacttg

SEQ ID NO: 312 - RNA, 20 nt, synthetic construct
  source 1..20: mol_type = other RNA
cattaccttattaatatccc

SEQ ID NO: 313 - RNA, 20 nt, synthetic construct
  source 1..20: mol_type = other RNA
tttcttcctaattacagcgc

SEQ ID NO: 314 - RNA, 20 nt, synthetic construct
  source 1..20: mol_type = other RNA
attacagcgcggtgtggagg

SEQ ID NO: 315 - RNA, 20 nt, synthetic construct
  source 1..20: mol_type = other RNA
aacctgcagtctcacctgat

SEQ ID NO: 316 - RNA, 20 nt, synthetic construct
  source 1..20: mol_type = other RNA
ttctttcaatttcaaaaggt

SEQ ID NO: 317 - RNA, 20 nt, synthetic construct
  source 1..20: mol_type = other RNA
actcacactcacccaaaaat

SEQ ID NO: 318 - RNA, 20 nt, synthetic construct
  source 1..20: mol_type = other RNA
caatgatccttaagatcaca

SEQ ID NO: 319 - RNA, 20 nt, synthetic construct
  source 1..20: mol_type = other RNA
atacttaccaatatgggatg

SEQ ID NO: 320 - RNA, 20 nt, synthetic construct
  source 1..20: mol_type = other RNA
accttaccttgtgcttggat

SEQ ID NO: 321 - RNA, 20 nt, synthetic construct
  source 1..20: mol_type = other RNA
atcacagattatggcctgta

SEQ ID NO: 322 - RNA, 20 nt, synthetic construct
  source 1..20: mol_type = other RNA
gttctgtgggatattaataa

SEQ ID NO: 323 - RNA, 20 nt, synthetic construct
  source 1..20: mol_type = other RNA
cagtttcctccacaccgcgc

SEQ ID NO: 324 - RNA, 20 nt, synthetic construct
  source 1..20: mol_type = other RNA
tcgctctctaaatcaggtga

SEQ ID NO: 325 - RNA, 20 nt, synthetic construct
  source 1..20: mol_type = other RNA
catcacccacttctttcttc

SEQ ID NO: 326 - RNA, 20 nt, synthetic construct
  source 1..20: mol_type = other RNA
gaagaagtgcttttgtgatc

SEQ ID NO: 327 - RNA, 20 nt, synthetic construct
  source 1..20: mol_type = other RNA
tctccaatacaggccataat

SEQ ID NO: 328 - RNA, 20 nt, synthetic construct
  source 1..20: mol_type = other RNA
taggatgatgttcatggctt

SEQ ID NO: 329 - RNA, 20 nt, synthetic construct
  source 1..20: mol_type = other RNA
aaaaataaattttcttacct

SEQ ID NO: 330 - RNA, 20 nt, synthetic construct
  source 1..20: mol_type = other RNA
attctagatggaagctgtat

SEQ ID NO: 331 - RNA, 20 nt, synthetic construct
  source 1..20: mol_type = other RNA
gtgacaatcttcctcctacc

SEQ ID NO: 332 - RNA, 20 nt, synthetic construct
  source 1..20: mol_type = other RNA
atcatcccctttctttctca

SEQ ID NO: 333 - RNA, 20 nt, synthetic construct
  source 1..20: mol_type = other RNA
gttacctgagttgtggcggc

SEQ ID NO: 334 - RNA, 20 nt, synthetic construct
  source 1..20: mol_type = other RNA
catttcttattttaggatga

SEQ ID NO: 335 - RNA, 20 nt, synthetic construct
  source 1..20: mol_type = other RNA
aacgaacctctcaaaatgcc

SEQ ID NO: 336 - RNA, 20 nt, synthetic construct
  source 1..20: mol_type = other RNA
ctctctccaggtgatgatgc

SEQ ID NO: 337 - RNA, 20 nt, synthetic construct
  source 1..20: mol_type = other RNA
ccaaaactcacagtggctgg

SEQ ID NO: 338 - RNA, 20 nt, synthetic construct
  source 1..20: mol_type = other RNA
aacttttctttccccagatt

SEQ ID NO: 339 - RNA, 20 nt, synthetic construct
  source 1..20: mol_type = other RNA
tccagttacctggaaagcta

SEQ ID NO: 340 - RNA, 20 nt, synthetic construct
  source 1..20: mol_type = other RNA
tcttttcacttggttagggg

SEQ ID NO: 341 - RNA, 20 nt, synthetic construct
  source 1..20: mol_type = other RNA
ttgcagcattttccaggtaa

SEQ ID NO: 342 - RNA, 20 nt, synthetic construct
  source 1..20: mol_type = other RNA
ataatattgcagcattttcc

SEQ ID NO: 343 - RNA, 20 nt, synthetic construct
  source 1..20: mol_type = other RNA
cccgttcccagggactgaca

SEQ ID NO: 344 - RNA, 20 nt, synthetic construct
  source 1..20: mol_type = other RNA
gtttcaaaatttttcatcct

SEQ ID NO: 345 - RNA, 20 nt, synthetic construct
  source 1..20: mol_type = other RNA
caaagggcattttgagaggt

SEQ ID NO: 346 - RNA, 20 nt, synthetic construct
  source 1..20: mol_type = other RNA
acagcctccctggcatcatc

SEQ ID NO: 347 - RNA, 20 nt, synthetic construct
  source 1..20: mol_type = other RNA
gggcttagctttccaggtaa

SEQ ID NO: 348 - RNA, 20 nt, synthetic construct
  source 1..20: mol_type = other RNA
ggatagcaataacctgtgaa

SEQ ID NO: 349 - RNA, 20 nt, synthetic construct
  source 1..20: mol_type = other RNA
aaaatgctcccccggctaat

SEQ ID NO: 350 - RNA, 20 nt, synthetic construct
  source 1..20: mol_type = other RNA
ttcgacaaaatacctctatc

SEQ ID NO: 351 - RNA, 20 nt, synthetic construct
  source 1..20: mol_type = other RNA
tccgaggtaaaaccacctct

SEQ ID NO: 352 - RNA, 20 nt, synthetic construct
  source 1..20: mol_type = other RNA
ctcaaaacaggtttccagtt

SEQ ID NO: 353 - RNA, 20 nt, synthetic construct
  source 1..20: mol_type = other RNA
cttaccatcctcctgaagat

SEQ ID NO: 354 - RNA, 20 nt, synthetic construct
  source 1..20: mol_type = other RNA
ttgaagcctgctataactgc

SEQ ID NO: 355 - RNA, 20 nt, synthetic construct
  source 1..20: mol_type = other RNA
acaccttacctatagcactt

SEQ ID NO: 356 - RNA, 20 nt, synthetic construct
  source 1..20: mol_type = other RNA
atttcttttttaggtgtggt

SEQ ID NO: 357 - RNA, 20 nt, synthetic construct
  source 1..20: mol_type = other RNA
cgggcccgctccgacgcgga

SEQ ID NO: 358 - RNA, 20 nt, synthetic construct
  source 1..20: mol_type = other RNA
gttttacctcggatagaggt

SEQ ID NO: 359 - RNA, 20 nt, synthetic construct
  source 1..20: mol_type = other RNA
agaagccgtggcagaggtgg

SEQ ID NO: 360 - RNA, 20 nt, synthetic construct
  source 1..20: mol_type = other RNA
gaatgagaagccgtggcaga

SEQ ID NO: 361 - RNA, 20 nt, synthetic construct
  source 1..20: mol_type = other RNA
tgatcttcaggaggatggta

SEQ ID NO: 362 - RNA, 20 nt, synthetic construct
  source 1..20: mol_type = other RNA
ccgcagttatagcaggcttc

SEQ ID NO: 363 - RNA, 20 nt, synthetic construct
  source 1..20: mol_type = other RNA
caccaaagtgaagtgctata

SEQ ID NO: 364 - RNA, 20 nt, synthetic construct
  source 1..20: mol_type = other RNA
accagtttcaccacacctaa

SEQ ID NO: 365 - RNA, 20 nt, synthetic construct
  source 1..20: mol_type = other RNA
catgaccagtttcaccacac

SEQ ID NO: 366 - RNA, 20 nt, synthetic construct
  source 1..20: mol_type = other RNA
attttagcagcacctgagca

SEQ ID NO: 367 - RNA, 20 nt, synthetic construct
  source 1..20: mol_type = other RNA
agattaatgacatacgcatt

SEQ ID NO: 368 - RNA, 20 nt, synthetic construct
  source 1..20: mol_type = other RNA
gctttgctcaggtgctgcta

SEQ ID NO: 369 - RNA, 40 nt, synthetic construct
  source 1..40: mol_type = other RNA
aggagagaaaagaagagtaagaagaaatataagagccacc

SEQ ID NO: 370 - RNA, 101 nt, synthetic construct
  source 1..101: mol_type = other RNA
ttaattaagctgccttctgcggggcttgccttctggccatgcccttcttctctcccttgcacctgtacctcttggtctttgaataaagcctgagtaggaag

SEQ ID NO: 371 - RNA, 1708 nt, synthetic construct
  source 1..1708: mol_type = other RNA
aggagagaaaagaagagtaagaagaaatataagagccaccatggccccaaagaagaagcggaaggtcggtatccacggagtcccagcagccatgtccgtcttaacccgaaaggtgcaactgatacctgtgggggacaaggaggagagggaccgagtgtacaagtacctccgtgatggcatagaggcccaaaacagggctatgaacctctacatgtctggcctatacttcgccgccataaacgaggcatccaaggaggacagaaaggagctgaaccaactctacagcagaatagccacctccagcaagggctctgcctacactactgacattgaattccctacaggtctggccagcacctctaccctcagcatggccgtgaggcaagactttactaagagcttaaaagacggcttgatgtacgggcgtgtgtccctacccacctatcgaaaggacaaccctctgttcgtggacgtcagattcgttgccttacgggggacgaagcaaaagtacaacggcttgtaccatgagtacaagtcccacaccgagtttctggacaacctgtacagctctgacctgaaggtctacatcaagtttgccaacgacattacgtttcaggtcatcttcgggaacccacgcaaaagctccgctcttaggtcagagttccagaatatcttcgaggagtactacaaggtgtgtcagtcgtccattcagttctctgggaccaaaatcatcctgaacatggcgatgagaatcccagataaggagatcgagttggatgaagatgtgtgcgtcggagttgatctgggcattgctattcccgcggtgtgcgctcttaacaaaaatagatactcacgtgtcagcattggttcaaaagaggactttctgagggtgcgcactaaaatcaggaatcaacgcaaacggctgcagacaaacctgaaatcgtcaaacggtggacacggacggaagaagaagatgaagcccatggatcggtttagagattatgaagcgaattgggtgcagaattataaccactatgttagtcggcaggtagttgattttgcagttaaaaataaggccaaatatattaatctcgaaaatctcgaaggcatccgcgatgatgtaaagaatgaatggcttcttagtaactggagttattatcagttgcagcagtatattacatataaagctaaaacatatgggatcgaagtaagaaaaattaatccgtatcatacatcacagcgctgtagttgctgcggatatgaagatgctggaaatagacccaaaaaggaaaaaggacaggcatatttcaaatgtttaaagtgtggtgaggagatgaatgcagattttaatgctgcacggaacatcgcaatgtctacagaatttcagagtggcaagaaaactaagaaacagaaaaaggaacagcacgaaaataaaaaaaggccggcggccacgaaaaaggccggccaggcaaaaaagaaaaagtgattaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaat

SEQ ID NO: 372 - RNA, 41 nt, synthetic construct
  source 1..41: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 39: mod_base = OTHER; note = 2'-O-methyl modified nucleotide with phosphorothioate linkage
  modified_base 40: mod_base = OTHER; note = 2'-O-methyl modified nucleotide with phosphorothioate linkage
  modified_base 41: mod_base = OTHER; note = 2'-O-methyl modified nucleotide
atagattgctccttacgaggagacgagcaacggcggaaggt

SEQ ID NO: 373 - protein, 166 aa, synthetic construct
SEVEFSHEYWMRHALTLAKRARDERAGPVGAVLVLNNRVIGEGWNRAIGLHDPTAHAEIMALRQGGLVMQNHRLIDATLYVTFEPCVMCAGAMINSRIGRVVFGVRNSKRGAAGSLMNVLNYPGMNHRVEITEGILADECAALLCDFYRMPRHVFNSQKKAQSSIN

SEQ ID NO: 374 - protein, 166 aa, synthetic construct
SEVEFSHEYWMRHALTLAKRARDEGEAPVGAVLVLNNRVIGEGWNRRIGLHDPTAHAEIMALRQGGLVMQNSRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 375 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFAAARNIAMSTEFQSGKKTKKQKKEQHENK

SEQ ID NO: 376 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARAIAMSTEFQSGKKTKKQKKEQHENK

SEQ ID NO: 377 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYAKCLKCGEEMNADFNAARNIAMSTEFQSGKKTKKQKKEQHENK

SEQ ID NO: 378 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRASCCGYEDAGNRPKKEKGQAYFKCLKCGEEMNADFNAARNIAMSTEFQSGKKTKKQKKEQHENK

SEQ ID NO: 379 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTAQRCSCCGYEDAGNRPKKEKGQAYFKCLKCGEEMNADFNAARNIAMSTEFQSGKKTKKQKKEQHENK

SEQ ID NO: 380 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHASQRCSCCGYEDAGNRPKKEKGQAYFKCLKCGEEMNADFNAARNIAMSTEFQSGKKTKKQKKEQHENK

SEQ ID NO: 381 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENAEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 382 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLEALEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 383 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPAHTSQRCSCCGYEDAGNRPKKEKGQAYFKCLKCGEEMNADFNAARNIAMSTEFQSGKKTKKQKKEQHENK

SEQ ID NO: 384 - protein, 448 aa, synthetic construct; identical to SEQ ID NO: 376

SEQ ID NO: 385 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 386 - RNA, 20 nt, synthetic construct
  source 1..20: mol_type = other RNA
agtgtataagcatatcaata

SEQ ID NO: 387 - protein, 6 aa, synthetic construct
HHHHHH

SEQ ID NO: 388 - protein, 166 aa, synthetic construct
SEVEFSHEYWMRHALTLAKRARDEGAGPVGAVLVLNNRVIGEGWNRAIGLHDPTAHAEIMALRQGGLVMQNYRLFDATLYVTFEPCVMCAGAMINSRIGRVVFGVRNSKRGAAGSLMNVLNYPGMNHRVEITEGILADECAALLCDFYRIPRQVFNSQKKAQSSIN

SEQ ID NO: 389 - protein, 166 aa, synthetic construct
SEVEFSHEYWMRHALTLAKRARDERKAPVGAVLVLNNRVIGEGWNRAIGLHDPTAHAEIIALRQGGLVMQNYRLIDATLYVTFEPCVMCAGAMINSRIGRVVFGVRNSKRGAAGSLMNVLNYPGMNHRVEITEGILADECAALLCDFYRMPRQVFNAQKKAQSSIN

SEQ ID NO: 390 - protein, 166 aa, synthetic construct
SEVEFSHEYWMRHALTLAKRARDERSVPVGAVLVLNNRVIGEGWNRAIGLHDPTAHAEIMALRQGGLVMQNYRLYDATLYTTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 391 - protein, 222 aa, synthetic construct
DLASLRAQQIELASSVIREDRLDKDPPDLIAGADVGFEQGGEVTRAAMVLLKYPSLELVEYKVARIATTMPYIPGFLSFREYPALLAAWEMLSQKPDLVFVDGHGISHPRRLGVASHFGLLVDVPTIGVAKKRLCGKFEPLSSEPGALAPLMDKGEQLAWVWRSKARCNPLFIATGHRVSVDSALAWVQRCMKGYRLPEPTRWADAVASERPAFVRYTANQP

SEQ ID NO: 392 - protein, 409 aa, synthetic construct
EAENAGSYSLQQAQAFYTFPFQQLMAEAPNMAVVNEQQMPEEVPAPAPAQEPVQEAPKGRKRKPRTTEPKQPVEPKKPVESKKSGKSAKPKEKQEKITDTFKVKRKVDRFNGVSEAELLTKTLPDILTFNLDIVIIGINPGLMAAYKGHHYPGPGNHFWKCLFMSGLSEVQLNHMDDHTLPGKYGIGFTNMVERTTPGSKDLSSKEFREGGRILVQKLQKYQPRIAVFNGKCIYEIFSKEVFGVKVKNLEFGLQPHKIPDTETLCYVMPSSSARCAQFPRAQDKVHYYIKLKDLRDQLKGIERNMDVQEVQYTFDLQLAQEDAKKMAVKEEKYDPGYEAAYGGAYGENPCSSEPCGFSSNGLIESVELRGESAFSGIPNGQWMTQSFTDQIPSFSNHCGTQEQEEESHA

SEQ ID NO: 393 - protein, 311 aa, synthetic construct
CSPQESGMTALSARMLTRSRSLGPGAGPRGCREEPGPLRRREAAAEARKSHSPVKRPRKAQRLRVAYEGSDSEKGEGAEPLKVPVWEPQDWQQQLVNIRAMRNKKDAPVDHLGTEHCYDSSAPPKVRRYQVLLSLMLSSQTKDQVTAGAIQRLRARGLAVDSILQTDDATLGKLIYPVGFWRSKVKYIKQTSAILQQHYGGDIPASVAELVALPGVGPKMAHLAMAVAWGTVSGIAVDTHVHRIANRLRWTKKATKSPEETRAALEEWLPRELWHEINGLLVGFGQQTCLPVHPRCHACLNQALCPAAQGL

SEQ ID NO: 394 - RNA, 20 nt, synthetic construct
  source 1..20: mol_type = other RNA
accaaaaaatatacgctata

SEQ ID NO: 395 - RNA, 20 nt, synthetic construct
  source 1..20: mol_type = other RNA
gccttaacaagctgctcttc

SEQ ID NO: 396 - RNA, 36 nt, synthetic construct
  source 1..36: mol_type = other RNA
cttttggaagtatataaatttacataggtacaaaac

SEQ ID NO: 397 - RNA, 13 nt, synthetic construct
  source 1..13: mol_type = other RNA
ataggtacaaaac

SEQ ID NO: 398 - RNA, 53 nt, synthetic construct
  source 1..53: mol_type = other RNA
tggctgattttgcagccataaggtgaggaaaattcactcaccaaagcctatgt

SEQ ID NO: 399 - RNA, 65 nt, synthetic construct
  source 1..65: mol_type = other RNA
acagcttatttggaagctgaaatgtgaggtttataacactcacaagaatcctaaggatgccaaac

SEQ ID NO: 400 - RNA, 66 nt, synthetic construct
  source 1..66: mol_type = other RNA
tggctgattttgcagccataaggtgaggaaaattcactcaccaaagcctatgtataggtacaaaac

SEQ ID NO: 401-403 - skipped (3 entries)